SEQUENCE LISTING (4 sequences)

SEQ ID NO: 1 - protein, 14 aa, Artificial sequence
  Synthetic Peptide
EGKSSGSGSESKVD

SEQ ID NO: 2 - protein, 18 aa, Artificial sequence
  Synthetic Peptide
KESGSVSSEQLAQFRSLD

SEQ ID NO: 3 - protein, 5 aa, Artificial sequence
  Synthetic Peptide
GGGGS

SEQ ID NO: 4 - protein, 233 aa, Human
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

(19) United States
(12) Patent Application Publication
(10) Pub. No.: US 2006/0263368 A1
(43) Pub. Date: Nov. 23, 2006

(54) Targeted chimeric molecules for cancer therapy

(75) Inventors: Michael G. Rosenblum, Sugar Land, TX (US); Andrew D. Ellington, Austin, TX (US)

Correspondence Address: FULBRIGHT & JAWORSKI, LLP, 1301 MCKINNEY, SUITE 5100, HOUSTON, TX, 77010-3095, US

(73) Assignee: Research Development Foundation, Carson City, NV (US)

(21) Appl. No.: 11/329,362

(22) Filed: Jan. 10, 2006

Related U.S. Application Data
(60) Provisional application No. 60/643,337, filed on Jan. 10, 2005.

(51) Int. Cl. A61K 39/395

(52) U.S. Cl. 424/155.1

(57) ABSTRACT

The present invention concerns chimeric cancer therapeutic molecules comprising a targeting moiety and an anti-cell proliferation moiety. The anti-cell proliferation moiety may comprise a cytotoxic agent or an apoptosis-inducing factor, in specific embodiments. In particular embodiments, the anti-cell proliferation mechanism of the chimeric molecules comprises apoptotic pathways. In additional embodiments, the chimeric molecules of the present invention provide sensitivity to chemotherapy in a cell that is resistant to the chemotherapy.

### CROSS-REFERENCE TO RELATED APPLICATIONS

The present application claims priority to U.S. Provisional Patent Application Ser. No. 60/643,337, filed Jan. 10, 2005, which is incorporated by reference herein in its entirety.

The present invention was developed at least in part through Department of Defense Grants DAMD17-99-1-9259-3 and DAMD17-02-1-0457-1. The United States Government may have certain rights in the invention.

### FIELD OF THE INVENTION

The present invention is directed to the fields of cell biology, molecular biology, cancer biology, and medicine. More particularly, the present invention regards targeted chimeric molecules for cancer treatment, including targeted cytotoxic agents, such as TNF, for example, and targeted pro-apoptotic molecules, such as Granzyme B, for example.

### BACKGROUND OF THE INVENTION

The considerable morbidity and mortality with cancer fuels the need for improved therapies, not only for new incidences of cancer but also for those having been treated and having developed resistance to one or more chemotherapies. Other advantageous characteristics for an effective cancer treatment include having minimal side effects from the therapy, such as by targeting the composition specifically to cancerous cells while sparing normal cells from harm. That is, a beneficial composition would be able to provide effective treatment for one or more cancer cells, yet avoid deleterious effects on normal cells. In particular, useful compositions include those that provide separate targeting moieties and anti-cell proliferative moieties. The present invention provides such beneficial compositions useful for any type of cancer.

Melanoma and Targeted Cytotoxic Molecules

Through molecular engineering, proteins can be modified to significantly enhance their biological activities. Fusion proteins designed to combine antibodies with cytokines, antibodies with cytokine receptors, or cytokines with toxins, for example, are currently being evaluated in preclinical and clinical studies (Davis and Gillies, 2003; Veenendaal et al., 2002; Liu et al., 2000). Single-chain recombinant antibodies (scFvs) comprise the antibody V_{L} and V_{H} domains linked by a designed flexible peptide tether (Bird et al., 1988). Compared to intact IgGs, scFvs have the advantages of smaller size and structural simplicity with comparable antigen-binding affinities, and they can be more stable than the analogous 2-chain Fab fragments (Cocher et al., 1990; Kantor et al., 1982). Several studies have shown that the smaller size of scFvs provides better penetration into tumor tissue, improved pharmacokinetics, and a reduction in the immunogenicity observed with i.v. administered Fabs compared to that of intact murine antibodies (Cocher et al., 1990; Kantor et al., 1982; Macey et al., 1998; Aggarwal and Natarajan, 1996). The scFvMEL single-chain antibody, for example, retains the same binding affinity and specificity of the parental ZME-018 antibody that recognizes the surface domain of the gp240 antigen present on human melanoma cells (Burger and Dayer, 2002; Boris and Steinke, 2003).

Tumor necrosis factor (TNF) is a cytotoxic polypeptide secreted primarily by activated macrophages, and it shares some sequence homology (30%) with another peptide hormone, lymphotoxin (LT or TNF-beta) secreted by activated lymphocytes (Zouboulis et al., 1990). Purified recombinant human TNF-alpha is a single-chain, non-glycosylated polypeptide of molecular weight 17 kDa, although in solution it polymerizes into a compact, non-disulfide linked trimer. TNF mediates a wide spectrum of systemic and cellular responses, including fever, shock, tissue injury, tumor necrosis, induction of other cytokines and immunoregulatory molecules, cell proliferation, differentiation, and apoptosis (Shiohara et al., 1997; Cosman, 1994). In vitro, TNF is cytostatic or cytotoxic to a number of human tumor cells, including SKBR-3 breast carcinoma and A-375M human melanoma (Smith and Baglioni, 1987; Cappello et al., 2002), for example. All the responses either in vitro or in vivo are elicited by TNF-induced trimerization of two distinct cell surface receptors, TNFR1 and TNFR2, at least one of which is present in almost every cell type (Rao, 2001; Cowan and Storey, 2003). Although these two receptors induce both distinct and overlapping responses, the majority of TNF effects, including the initiation of cell death cascades and host responses against a variety of pathogens, appear to be mediated by TNFR1 (Tsujimoto et al., 1985). The ability of both TNFR1 and TNFR2 to transduce signals is dependent upon the interaction of their cytoplasmic tails with downstream regulatory proteins. The intracellular domain of TNFR1, in contrast to TNFR2, contains a so-called “death domain”, which binds adapter proteins such as TRADD (TNFR-associated death domain protein). TRADD binds two additional transducers, TRAF2 (TNFR-associated factor-2) and receptor-interacting protein. These proteins, in turn, induce the kinase cascades ultimately resulting in the activation of the transcription factor NF-κB and/or of the cell death pathway. MADD (MAP kinase-activating death domain protein) is another protein that binds to the death domain of TNFR1. However, in contrast, MADD does not cause cell death or NF-κB activation, but specifically stimulates members of the mitogen-activated protein kinase (MAPK) family (Sugarman et al., 1985). Currently, the MAPK family comprises three subfamilies, namely: (a) the extracellular signal-regulated kinase (ERK); (b) the c-Jun NH2-terminal kinase/stress-activated protein kinases (JNK/SAPK); and (c) the p38 MAPK subfamily (Niitsu et al., 1985).

Several groups have demonstrated that human tumor cells can display between 100 and 5000 TNF receptor sites per cell (Rosenblum et al., 1991; Rosenblum et al., 1995). However, no apparent correlation has been observed between receptor number (or affinity) and the cellular response to the cytotoxic effects of TNF, suggesting that post-receptor signaling events may primarily modulate TNF biochemical effects (Koizumi et al., 1988).

Previous studies of the present inventors were first to demonstrate that immunocytokines consisting of chemical conjugates of human TNF and monoclonal antibodies can display impressive targeted cytotoxic properties against tumor cells in culture that appear to be far superior to native TNF and were active against TNF-resistant tumor cells (Rosenblum et al., 1991; Mujoo et al., 1995; Hanada and Yoshimura, 2002). In addition, studies in xenograft models suggested that antibody-TNF conjugates readily accumulate specifically in tumor tissues and demonstrate superior in vivo anti-tumor activity compared with native TNF (Tamanini et al., 2003). Based on these original observations, the present inventors designed and constructed a second-generation recombinant fusion toxin composed of the recombinant single-chain anti-gp240 antibody scFvMEL targeting human melanoma cells and containing human TNF as a cytotoxic effector molecule. The fusion protein was shown to enhance the in vitro killing of both TNF-sensitive and TNF-resistant human melanoma cells compared to native TNF. Further studies confirmed that the observed effects were antibody-mediated since competition with free antibody reduced the apparent cytotoxicity of the construct (Mujoo et al., 1995).

HER-2/neu Cancers and Targeted Cytotoxic Molecules

The HER-2/neu protooncogene is a 185-kDa transmembrane receptor tyrosine kinase that belongs to the epidermal growth factor family (Bargman et al., 1986; Coussens et al., 1985; Yamamoto et al., 1986) and is overexpressed in 20-30% of human breast cancers and ovarian cancers (Slamon et al., 1989; Tyson et al., 1991). HER-2/neu over-expression enhances proliferative, prosurvival, and metastatic signals in breast cancer cell lines (Hung and Lau, 1999; Ignatoski et al., 2000; Tzahar and Yarden, 1998) and has been associated with poor prognosis in ovarian, node-positive, and node-negative breast carcinomas (Berchuck et al., 1990; Slamon et al., 1987; Ro et al., 1989; Ross and Fletcher, 1998). One of the key roles this oncogene appears to play is in modulation of cellular response to cytotoxic cytokines such as TNF (Lichtenstein et al., 1991; Tang et al., 1994). A variety of research groups have demonstrated that both naturally HER-2/neu-overexpressing cells and HER-2/neu-transfected cells are resistant to the cytotoxic effects of TNF (Lichtenstein et al., 1990; Hudziak et al., 1988). Since TNF plays a central role in immune surveillance functions (Saks and Rosenblum, 1992), resistance to its cytotoxic effects mediated by HER-2/neu over-expression in breast cancer may allow transformed cells a growth advantage by escaping host defense mechanisms.

With the advent of molecular engineering approaches to build unique, targeted therapeutic agents, a second-generation recombinant construct comprising TNF tethered to a single-chain antibody recognizing HER-2/neu (scFv23) was generated. Previous studies demonstrated the production, purification and biological characterization of the scFv23/TNF construct against human breast tumor cells in culture (Rosenblum et al., 2000) and found that the fusion construct targeting HER-2/neu was active against cells resistant to TNF itself.

The association of targeted molecules with pancreatic cancer, for example, is also noteworthy. Pancreatic cancer remains one of the leading causes of cancer-related deaths in the United States and Europe (Haycox et al., 1998; Ward et al., 2001; Gumbs et al., 2002; Magee et al., 2002; Kulke, 2002) This is a highly aggressive and metastatic tumor type virtually resistant to all chemotherapeutic (Permert et al., 2001) and radiotherapeutic intervention (Boz et al., 2001; Matsuno et al., 2001). The current front-line approach for chemotherapeutic intervention is 5-fluorouracil (FU) or 5-FU containing regimens (Lokich and Skarin, 1972; Frey et al., 1981; Takada et al., 1992; Ducreux and Rougier, 1996). However, recent studies have demonstrated some clinical benefit to treatment with gemcitabine and gemcitabine-containing regimens (van Moorsel et al., 1997; Carnicheal, 1997; Michael and Moore, 1997; Cascinu et al., 1999).

In pancreatic tumor biopsy specimens, there are numerous oncogenes such as HER-2/neu and HER-1 (Tomaszewska et al., 1998; Sakorafas et al., 1995; Williams et al., 1991; Tamanaka, 1992; Lemoine et al., 1992; Ozawa et al., 1988) that are overexpressed, as well as there being mutations in various genes, such as p53, Ki-ras, and p-21 (Yokoyama et al., 1994; Hahn and Kern, 1995; Dergham et al., 1997). Many of these genetic abnormalities play a major role in the development of the aggressive, metastatic and therapy-resistant phenotype presented clinically. Approaches to use vaccines (Gjertsen and Gaudernack, 1998; Gunzburg and Salmons, 2001; Jaffee et al., 2001; Kaufman et al., 2002) or antibodies to target oncogene protein products (Buchler et al., 2001; Xiong and Abbreuzzese, 2002; Buchsbaum et al., 2002) are underway or have been completed to provide more focused control of tumor growth.

WO 93/21232 describes conjugates of a cellular targeting moiety and a cytotoxic moiety for the treatment of a neoplastic condition. Specific examples include a c-erbB-2 protein antibody and gelonin.

Targeted Pro-Apoptotic Molecules

The selective destruction of an individual cell is often desirable in a variety of clinical settings. A multitude of signal transduction pathways in the cell are linked to its death and survival, and delivery of a limiting and/or crucial component of the pathway can be productive in terms of its destruction. A classic example of such a signal transduction pathway is apoptosis, and a variety of elements of apoptotic pathways are useful to target a cell for death. Apoptosis, or programmed cell death, is a fundamental process controlling normal tissue homeostasis by regulating a balance between cell proliferation and death (Vaux et al., 1994; Jacobson et al., 1997).

The serine protease granzyme B (GrB) (Lobe et al., 1986; Schmid and Weissman, 1987; Trapani et al., 1988) is integrally involved in apoptotic cell death induced in target cells upon their exposure to the contents of lysosome-like cytoplasmic granules (or cytolytic granules) found in cytotoxic T-lymphocytes (CTL) and natural killer (NK) cells (Henkart, 1985; Young and Cohn, 1986; Smyth and Trapani, 1995). Cytotoxic lymphocyte granules contain perforin, a pore-forming protein, and a family of serine proteases, termed granzymes. Perforin has some structural and functional resemblance to the complement proteins C6, C7, C8 and C9, members of complement membrane attack complex (Shinkai et al., 1988). In lymphocyte-mediated cytolysis, perforin is inserted into the target cell membranes and appears to polymerize to form pores (Podack, 1992; Yagita et al., 1992), which mediates access of granzyme B to the target cell cytoplasm. Once inside, granzyme B induces apoptosis by directly activating caspases and inducing rapid DNA fragmentation (Shi et al., 1992).

The granzymes are structurally related, but have diverse substrate preference. Through its unique ability to cleave after aspartate residues, granzyme B can cleave many procaspases in vitro, and it has been an important tool in analyzing the maturation of caspase-3 (Darmon et al., 1995; Quan et al., 1996; Martin et al., 1996), caspase-7 (Chinnaiyan et al., 1996; Gu et al., 1996; Femandes-Alnemri et al., 1995), caspase-6 (Orth et al., 1996; Femandes-Alnemri et al., 1995), caspase-8 (Muzio et al., 1996), caspase-9 (Duan et al., 1996), and caspase-10a/b (Femandes-Alnemri et al., 1996; Vincenz and Dixit, 1997). Furthermore, it is highly toxic to target cells (Shi et al., 1992). It has been assumed until now that granzyme B kills cells by direct caspase activation, supplemented under certain circumstances by direct damage to downstream caspase substrates (Andrade et al., 1998). Having gained access to the cytosol, granzyme B is rapidly translocated to the nucleus (Jans et al., 1996; Trapani et al., 1996) and can cleave poly (ADP-ribose) polymerase and nuclear matrix antigen, sometimes using different cleavage sites than those preferred by caspases (Andrade et al., 1998). Although many procaspases are efficiently cleaved in vitro, granzyme B-induced caspase activation occurs in a hierarchical manner in intact cells, commencing at the level of executioner caspases such as caspase-3, followed by caspase-7 (Yang et al., 1998). This is in contrast to FasL-mediated killing, which relies on a membrane signal generated through apical caspases such as caspase-8 (Muzio et al., 1996; Sarin et al., 1997). In addition, some studies showed that granzyme B can also induce death through a caspase-independent mechanism that involves direct damage to normuclear structures, although the key substrates in this pathway have yet to be elucidated (Sarin et al., 1997; Trapani et al., 1998; Heibein et al., 1999; Beresford et al., 1999).

Studies by Froelich and co-workers suggest that GrB is internalized by receptor-mediated endocytosis, and that the role of perforin is to mediate release of granzyme B from endocytic vesicles. In fact, perforin can be replaced by other vesicle-disrupting factors such as those produced by adenovirus (Froelich et al., 1996; Pinkoski et al., 1998; Browne et al., 1999).

Granzymes in general are highly homologous, with 38-67% homology to GrB (Haddad et al., 1991), and they contain the catalytic triad (His-57, Asp-102, and Ser-195) of trypsin family serine proteases. Other features include the mature, N-terminal Ile-Ile-Gly-Gly sequence, three or four disulfide bridges, and a conserved motif (PHSRPYMA), which also appears in neutrophil cathepsin G and mast cell chymases. The carbohydrate moieties of granzymes are Asn-linked (Griffiths and Isaaz, 1993). The granzyme mRNA transcripts are translated as pre-pro-proteases. The pre- or leader sequence is cleaved by signal peptidase at the endoplasmic reticulum. When the propeptides are removed, the inactive progranzymes (zymogens) become active proteases. The granzyme propeptides sequences start after the leader peptide and end before the N-terminal Ile needed for the protease to fold into a catalytic conformation (Kam et al., 2000).

Among the various apoptotic factors identified so far, members of the Bcl-2 family represent some of the most well-defined regulators of this death pathway. Some members of the Bcl-2 family, including Bcl-2, Bcl-XL, Ced-9, Bcl-w and so forth, promote cell survival, while other members including Bax, Bcl-Xs, Bad, Bak, Bid, Bik and Bim have been shown to potentiate apoptosis (Adams and Cory, 1998). A number of diverse hypotheses have been proposed so far regarding the possible biological functions of the Bcl-2 family members. These include dimer formation (Oltvai et al., 1993), protease activation (Chinnaiyan et al., 1996), mitochondrial membrane depolarization (6), generation of reactive oxygen intermediates (Hockenbery et al., 1993), regulation of calcium flux (Lam et al., 1994; Huiling et al., 1997), and pore formation (Antonsson et al., 1997; Marzo et al., 1998).

Bax, a 21 kDa death-promoting member of the Bcl-2 family, was first identified as a protein that co-immunoprecipated with Bcl-2 from different cell lines (Oltvai et al., 1993). Overexpression of Bax accelerates cell death in response to a wide range of cytotoxic results. Determination of the amino acid sequence of the Bax protein showed it to be highly homologous to Bcl-2. The Bax gene consists of six exons and produces alternative transcripts, the predominant form of which encodes a 1.0 kb mRNA and is designated Bax.alpha. Like Bcl-2 and several other members of the Bcl-2 family, the Bax protein has highly conserved regions, BH1, BH2 and BH3 domains, and hydropathy analysis of the sequences of these proteins indicates the presence of a hydrophobic transmembrane segment at their C-terminal ends (Oltvai et al., 1993).

Bax is widely expressed without any apparent tissue specificity. However, on the induction of apoptosis, Bax translocates into mitochondria, resulting in mitochondria dysfunction and release of cytochrome c, which subsequently activates caspase pathways (Hsu and Youle, 1997; Wolter et al., 1997; Gross et al., 1998). This translocation process is rapid and occurs at an early stage of apoptosis (Wolter et al., 1997). Selective overexpression of Bax in human ovarian cancer through adenoviral gene transfer resulted in significant tumor cell kill in vivo (Tai et al., 1999). Overexpression of the Bax gene by a binary adenovirus system in cultured cell lines from human lung carcinoma results in caspase activation, apoptosis induction, and cell growth suppression. Moreover, intratumoral injection of adenovirus vector expressing the Bax gene suppressed growth of human lung cancer xenografts established in nude mice (Kagawa et al., 2000; Kagawa et al., 2000).

WO 99/45128 and Aqeilan et al. (1999) are directed to chimeric proteins having cell-targeting specificity and apoptosis-inducing activities, particularly the recombinant chimeric protein IL-2-Bax, which specifically targets IL2 receptor-expressing cells and induces cell-specific apoptosis.

WO 99/49059 relates to a chimeric toxin comprised of gonadotropin releasing hormone (GnRH) and Pseudomonas exotoxin A (PE) to detect a tumor-associated epitope expressed by human adenocarcinoma.

WO 97/46259 concerns targeted chimeric toxins comprising cell targeting moieties and cell killing moieties directed to neoplastic cells. In a specific example, the chimeric toxin comprises gonadotropin releasing hormone homologs and Pseudomonas Exotoxin A.

WO 97/22364 addresses targeted treatment of allergy responses, whereby a chimeric cytotoxin Fc2′-3-PE₄₀ is directed to targeted elimination of cells expressing the FcεRI receptor.

While some chimeric protein compositions have been described, other methods and compositions are needed for improved therapies involving the killing of cells.

### SUMMARY OF THE INVENTION

The present invention generally concerns treatment of cancer using chimeric molecules comprising a targeting moiety and an anti-cell proliferation factor, for example. The anti-cell proliferation factor may be further defined, for example, as a cytotoxic agent or, alternatively, a pro-apoptotic inducing moiety. Thus, the chimeric polypeptide is comprised of at least two moieties: one moiety is the effectual component for killing of the cell (the cytotoxin or the pro-apoptotic moiety), for example; the second moiety is the delivery component of the chimeric polypeptide to target the killing component to the cell of interest, for example. In specific embodiments, any cell of interest may be targeted with an appropriate targeting moiety, although in specific embodiments the cell targeting moiety targets a cancer cell. In some embodiments of the present invention, at least one of the moieties, and preferably both, are of human origin, which eliminates an immune response from the individual to whom the chimeric polypeptide is administered. The bipartite components of the chimeric molecules may be associated in any suitable manner, but in particular embodiments they are conjugated together. In some embodiments, the two components are conjugated to one another, while in other embodiments the polypeptides are engineered recombinantly to produce a fusion protein. Conjugated compounds may be attached to one another by a linker, for example.

In a particular embodiment of the invention, the cytotoxic agent is TNF-α. Although this molecule has been described before in the context of targeted chimeric molecules, the present invention utilizes them in novel therapeutic methods, including for example, restoring chemosensitivity to chemotherapy-resistant cancer cells; treating Her-2/neu- and Nf-κB-overexpressing cancers; inducing apoptosis in TNF-resistant cancers; and administering the composition with a chemotherapeutic agent that acts via interruption of Nf-κB signalling.

In another particular embodiment of the present invention, the cell-killing moiety is a pro-apoptotic factor. Although any suitable pro-apoptotic factor may be utilized in the invention, in particular embodiments the pro-apoptotic factor is a granzyme, such as Bax, granzyme A, or granzyme B, for example.

Any suitable cell-specific targeting moiety may be employed in the chimeric molecule of the invention, although in particular embodiments it comprises an antibody to one or more particular cell-surface antigens, cell markers, growth factors, hormones, or cytokines, for example. In one aspect, the targeting moiety of the chimeric composition is an antibody, such as a single chain antibody, an antibody fragment, a Fab, novel constructs such as mini-bodies, diabodies, triabodies, and so forth, for example. Specific examples of targeting molecules include scFv23, C6.5 or ML3-9, comprising a single chain antibody recognizing the cell surface domain of HER-2/neu; scFvMEL, comprising an anti-gp240 antigen single chain Fv; scFvAF20 comprising a single-chain antibody to the AF-20 antigen; and the human/mouse chimeric antibody HuM195 recognizing the CD-33 antigen, for example.

The compositions of the present invention may be directed to any suitable cancer, including lung, breast, brain, prostate, spleen, pancreatic, cervical, ovarian, head and neck, esophageal, liver, skin, kidney, leukemia, bone, testicular, colon, bladder, and so forth, for example. In particular embodiments of the invention they are directed to cancers having particular molecular etiologies and/or genotypes. In a specific embodiment, the cancer etiology renders it at least initially refractory to particular therapies and/or renders it able to develop resistance to one or more therapies. In further specific embodiments, the cancers to be treated are Her-2/neu-overexpressing; are TNF-α-resistant; are Nf-κB-overexpressing; are Nf-κB-signaling defective; comprise upregulation of EGF receptor; comprise upregulation of Multidrug resistance proteins (MDR or MRP); and/or are resistant to one or more conventional chemotherapies, such as 5-fluorouracil, for example. Moreover, the compositions of the present invention may act through distinct mechanisms, such as by down-regulating Akt phosphorylation; by inducing apoptosis, such as through cleavage of caspase-8, caspase-3, and/or poly ADP-ribose polymerase, for example; by inducing activation of caspase-3; by down-regulating an anti-apoptotic protein, such as Bcl-2; by degrading IκBα; by activating p38 MAP kinase; by activating SAPK/JNK pathway; and/or by inducing apoptotic nuclei; and so forth.

In particular embodiments of the invention, the immunocytokine scFv23/TNF, comprised of TNF tethered to the single chain antibody scFv23 that recognizes the cell-surface domain of HER-2/neu, is utilized. The present inventors demonstrate that scFv23/TNF is effective against cancers that are highly resistant to chemotherapy and that over-express HER-2/neu, for example, such as pancreatic tumors and breast tumors. More particularly, using a panel of human pancreatic cell lines, the present inventors characterized the relative expression of HER-2/neu, HER-1, TNFR-1, TNFR-2 and p-Akt and evaluated the in vitro response of cells to scFv23/TNF, TNF and several classes of chemotherapeutic agents. There was a correlation between the expression levels of HER-2/neu and TNFR-1 and cellular response to the tested agents. For example, pancreatic L3.6pl cells expressing the highest levels of HER-2/neu and TNFR-1 were the most sensitive to the conventional chemotherapeutic agents, whereas Capan-2 cells expressing comparatively lower levels of HER-2/neu and TNFR-1 were the most resistant to the tested drugs. Doxorubicin, gemcitabine and scFv23/TNF were the most active cytotoxic agents, whereas all cell lines were relatively resistant to 5-fluorouracil, cisplatin, etoposide, and TNF. Combination studies demonstrated a uniform synergistic effect of scFv23/TNF with 5-fluorouracil and an antagonistic effect of scFv23/TNF with doxorubicin in all pancreatic cell lines. Mechanistic studies demonstrated that the scFv23/TNF and 5-FU combination specifically resulted in a down-regulation of both the survival protein phospho-Akt and the anti-apoptotic protein Bcl-2. In addition, the combination induced apoptosis through cleavage of caspase-8, caspase-3, and poly ADP-ribose polymerase, for example. Therefore, targeting HER-2/neu expressing tumor cells using the scFv23/TNF fusion toxin is effective therapy for cancer, such as pancreatic cancer, particularly when utilized in combination with a chemotherapeutic agent such as 5-fluorouracil, for example.

To analyze a correlation between HER-2/neu expression and TNF resistance in breast cancer, the unique signaling pathways associated with the cytotoxic effects of scFv23/TNF were also characterized. SKBR-3/H cells expressed a 3.3 fold higher level of HER-2/neu, whereas SKBR-3/L cells expressed 2.3 fold and 4 fold higher levels of TNF receptor-1 and TNF respector-2, respectively. Compared with low-HER-2/neu-expressing SKBR-3/L cells, HER-2/neu-overexpressing SKBR-3/H cells were completely resistant to TNF itself but were sensitive to scFv23/TNF. Treatment of SKBR-3/H cells with scFv23/TNF resulted in down-regulation of Akt phosphorylation and induced apoptosis at least through cleavage of caspase-8, caspase-3, and poly ADP-ribose polymerase. ScFv23/TNF-induced cytotoxicity was dependent on activation of caspase-3, in some embodiments of the invention. On the other hand, scFv23 and TNF alone activated phosphorylation of Akt but had no effect on caspase activation and apoptosis. Therefore, scFv23/TNF has a distinct mechanism of action compared to TNF and is effective against HER-2/neu-overexpressing cancer cells resistant to TNF.

Other exemplary fusion toxins of the present invention include the fusion construct scFvMEL/TNF, which comprises an anti-gp240 antigen single-chain Fv tethered to human TNF. The present inventors characterized the molecular mechanisms of the cytotoxic effects of the antimelanoma fusion toxin scFvMEL/TNF in comparison to TNF against human melanoma cells. In particular, mechanisms underlying the ability of the construct to overcome cellular resistance to TNF itself were identified, particularly molecular pathways involved in TNF-induced signaling such as NF-κB and JNK, as well as apoptosis mediators including caspase-3 and PARP. Furthermore, cDNA microarray analysis of cells treated with both TNF and scFvMEL/TNF identified unique differences in the mechanism of cytotoxic activity between TNF and scFvMEL/TNF.

In particular, the present inventors demonstrate that the scFvMEL/TNF fusion construct was more cytotoxic to antigen positive A375-M cells compared to TNF alone (I.C.₅₀˜0.1 nM vs. 1.4 nM, respectively), and was also cytotoxic to AAB-527 cells (I.C.₅₀˜20 nM) completely resistant to TNF. Treatment with TNF or with scFvMEL/TNF induced degradation of IκBα and activation of p38 MAP kinase in a time-dependent manner in both A375-M and AAB-527 cells. Rapid activation of the SAPK/JNK pathway was observed in TNF-resistant AAB-527 cells treated with TNF, but not after treatment with scFvMEL/TNF. Therefore, activation of SAPK/JNK contributes to the observed cellular resistance to TNF, in specific embodiments of the invention. In agreement with the cytotoxicity data, scFvMEL/TNF induced PARP cleavage and apoptotic effects in both A375-M and AAB-527 cells. However, TNF induced PARP cleavage and apoptosis only in A375-M cells.

Based on these studies and the demonstration that there are many genes down-regulated or up-regulated in melanoma cells by scFvMEL/TNF but not by TNF treatment, including genes involved primarily in cell surface receptor-linked signaling, intracellular signaling cascades, cell cycle events, and nucleotide metabolism regulation, scFvMEL/TNF has a unique mechanism of action compared to TNF. More specifically, the fusion construct can overcome TNF resistance because it induces apoptosis, blocks the SAPK/JNK cell survival pathway and activates a unique complement of genes compared to that observed after TNF exposure.

In another embodiment of the present invention, the chimeric molecules comprise an apoptosis-inducing agent and a targeting moiety. Although any particular apoptosis-inducing agent capable of killing a cell may be employed in the invention, in specific embodiments the apoptosis-inducing agent is a granzyme, such as granzyme B, for example. The targeting moiety may be of any suitable kind such that the moiety substantially targets the chimeric molecule to a cancer cell; in particular embodiments the targeting moiety is an antibody to one or more particular cell markers, a growth factor, a hormone, or a cytokine, for example. In one aspect, the targeting moiety of the chimeric composition is an antibody, such as a single chain antibody, an antibody fragment, a Fab, and so forth.

In particular embodiments, the present inventors utilized the novel fusion construct GrB/scFvMEL, comprising the human pro-apoptotic serine protease Granzyme B (GrB) and the single-chain antibody scFvMEL recognizing the gp240 antigen that is the high-molecular-weight glycoprotein present on a majority (80%) of melanoma cell lines and fresh tumor samples. Specifically, the expression of gp240 antigen on different melanoma cells was examined by using ELISA and flow cytometry. The gp240 presents on A375, TXM-18L, TXM-13 and MEL-526 melanoma cells, however, there was very low level of expression on TXM-1 melanoma cells. The GrB/scFvMEL fusion construct bound to high-level gp240 antigen A375, TXM-18L, TXM-13 and MEL-526 but not to TXM-1 cells as detected by an anti-scFvMEL antibody. The fusion construct demonstrated an I.C.₅₀ of ˜20 nM against log-phase A375 cells, ˜50 nM against MEL-526 cells, ˜100 nM against TXM-18L, ˜200 nM against TXM-13 cells and minimal cytotoxicity to TXM-1 and non-target SKBR3 cells at doses of up to 1 μM.

By comparison, the cytotoxic effects of GrB/scFvMEL were approximately the same as that of another fusion toxin, scFvMEL/rGel, on these melanoma cells. Co-administration of GrB/scFvMEL and chemotherapeutic agents (doxorubicin, vincristine sulfate, etoposide, cisplatin or cytorabine, for example) to A375 cells for 72 hours demonstrated synergistic antitumor activity with doxorubicin, vincristine or cisplatin and additive effects in combination with etoposide or cytorabine. Pre-treatment with GrB/scFvMEL for 6 h followed by co-exposure to these chemotherapeutic agents for 72 hours showed significantly inhibited growth as compared to pre-treatment with drugs followed by co-exposure with the fusion construct. Moreover, the cytotoxicities of various chemotherapeutic agents significantly increased when A375 cells were pre-treated with GrB/scFvMEL for 6 h followed by treatment with chemotherapeutic agents for 72 h compared to without GrB/scFvMEL pretreatment (p<0.01). The effects of chemotherapeutic agents could be sensitized by pretreatment with GrB/scFvMEL for 6 h on gp240 antigen-positive targeted cells. Tumor tissue displayed apoptotic nuclei in GrB/scFvMEL treatment group after 24 h administration on mice bearing A375 xenograft tumors, as assessed by TUNEL assay. Localization or internalization of GrB/scFvMEL was observed in tumor tissue as assessed by immunohistochemical staining detected by either anti-GrB or anti-scFvMEL antibody. Mice bearing A375 tumors were administered (iv tail vein, 37.5 mg/kg) for 5 times every other day with either GrB/scFvMEL or saline, and tumor volumes were measured for 42 days. The saline-treated control tumors increased from 50 mm³ to 1200 mm³ over this period. Tumors treated with GrB/scFvMEL increased from 50 mm³ to 200 mm³. Thus, the GrB/scFvMEL fusion construct demonstrates impressive antitumor activity and enhances the sensitivity of human melanoma cells to chemotherapy.

In one embodiment of the present invention, there is a method of conferring or restoring chemosensitivity to one or more chemotherapy-resistant cancer cells in an individual, comprising delivering to the individual a therapeutically effective amount of a chimeric molecule comprising a cell-specific targeting moiety and an anti-cell proliferation moiety. In specific embodiments, the cell-specific targeting moiety is further defined as a cancer cell-targeting moiety. In further specific embodiments, the cancer cell-targeting moiety is further defined as an antibody, a growth factor, a hormone, a peptide, an aptamer, a cytokine, interferon, vitamin, or a mixture thereof, for example. The antibody may be further defined as a full-length antibody, chimeric antibody, Fab′, Fab, F(ab′)2, single domain antibody (DAB), Fv, single chain Fv (scFv), minibody, diabody, triabody, or a mixture thereof, for example. In particular embodiments, the antibody is an anti-HER-2/neu antibody, such as scFv23, for example. In additional specific embodiments, the antibody is an anti-gp240 antigen antibody, such as one that comprises scFvMEL, for example.

In other specific embodiments, the cancer cell-targeting moiety comprises one or more growth factors, such as, for example, transforming growth factor, epidermal growth factor, insulin-like growth factor, fibroblast growth factor, heregulin, platelet-derived growth factor, vascular endothelial growth factor, or hypoxia inducible factor. In an additional specific embodiment, the cancer cell-targeting moiety comprises one or more hormones, such as human chorionic gonadotropin, gonadotropin releasing hormone, an androgen, an estrogen, thyroid-stimulating hormone, follicle-stimulating hormone, luteinizing hormone, prolactin, growth hormone, adrenocorticotropic hormone, antidiuretic hormone, oxytocin, thyrotropin-releasing hormone, growth hormone releasing hormone, corticotropin-releasing hormone, somatostatin, dopamine, melatonin, thyroxine, calcitonin, parathyroid hormone, glucocorticoids, mineralocorticoids, adrenaline, noradrenaline, progesterone, insulin, glucagon, amylin, erythropoitin, calcitriol, calciferol, atrial-natriuretic peptide, gastrin, secretin, cholecystokinin, neuropeptide Y, ghrelin, PYY3-36, insulin-like growth factor-1, leptin, thrombopoietin, angiotensinogen, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, and/or, IL-36, for example.

In another embodiment of the present invention, the cancer cell-targeting moiety comprises one or more cytokines, such as IL1, IL2, IL3, IL4, IL5, IL6, IL7, IL8, IL9, IL10, IL11, IL12, IL13, IL14, IL15, IL-16, IL-17, IL-18, granulocyte-colony stimulating factor, macrophage-colony stimulating factor, granulocyte-macrophage colony stimulating factor, leukemia inhibitory factor, erythropoietin, granulocyte macrophage colony stimulating factor, oncostatin M, leukemia inhibitory factor, IFN-γ, IFN-α, IFN-β, LT-β, CD40 ligand, Fas ligand, CD27 ligand, CD30 ligand, 4-1BBL, TGF-β, IL 1α, IL-1β, IL-1 RA, MIF, IGIF, and/or a mixture thereof.

In particular embodiments of the invention, the anti-cell proliferation moiety is further defined as an apoptosis-inducing moiety or a cytotoxic agent. The apoptosis-inducing moiety may be a granzyme, a Bcl-2 family member, cytochrome C, a caspase, or a combination thereof, for example. Exemplary granzymes include granzyme A, granzyme B, granzyme C, granzyme D, granzyme E, granzyme F, granzyme G, granzyme H, granzyme I, granzyme J, granzyme K, granzyme L, granzyme M, granzyme N, or a combination thereof, for example. In other specific embodiments, the Bcl-2 family member is, for example, Bax, Bak, Bcl-Xs, Bad, Bid, Bik, Hrk, Bok, or a combination thereof, for example.

In additional specific embodiments, the caspase is caspase-1, caspase-2 caspase-3, caspase-4, caspase-5, caspase-6, caspase-7, caspase-8, caspase-9, caspase-10, caspase-11, caspase-12, caspase-13, caspase-14, or a combination thereof, for example. In specific embodiments, the cytotoxic agent is TNF-α, gelonin, Prodigiosin, a ribosome-inhibiting protein (RIP), Pseudomonas exotoxin, Clostridium difficile Toxin B, Helicobacter pylori VacA, Yersinia enterocolitica YopT, Violacein, diethylenetriaminepentaacetic acid, irofulven, Diptheria Toxin, mitogillin, ricin, botulinum toxin, cholera toxin, saporin 6, or a combination thereof, for example.

The cytotoxic agent may be recombinant, in specific embodiments of the invention.

In particular embodiments of the invention, the cell-specific targeting moiety and the anti-cell proliferation moiety are chemically conjugated. In further embodiments, the cell-specific targeting moiety and the anti-cell proliferation moiety are comprised in a fusion polypeptide, and in specific embodiments they are connected by a linker, for example.

In specific embodiments of the invention, the chemotherapy-resistant cancer cell is further defined as HER-2/neu overexpressing, resistant to TNF-α, Nf-κB-overexpressing, Nf-κB signaling-defective, or a combination thereof, and in specific embodiments they may be resistant to one or more classes of chemotherapeutic agents, such as alkylating agents, nitrosoureas, antimetabolites, antitumor antibiotics, plant alkyloids, taxanes, hormonal agents, or a combination thereof, for example. In specific embodiments, the chemotherapy-resistant cells are resistant to one or more of 5-fluorouracil, cisplatin, etoposide, doxorubicin, gemcitabine, or a combination thereof, for example.

In particular embodiments of the invention, the method further comprises an additional cancer therapy for the individual, such as chemotherapy, surgery, radiation, gene therapy, hormone therapy, immunotherapy, or a combination thereof.

In particular embodiments, the additional therapy and the chimeric molecule are administered concomitantly or are administered in succession. For example, the chimeric molecule may be administered prior to the chemotherapy and/or the chimeric molecule may be administered subsequent to the chemotherapy. In specific embodiments, the chemotherapy and the chimeric molecule provide a synergistic effect on the cancer cell or they provide an additive effect on the cancer cell. The chimeric molecule may be further defined as neoadjuvant surgical therapy or as postadjuvant surgical therapy, for example. In specific embodiments, the chimeric molecule is scFvMEL/GrB, scFv23/TNF-α, scFvMEL/TNF-α, or a combination thereof.

In an additional embodiment of the present invention, there is a method of sensitizing one or more cancer cells in an individual to a chemotherapy, comprising administering to the individual a therapeutically effective amount of a chimeric molecule, the chimeric molecule comprising a cell-targeting moiety and an anti-cell proliferation moiety. The cancer cell may be further defined as HER-2/neu overexpressing, TNF-α-resistant, Nf-κB-overexpressing, Nf-κB signaling-defective; or a combination thereof. The cancer cell may be resistant to chemotherapy, such as 5-fluorouracil, cisplatin, etoposide, doxorubicin, or gemcitabine, for example.

In another embodiment of the present invention, there is a method of inducing apoptosis in one or more TNF-resistant cancer cells in an individual, comprising administering to the individual a therapeutically effective amount of a chimeric molecule, the chimeric molecule comprising a cell-specific targeting moiety and an anti-cell proliferation moiety. Induction of apoptosis in one or more cells may be further defined as comprising one or more of the following: blockage of the SAPK/JNK signal pathway; inhibition of Akt phosphorylation; downregulation of Bcl-2; cleavage of caspase-8; cleavage of caspase-3; cleavage of poly ADP-ribose polymerase; degradation of IκB-α; and a combination thereof.

In an additional embodiment of the present invention, there is a method of inducing apoptosis in one or more HER-2/neu-overexpressing cancer cells in an individual, comprising administering to the individual a therapeutically effective amount of a chimeric molecule, the chimeric molecule comprising a cell-specific targeting moiety and an anti-cell proliferation moiety. Inducing apoptosis in one or more cells is further defined as comprising one or more of the following: blockage of the SAPK/JNK signal pathway; inhibition of Akt phosphorylation; downregulation of Bcl-2; cleavage of caspase-8; cleavage of caspase-3; cleavage of poly ADP-ribose polymerase; degradation of IκB-α; and a combination thereof.

In an additional embodiment, there is a method of inducing apoptosis in one or more gp240 antigen-positive cells in an individual, comprising administering to the individual a therapeutically effective amount of a chimeric molecule, the chimeric molecule comprising a cell-specific targeting moiety and an anti-cell proliferation moiety.

In another embodiment of the present invention, there is a method of treating cancers in an individual that are Her-2/neu overexpressing and Nf-κB overexpressing, comprising administering to the individual a therapeutically effective amount of a chimeric molecule, the chimeric molecule comprising a cell-specific targeting moiety and an anti-cell proliferation moiety.

In an additional embodiment of the present invention, there is a method of treating cancer in an individual, comprising administering to the individual a therapeutically effective amount of at least one chemotherapeutic agent, wherein said agent acts by interrupting NF-κB signaling, and a chimeric molecule, said chimeric molecule comprising a cell-specific targeting moiety and an anti-cell proliferation moiety. In a specific embodiment, the chemotherapeutic agent is an antimetabolite, such as 5-fluorouracil, 6-mercaptopurine, capecitabine, cytosine arabinoside, floxuridine, fludarabine, gemcitabine, methotrexate, or thioguanine, for example.

In an additional embodiment of the invention, there is a composition comprising an aptamer that targets a cell, such as one or more proteins of the cell, including one or more proteins on a surface of the cell. The aptamer may be considered a cell-specific targeting moiety, and in particular embodiments it is associated with an anti-cell proliferation moiety, such as linked or conjugated thereto. The aptamer may be associated with, such as linked to or conjugated to, a chimeric molecule of the invention, wherein the chimeric molecule itself comprises a cell-specific targeting moiety and an anti-cell proliferation moiety.

In further embodiments, a therapeutically effective amount of the aptamer composition is administered to an individual to confer or restore chemosensitivity to one or more chemotherapy-resistant cancer cells in the individual. The aptamer composition may also be utilized to sensitize one or more cancer cells in an individual to a chemotherapy by administering a therapeutically effective amount of the composition to the individual. The aptamer composition may also be employed to induce apoptosis in one or more TNF-resistant cancer cells in an individual, in one or more HER-2/neu overexpressing cancer cells in an individual, and/or in one or more gp240 antigen-positive cells in an individual. In additional embodiments, the aptamer composition is utilized for administering to an individual that has cancer that is HER-2/neu overexpressing and NF-κB overexpressing.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings.

FIG. 1 shows expression pattern of HER-2/neu, HER-1, TNFR-1, TNFR-2, and p-Akt in four Human pancreatic cell lines. Four pancreatic cancer cell lines (AsPc-1, Capan-1, Capan-2, and L3.6pl) were seeded at 5×10⁵ cells/φ60 mm petri-dish and incubated for 24 hr after which cell lysates were collected. Whole cell lysates (50 μg) were analyzed by SDS-PAGE and immunoblotting with anti-HER-2/neu, TNF receptor-1, TNF receptor-2, and p-Akt antibodies, followed by incubation with an anti-mouse or anti-rabbit horseradish peroxidase-labeled antibody and chemiluminescent detection. Actin was used as a loading control for protein loading.

FIGS. 2A-2D provide dose-response curves of TNF, scFv23/TNF, 5-fluorouracil, cisplatin, ectoposide, doxorubicin, and gemcitabine on four pancreatic cancer cell lines. AsPc-1 (FIG. 2A), Capan-1 (FIG. 2B), Capan-2 (FIG. 2C), and L3.6pl (FIG. 2D). Cells were treated with different drugs for 72 hr and then assessed growth inhibition by crystal violet staining. Values are means±SD from at least four independent exposures.

FIG. 3 shows effects of scFv23, TNF, and scFv23/TNF on the expression of Akt and phospho-Akt. L3.6pl cells were treated with IC₂₅ of 5-FU, scFv23, and 5-FU plus scFv23/TNF combination. After treatment, cell lysates (50 μg) were analyzed by SDS-PAGE and immunoblotting with anti-Akt and phospho-Akt antibodies, followed by incubation with an anti-rabbit horseradish peroxidase-labeled antibody and chemiluminescent detection. Actin was used as a loading control.

FIG. 4 demonstrates effects of scFv23, TNF, and scFv23/TNF on the expression of Bcl-2. L3.6pl cells were treated with IC₂₅ of 5-FU, scFv23, and 5-FU plus scFv23/TNF combination. After treatment, cell lysates (50 μg) were analyzed by SDS-PAGE and immunoblotting with anti-Bcl-2 antibody, followed by incubation with an anti-rabbit horseradish peroxidase-labeled antibody and chemiluminescent detection. Actin was used as a loading control.

FIG. 5 shows effects of 5-fluorouracil, scFv23/TNF, and combination on the activation of caspase-8, caspase-3, and PARP cleavage. L3.6pl cells were treated with IC₂₅ of 5-FU, scFv23/TNF, and combination for 48 hr. After treatment, cell lysates (50 μg) were analyzed by SDS-PAGE and immunoblotting with anti-caspase-8, caspase-3, and PARP antibodies, followed by incubation with an anti-mouse horseradish peroxidase-labeled antibody and chemiluminescent detection. Actin was used as a loading control.

FIG. 6 shows the influence of caspase-3 inhibitor on the viability of combination 5-FU+scFv23/TNF-treated L3.6pl cells. L3.6pl cells pre-treated with or without 100 μM caspase-3 inhibitor (Ac-DEVD-CHO) for 3 hr and then treated with IC₂₅ of 5-FU, scFv23/TNF, and combination. After 72 hr of exposure, viability was determined using an XTT assay.

FIGS. 7A-7B demonstrate the effect of scFv23/TNF on SKBR-3 breast cancer cell lines. Expression pattern of HER-2/neu, TNF receptor 1 and TNF receptor 2 is shown. SKBR-3/H and SKBR-3/L cell lines were seeded at 5×10⁵ cells/φ60 mm petri-dish and incubated for 24 hr after which cell lysates were collected. Whole cell lysates (50 μg) were analyzed by SDS-PAGE and immunoblotting with anti-HER-2/neu, TNF receptor-1, and TNF receptor-2 antibodies, followed by incubation with an anti-mouse or anti-rabbit horseradish peroxidase-labeled antibody and chemiluminescent detection. Actin was used as a loading control (FIG. 7A). Growth inhibition of either TNF or scFv23/TNF in SKBR-3/H and SKBR-3/L cells. SKBR-3/H and -3/L cells were treated with various concentration of TNF or scFv23/TNF. After 72 hr of exposure, viability was determined using XTT assay (FIG. 7B).

FIG. 8 shows neutralizing effect of TNF receptor-1 antibody on scFv23/TNF-induced growth inhibition. SKBR-3 cells exposed to anti-TNF receptor-1 Ab (25 and 50 μg/ml) 2 hr before 200 nM scFv23/TNF treatment. After 72 hr of exposure, viability was determined using an XTT assay.

FIG. 9 provides the effects of scFv23, TNF, and scFv23/TNF on the expression of IκB-α, TRADD, and TRAF2. SKBR-3/H cells were treated for the indicated times with 200 nM scFv23, 200 nM TNF or 200 nM scFv23/TNF for indicated time courses. After treatment, cell lysates (50 μg) were analyzed by SDS-PAGE and immunoblotting with anti-IκB-α, TRADD, and TRAF2 antibodies, followed by incubation with an anti-rabbit or anti-mouse horseradish peroxidase-labeled antibody and chemiluminescent detection. Actin was used as a loading control.

FIG. 10 shows effects of scFv23, TNF, and scFv23/TNF on the expression of Akt and phospho-Akt. SKBR-3/H cells were treated for the indicated times with 200 nM scFv23, 200 nM TNF, or 200 nM scFv23/TNF. After treatment, cell lysates (50 μg) were analyzed by SDS-PAGE and immunoblotting with anti-Akt and phospho-Akt antibodies, followed by incubation with an anti-rabbit horseradish peroxidase-labeled antibody and chemiluminescent detection. Actin was used as a loading control (FIG. 10A and FIG. 10B).

FIGS. 11A-11B demonstrate effects of TNF and scFv23/TNF on the apoptosis in HER-2/neu-over-expressing SKBR-3/H cells. Microscopic analysis of apoptotic cells is demonstrated. SKBR-3/H cells exposed to 200 nM TNF or 200 nM scFv23/TNF for 24 hr and 48 hr. After treatment, the cells were washed with PBS, permeabilized in permeabilization solution (0.1% Triton X-100, 0.1% sodium citrate), and then fixed in 4% paraformaldehyde. Fixed cells were stained with in situ cell death detection kit (Roche). Cells undergoing apoptosis were determined by fluorescence microscope (×200) (FIG. 11A). DNA Fragmentation of apoptotic cells. SKBR-3/H cells exposed to 200 nM TNF or 200 nM scFv23/TNF for 24 hr and 48 hr were lysed. DNA was extracted, fractionated by electrophoresis and stained by ethidium bromide (FIG. 11B).

FIG. 12 shows effects of scFv23, TNF, and scFv23/TNF on the activation of caspase-8, caspase-3, and PARP cleavage. SKBR-3/H cells were treated for the indicated times with 200 nM scFv23, 200 nM TNF, or 200 nM scFv23/TNF for 24 hr and 48 hr. After treatment, cell lysates (50 μg) were analyzed by SDS-PAGE and immunoblotting with anti-caspase-8, caspase-3, and PARP antibodies, followed by incubation with an anti-mouse horseradish peroxidase-labeled antibody and chemiluminescent detection. Actin was used as a loading control.

FIG. 13 demonstrates influence of caspase-3 inhibitor on the viability of scFv23/TNF-treated SKBR-3/H cells. SKBR-3/H cells pre-treated with or without 100 μM caspase-3 inhibitor (Ac-DEVD-CHO) for 3 hr and then treated with various concentrations of scFv23/TNF. After 72 hr of exposure, viability was determined using an XTT assay.

FIG. 14 shows influence of caspases inhibitors on the viability of scFv23/TNF-treated SKBR-3-LP cells. SKBR-3-LP cells pre-treated with or without 200 μM general caspase inhibitor (Z-VAD-FMK), 200 μM caspase-8 inhibitor (Z-IETD-FMK), or 200 μM caspase-3 inhibitor (Z-DEVD-FMK) for 2 hr and then treated with various concentrations of scFv23/TNF. After 72 hr of exposure, viability was determined using an XTT assay.

FIGS. 15A-15B demonstrate expression of signaling proteins and comparative sensitivity on SKBR-3 breast cancer cell lines. In FIG. 15A, there is Western blot analysis of HER-2/neu, TNF-R1, and TNF-R2 in SKBR-3-LP and -HP cell lines. SKBR-3-LP and SKBR-3-HP cell lines were seeded at 5×105 cells/φ60 mm petri-dish and incubated for 24 hr after which cell lysates were collected. Whole cell lysates (50 μg) were analyzed by SDS-PAGE and immunoblotting with anti-HER-2/neu, TNF receptor-1, and TNF receptor-2 antibodies, followed by incubation with an anti-mouse or anti-rabbit horseradish peroxidase-labeled antibody and chemiluminescent detection. Actin was used as a loading control. In FIG. 15B, there is growth inhibition of Herceptin, scFv23/TNF, and TNF in SKBR-3-LP and SKBR-3-HP cells. SKBR-3-LP and -3-HP cells were treated with various concentration of TNF or scFv23/TNF. After 72 hr of exposure, viability was determined using XTT assay.

FIG. 16 demonstrates the role of TNF receptor on scFv23/TNF-induced growth inhibition. To determine whether the cytotoxic effects of the scFv23/TNF, Herceptin or TNF were mediated entirely through interaction with cell-surface TNF receptor-1, the binding of scFv23/TNF to TNF receptor-1 was blocked using TNFR1:Fc fusion protein (1 and 10 φg/ml). After 72 hr of exposure, viability was determined using an XTT assay.

FIGS. 17A-17B show the effect of scFv23/TNF on modulation of TNF receptor-1 expression. To determine whether scFv23/TNF can modulate the expression of TNF-R1, we treated SKBR-3-LP and L3.6pl cells with scFv23, TNF, scFv23/TNF (FIG. 17A), or Herceptin (FIG. 17B). Whole cell lysates (50 μg) were analyzed by SDS-PAGE and immunoblotting with anti-TNF receptor-1, and TNF receptor-2 antibodies, followed by incubation with an anti-mouse or anti-rabbit horseradish peroxidase-labeled antibody and chemiluminescent detection. Actin was used as a loading control.

FIG. 18 demonstrates modulation of TNF sensitivity in SKBR-3-LP cells. To determine whether the over-expression of TNF-R1 can modulate the TNF sensitivity in HER-2/neu-overexpressing SKBR-3-LP cells, SKBR-3-LP cells were treated with TNF, scFv23, scFv23/TNF or TNF in combination with scFv23. After 72 hr of exposure, viability was determined using XTT assay.

FIG. 19 shows effects of scFv23, TNF, and scFv23/TNF on the expression of TRADD, TRAF-2, Iκ-B, Akt, and p-Akt. SKBR-3-LP cells were treated for the indicated times with 200 nM scFv23, 200 nM TNF, or 200 nM scFv23/TNF. After treatment, cell lysates (50 μg) were analyzed by SDS-PAGE and immunoblotting with anti-TRADD, TRAF-2, Iκ-B, Akt or phospho-Akt antibodies, followed by incubation with an anti-rabbit horseradish peroxidase-labeled antibody and chemiluminescent detection. Actin was used as a loading control.

FIG. 20 provides a summary of signal transduction effects of TNF and scFv23/TNF on HER-2/neu-overexpressing SKBR-3-LP cells.

FIG. 21 shows that the scFvMEL genes were fused to human TNF genes linked via a flexible tether G4S. The fusion construct scFvMEL/TNF was cloned into bacterial expression vector pET32a (+) at Nco I and HindIII in multiple cloning sites.

FIGS. 22A-22C demonstrate SDS-PAGE and Western blotting analysis of expression of scFvMEL/TNF fusion protein. FIG. 22A provides a 8.5% SDS-PAGE and coomassie blue staining under reducing conditions. Lane 1, protein marker. Lane 2, non-induced total lysate. Lane 3, induced total lysate. Lane 4, purified by IMAC. Lane5, after rEK digestion. Lane 6, final purified scFvMEL/TNF. FIGS. 23B and 23C show western blotting detected by rabbit anti-huTNF antibody; (FIG. 22B) or rabbit anti-scFvMEL antibody; (FIG. 22C) Lane 1, ZME-018. Lane 2, expressed protein with his-tag purified by IMAC. Lane 3, final purified expressed protein after rEK digestion. Lane 4, recombinant huTNF.

FIGS. 23A-23B show western blotting analysis IκB-α degradation. In FIG. 24A, cells (2×10⁵ cells/well) were set up in 6-well plates and treated with I.C.₅₀ concentrations of TNF or scFvMEL/TNF for 2, 5, 15, 30, 45 and 60 min time course. The cell lysate was extracted and 30 μg of total protein was loaded onto 8.5% SDS-PAGE and standard Western blotting was performed detected by IκBα antibody (1:3000 dilution). In FIG. 23B, cells were pretreated with ZME-018 (40 μg/ml) for 4 h, and then treated with I.C.₅₀ concentrations of scFvMEL/TNF for indicated time course. The same amount of cell lysate was analyzed as in FIG. 23A to detect IκB-α degradation.

FIG. 24 demonstrates western blotting analysis of p38 MAP kinase pathway. Cell lysates from different time course treatment with scFvMEL/TNF or TNF were extracted. The amount of 50 μg total protein was loaded onto 12% SDS-PAGE and analyzed by Western blotting probed with MKK3, phospho-MKK3 MKK6, p38 MAP Kinase, phospho-p38 MAP kinase (Thr 180/Tyr 182), ATF-2 or phospho-ATF-2 antibodies.

FIG. 25 shows western blotting analysis of SAPK/JNK pathway. The amount of 50 μg total protein of cell lysates from different time course treatment with scFvMEL/TNF or TNF was loaded onto 12% SDS-PAGE and analyzed by Western blotting probed with MKK4, phospho-SEK1/MKK4, SAPK/JNK, phospho-p54/46 SAJK/JNK (Thr 183/Tyr 185), c-Jun or phospho-c-Jun antibodies.

FIGS. 26A-26C show apoptotic profiles: In FIG. 26A, PARP cleavage is demonstrated. Cells (2×10⁶ cells/ml) were treated with TNF at 1 nM on A375-M and 200 nM on AAB-527, respectively, or with scFvMEL/TNF at 0.1 nM on A375-M and 20 nM on AAB-527 cells for 24 h. The amount of 50 μg total protein of cell lysate was loaded onto 7.5% SDS-PAGE and analyzed by Western blotting probed with anti-PARP antibody which recognized both cleaved (86 kDa) and uncleaved (116 kDa) proteins. In FIG. 26B, cleaved caspase-3 is shown. Cells were treated with scFvMEL/TNF or TNF for 1, 4, 8, 16 and 24 h. The amount of 50 μg total protein of cell lysate was loaded onto 12% SDS-PAGE and analyzed by Western blotting probed with cleaved caspase-3 monoclonal antibody. FIG. 26C demonstrates in situ cell death (TUNEL) analysis of apoptotic cells. 10,000 cells per well in 16-well chamber slide were treated with scFvMEL/TNF or TNF at I.C.₅₀ concentration for 24 h and washed briefly with PBS. Cells were fixed by 3.7% formaldehyde at room temperature for 10 min and permeabilized by 0.1% Triton X-100, 0.1% sodium citrate on ice for 2 min. Cells were incubated with TUNEL reaction mixture at 37° C. for 60 min. After final washing, the cells were analyzed under a Nikon Eclipse TS-100 fluorescence microscope with 400× magnification.

FIG. 27 shows western blotting analysis of TNF receptors and TNF receptor signaling related proteins on melanoma cells. The same amount of total proteins (30 μg) of cell lysate from different time course treatment with scFvMEL/TNF or TNF were loaded onto 10% SDS-PAGE and analyzed by Western blotting probed with anti-TNFR1, anti-TNFR2, anti-TRADD, anti-TRAF2, anti-RIP and anti-β-actin antibodies.

FIG. 28 demonstrates neutralizing cytotoxicity of scFvMEL/TNF by anti-TNFR1 Ab. A375-M cells or SKBR3-HP cells (4000 cells per well) were pre-treated with 25 μg/ml of anti-TNFR1 Ab for 2 h before the cells were treated with scFvMEL/TNF or TNF at the I.C. concentration for 72 h. The effect of scFvMEL/TNF and TNF on the growth of tumor cells in culture was determined using crystal violet staining, then the optical densities at 595 nm were measured.

FIG. 29 shows pharmacokinetics of ¹²⁵I-labeled scFvMEL/TNF in mice. The radiolabeled fusion construct was administered (i.v., tail vein) to Balb/c mice. Groups (5/group) were sacrificed at various times after administration. The radioactivity in plasma was assessed, and the results were analyzed by a least-square nonlinear regression (PK Analyst; MicroMath, Inc.). The data demonstrated a triphasic curve fit with calculated half-lives of 0.38 h, 3.9 h and 17.6 h for the α-, β- and γ-phases, respectively.

FIG. 30 provides a group average terminal body weight (TBW) and relative organ weights. Five mice per group were injected intravenously daily for 5 days with 0.2, 0.4, 0.6, and 0.8 mg/kg/day of scFvMEL/TNF (Groups 2-5). The total dose delivered was 1, 2, 3, 4 mg/kg which corresponds to 25, 50, 75, and 100% of an established MTD. The vehicle control group (Group 1) consisted of saline. Seven days after the last injection (Day 12), mice were sacrificed by exposure to CO₂. The Terminal Body Weight (TBW) of each mouse was measured before a complete necropsy including liver, kidneys, spleen, etc. was performed. The organ weight of individual animal was measured before organs were fixed by immersion in neutral-buffered 10% Formalin solution. The relative (to body weight) organ weights were calculated as a percent of control (Organ WT/TBW×100). ScFvMEL/TNF causes a dose-related increase in the relative spleen weights (relative to body weight).

FIG. 31 shows antitumor activity of scFvMEL/TNF on A375GFP tumor xenografts monitored by Xenogen IVIS 200 Imaging System. Nude mice bearing established (50 mm³) human melanoma (A375GFP) tumors stably transfected with green-fluorescent protein (GFP) growing in the right flank were treated (i. v. tail vein) with either saline (controls) or scFvMEL at 2.5 mg/kg or scFvMEL (0.2 mg/kg) plus TNF (0.2 mg/kg), or scFvMEL/TNF at 2.5 mg/kg (total dose) for 5 consecutive days. Tumors were monitored using a Xenogen IVIS 200 Imaging System after mice were anesthesthetized with Nembutal at 50 mg/kg (i. p.) once a week after treatment.

FIG. 32 shows nude mice bearing human melanoma (A375GFP) tumors were treated i. v. (tail vein) with either saline (controls) or scFvMEL at 2.5 mg/kg or scFvMEL (0.2 mg/kg) plus human recombinant TNF (0.2 mg/kg), or scFvMEL/TNF at 2.5 mg/kg (total dose) for 5 consecutive days (arrows). Treatment of mice bearing established (50 mm3) tumors with scFvMEL/TNF at a dose of 2.5 mg/kg resulted in potent tumor suppression and complete tumor regression of all lesions (5/5 mice tumor free on day 43). In contrast, all mice treated with either saline, scFvMEL alone, or scFvMEL plus TNF showed rapid tumor growth. Mice bearing larger tumors (150 mm3) also showed tumor regression (3/5 tumor free on day 44).

FIG. 33 shows the expression of gp240 antigen on different melanoma cells detected by ELISA. To examine the expression of gp240 antigen on melanoma A375-M, TXM-18, TXM-13, MEL 526 and TXM-1 cells, parental monoclonal antibody ZME-018 IgG2a that specifically binds to gp240 antigen was used in ELISA. Ninety-six well ELISA plates containing adherent melanoma cells (5×10⁴ cells per well) were blocked by addition of a solution containing 5% bovine serum albumin (BSA) for 1 h. Cells were incubated with monoclonal antibody ZME-018 IgG2a (produced by our core lab) followed by incubation with goat anti-mouse/horseradish peroxidase conjugate (HRP-GAM). The substrate solution ABTS containing 1 ml/ml 30% H₂O₂ was added to the wells. Absorbance at 405 nm was measured after 30 min. The results demonstrated that gp240 antigen presents on A375-M, TXM-18L, TXM-13, and MEL-526 cells, however, very low level expressing of gp240 antigen on TXM-1 cells was observed.

FIG. 34 demonstrates the gp240 expression on melanoma cell lines detected by FACS assay. Melanoma cells consisting of 1×10⁶ cells were first treated with monoclonal antibody ZME-018 IgG2a for 20 min at 4° C., then stained with allophycocyanin (APC)-conjugated Goat-Anti-Mouse antibody (BD Immunocytometry System, CA) for another 20 min at 4° C., both resuspended in 100 ml FACS staining buffer (2% FCS/DPBS). As a negative staining control, cells were stained with an isotype-matched control antibody of irrelevant specificity (Mouse IgG2a, PharMingen, San Diego, Calif.) at the same concentration as the antibody against gp240. Following staining, cells were washed twice with DPBS, then resuspended in 500 ml of 1% paraformaldehyde solution and stored on ice in the dark. FACS analysis was carried out right afterward on a FACS Caliber cytometer (Becton Dickinson, San Jose, Calif.). APC fluorescence was detected in the FL-4 channel. For each cell line, 10,000 events were acquired. Analysis was performed with the CellQuest Pro™ software ((Becton Dickinson). Expression of gp240 was indicated as the solid lines for all five melanoma cell lines (A375M, TXM13, TXM18, MEL526 and TXM1) and also they were overlayed by the dot lines representing the isotype control.

FIGS. 35A and 35B show binding activity of scFvMEL moiety of GrB/scFvMEl fusion protein by ELISA. Ninety-six well ELISA plates containing adherent melanoma cells (5×10⁴ cells per well) were blocked by addition of a solution containing 5% bovine serum albumin (BSA) for 1 h. To detect the binding activity of GrB/scFvMEL, cells were incubated with purified GrB/scFvMEL at various concentrations for 1 h at room temperature (RT). After they were washed, the cells were incubated with rabbit anti-scFvMEL antibody, followed by addition of goat anti-rabbit/HRP conjugate (HRP-GAR) antibody. Finally, the substrate solution ABTS containing 1 ml/ml 30% H₂ O₂ was added to the wells. Absorbance at 405 nm was measured after 30 min. In FIG. 35A, the profile of absorbance indicated the binding activity of GrB/scFvMEL at different concentrations on different melanoma cell lines. In FIG. 35B, there is the absorbance of GrB/scFvMEL at the same IC₅₀ concentration on different melanoma cell lines. The results demonstrated that GrB/scFvMEL bound to high-level gp240 antigen expressing melanoma A375-M, TXM-18L, TXM-13 and MEL-526 cells. Moreover, the binding activity was stronger in A375-M and MEL-526 followed by TXM-18L and TXM-13. However, the protein did not bind to TXM-1 in which has very low-level expression of gp240 antigen.

FIG. 36 shows cytotoxicity assays in vitro against melanoma cells. Samples (GrB/scFvMEL or MEL sFv/rGel) were assayed using a standard 72-h cell proliferation assay with melanoma cell monolayers and using crystal violet staining. The percent of control refers to the percentage of cells in the drug-treated wells compared to that of control (untreated) wells. The Bar showed the IC₅₀ value on different melanoma cell lines. There are cytotoxic effects on A375M, MEL526, TXM-18L and TXM13 that are high expressed gp240 antigen. However, no cytotoxic effects were found on TXM-1 cells at doses of up to 1 μM.

FIG. 37 provides studies of GrB/scFvMEL in combination with various chemotherapeutic agents. Antigen-positive (A375M) cells were pretreated (at IC₂₅ doses) with various chemotherapeutic agents for 6 h followed by addition of GrB/scFvMEL (IC₂₅). The cells were then incubated for a total of 72 h (sequence C1). Alternatively, cells were first treated with GrB/scFvMEL for 6 h, and then various chemotherapeutic agents were added for 72 h (sequence C2). Chemotherapeutic agents include doxorubicin (DOX), vincristine (VCR), etoposide (VP-16), cisplantin (CDDP), cytarabine (Ara C) and 5-Fu. Co-administration GrB/scFvMEL and chemotherapeutic agents to A375 cells for 72 hours, demonstrated synergistic antitumor activity with DOX, VCR or CDDP and additive effects in combination with VP-16 or Ara C. Pre-treatment with GrB/scFvMEL for 6 h followed by co-exposure to these chemotherapeutic agents for 72 hours (C2) showed significantly inhibited growth as compared to pre-treatment with drugs followed by co-exposure the fusion construct (C1).

FIG. 38 provides antitumor activity of GrB/scFvMEL in vivo. Athymic (nu/nu) mice, female, 6-8 weeks of age, were injected subcutaneously, right flank with 3×10 6 log-phase A375-M cells and tumors were allowed to establish. Once tumors reached measurable size (˜30-50 mm³), animals were treated via i. v. tail vein with either saline (control) or GrB/scFvMEL fusion construct (37.5 mg/kg total dose) for 5 times every other day. Animals were monitored and tumors were measured for an additional 28 days. The saline-treated control tumors increased 24 fold (from 50 mm3 to 1200 mm³) over 28 days. In contrast, GrB/scFvMEL (37.5 mg/kg) treated tumors increased 4 fold (from 50 mm³ to 200 mm³).

FIG. 39 demonstrates that GrB/scFvMEL specifically binds to gp240 antigen positive melanoma cell lines as assessed by ELISA. Ninety-six well ELISA plates containing adherent melanoma cells (5×10⁴ cells per well) were blocked by addition of a solution containing 5% bovine serum albumin (BSA) for 1 h. To detect the binding activity of GrB/scFvMEL, cells were incubated with purified GrB/scFvMEL at various concentrations for 1 h at room temperature. After they were washed, the cells were incubated with rabbit anti-scFvMEL antibody, followed by addition of goat anti-rabbit/HRP conjugate (HRP-GAR) antibody. Finally, the substrate solution ABTS containing 1 μl/ml 30% H₂ O₂ was added to the wells. Absorbance at 405 nm was measured after 30 min. GrB/scFvMEL bound to high-level gp240 antigen expressing melanoma A375-M, MEL-526, TXM-18, but not to TXM-1 which has very low-level expression of gp240 antigen.

FIG. 40 shows that cellular resistance to doxorubicin is associated with a marginal cross-resistance to GrB/scFvMEL. A375-doxorubicin resistant (A375DR) cells are 400-fold resistant to doxorubicin compared to the parental A375 cells. Log phase A375DR cells were treated with various doses of GrB/scFvMEL for 72 hours. The percent of control refers to the percentage of cells in the drug-treated wells compared to that of control (untreated) wells. A375DR cells demonstrated only 4.5-fold resistant to GrB/scFvMEL compared to the parental A375 cells (I.C.₅₀ of 63.6 vs. 14.5 nM).

FIGS. 41A-41C show treatment with GrB/scFvMEL sensitizes melanoma cells to ionizing radiation. Radiosensitization by GrB/scFvMEL was based on clonogenic cell survival assays. A375 (FIG. 41A), A375DR (FIG. 41B), and SKBR3-HP (FIG. 41C) cells were pretreated with GrB/scFvMEL (10 nM for 16 hours), and the drug was washed off and cells were irradiated at various doses and plated for clonogenic cell survival assay. The observed sensitizations were statistically significant at 2, 4, and 6 Gy dosage groups on A375 cells (p<0.05) and at 4 and 6 Gy dosage groups on A375DR cells (p<0.05 and 0.005, respectively). No statistically-significant sensitization was observed in gp240 antigen negative SKBR3 cells (p>0.05).

FIGS. 42A and 42B demonstrate that GrB/scFvMEL inhibits A375DR cells invasion of Matrigel. A375DR cell aggregates were prepared as described under Materials and Methods section. Cell aggregates were transferred over the Matrigel cushion and then overlaid with additional 100 μl of Matrigel. The aggregates into Matrigel were covered with 400 μl culture medium in the absence or presence of GrB/scFvMEL (50 nM). The aggregates were then observed daily under a light microscope (FIG. 42A). A375DR cells actively leave the aggregate and invade the Matrigel preparation at 4 and 6 days. The treatment of A375DR cells with GrB/scFvMEL inhibits A375DR invasion of Matrigel at 4 and 6 days. The densities of cells invaded into matrigel surrounding the aggregates was analyzed by AlphaEase®FC software and the percent of invasion was calculated based on the cell densities of two groups and standardized by the value of non-treatment control group as 100% invasion (FIG. 42B).

FIG. 43 shows that GrB/scFvMEL demonstrates anti-tumor activity on xenograft melanoma model by inducing apoptosis in tumor tissue. Anti-tumor effect of GrB/scFvMEL on A375-M xenograft tumors. Athymic (nu/nu) mice, female, 6-8 weeks of age, were injected subcutaneously, right flank with 3×10⁶ log-phase A375-M cells and tumors were allowed to establish. Once tumors reached measurable size (˜50 mm³), animals were administered intravenously with either saline (control) or GrB/scFvMEL fusion construct for 5 times every other day (37.5 mg/kg total dose). Animals were monitored and tumors were measured for an additional 28 days. The saline-treated control tumors increased 24 fold (from 50 mm³ to 1200 mm³) over 28 days. In contrast, GrB/scFvMEL treated tumors increased 4 fold (from 50 mm³ to 200 mm³).

### DETAILED DESCRIPTION OF THE INVENTION

As used herein the specification, “a” or “an” may mean one or more. As used herein in the claim(s), when used in conjunction with the word “comprising”, the words “a” or “an” may mean one or more than one. As used herein “another” may mean at least a second or more. Some embodiments of the invention may consist of or consist essentially of one or more elements, method steps, and/or methods of the invention. It is contemplated that any method or composition described herein can be implemented with respect to any other method or composition described herein.

I. Definitions

The term “blockage of the SAPK/JNK signal pathway” as used herein refers to inhibiting at least in part the function of one or more components of the pathway, reducing the half-life and/or biological activity of one or more components of the pathway, reducing expression of one or more components of the pathway, and so forth. Components of the SAPK/JNK signal pathway are known in the art, although exemplary embodiments include SAPK/JNK, c-Jun, MKK4, SEK1, p54/46 ZAP-70, NfAT, MEKK1, GrB2, MEKK 4/7, and Vinculin, for example.

The term “chemosensitivity” as used herein refers to the ability of one or more cells to be effectively treated by a cancer therapy. In particular, it refers to the ability of one or more cells to be destroyed or to have at least reduced proliferation by one or more chemotherapeutics.

The term “chemotherapy resistance” as used herein refers to the ability of a cancer cell to be refractory to therapy by one or more chemotherapy agents. In one specific embodiment, the one or more chemotherapies were initially effective (sensitive) to the one or more agents, whereas in alternative embodiments the one or more chemotherapies were never substantially effective on the cancer cell.

The term “cytotoxic” refers to an agent having a toxic or destructive effect on one or more cells, such as cancer cells, including in a tumor, such as a solid tumor. In an alternative embodiment, the agent is destructive to a cancer cell that is not in a tumor, such as in a non-solid tumor, including leukemia or lymphoma.

The term “inhibition of Akt phosphorylation” refers to blocking at least in part the phosphorylation of Akt on one or more molecules. The inhibition may be determined by any suitable method in the art, such as by western blot with antibodies to phospho-Akt, for example.

The term “downregulation of Bcl-2” refers to decreasing the expression level of Bcl-2. The level may be determined by any suitable method in the art, including by Westerns or Northerns, for example.

The term “granzyme” as used herein is defined as an enzyme from the granules of cytotoxic lymphocytes that upon entry into the cytosol of a cell induce apoptosis and/or nuclear DNA fragmentation. In a specific embodiment, the granzyme is a lymphocyte serine protease. In some embodiments, the granzyme is full-length, whereas in other embodiments the granzyme is partial.

The term “HER-2/neu overexpressing” as used herein refers to expression of HER-2/neu in a particular cell being greater than 2-fold higher than corresponding non-cancerous cells of the same tissue. In addition, a “cancer associated with overexpression of HER-2/neu” is used herein to refer to cancerous tissue comprising more HER-2/neu than non-cancerous tissue from the same portion of the body. The expression level of HER-2/neu may be determined by any suitable method in the art, such as, for example, by Western blot, northern blot, or quantitative FISH.

The term “immunocytokine” as used herein refers to a class of recombinant agents comprising cytokines fused to antibodies, and these constructs are useful for re-directing their biological effects to target specific cells and to prevent non-target toxicity.

The term “NF-κB overexpressing” as used herein refers to expression of NF-κB in a particular cell being greater than 2-fold higher than corresponding non-cancerous cells of the same tissue. In addition, a “cancer associated with overexpression of NF-κB” is used herein to refer to cancerous tissue comprising more NF-κB than non-cancerous tissue from the same portion of the body. The expression level of NF-κB may be determined by any suitable method in the art, such as, for example, by Western blot, northern blot, or quantitative FISH.

The term “sensitivity to chemotherapy” as used herein refers to being treatable by one or more chemotherapeutics, and in specific embodiments is treatable by one or more particular chemotherapeutics.

II. The Present Invention

Traditional chemotherapy can be an extremely ineffective means of treating particular cancers due to the often drug-resistant characteristic of the disease. A growing understanding of the molecular events that mediate tumor growth and metastases has led to the development of rationally designed targeted therapeutics that offer the dual hope of maximizing efficacy and minimizing toxicity to normal tissue (Awada et al., 2003). In recent years, a strategy in cancer therapy in general has been the use of maximum tolerated doses of toxic non-specific agents as well as the investigation of a range of new agents that specifically target tumor-related molecules through a variety of biological pathways. Approaches to directly modulate apoptosis pathways are of particular interest in terms of new drug development in melanoma, for example. The activation of apoptotic mechanisms in melanoma cells has been directly implicated in the response of patient tumors to chemotherapy, response to radiotherapy and propensity to metastasize.

The present invention concerns chimeric molecules and their use for treatment and/or prevention of cancer. More particularly, the chimeric molecules are utilized for cancers that are refractory to treatment or that develop resistance to a chemotherapy. These kinds of cancers may be of any kind, but in particular embodiments they are Her-2/neu overexpressing and/or are resistant to TNF-α when used alone. In specific embodiments, the cancer is melanoma, pancreatic cancer, or breast cancer, for example.

The chimeric molecules comprise at least two components, including a targeting moiety and either a cytotoxic moiety or an apoptosis-inducing moiety. In specific embodiments, the targeting moiety is an antibody fragment. In additional specific embodiments, the cytotoxic moiety comprises TNF-α, and the apoptosis-inducing moiety comprises a granzyme, such as granzyme A or granzyme B.

The present invention is particularly useful for conferring or restoring chemosensitivity to a chemotherapy-resistant cell, and in specific embodiments the chimeric molecules are used in conjunction with a conventional chemotherapeutic agent. In specific embodiments, the chimeric molecules act through a particular mechanism, such as by evoking apoptotic pathways regardless of whether or not the anti-cell proliferation factor component of the chimeric molecule is a cytotoxic agent or a pro-apoptotis inducing moiety.

In particular, the chimeric molecules of the invention are useful for treating cancers that comprise particular cellular etiologies, such as those that are HER-2/neu overexpressing, resistant to TNF-α, NF-κB defective, or a combination thereof, for example.

III. Resistance to Chemotherapy

Of particular concern in chemotherapy treatment is the development of cancer resistance to a specific chemotherapeutic or class of chemotherapeutics during treatment. The resistance may manifest during the first treatment of chemotherapy or during subsequent chemotherapy treatments. In alternative embodiments, one or more chemotherapies are never detectably effective against a cancer.

In specific embodiments of the present invention, during a particular chemotherapeutic treatment some of the cells that are not killed by the chemotherapy mutate and become resistant to the particular drug. Once these cells multiply, there may be more resistant cells than cells that are sensitive to the chemotherapy. In other embodiments of chemotherapeutic resistance, gene amplification is involved, such as when a cancer cell produces many copies of a particular gene, which triggers an overproduction of protein that renders the anticancer drug ineffective. In other embodiments, cancer cells may pump the drug out of the cell substantially as fast as the drug is going in, or cancer cells may even stop taking in the drug(s) because the protein that transports the drug across the cell wall ceases to function. In additional embodiments of chemotherapy-resistance mechanisms, the cancer cells learn how to repair DNA breaks caused by some anti-cancer drugs and/or the cancer cells develop a mechanism that inactivates the drug, for example.

In many instances, the development of drug resistance is one reason that drugs are often given in combination, which may reduce the incidence of developing resistance to any one drug. It is known that upon development of resistance to one drug or group of drugs, it is more likely that the cancer may be resistant to other drugs. In particular aspects of the invention, the compositions and methods of the present invention employ more than one cancer-treating agent. For example, more than one chimeric molecule of the invention may be administered to an individual with cancer, such as an individual suspected of having cancer cells that develop resistance to cancer, or a chimeric molecule of the present invention may be used in combination with conventional chemotherapeutics, such as 5-fluorouracil, for example.

IV. Chemotherapeutic Agents

In embodiments of the present invention, the chimeric molecules comprising a cell-specific targeting moiety and either an apoptotic-inducing moiety or a cytoxic agent are effective when used in combination with one or more chemotherapeutic agents, are effective in sensitizing a cancer to one or more chemotherapeutic agents, are effective in conferring or restoring sensitivity to a chemotherapy-resistant cancer, or a combination thereof. In particular, the chimeric molecule and the chemotherapeutic agent may act additively or synergistically against the cancer. The term “synergistically” as used herein refers to the combined effect of the chimeric molecule and chemotherapeutic(s) being greater than the sum of their individual effects.

The term “chemotherapeutic agent” as used herein concerns at least conventional chemotherapeutic agents of the following exemplary classes: alkylating agents; nitrosoureas; antimetabolites; antitumor antibiotics; plant alkyloids; taxanes; hormonal agents; and miscellaneous agents. Although the chimeric molecules of the present invention are effective for cancer therapy, in the context of this invention they do not refer to the conventional term “chemotherapeutic agent” as used herein.

Alkylating agents are so named because of their ability to add alkyl groups to many electronegative groups under conditions present in cells, thereby interfering with DNA replication to prevent cancer cells from reproducing. Most alkylating agents are cell cycle non-specific. In specific embodiments, they stop tumor growth by cross-linking guanine bases in DNA double-helix strands. Examples include busulfan, carboplatin, chlorambucil, cisplatin, cyclophosphamide, dacarbazine, ifosfamide, mechlorethamine hydrochloride, melphalan, procarbazine, thiotepa, and uracil mustard, for example.

Nitrosoureas are referred to as alkylating agents because they act by the process of alkylation to inhibit DNA repair. These alkylating agents are metabolites that interfere with enzymes needed for DNA repair. The nitrosoureas are able to cross the blood-brain barrier, so they are used to treat brain tumors as well as non-Hodgkin's lymphoma, multiple myeloma, and malignant melanoma, for example. Most nitrosourea drugs are cell cycle-nonspecific. Examples include carmustine, lumustine, and streptozocin.

Anti-metabolites prevent incorporation of bases into DNA during the synthesis (S) phase of the cell cycle, prohibiting normal development and division. Antimetabolites include drugs such as 5-fluorouracil, 6-mercaptopurine, capecitabine, cytosine arabinoside, floxuridine, fludarabine, gemcitabine, methotrexate, and thioguanine.

There are a variety of antitumor antibiotics that generally prevent cell division by interfering with enzymes needed for cell division or by altering the membranes that surround cells. Included in this class are the anthracyclines, such as doxorubicin, which act to prevent cell division by disrupting the structure of the DNA and terminate its function. These agents are cell cycle non-specific. Antitumor antibiotics include dactinomycin, daunorubicin, doxorubicin, idarubicin, mitomycin-C, and mitoxantrone.

Plant alkaloids inhibit or stop mitosis or inhibit enzymes that prevent cells from making proteins needed for cell growth. Frequently used plant alkaloids include vinblastine, vincristine, vindesine, and vinorelbine.

The taxanes affect cell structures called microtubules that are important in cellular functions. In normal cell growth, microtubules are formed when a cell starts dividing, but once the cell stops dividing, the microtubules are disassembled or destroyed. Taxanes prohibit the microtubules from breaking down such that the cancer cells become so clogged with microtubules that they cannot grow and divide. Exemplary taxanes include paclitaxel and docetaxel.

Hormonal agents and hormone-like drugs are utilized for certain types of cancer, including, for example, leukemia, lymphoma, and multiple myeloma. They are often employed with other types of chemotherapy drugs to enhance their effectiveness. Sex hormones are used to alter the action or production of female or male hormones and are used to slow the growth of breast, prostate, and endometrial cancers, for example. Inhibiting the production (aromatase inhibitors) or action (tamoxifen) of these hormones can often be used as an adjunct to therapy. Some other tumours are also hormone dependent. Tamoxifen is an example of a hormonal agent that interferes with the activity of estrogen, which promotes the growth of breast cancer cells.

Miscellaneous agents include chemotherapeutics such as bleomycin, hydroxyurea, L-asparaginase, and procarbazine, for example.

V. Generation of Chimeric Molecules

The chimeric molecules may be produced by any suitable manner such that the targeting moiety and the anti-cell proliferation moiety are associated. While the chimeric proteins of the present invention may be produced by chemical synthetic methods or by chemical linkage between the two moieties, for example, in particular embodiments they are produced by fusion of a coding sequence of a cell-specific targeting moiety and a coding sequence of an apoptosis-inducing protein under the control of a regulatory sequence that directs the expression of the fusion polynucleotide in an appropriate host cell. In preferred embodiments, each of the components of the chimeric protein comprise functional activity for their respective parts being a cell-specific targeting moiety and a signal transduction pathway factor (such as an apoptosis-inducing protein). For embodiments wherein chemical linkers are employed to associate the cell-targeting moiety and the anti-cell proliferation moiety, any suitable linker may be utilized in the invention. Specific examples include SPDP, SMPT, and/or Avidin/streptavidin:biotin, for example.

The fusion of two full-length coding sequences can be achieved by methods well known in the art of molecular biology. It is preferred that a fusion polynucleotide contain only the AUG translation initiation codon at the 5′ end of the first coding sequence without the initiation codon of the second coding sequence to avoid the production of two separate encoded products. In addition, a leader sequence may be placed at the 5′ end of the polynucleotide in order to target the expressed product to a specific site or compartment within a host cell to facilitate secretion or subsequent purification after gene expression. The two coding sequences can be fused directly without any linker or by using a flexible polylinker, such as one comprised of the pentamer Gly-Gly-Gly-Gly-Ser repeated 1 to 3 times. Such a linker has been used in constructing single chain antibodies (scFv) by being inserted between VH and VL (Bird et al., 1988; Huston et al., 1988). The linker is designed to enable the correct interaction between two beta-sheets forming the variable region of the single chain antibody. Other linkers that may be used include Glu-Gly-Lys-Ser-Ser-Gly-Ser-Gly-Ser-Glu-Ser-Lys-Val-Asp (SEQ ID NO:1) (Chaudhary et al., 1990) and Lys-Glu-Ser-Gly-Ser-Val-Ser-Ser-Glu-Gln-Leu-Ala-Gln-Phe-Arg-Ser-Leu-Asp (SEQ ID NO:2) (Bird et al., 1988), for example.

A. Cell-Specific Targeting Moieties

The chimeric proteins of the invention are comprised of a cell-specific targeting moiety and an anti-cell proliferation moiety. The cell-specific targeting moiety confers cell-type specific binding to the molecule, and it is chosen on the basis of the particular cell population to be targeted. A wide variety of proteins are suitable for use as cell-specific targeting moieties, including but not limited to, ligands for receptors such as growth factors, hormones and cytokines, and antibodies or antigen-binding fragments thereof.

1. Antibodies

In some embodiments of the invention, one or more antibodies are employed as a cell-specific targeting moiety. As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting.

The term “antibody” is used to refer to any antibody-like molecule that has an antigen binding region, and includes antibody fragments such as Fab′, Fab, F(ab′)2, single domain antibodies (DABs), Fv, scFv (single chain Fv), and the like. The techniques for preparing and using various antibody-based constructs and fragments are well known in the art. Means for preparing and characterizing antibodies are also well known in the art (See, e.g., Antibodies: A Laboratory Manual, Cold Spring Harbor Laboratory, 1988; incorporated herein by reference).

Antibodies are extremely versatile and useful cell-specific targeting moieties because they can be generated against any cell surface antigen of interest. Monoclonal antibodies have been generated against cell surface receptors, tumor-associated antigens, and leukocyte lineage-specific markers such as CD antigens. Antibody variable region genes can be readily isolated from hybridoma cells by methods well known in the art.

Over the past few years, several monoclonal antibodies have been approved for therapeutic use and have achieved significant clinical and commercial success. Much of the clinical utility of monoclonal antibodies results from the affinity and specificity with which they bind to their targets, as well as long circulating life due to their relatively large size. Monoclonal antibodies, however, are not well suited for use in indications where a short half-life is advantageous or where their large size inhibits them physically from reaching the area of potential therapeutic activity.

Moreover, antibodies in their native form, consisting of two different polypeptide chains that need to be generated in approximately equal amounts and assembled correctly, are not optimal candidates for therapeutic purposes. However, it is possible to create a single polypeptide that can retain the antigen binding properties of a monoclonal antibody.

Single chain antibodies (SCAs) are genetically engineered proteins designed to expand on the therapeutic and diagnostic applications possible with monoclonal antibodies. SCAs have the binding specificity and affinity of monoclonal antibodies and, in their native form, are about one-fifth to one-sixth of the size of a monoclonal antibody, typically giving them very short half-lives. Human SCAs offer many benefits compared to most monoclonal antibodies, including more specific localization to target sites in the body, faster clearance from the body, and a better opportunity to be used orally, intranasally, transdermally or by inhalation, for example. In addition to these benefits, fully-human SCAs can be isolated directly from human SCA libraries without the need for costly and time consuming “humanization” procedures. SCAs are also readily produced through intracellular expression (inside cells) allowing for their use in gene therapy applications where SCA molecules act as specific inhibitors of cell function.

Single-chain recombinant antibodies (scFvs) consist of the antibody VL and VH domains linked by a designed flexible peptide tether (Atwell et al., 1999). Compared to intact IfGs, scFvs have the advantages of smaller size and structural simplicity with comparable antigen-binding affinities, and they can be more stable than the analogous 2-chain Fab fragments (Colcher et al., 1998; Adams and Schier, 1999). Several studies have shown that the smaller size of scFvs provides better penetration into tumor tissue, improved pharmacokinetics, and a reduction in the immunogenicity observed with i.v. administered Fabs compared to that of intact murine antibodies (Bird et al., 1988; Cocher et al., 1990; Colcher et al., 1998; Adams and Schier, 1999). For example, the scFvMEL single-chain antibody retains the same binding affinity and specificity of the parental ZME-018 antibody that recognizes the surface domain of the gp240 antigen present on human melanoma cells (Kantor et al., 1982; Macey et al., 1998).

Recombinant single-chain Fv antibody (scFv)-based agents have been used in pre-clinical studies for cell-targeted delivery of cytokines (Liu et al., 2004) and intracellular delivery of highly cytotoxic n-glycosidases such as recombinant gelonin (rGel) toxin (Rosenblum et al., 2003). The smaller size of these antibody fragments may allow better penetration into tumor tissue, improved pharmacokinetics, and a reduction in the immunogenicity observed with intravenously administered murine antibodies. Initially, to target melanoma cells, we chose a recombinant single-chain antibody designated scFvMEL which recognizes the high-molecular-weight glycoprotein gp240, found on a majority (80%) of melanoma cell lines and fresh tumor samples (Kantor et al., 1982). It has been used extensively by the present inventors to target gp240 bearing cells in vitro and using xenograft models (Rosenblum et al,. 2003; Liu et al., 2003; Rosenblum et al., 1991; Rosenblum et al. 1994; Rosenblum et al., 1995; Rosenblum et al., 1996; Rosenblum et al., 1999). This antibody binds to target cells and is efficiently internalized making this an excellent carrier to deliver toxins or other therapeutic payloads.

Antibodies designated ZME-018 or 225.28 S that is the parental antibody of scFvMEL targeting the gp240 antigen have been extensively studied in melanoma patients and have demonstrated an impressive ability to localize in metastatic tumors after systemic administration (Rosenblum et al., 1994; Kantor et al,. 1986; Macey et al., 1988; Rosenblum et al., 1991). This antibody possesses high specificity for melanoma and is minimally reactive with a variety of normal tissues, making it a promising candidate for further study (Rosenblum et al,. 1995; Macey et al., 1988; Rosenblum et al., 1991; Mujoo et al,. 1995). More importantly, the gp240 antigen is not expressed on normal cells thus making this an interesting target for therapeutic intervention.

The variable regions from the heavy and light chains (VH and VL) are both approximately 110 amino acids long. They can be linked by a 15 amino acid linker with the sequence (SEQ ID NO:3)₃, for example, which has sufficient flexibility to allow the two domains to assemble a functional antigen binding pocket. In specific embodiments, addition of various signal sequences allows the scFv to be targeted to different organelles within the cell, or to be secreted. Addition of the light chain constant region (Ck) allows dimerization via disulfide bonds, giving increased stability and avidity. Thus, for a single chain Fv (scFv) SCA, although the two domains of the Fv fragment are coded for by separate genes, it has been proven possible to make a synthetic linker that enables them to be made as a single protein chain scFv (Bird et al., 1988; Huston et al., 1988) by recombinant methods. Furthermore, they are frequently used due to their ease of isolation from phage display libraries and their ability to recognize conserved antigens (for review, see Adams and Schier, 1999). For example, scFv is utilized to target suicide genes to carcinoembryonic antigen (CEA)-expressing tumor cells by a retrovector displaying anti-CEA scFv (Kuroki et al., 2000).

Furthermore, the Fc portion of the heavy chain of an antibody may be used to target Fc receptor-expressing cells such as the use of the Fc portion of an IgE antibody to target mast cells and basophils. The use of antibodies to target a polypeptide or peptide of interest by antibody-directed therapy or immunological-directed therapy is currently approved and in use in the present therapeutic market.

A Fab antibody fragment may be utilized in the invention. An Fab fragment comprises a light chain and the N-terminal portion of the heavy chain that are linked together by disulfide bonds. It typically has a molecular weight of approximately 50 kD and comprises a single antigen binding site. Fab fragments may be obtained from F(ab′)₂ fragments by limited reduction, or from whole antibody by digestion with papain in the presence of reducing agents.

2. Moieties Other than Antibodies

Molecules other than antibodies or antibody fragments may be employed as cell-specific targeting moieties. Since a large number of cell surface receptors have been identified in hematopoietic cells of various lineages, ligands or antibodies specific for these receptors may be used as cell-specific targeting moieties. IL2 may be used as a cell-specific targeting moiety in a chimeric protein to target IL2R+ cells. Alternatively, other molecules such as B7-1, B7-2 and CD40 may be used to specifically target activated T cells (The Leucocyte Antigen Facts Book, 1993, Barclay et al. (eds.), Academic Press). Furthermore, B cells express CD19, CD40 and IL4 receptor and may be targeted by moieties that bind these receptors, such as CD40 ligand, IL4, IL5, IL6 and CD28. The elimination of immune cells such as T cells and B cells is particularly useful in the treatment of autoimmunity, hypersensitivity, transplantation rejection responses and in the treatment of lymphoid tumors. Examples of autoimmune diseases are multiple sclerosis, rheumatoid arthritis, insulin-dependent diabetes mellitus, systemic lupus erythemotisis, scleroderma, and uviatis. More specifically, since myelin basic protein is known to be the major target of immune cell attack in multiple sclerosis, this protein may be used as a cell-specific targeting moiety for the treatment of multiple sclerosis (WO 97/19179; Becker et al., 1997).

Other cytokines that may be used to target specific cell subsets include the interleukins (IL1 through IL15), granulocyte-colony stimulating factor, macrophage-colony stimulating factor, granulocyte-macrophage colony stimulating factor, leukemia inhibitory factor, tumor necrosis factor, transforming growth factor, epidermal growth factor, insulin-like growth factors, and/or fibroblast growth factor (Thompson (ed.), 1994, The Cytokine Handbook, Academic Press, San Diego).

A skilled artisan recognizes that there are a variety of known cytokines, including hematopoietins (four-helix bundles) (such as Epo (erythropoietin), IL-2 (T-cell growth factor), IL-3 (multicolony CSF), IL-4 (BCGF-1, BSF-1), IL-5 (BCGF-2), IL-6 IL-4 (IFN-β2, BSF-2, BCDF), IL-7, IL-8, IL-9, IL-11, IL-13 (P600), G-CSF, IL-15 (T-cell growth factor), GM-CSF (granulocyte macrophage colony stimulating factor), OSM (OM, oncostatin M), and LIF (leukemia inhibitory factor)); interferons (such as IFN-γ, IFN-α, and IFN-β); immunoglobin superfamily (such as B7.1 (CD80), and B7.2 (B70, CD86)); TNF family (such as TNF-α (cachectin), TNF-β (lymphotoxin, LT, LT-α), LT-β, CD40 ligand (CD40L), Fas ligand (FasL), CD27 ligand (CD27L), CD30 ligand (CD30L), and 4-1BBL)); and those unassigned to a particular family (such as TGF-β, IL 1α, IL-1β, IL-1 RA, IL-10 (cytokine synthesis inhibitor F), IL-12 (NK cell stimulatory factor), MIF, IL-16, IL-17 (mCTLA-8), and/or IL-18 (IGIF, interferon-γ inducing factor)).

Additionally, certain cell surface molecules are highly expressed in tumor cells, including hormone receptors such as human chorionic gonadotropin receptor and gonadotropin releasing hormone receptor (Nechushtan et al., 1997). Therefore, the corresponding hormones may be used as the cell-specific targeting moieties in cancer therapy. Examples of hormones that may be employed as cell-specific targeting moieties include proteins, peptides, and modified amino acids, or steroids, for example. Specific hormones include human chorionic gonadotropin, gonadotropin releasing hormone, androgens, such as testosterone, for example, or estrogens, such as estradiol, for example. Additional specific hormones include thyroid-stimulating hormone, follicle-stimulating hormone, luteinizing hormone, prolactin, growth hormone, adrenocorticotropic hormone, antidiuretic hormone, oxytocin, thyrotropin-releasing hormone, growth hormone releasing hormone, corticotropin-releasing hormone, somatostatin, dopamine, melatonin, thyroxine, calcitonin, parathyroid hormone, glucocorticoids (such as cortisol, for example), mineralocorticoids (such as aldosterone, for example), adrenaline, noradrenaline, progesterone, insulin, glucagon, amylin, erythropoitin, calcitriol, calciferol, atrial-natriuretic peptide, gastrin, secretin, cholecystokinin, neuropeptide Y, ghrelin, PYY₃₋₃₆, Insulin-like growth factor-1, leptin, thrombopoietin, or angiotensinogen, for example.

In addition, interferons may be employed as cell targeting moieties, such as, for example. Interferons (IFNs) belong to the large class of glycoproteins referred to as cytokines and are proteins generated by the immune system cells in response to challenges by foreign agents including viruses, bacteria, parasites and tumor cells, for example. Three major classes exist in humans: type I, type II, and type III. Type I IFNs comprise at least thirteen different alpha isoforms IFNA (1, 2, 4, 5, 6, 7, 8, 10, 13, 14, 16, 17, 21); a beta (IFNB1); an omega (IFNW1); an epsilon (IFNE1); and kappa (IFNK) isoforms. Type II IFNs comprise IFN gamma (IFNG). A third class comprises IFN-lambda having at least 3 different isoforms (IL29. IL28A, and IL28B).

Furthermore, vitamins may be utilized as cell-targeting moieties, including folate, vitamin D₃, vitamin K₁, vitamin E, and/or vitamin A, for example.

Thus, in some embodiments of the invention, no antibodies are utilized in the chimeric polypeptides.

B. Anti-Cell Proliferation Moiety

The compositions and methods of the present invention utilize a chimeric molecule having an anti-cell proliferation moiety that at least in part is responsible for indirectly or directly impairing proliferation of a cell, damaging the ability of the cell to proliferate, reducing the rate and/or extent of proliferation of a cell, rendering a cell dormant; rendering a cell non-proliferative; and/or killing, destroying, and/or eradicating a cell. In specific embodiments, the cell is a cancer cell, which may or may not be in a solid tumor. In particular embodiments, the anti-cell proliferation moiety comprises one or more apoptosis-inducing moieties or one or more cytotoxic moieties.

1. Apoptosis-Inducing Moieties

Traditional chemotherapeutic approaches for treatment of neoplastic disease have generally relied upon the targeting of rapidly proliferating cells by inhibiting DNA replication or cell division. Although this strategy has been effective, its innate lack of selectivity for tumor cells has resulted in diminishing returns, approaching the limits of acceptable toxicity. Apoptosis also contributes to cell death in tumors treated with various anticancer agents (Debatin, 1999). Development of resistance of cancer cells to chemotherapeutic agents has been correlated with a blockade of apoptotic signaling in resistant cells (Serrone and Hersey, 1999; Lin et al., 2003). Metastatic spread of breast cancer, for example, also appears to directly involve the apoptotic pathway. Some studies have shown that high metastatic potential is strictly associated with increased resistance to apoptosis (Lin et al., 2003; Bucci et al., 2001; Sierra et al., 2000; Lipponen, 1999). Suppression of drug-induced apoptosis by over-expression of oncogenes such as bcl-2, growth factors or their receptors (Wu et al., 2004; Botti et al., 2004; Abramovitch and Werner, 2003; Rowinsky, 2003) may be an important cause of both metastases and intrinsic chemo- and radiation-resistance.

Recent clinical studies (Martinez-Arribas et al., 2003) in patients with breast cancer, for example, suggest a direct link between tumor apoptosis, labeling index (Ki-67) and patient response to chemotherapeutic regimens. The studies assess the in vivo relationship of apoptosis to proliferation and Bcl-2 protein in human breast tumors both prior to chemotherapy and in the residual resistant cell population at the completion of treatment by evaluation of apoptotic index (AI), Ki67 and Bcl-2 protein expression in the tissue of patients with operable breast cancer immediately before ECF [6 cycles of epirubicin (50 mg/m², iv, Day 1), cisplatin (50 mg/m², iv, Day 1) and continuous infusional 5 Fu (200 mg/m²/24 hrs)] preoperative chemotherapy. There was a significant positive association between AI and Ki67 both before and after chemotherapy. In the residual specimens AI and Ki67 were significantly reduced compared with pre-treatment biopsies, while Bcl-2 expression showed a significant increase. These data suggest that apoptosis and proliferation are closely related in vivo. It is possible that the phenotype of reduced apoptosis and increased Bcl-2 may be associated with breast cancer cells resistant to cytotoxic chemotherapy (Ellis et al., 1998). Several studies have suggested a direct correlation between response to chemotherapy and activation of apoptotic pathways (Park et al., 2004; Archer et al., 2003; Simoes-Wust et al., 2002; Serrano et al., 2002). Chemo-resistance appears to frequently accompany clinical progression of breast cancers from a hormone-dependent, non-metastatic, antiestrogen-sensitive phenotype to a hormone-independent, invasive, metastatic, antiestrogen-resistant phenotype (Campbell et al., 2001).

Thus, in some embodiments, the present invention addresses delivery of certain pro-apoptotic proteins that are central mediators of this effect to the interior of target cells, which will result in cell death through apoptotic mechanisms. The apoptosis-inducing moiety induces programmed cell death upon entry into the target cell of the chimeric polypeptide, which is delivered for binding to the target cell by the cell-specific targeting moiety.

Any suitable pro-apoptotic moiety may be employed in the invention. The pro-apoptotic moieties include granzymes, such as granzyme A or granzyme B, or a Bcl-2 family member.

The pro-apoptotic proteins in the BCL2 family may also be utilized as the apoptosis-inducing moieties in the present invention. Such human proteins are expected to have reduced immunogenicity over many immunotoxins composed of bacterial toxins. Although Bax is a useful apoptosis-inducing moiety in one embodiment of the present invention, other members in this family are suitable for use in the present invention and include Bak (Farrow et al., 1995; Chittenden et al., 1995; Kiefer et al., 1995), Bcl-Xs (Boise et al., 1993; Fang et al., 1994), Bad (Yang et al., 1995), Bid (Wang et al., 1996), Bik (Boyd et al., 1995), Hrk (Inohara et al., 1997) and/or Bok (Hsu et al., 1997). The nucleotide sequences encoding these proteins are known in the art and are readily obtainable from databases such as GenBank, and thus cDNA clones can be readily obtained for fusion with a coding sequence for a cell-specific targeting moiety in an expression vector.

Specific domains of particular members of the Bcl-2 family have been studied regarding their apoptosis-inducing activities. For example, the GD domain of Bak is involved in the apoptosis function (U.S. Pat. No. 5,656,725). In addition, Bax and Bipla share a homologous domain. Therefore, any biologically active domains of the Bcl-2 family may be used as an apoptosis-inducing moiety for the practice of the present invention.

Caspases also play a central role in apoptosis and may well constitute part of the consensus core mechanism of apoptosis. Caspases are implicated as mediators of apoptosis. Since the recognition that CED-3, a protein required for developmental cell death, has sequence identity with the mammalian cysteine protease interleukin-1 beta-converting enzyme (ICE), a family of at least 10 related cysteine proteases has been identified. These proteins are characterized by almost absolute specificity for aspartic acid in the P1 position. All the caspases (ICE-like proteases) contain a conserved QACKG (where X is R, Z or G) pentapeptide active-site motif. Caspases are synthesized as inactive proenzymes comprising an N-terminal peptide (Prodomain) together with one large and one small subunit. The crystal structures of both caspase-1 and caspase-3 show that the active enzyme is a heterotetramer, containing two small and two large subunits. Activation of caspases during apoptosis results in the cleavage of critical cellular substrates, including poly (ADP-riose) polymerase and lamins, so precipitating the dramatic morphological changes of apoptosis (Cohen, 1997, Biochem. J. 326:1-16). Therefore, it is also within the scope of the present invention to use a caspase as an apoptosis-inducing moiety.

Recently a few new proteins were cloned and identified as factors required for mediating activity of proteins, mainly caspases, involved in the apoptosis pathway. One factor was identified as the previously known electron transfer protein, cytochrome c (Lin et al., 1996, Cell 86:147-157), designed as Apaf-2. In addition to cytochrome c the activation of caspase-3 requires two other cytosolic factors-Apaf-1 and Apaf-3. Apaf-1 is a protein homologous to C. elegans CED-4, and Apaf-3 was identified as a member of the caspase family, caspase-9. Both factors bind to each other via their respective NH2-terminal CED-3 homologous domains, in the presence of cytochrome c, an event that leads to caspase-9 activation. Activated caspase-9 in turn cleaves and activates caspase-3 (Liu et al., 1996; Zou et al., 1997; Li et al., 1997). Another protein involved in the apoptotic pathway is DNA fragmentation factor (DFF), a heterodimer of 45 and 40 kd subunits that functions downstream of caspase-3 to trigger fragmentation of genomic DNA into nucleosomal segments (Liu et al., 1997).

2. Cytotoxic Agents

An additional form of anti-cell proliferation moiety useful in the invention includes one or more cytotoxic agents, which may also be referred to as cytotoxins. Cytotoxic agents are employed in compositions and methods of in the invention in conjunction with a cell-targeting moiety, and in particular embodiments the cytotoxic agent is the component of the chimeric molecules substantially responsible for killing and/or reducing proliferation of one or more cells, such as one or more cancer cells. In an alternative embodiment of the invention, the cytotoxic agent is instead a cytostatic agent capable of retarding, inhibiting, suppressing, and so forth the cellular activity and/or replicative capability of the cell.

Any useful cytotoxic agent may be used in the chimeric molecules, and the primary types of directly cytotoxic enzymes include, for example, the class of ribosome-inhibiting proteins (RIPs); pseudomonas exotoxin (PE); the highly potent plant n-glycosidase gelonin, which may be recombinant (rGel); and TNF-α. Toxins such as pseudomonas exotoxin (PE) and gelonin (rGel) have been successfully utilized because only a few molecules are needed to irretrievably be toxic to a target cell (Rosenblum et al., 2003; Veenendaal et al., 2002). Recently, Newton et al. described a new class of immunoconjugates containing human RNase that has in vitro and in vivo cytotoxic activity against human tumor cell lines and xenografts (Newton et al., 2001) primarily through degradation of RNA. Other exemplary embodiments of cytotoxic agents include nephrotoxins, neurotoxins, enterotoxins, Clostridium difficile Toxin B, Helicobacter pylori VacA, Yersinia enterocolitica YopT.

Although in particular embodiments of the invention, the cytotoxic agent of the present invention is TNF-α, in alternative embodiments the chimeric molecules comprise rGel fusion constructs that kill cells through a necrotic rather than an apoptotic process.

In some embodiments of the invention, recombinant cytotoxic agents are employed in the chimeric molecule. These may be referred to as “designer toxins.” These recombinant cytotoxic agents may be provided that is altered with respect to the native sequence, such as by having amino acids replaced or removed as compared to the native protein sequence. The recombinant cytotoxic agent may comprise the whole sequence or a partial sequence, and the partial sequence may be associated with a heterologous sequence.

For example, as indicated in U.S. patent application Ser. No. 10/074,596, which is incorporated by reference herein in its entirety, a recombinant gelonin toxin is provided that is altered with respect to the native gelonin sequence. The recombinant gelonin toxin or the present invention does not have all of the amino acids of the native gelonin, but in some embodiments, comprises a core toxin region defined as amino acid residues 110-210 of a particular sequence therein.

As an exemplary embodiment only, a recombinant gelonin toxin of the invention may include a gelonin toxin that is truncated with respect to the native sequence, such that the toxin is lacking at least 5, 10, 20, 30, 40, 50, or more amino acids. In some embodiments of the invention, the toxin contains the core toxin region, but is missing amino acids anywhere outside the core toxin region. In addition to deletions, the recombinant gelonin toxin of the invention may have an amino acid in place of a removed amino acid. For example, the glycine residue at position 7 in the gelonin protein sequence may be replaced with a non-glycine amino acid residue or a modified amino acid. If the glycine residue at position 7 is merely removed, the alanine at position 8 in SEQ ID NO: 1 becomes position 7, but is not considered a replacement because the positions of the amino acids are simply shifted by 1 position. It is contemplated that at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, or more amino acids may be replaced in the exemplary gelonin embodiment of the cytotoxic agent.

VI. Chimeric Molecule Production

The chimeric molecule of the present invention may be produced in any suitable manner in the art, although in particular embodiments the chimeric molecule is generated as a fusion polypeptide or is chemically conjugated, such as by a linker.

A. Chimeric Chemical Conjugates/Chimeric Conjugates with Linkers

In embodiments wherein the chimeric molecule is produced by conjugation, such as chemical conjugation or by a linker, the singular components are provided or obtained and are then associated by the chemical conjugation or linking method.

For example, the chimeric molecule components may be joined via a biologically-releasable bond, such as a selectively-cleavable linker or amino acid sequence. For example, peptide linkers that include a cleavage site for an enzyme preferentially located or active within a tumor environment are contemplated. Exemplary forms of such peptide linkers are those that are cleaved by urokinase, plasmin, thrombin, Factor IXa, Factor Xa, or a metallaproteinase, such as collagenase, gelatinase, or stromelysin. Alternatively, peptides or polypeptides may be joined to an adjuvant.

Amino acids such as selectively-cleavable linkers, synthetic linkers, or other amino acid sequences may be used to separate proteinaceous moieties. Additionally, while numerous types of disulfide-bond containing linkers are known that can successfully be employed to conjugate the toxin moiety with the targeting agent, certain linkers will generally be preferred over other linkers, based on differing pharmacologic characteristics and capabilities. For example, linkers that contain a disulfide bond that is sterically “hindered” are to be preferred, due to their greater stability in vivo, thus preventing release of the toxin moiety prior to binding at the site of action.

Additionally, any other linking/coupling agents and/or mechanisms known to those of skill in the art can be used to combine the components of the present invention, such as, for example, antibody-antigen interaction, avidin biotin linkages, amide linkages, ester linkages, thioester linkages, ether linkages, thioether linkages, phosphoester linkages, phosphoramide linkages, anhydride linkages, disulfide linkages, ionic and hydrophobic interactions, bispecific antibodies and antibody fragments, or combinations thereof.

It is contemplated that a cross-linker having reasonable stability in blood will be employed. Numerous types of disulfide-bond containing linkers are known that can be successfully employed to conjugate targeting and therapeutic/preventative agents. Linkers that contain a disulfide bond that is sterically hindered may prove to give greater stability in vivo, preventing release of the targeting peptide prior to reaching the site of action. These linkers are thus one group of linking agents.

Another cross-linking reagent is SMPT, which is a bifunctional cross-linker containing a disulfide bond that is “sterically hindered” by an adjacent benzene ring and methyl groups. It is believed that steric hindrance of the disulfide bond serves a function of protecting the bond from attack by thiolate anions such as glutathione which can be present in tissues and blood, and thereby help in preventing decoupling of the conjugate prior to the delivery of the attached agent to the target site.

The SMPT cross-linking reagent, as with many other known cross-linking reagents, lends the ability to cross-link functional groups such as the SH of cysteine or primary amines (e.g., the epsilon amino group of lysine). Another possible type of cross-linker includes the hetero-bifunctional photoreactive phenylazides containing a cleavable disulfide bond such as sulfosuccinimidyl-2-(p-azido salicylamido)ethyl-1,3′-dithiopropionate. The N-hydroxy-succinimidyl group reacts with primary amino groups and the phenylazide (upon photolysis) reacts non-selectively with any amino acid residue.

In addition to hindered cross-linkers, non-hindered linkers also can be employed in accordance herewith. Other useful cross-linkers, not considered to contain or generate a protected disulfide, include SATA, SPDP and 2-iminothiolane (Wawrzynczak & Thorpe, 1987). The use of such cross-linkers is well understood in the art. Another embodiment involves the use of flexible linkers.

U.S. Pat. No. 4,680,338, describes bifunctional linkers useful for producing conjugates of ligands with amine-containing polymers and/or proteins, especially for forming antibody conjugates with chelators, drugs, enzymes, detectable labels and the like. U.S. Pat. Nos. 5,141,648 and 5,563,250 disclose cleavable conjugates containing a labile bond that is cleavable under a variety of mild conditions. This linker is particularly useful in that the agent of interest may be bonded directly to the linker, with cleavage resulting in release of the active agent. Preferred uses include adding a free amino or free sulfhydryl group to a protein, such as an antibody, or a drug.

U.S. Pat. No. 5,856,456 provides peptide linkers for use in connecting polypeptide constituents to make fusion proteins, e.g., single chain antibodies. The linker is up to about 50 amino acids in length, contains at least one occurrence of a charged amino acid (preferably arginine or lysine) followed by a proline, and is characterized by greater stability and reduced aggregation. U.S. Pat. No. 5,880,270 discloses aminooxy-containing linkers useful in a variety of immunodiagnostic and separative techniques.

B. Chimeric Fusion Molecules

In embodiments wherein the chimeric molecule comprises a fusion protein, a polynucleotide that encodes a chimeric protein, mutant polypeptide, biologically active fragment of chimeric protein, or functional equivalent thereof, may be used to generate recombinant DNA molecules that direct the expression of the chimeric protein, chimeric peptide fragments, or a functional equivalent thereof, in appropriate host cells.

Due to the inherent degeneracy of the genetic code, other DNA sequences that encode substantially the same or a functionally equivalent amino acid sequence, may be used in the practice of the invention of the cloning and expression of the chimeric protein. Such DNA sequences include those capable of hybridizing to the chimeric sequences or their complementary sequences under stringent conditions. In one embodiment, the phrase “stringent conditions” as used herein refers to those hybridizing conditions that (1) employ low ionic strength and high temperature for washing, for example, 0.015 M NaCl/0.0015 M sodium citrate/0.1% SDS at 50° C.; (2) employ during hybridization a denaturing agent such as formamide, for example, 50% (vol/vol) formamide with a 0.1% bovine serum albumin/0.1% Ficoll/0.1% polyvinylpyrrolidone/50 mM sodium phosphate buffer at pH 6.5 with 750 mM NaCl, 75 mM sodium citrate at 42° C.; or (3) employ 50% formamide, 5×SSC (0.75 M NaCl, 0.075 M Sodium pyrophosphate, 5× Denhardt's solution, sonicated salmon sperm DNA (50 μg/ml), 0.1% SDS, and 10% dextran sulfate at 42° C., with washes at 42° C. in 0.2×SSC and 0.1% SDS.

Altered DNA sequences that may be used in accordance with the invention include deletions, additions or substitutions of different nucleotide residues resulting in a sequence that encodes the same or a functionally equivalent fusion gene product. The gene product itself may contain deletions, additions or substitutions of amino acid residues within a chimeric sequence, which result in a silent change thus producing a functionally equivalent chimeric protein. Such amino acid substitutions may be made on the basis of similarity in polarity, charge, solubility, hydrophobicity, hydrophilicity, and/or the amphipathic nature of the residues involved. For example, negatively charged amino acids include aspartic acid and glutamic acid; positively charged amino acids include lysine, histidine and arginine; amino acids with uncharged polar head groups having similar hydrophilicity values include the following: glycine, asparagine, glutamine, serine, threonine, tyrosine; and amino acids with nonpolar head groups include alanine, valine, isoleucine, leucine, phenylalanine, proline, methionine, tryptophan.

The DNA sequences of the invention may be engineered in order to alter a chimeric coding sequence for a variety of ends, including but not limited to, alterations that modify processing and expression of the gene product. For example, mutations may be introduced using techniques which are well known in the art, e.g., site-directed mutagenesis, to insert new restriction sites, to alter glycosylation patterns, phosphorylation, etc.

In an alternate embodiment of the invention, the coding sequence of the chimeric protein could be synthesized in whole or in part, using chemical methods well known in the art. (See, for example, Caruthers et al., 1980; Crea and Horn, 1980; and Chow and Kempe, 1981). Fo example, active domains of the moieties can be synthesized by solid phase techniques, cleaved from the resin, and purified by preparative high performance liquid chromatography followed by chemical linkage to form a chimeric protein. (e.g., see Creighton, 1983, Proteins Structures And Molecular Principles, W.H. Freeman and Co., N.Y. pp. 50-60). The composition of the synthetic peptides may be confirmed by amino acid analysis or sequencing (e.g., the Edman degradation procedure; see Creighton, 1983, Proteins, Structures and Molecular Principles, W.H. Freeman and Co., N.Y. pp. 34-49). Alternatively, the two moieties of the chimeric protein produced by synthetic or recombinant methods may be conjugated by chemical linkers according to methods well known in the art (Brinkmann and Pastan, 1994).

In order to express a biologically active chimeric protein, the nucleotide sequence coding for a chimeric protein, or a functional equivalent, is inserted into an appropriate expression vector, i.e., a vector which contains the necessary elements for the transcription and translation of the inserted coding sequence. The chimeric gene products as well as host cells or cell lines transfected or transformed with recombinant chimeric expression vectors can be used for a variety of purposes. These include but are not limited to generating antibodies (i.e., monoclonal or polyclonal) that bind to epitopes of the proteins to facilitate their purification.

Methods that are well known to those skilled in the art can be used to construct expression vectors containing the chimeric protein coding sequence and appropriate transcriptional/translational control signals. These methods include in vitro recombinant DNA techniques, synthetic techniques and in vivo recombination/genetic recombination. See, for example, the techniques described in Sambrook et al., 1989, Molecular Cloning A Laboratory Manual, Cold Spring Harbor Laboratory, N.Y. and Ausubel et al., 1989, Current Protocols in Molecular Biology, Greene Publishing Associates and Wiley Interscience, N.Y.

A variety of host-expression vector systems may be utilized to express the chimeric protein coding sequence. These include but are not limited to microorganisms such as bacteria transformed with recombinant bacteriophage DNA, plasmid DNA or cosmid DNA expression vectors containing the chimeric protein coding sequence; yeast transformed with recombinant yeast expression vectors containing the chimeric protein coding sequence; insect cell systems infected with recombinant virus expression vectors (e.g., baculovirus) containing the chimeric protein coding sequence; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing the chimeric protein coding sequence; or animal cell systems. It should be noted that since most apoptosis-inducing proteins cause programmed cell death in mammalian cells, it is preferred that the chimeric protein of the invention be expressed in prokaryotic or lower eukaryotic cells.

The expression elements of each system vary in their strength and specificities. Depending on the host/vector system utilized, any of a number of suitable transcription and translation elements, including constitutive and inducible promoters, may be used in the expression vector. For example, when cloning in bacterial systems, inducible promoters such as pL of bacteriophage λ, plac, ptrp, ptac (ptrp-lac hybrid promoter; cytomegalovirus promoter) and the like may be used; when cloning in insect cell systems, promoters such as the baculovirus polyhedrin promoter may be used; when cloning in plant cell systems, promoters derived from the genome of plant cells (e.g., heat shock promoters; the promoter for the small subunit of RUBISCO; the promoter for the chlorophyll α/β binding protein) or from plant viruses (e.g., the 35S RNA promoter of CaMV; the coat protein promoter of TMV) may be used; when cloning in mammalian cell systems, promoters derived from the genome of mammalian cells (e.g., metallothionein promoter) or from mammalian viruses (e.g., the adenovirus late promoter; the vaccinia virus 7.5K promoter) may be used; when generating cell lines that contain multiple copies of the chimeric DNA, SV40-, BPV- and EBV-based vectors may be used with an appropriate selectable marker.

In bacterial systems a number of expression vectors may be advantageously selected depending upon the use intended for the chimeric protein expressed. For example, when large quantities of chimeric protein are to be produced, vectors that direct the expression of high levels of protein products that are readily purified may be desirable. Such vectors include but are not limited to the pHL906 vector (Fishman et al., 1994); the E. coli expression vector pUR278 (Ruther et al., 1983), in which the chimeric protein coding sequence may be ligated into the vector in frame with the lacZ coding region so that a hybrid AS-lacZ protein is produced; pIN vectors (Inouye and Inouye, 1989; Van Heeke and Schuster, 1989); and the like.

An alternative expression system that could be used to express chimeric protein is an insect system. In one such system, Autographa californica nuclear polyhidrosis virus (AcNPV) is used as a vector to express foreign genes. The virus grows in Spodoptera frugiperda cells. The chimeric protein coding sequence may be cloned into non-essential regions (for example the polyhedrin gene) of the virus and placed under control of an AcNPV promoter (for example the polyhedrin promoter). Successful insertion of the chimeric protein coding sequence will result in inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted gene is expressed. (e.g., see Smith et al., 1983; U.S. Pat. No. 4,215,051).

Specific initiation signals may also be required for efficient translation of the inserted chimeric protein coding sequence. These signals include the ATG initiation codon and adjacent sequences. In cases where the entire chimeric gene, including its own initiation codon and adjacent sequences, is inserted into the appropriate expression vector, no additional translational control signals may be needed. However, in cases where the chimeric protein coding sequence does not include its own initiation codon, exogenous translational control signals, including the ATG initiation codon, must be provided. Furthermore, the initiation codon must be in phase with the reading frame of the chimeric protein coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., 1987).

In addition, a host cell strain may be chosen that modulates the expression of the inserted sequences, or modifies and processes the gene product in the specific fashion desired. Such modifications (e.g., glycosylation) and processing (e.g., cleavage) of protein products may be important for the function of the protein. The presence of consensus N-glycosylation sites in a chimeric protein may require proper modification for optimal chimeric protein function. Different host cells have characteristic and specific mechanisms for the post-translational processing and modification of proteins. Appropriate cell lines or host systems can be chosen to ensure the correct modification and processing of the chimeric protein. To this end, eukaryotic host cells that possess the cellular machinery for proper processing of the primary transcript, glycosylation, and phosphorylation of the chimeric protein may be used. Such mammalian host cells include but are not limited to CHO, VERO, BHK, HeLa, COS, MDCK, 293, W138, and the like.

For long-term, high-yield production of recombinant chimeric proteins, stable expression is preferred. For example, cell lines that stably express the chimeric protein may be engineered. Rather than using expression vectors that contain viral originals of replication, host cells can be transformed with a chimeric coding sequence controlled by appropriate expression control elements (e.g., promoter, enhancer, sequences, transcription terminators, polyadenylation sites, etc.), and a selectable marker. Following the introduction of foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci that in turn can be cloned and expanded into cell lines.

A number of selection systems may be used, including but not limited to the herpes simplex virus thymidine kinase (Wigler et al., 1977), hypoxanthine-guanine phosphoribosyltransferase (Szybalski and Szybalski, 1962), and adenine phosphoribosyltransferase (Lowy et al., 1980) genes can be employed in tk-, hgprt- or aprt-cells, respectively. Also, antimetabolite resistance can be used as the basis of selection for dhfr, which confers resistance to methotrexate (Wigler et al., 1980; O'Hare et al., 1981); gpt, which confers resistance to mycophenolic acid (Mulligan and Berg, 1981); neo, which confers resistance to the aminoglycoside G-418 (Colbere-Garapin et al., 1981); and hygro, which confers resistance to hygromycin (Santerre et al., 1984) genes. Additional selectable genes have been described, namely trpB, which allows cells to utilize indole in place of tryptophan; hisD, which allows cells to utilize histinol in place of histidine (Hartman and Mulligan, 1988); and ODC (ornithine decarboxylase) which confers resistance to the ornithine decarboxylase inhibitor, 2-(difluoromethyl)-DL-ornithine, DFMO (McConlogue L, 1987, In: Current Communications in Molecular Biology, Cold Spring Harbor Laboratory ed.).

VII. Muteins

In particular embodiments, an altered molecule, such as an altered TNF molecule, including an altered TNF-α molecule, is employed in the chimeric molecules of the present invention. Other molecules may be employed as muteins, and TNF is described herein merely as an exemplary embodiment. The altered TNF molecule may be further defined as a mutant of TNF, which may be even further defined as a TNF mutein. A TNF mutein comprises a TNF molecule having one or more mutations, wherein the TNF molecule retains TNF function, which in specific embodiments refers to being cytotoxic to a cancer cell. In specific embodiments, the TNF mutein comprises substantially the same or greater activity than wild-type TNF, such as concerning anti-cancer activity and low toxicity. The alteration may affect the binding affinity of TNF to p75-TNF-receptor and/or to p55-TNF-receptor.

In embodiments of the invention, the mutein is altered by substitution of one or more amino acids and in specific embodiments is by naturally occuring amino acids.

The one or more mutations may be in the N-terminus and/or the C-terminus, for example. The mutation may be a point mutation, a frame shift mutation, a deletion, an inversion, or a splicing mutant, for example. The mutation may be in a particular region of TNF, such as a functional domain of TNF. In specific embodiments, the mutation is in the trimerization domain. An exemplary TNF molecule for alteration to a TNF mutein is provided in SEQ ID NO:4 (GenBank Accession No. AAA61200). TNF-muteins may be designed based on the 3-D structure of the protein and molecular modelling approaches.

Specific examples of TNF muteins include those identified, for example, in U.S. Pat. No. 5,773,582; U.S. Pat. No. 5,422,104; U.S. Pat. No. 5,247,070; U.S. Pat. No. 5,606,023; U.S. Pat. No. 5,652,353; U.S. Pat. No. 4,677,064; U.S. Pat. No. 5,519,119; and U.S. Pat. No. 5,652,353, all of which are incorporated by reference herein in their entirety.

VIII. Polypeptide Purification

The chimeric proteins of the invention can be purified by art-suitable techniques such as high performance liquid chromatography, ion exchange chromatography, gel electrophoresis, affinity chromatography, and the like. The actual conditions used to purify a particular protein will depend, in part, on factors such as net charge, hydrophobicity, hydrophilicity, etc., and will be apparent to those having skill in the art.

For affinity chromatography purification, any antibody that specifically binds the protein may be used. For the production of antibodies, various host animals, including but not limited to rabbits, mice, rats, etc., may be immunized by injection with a chimeric protein or a fragment thereof. The protein may be attached to a suitable carrier, such as bovine serum albumin (BSA), by means of a side chain functional group or linkers attached to a side chain functional group. Various adjuvants may be used to increase the immunological response, depending on the host species, including but not limited to, Freund's (complete and incomplete), mineral gels such as aluminum hydroxide, surface active substances such as lysolecithin, pluronic polyols, polyanions, peptides, oil emulsions, keyhold limpet hemocyanin, dinitrophenol, and potentially useful human adjuvants such as BCG (bacilli Calmetter-Guerin) and Corynebacterium parvum.

Monoclonal antibodies to a chimeric protein may be prepared using any technique which provides for the production of antibody molecules by continuous cell lines in culture. These include but are not limited to the hybridoma technique originally described by Koehler and Milstein (1975), the human B-cell hybridoma technique, (Kosbor et al., 1983; Cote et al., 1983) and the EBV-hybridoma technique (Cole et al., 1985). In addition, techniques developed for the production of “chimeric antibodies” (Morrison et al., 1984; Neuberger et al., 1984; Takeda et al., 1985) by splicing the genes from a mouse antibody molecule of appropriate antigen specificity together with genes from a human antibody molecule of appropriate biological activity can be used. Alternatively, techniques described for the production of single chain antibodies (U.S. Pat. No. 4,946,778) can be adapted to produce chimeric protein-specific single chain antibodies for chimeric protein purification and detection.

IX. Polypeptide Production

In particular aspects of the invention, a chimeric molecule in the form of a polypeptide is produced, and in specific aspects the polypeptide is a protein. The polypeptide may be considered to be manufactured, such as by the exemplary procedure described below. A scaled-up protocol for chimeric polypeptide production pursuant to the invention will facilitate its production and use, such as for cancer therapy.

In particular aspects, the following reagents may be employed: equilibration (EQ) Buffer (20 mM Tris, pH 8.0, 200 mM NaCl); and Elution Buffer (20 mM Tris, pH 8.0, 500 mM NaCl, 300 mM Imidazole). Unless noted, all buffers may be stored at 4-8° C. for protein stability. Unless noted, all manipulations may be performed at 4-8° C. for protein stability.

A. Small Scale Analysis of Fermentation Batch

The following protocol may be employed for small scale analysis of fermentation batch:

Use 10 ml of cells @ OD₆₀₀=10 for analysis.

Spin down cells and discard supernatant.

Resuspend cells in 4 ml of EQ Buffer.

Lyse by sonication, 3×20 sec, 2 minute intervals.

Spin down in microfuge, 15 min @ 14,000 rpm.

Carefully remove 3.2 ml supernatant and transfer to fresh tube.

Check pH to ensure it is approximately 8.0

Add 100 μl of metal affinity resin chelated with Cobalt Chloride

Incubate for 15 min at room temperature; rotate to keep resin as suspension.

Transfer suspension to 10 ml column (Bio-Rad, # 731-1550) and collect flow through.

Add 650 μl of Elution Buffer and collect eluate in fresh eppendorf tube.

Run 20 μl aliquot on a 10% SDS-PAGE gel

Estimate yield and extrapolate to obtain expected batch yield.

B. Large Scale Purification of Chimeric Molecule

The following protocol concerning large scale purification of a chimeric molecule may be employed.

1. Bacterial Lysis and Recovery

1. Thaw the bacterial cell culture suspension (approx. 100 g wet weight bacterial paste). Adjust to final volume of 1 L in EQ Buffer.

2. Aliquot cell culture suspension into 5×250 ml Centrifuge tubes, 200 ml per tube.

3. Sonicate the cell suspension in each centrifuge tube 5×1 min per sonication, with a pause of 10 min on ice between each burst.

4. Centrifuge the cell extract at 40,000 rpm for 1 h at 4° C.

5. Carefully transfer the supernatant to a 1 L measuring cylinder or beaker. Note: supernatant can be stored overnight at 4° C. For longer storage, store at −20° C. and, after thawing, centrifuge supernatant as above prior to next step.

6. Using a 0.45 μm filter, remove particulate matter from the lysate prior to loading onto the column.

2. Purification Process of Bacterial Lysates

I) Immobilized Metal Affinity Chromatography (IMAC) Column Preparation for Purification of Chimeric Polypeptide

(Column dimensions: width, 2.5 cm; height 20 cm (Bio-Rad, # 737-2521); Column bed height: 8 cm; Column bed volume: 40 ml.)

7. Prepare a 40 ml bed volume Chelating Sepharose Fast Flow Resin (Amersham Pharmacia Biotech) column. Adjust column flow rate to 4 ml/minute and equilibrate with distilled water.

8. Wash the column with at least 2 column volumes (CV) of distilled water

9. Add approximately 0.5 CV of Cobalt Chloride solution (0.2 M in distilled water).

10. Wash the column with at least 5 CV of distilled water to remove excess of metal ions.

11. Continue washing the column with at least 5 CV of Sodium Acetate solution (0.02 M sodium acetate, 0.5 M NaCl, pH 3.0), or until the pH of the effluent is 3.0 (usually around 1 CV). This will elute loosely bound ions that might otherwise leak out during adsorption/desorption phase of the actual chromatographic step.

12. Equilibrate the column with 5 CV of EQ Buffer.

13. Run the lysate through the column and collect the flow through.

14. Wash the column with 2 CV of EQ Buffer.

15. Wash the column with 3 CV of 20 mM Tris, pH 8.0, 0.5 M NaCl.

16. Wash the column with 3 CV of 20 mM Tris, pH 8.0, 200 mM NaCl, 20 mM Imidazole. Collect 15 ml fractions.

17. Elute the protein with 5 CV of 20 mM Tris, pH 8.0, 0.5 M NaCl, 300 mM Imidazole. Collect 15 ml fractions.

18. Run the fractions on a 10% SDS-PAGE gel (20 μl of each fraction). Protein molecular weight is 62 kDa (monomer).

II) Digestion of the Polyhistidine Tag

19. Pool fractions containing the chimeric polypeptide and buffer exchange by overnight dialysis using “rEK Buffer” (20 mM Tris, pH 8, 150 mM NaCl).

20. Spin down overnight dialysate to remove any precipitate. Analyze sample on SDS-PAGE gel and estimate total chimeric polypeptide.

21. Remove the thioredoxin/polyhistidine tag by addition 4 Units recombinant Enterokinase (rEK, Novagen) per mg chimeric polypeptide and allow digestion to proceed overnight at room temperature.

22. Remove a 20 μl aliquot for electrophoretic analysis of digestion efficiency.

23. Buffer exchange the digested chimeric polypeptide by overnight dialysis against 0.5×PBS.

III) Protein Purification Using Ion Exchange Chromatography with Blue Sepharose

(Column dimensions: width, 1.5 cm; height 15 cm (Bio-Rad, #737-1516); Column bed height: 6 cm; Column bed volume: 10 ml.)

24. Prepare a 10 ml bed volume Blue Sepharose 6 Fast Flow Resin (Amersham Pharmacia Biotech) column. Adjust column flow rate to 5 ml/minute.

25. Wash the column with 10 CV of 10 mM Na₂HPO₄, pH 7.2 (“Phosphate buffer”).

26. Wash the column with 3 CV of PBS/2M NaCl.

27. Wash the column with 10 CV Phosphate buffer.

28. Spin down the overnight dialyzed protein sample to remove any precipitation. Dilute the protein sample 1:1 with phosphate buffer so that the [NaCl] is 35 mM.

29. Run the protein sample through the Blue Sepharose column and collect the flow through. Reload the flow through on the column.

30. Wash the column with 5 CV Phosphate Buffer.

31. Wash the column with 6 CV PBS/150 mM NaCl. Collect 6 ml fractions.

32. Elute the VEGF121/rGel with 9 CV PBS/2M NaCl. Collect 6 ml fractions.

33. Remove 10 μl aliquots for electrophoretic analysis. VEGF121/rGel molecular weight is 43 kDa (monomer).

34. Pool fractions containing VEGF121/rGel and buffer exchange by overnight dialysis against PBS.

IV) Endotoxin Removal

(Column dimensions: width, 1.5 cm; height 15 cm; Column bed height: 3 cm; Column bed volume: 5 ml)

35. Prepare a 5 ml bed volume of Detoxi-Gel Endotoxin Removing Gel (Pierce). Column flow is by gravity. Follow manufacturer's instructions for preparation and use of the Endo-Gel (Steps 30-32)

36. Wash the column with 5 CV 1% sodium deoxycholate.

37. Wash the column with 3-5 CV of pyrogen-free water

38. Wash the column with 5 CV PBS

39. Run the chimeric polypeptide through the column and collect the flow-through. Wash the column with 3 ml PBS; combine this eluate to the flow-through (which contains the chimeric polypeptide).

40. Determine endotoxin levels using the Limulus Amebocyte Lysate assay (Cambrex)

41. Repeat Steps 30-33 to regenerate the column and remove any bound endotoxin until endotoxin levels are below 0.65 EU/mg.

V) Buffer Exchange and Concentration

42. Buffer exchange into sterile DPBS (pH 7.4)

43. Concentrate the chimeric molecule to 1 mg/ml.

44. Assess other release specifications

45. Filter sterilize using 0.22 μm filters and store the protein at −20° C. (prefer flexi-bag).

Following and/or during this exemplary procedure, one may monitor the purity and yield, such as % purity and/or % yield. Different fractions may be subjected to electrophoresis to assist the monitoring.

C. Fed-Batch Fermentation of Chimeric Polypeptide in Exemplary E. coli

In an alternative embodiment, one may employ the following procedure of fed-batch fermentation of vector and E. coli to produce recombinant fusion protein. In specific embodiments, a pET vector and E. coli AD494 (DE3) pLysS-host systems are employed, for example. AD494 strains are K-12 derived thioredoxin reductase (trxB) mutants that enable disulfide bond formation in the cytoplasm. The trxB mutation is selectable on kanamycin; therefore, this strain is recommended for use with plasmids carrying the ampicillin resistance marker bla. The pLysS version comprises a chloramphenical-resistant plasmid that encodes T7 lysozyme, which provides better control of basal expression levels.

1. Vector and Host System

In an exemplary embodiment, a pET vector encoding the chimeric polypeptide is comprised in E. coli AD494 (DE3) pLysS, for example.

2. Seed Cultivation

1. Prepare the seed medium in 250 mL of the autoclaved Erlenmeyer flask as follows: 50 mL of LB medium, ampicillin 200 mg/L, kanamycin 30 mg/L, and chloramphenicol 30 mg/L.

2. Inoculate a vial of the glycerol cell stock (1 mL) frozen at −80° C. into LB medium.

3. The conditions of the seed cultivation: 37° C. and 240 rpm at a shaking incubator.

4. After 5±0.5 h, inoculate 50 mL of the cell broth (OD₆₀₀ₙₘ=1.0±0.5) into the main fermentor (working volume=1.5 L).
For the large-scale fementation, this seed cultivation can be modified in two steps. For consistent inoculations, the final seed cultivation needs to be prepared in the same values for the following parameters: the final OD and the inoculumn size (3.0%, seed volume/working volume of main cultivation×100).

3. Main Fermentation in a 2-L Vessel

1. Calibrate the pH electrode connected to the fermentor controller with the standard solutions of pH 4 and pH 7.

2. Prepare the following the initial batch medium (iGYG): 24.0 g/L glycerol; 20.0 g/L yeast extract (Difco, USA); 7.0 g/L K₂HPO₄; 3.0 g/L KH₂PO₄; 0.5 g/L MgSO₄.7H₂O; 0.01 g/L Biotin (Sigma, USA); 0.5 g/L of glycine (6.6 mM). Glycerol and glycine are sterilized separately and added into the main fermentor containing the sterilized medium after cooling down the set temperature, 37° C. For 1-L of the feeding medium (fdGYG), 600.0 g of glycerol, 60.0 g of yeast extract, and 3.0 of glycine. Three components need to be autoclaved separately.

3. Calibrate the DO (dissolved oxygen) electrode at 37° C. and 600 rpm. For zeroing (DO=0%), purge the nitrogen gas into the fermentor at 1 vvm (volume of gas/volume of liquid/minute). For spanning (DO=100%), use the air gas.

4. Add the antibiotics: in the final concentrations, ampicillin 200 mg/L, kanamycin 30 mg/L, and chloramphenicol 30 mg/L.

5. Inoculate the seed into the fermentor and take a sample for analyzing cell density (OD₆₀₀ₙₘ).

6. The pH of the medium is controlled at 7.0 using the acid (42.5% of H₃PO₄) and base (50% of NH₄OH) solutions.

7. At OD=19.0 0±2.0 (or 12±1 h), initiate the fed-batch fermentation with the feeding solution in p (specific cell growth rate, h⁻¹)-controlled feeding strategy. The volumetric feed rate is determined by the following formula:

$$F = C \cdot ( \frac{μ \cdot X \cdot V}{Y \cdot {S}_{f}} ) \cdot exp ( μ \cdot t )$$

where F: volumetric feed rate of medium (L/h); C: correction factor (>0); μ: specific cell growth rate to be controlled (/h); X: cell concentration in culture broth (OD); V: working volume in fermentor (L); Y: cell yield on glycerol (OD/g/L); S_{f}: glycerol concentration in feed solution (g/L); t: time interval in feeding period (h); Fₛ=a·F+b where Fₛ: set value of pump for feeding; F: volumetric feed rate of medium (L/h); a (slope) and b (constant) are pre-determined by the calibration curve determined in F v. Fₛ.

8. At OD≧35.0±5.0, change the culture temperature from 37 to 23° C. and add the inducer (IPTG, 0.1 mM for OD10) for expression of the target protein. Change the set value of the pump (Fs) to reduce the feed rate down to 25 mL/h for 1.8 L of culture volume (15 g-glycerol/h or 8.3 g-glycerol/L/h).

9. Increase the agitation speed (RPM) up to 990 (or control the RPM to maintain the DO level over 20% through the cultivation).

10. After 10±1 h, the cell is harvested and centrifuged to separate the cell pellet.

11. Resuspend the cell pellet in Tris-buffer (20 mM Tris-HCl, pH 8) for storage in the freezer (−20° C.) or purification.

4. Expression Test in SDS-PAGE (6.5%) Stained with Coomassie Blue

1. Resuspend cell pellet (OD10, 10 mL) with 4 mL of 20 mM Tris buffer, pH 8.

2. Sonicate the cell suspension (20 sec, 2 or 3 times) in the ice bath.

3. Ultra centrifuge the disrupted cell at 40K, 15 min, and 4oC using Ti50.4 rotor.

4. Take 3.2 mL of supernatant (cell lysate) and add 0.2 mL of TALON resin (50% suspension in 20 mM Tris buffer, pH8) and 0.1 mL of 4M NaCl (the final concentration=200 mM).

5. Binding for 30 min at RT.

6. Transfer the sample into the column and collect the flow through (‘FT’).

7. Elute the target protein (V3825) with 1 mL of the elution buffer (20 mM Tris, 200 mM NaCl, and 500 mM Imidazole, pH 8) (‘E’).

8. Resuspend the TALON resin remained inside of the column with 0.2 mL of 20 mM Tris buffer, pH 8, and transfer it to the micro-centrifuge tube (‘TAL’).

9. Run the SDS-PAGE (6.5%) with standards (3-0.5 ug per each lane) and analyze the amounts of the target protein by the densitometer.

10. Calculations:

$${[ V3825 ]}_{sp} = X ( μ g ) \times ( \frac{1000 ( μ L )}{{V}_{1} ( μ L )} ) ( \frac{{V}_{2} ( m L )}{{V}_{3} ( m L )} ) {( \frac{1}{{V}_{4} ( m L )} ) \\ [ V 3825 ]}_{t} = {[ V 3825 ]}_{sp} \times ( \frac{OD}{10} )$$

Here,

[V3825]ₛₚ: Specific productivity of V3825 [mg/L/OD10]

X: Amount of target protein per each lane estimated by densitometry analysis [μg]

V₁: Loading volume of sample in each well of SDS-PAGE [μL]

V₂: Volume of 20 mM Tris buffer, pH 8 added to resuspend the cell pellet [mL]

V₃: Volume of supernatant (cell lysate) after cell lysis (sonication) and ultra-centrifugation [mL]

V₄: Original sampling volume=10 [mL]

[V3825]_{f}: Total productivity of V3825 [mg/L]

OD: Optical density (600 nm) of the original sample collected from the fermentor

The exemplary protocols described in this section, for example, may be employed for any chimeric molecule of the invention, and a skilled artisan is aware of aspects of the protocol to optimize pursuant to a specific chimeric molecule, for example.

X. Aptamers

In certain embodiments of the invention, compositions and methods utilize chimer molecules comprised with aptamers. The term “aptamer” as used herein refers to one or more small molecules that can bind to another molecule. In specific embodiments, aptamers can comprise nucleic acid, including RNA or DNA that may comprise oligonucleotides, and/or they can comprise peptides. In certain embodiments, aptamers comprise oligonucleotides, such as those that are chemically synthesized strands of oligonucleotides that can assume highly specific three-dimensional conformations. Aptamers may be designed to have appropriate binding affinities and specificities towards certain target molecules, including the same molecules to which the chimeric molelcules are targeted, for example. In alternative embodiments, aptamers are employed as the cell-targeting moiety for one or more anti-cell proliferation moieties.

An aptamer molecule may be conjugated to a desired molecule of the invention by any suitable methods in the art, although in particular aspects of the invention the desired molecule is conjugated to the aptamer by the heterobifunctional cross-linking agent n-succinimidyl-3-(2-pyridyldithio)propionate (SPDP).

An aptamer may be obtained, such as from knowledge in the art concerning already-known aptamers, or an aptamer may be screened for, such as by routine methods in the art, for example. Thus, in specific embodiments of the invention, an aptamer is linked to an anti-cell proliferation moiety. In other embodiments of the invention an aptamer is linked to a chimeric molecule of an anti-cell proliferation moiety linked to a cell targeting moiety, and in this case the aptamer/chimeric molecule employs two cell-targeting entities.

The following description regards an exemplary aptamer composition employing a gelonin conjugate comprising an aptamer that targets prostate-specific membrane antigen (PMSA), although one of skill in the art recognizes that alternative embodiments may be employed with part or all of any of the chimeric molecules of the invention and/or with alternative aptamers. PSMA is a transmembrane receptor whose expression increases on prostatic tumor cells during disease progression. This protein is also expressed on the endothelium of tumor vasculature but not normal vasculature. Aptamers are small nucleic acids selected to bind proteins such as cell surface tumor antigens with high affinity and specificity. Aptamers have the potential to serve as replacements for cell-targeting antibodies or other cell-targeting ligands.

The present inventors utilized a modified RNA aptamer known to bind the external domain of PSMA with high affinity (previously reported Ki=2 nM). The 22 kDa aptamer molecule was conjugated to recombinant gelonin (rGel) toxin using the heterobifunctional cross-linking agent SPDP. The aptamer/rGel conjugate was then purified by ion exchange and gel permeation chromatography. The final product was uncontaminated by free aptamer or free rGel and migrated as a single species (˜50 kDa) by SDS-PAGE. Analysis of the construct demonstrated that the rGel component was enzymatically active compared to free rGel. In addition, the conjugate was found to bind specifically to PSMA-expressing LNCAP cells. Cytotoxicity studies of the Aptamer/rGel conjugate demonstrated an I.C.₅₀ of 32 nM on antigen-positive LNCAP cells compared to an I.C.₅₀ of 350,000 nM on PC3 cells, which contain much less antigen; a targeting index of approximately 10,000 fold. Internalization studies should reveal the details of toxin conjugate entry, and animal model studies are ongoing. These data indicate that aptamers can be used to successfully deliver protein molecules such as toxins to tumor cells and provide a novel approach to development of targeted therapeutic agents. The fact that aptamers can be chemically synthesized and thereby site-specifically conjugated makes them especially interesting as targeting ligands. The exemplary PSMA-rGel construct is useful to target PSMA on prostate cells and on tumor vasculature.

XI. Administration of the Chimeric Molecules

In some embodiments, an effective amount of the chimeric molecules of the present invention is administered to a cell. In other embodiments, a therapeutically effective amount of the chimeric molecules of the present invention are administered to an individual for the treatment of disease. The term “effective amount” as used herein is defined as the amount of the chimeric molecules of the present invention that is necessary to result in a physiological change in the cell or tissue to which it is administered. The term “therapeutically effective amount” as used herein is defined as the amount of the chimeric molecules of the present invention that eliminates, decreases, delays, or minimizes adverse effects of a disease, such as cancer. A skilled artisan readily recognizes that in many cases the chimeric molecules may not provide a cure but may only provide partial benefit, such as alleviation or improvement of at least one symptom. In some embodiments, a physiological change having some benefit is also considered therapeutically beneficial. Thus, in some embodiments, an amount of chimeric molecules that provides a physiological change is considered an “effective amount” or a “therapeutically effective amount.”

In some embodiments of the present invention and as an advantage over known methods in the art, the chimeric molecules are delivered as proteins and not as nucleic acid molecules to be translated to produce the desired polypeptides. As an additional advantage, in some embodiments human sequences are utilized in the chimeric polypeptides of the present invention to circumvent any undesirable immune responses from a foreign polypeptide.

The chimeric proteins of the invention may be administered to a subject per se or in the form of a pharmaceutical composition for the treatment of cancer, autoimmunity, transplantation rejection, post-traumatic immune responses and infectious diseases, for example by targeting viral antigens, such as gp120 of HIV. More specifically, the chimeric polypeptides may be useful in eliminating cells involved in immune cell-mediated disorder, including lymphoma; autoimmunity, transplantation rejection, graft-versus-host disease, ischemia and stroke. Pharmaceutical compositions comprising the proteins of the invention may be manufactured by means of conventional mixing, dissolving, granulating, dragee-making, levigating, emulsifying, encapsulating, entrapping or lyophilizing processes. Pharmaceutical compositions may be formulated in conventional manner using one or more physiologically acceptable carriers, diluents, excipients or auxiliaries that facilitate processing of the proteins into preparations which can be used pharmaceutically. Proper formulation is dependent upon the route of administration chosen.

For topical administration the proteins of the invention may be formulated as solutions, gels, ointments, creams, suspensions, etc. as are well-known in the art.

Systemic formulations include those designed for administration by injection, e.g. subcutaneous, intravenous, intramuscular, intrathecal or intraperitoneal injection, as well as those designed for transdermal, transmucosal, inhalation, oral or pulmonary administration.

For injection, the proteins of the invention may be formulated in aqueous solutions, preferably in physiologically compatible buffers such as Hanks' solution, Ringer's solution, or physiological saline buffer. The solution may contain formulatory agents such as suspending, stabilizing and/or dispersing agents.

Alternatively, the proteins may be in powder form for constitution with a suitable vehicle, e.g., sterile pyrogen-free water, before use.

For transmucosal administration, penetrants appropriate to the barrier to be permeated are used in the formulation. Such penetrants are generally known in the art.

For oral administration, the proteins can be readily formulated by combining the proteins with pharmaceutically acceptable carriers well known in the art. Such carriers enable the proteins of the invention to be formulated as tablets, pills, dragees, capsules, liquids, gels, syrups, slurries, suspensions and the like, for oral ingestion by a patient to be treated. For oral solid formulations such as, for example, powders, capsules and tablets, suitable excipients include fillers such as sugars, e.g. lactose, sucrose, mannitol and sorbitol; cellulose preparations such as maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP); granulating agents; and binding agents. If desired, disintegrating agents may be added, such as the cross-linked polyvinylpyrrolidone, agar, or alginic acid or a salt thereof such as sodium alginate.

If desired, solid dosage forms may be sugar-coated or enteric-coated using standard techniques.

For oral liquid preparations such as, for example, suspensions, elixirs and solutions, suitable carriers, excipients or diluents include water, glycols, oils, alcohols, etc. Additionally, flavoring agents, preservatives, coloring agents and the like may be added.

For buccal administration, the molecules may take the form of tablets, lozenges, etc. formulated in conventional manner.

For administration by inhalation, the molecules for use according to the present invention are conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of gelatin for use in an inhaler or insufflator may be formulated containing a powder mix of the protein and a suitable powder base such as lactose or starch.

The chimeric molecules may also be formulated in rectal or vaginal compositions such as suppositories or retention enemas, e.g., containing conventional suppository bases such as cocoa butter or other glycerides.

In addition to the formulations described previously, the molecules may also be formulated as a depot preparation. Such long acting formulations may be administered by implantation (for example subcutaneously or intramuscularly) or by intramuscular injection. Thus, for example, the molecules may be formulated with suitable polymeric or hydrophobic materials (for example as an emulsion in an acceptable oil) or ion exchange resins, or as sparingly soluble derivatives, for example, as a sparingly soluble salt.

Alternatively, other pharmaceutical delivery systems may be employed. Liposomes and emulsions are well-known examples of delivery vehicles that may be used to deliver proteins of the invention. Certain organic solvents such as dimethylsulfoxide also may be employed, although usually at the cost of greater toxicity. Additionally, the molecules may be delivered using a sustained-release system, such as semipermeable matrices of solid polymers containing the therapeutic agent. Various of sustained-release materials have been established and are well known by those skilled in the art. Sustained-release capsules may, depending on their chemical nature, release the molecules for a few weeks up to over 100 days. Depending on the chemical nature and the biological stability of the chimeric molecules, additional strategies for molecule stabilization may be employed.

As the protein embodiments of the chimeric molecules of the invention may contain charged side chains or termini, they may be included in any of the above-described formulations as the free acids or bases or as pharmaceutically acceptable salts. Pharmaceutically acceptable salts are those salts that substantially retain the biologic activity of the free bases and which are prepared by reaction with inorganic acids. Pharmaceutical salts tend to be more soluble in aqueous and other protic solvents than are the corresponding free base forms.

A. Effective Dosages

The chimeric molecules of the invention will generally be used in an amount effective to achieve the intended purpose. For use to treat or prevent a disease condition, the molecules of the invention, or pharmaceutical compositions thereof, are administered or applied in a therapeutically effective amount. A therapeutically effective amount is an amount effective to ameliorate or prevent the symptoms, or prolong the survival of, the patient being treated. Determination of a therapeutically effective amount is well within the capabilities of those skilled in the art, especially in light of the detailed disclosure provided herein.

For systemic administration, a therapeutically effective dose can be estimated initially from in vitro assays. For example, a dose can be formulated in animal models to achieve a circulating concentration range that includes the IC₅ as determined in cell culture. Such information can be used to more accurately determine useful doses in humans.

Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the molecules which are sufficient to maintain therapeutic effect. Usual patient dosages for administration by injection range from about 0.1 to 5 mg/kg/day, preferably from about 0.5 to 1 mg/kg/day. Therapeutically effective serum levels may be achieved by administering multiple doses each day.

In cases of local administration or selective uptake, the effective local concentration of the proteins may not be related to plasma concentration. One having skill in the art will be able to optimize therapeutically effective local dosages without undue experimentation.

The amount of molecules administered will, of course, be dependent on the subject being treated, on the subject's weight, the severity of the affliction, the manner of administration and the judgment of the prescribing physician.

The therapy may be repeated intermittently while symptoms detectable or even when they are not detectable. The therapy may be provided alone or in combination with other drugs. In the case of autoimmune disorders, the drugs that may be used in combination with IL2-Bax of the invention include, but are not limited to, steroid and non-steroid anti-inflammatory agents.

B. Toxicity

Preferably, a therapeutically effective dose of the chimeric molecules described herein will provide therapeutic benefit without causing substantial toxicity.

Toxicity of the molecules described herein can be determined by standard pharmaceutical procedures in cell cultures or experimental animals, e.g., by determining the LD50 (the dose lethal to 50% of the population) or the LD₁₀₀ (the dose lethal to 100% of the population). The dose ratio between toxic and therapeutic effect is the therapeutic index. Proteins which exhibit high therapeutic indices are preferred. The data obtained from these cell culture assays and animal studies can be used in formulating a dosage range that is not toxic for use in human. The dosage of the proteins described herein lies preferably within a range of circulating concentrations that include the effective dose with little or no toxicity. The dosage may vary within this range depending upon the dosage form employed and the route of administration utilized. The exact formulation, route of administration and dosage can be chosen by the individual physician in view of the patient's condition. (See, e.g., Fingl et al., 1975, In: The Pharmacological Basis of Therapeutics, Ch. 1, p. 1).

C. Pharmaceutical Preparations

Pharmaceutical compositions of the present invention comprise an effective amount of one or more chimeric polypeptides or chimeric polypeptides and, in some embodiments, at least one additional agent dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains at least one chimeric polypeptide or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The chimeric molecules may comprise different types of carriers depending on whether it is to be administered in solid, liquid or aerosol form, and whether it needs to be sterile for such routes of administration as injection. The present invention can be administered intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, inhalation (e.g. aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The chimeric molecules may be formulated into a composition in a free base, neutral or salt form. Pharmaceutically acceptable salts, include the acid addition salts, e.g., those formed with the free amino groups of a proteinaceous composition, or that are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

In other embodiments, one may use eye drops, nasal solutions or sprays, aerosols or inhalants in the present invention. Such compositions are generally designed to be compatible with the target tissue type. In a non-limiting example, nasal solutions are usually aqueous solutions designed to be administered to the nasal passages in drops or sprays. Nasal solutions are prepared so that they are similar in many respects to nasal secretions, so that normal ciliary action is maintained. Thus, in preferred embodiments the aqueous nasal solutions usually are isotonic or slightly buffered to maintain a pH of about 5.5 to about 6.5. In addition, antimicrobial preservatives, similar to those used in ophthalmic preparations, drugs, or appropriate drug stabilizers, if required, may be included in the formulation. For example, various commercial nasal preparations are known and include drugs such as antibiotics or antihistamines.

In certain embodiments, the chimeric molecules is prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. Preferred carriers for oral administration comprise inert diluents, assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof.

In certain preferred embodiments an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

Additional formulations which are suitable for other modes of administration include suppositories. Suppositories are solid dosage forms of various weights and shapes, usually medicated, for insertion into the rectum, vagina or urethra. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

In particular embodiments, prolonged absorption of an injectable composition can be brought about by the use in the compositions of agents delaying absorption, such as, for example, aluminum monostearate, gelatin or combinations thereof.

XII. Combination Treatments/Cancer Therapies

In order to increase the effectiveness of a chimeric molecule of the present invention, or expression construct coding therefor, it may be desirable to combine these compositions with other agents effective in the treatment of hyperproliferative disease, such as anti-cancer agents. Indeed, in particular embodiments, the chimeric molecules of the present invention are employed with one or more chemotherapeutic agents, such as to render effective the chemotherapeutic agent on a resistant cell. The chimeric molecules alone or in conjunction with one or more chemotherpeutic agents may be administered to an individual with cancer in addition to another cancer therapy, such as radiation, surgery, gene therapy, and so forth.

An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, these other compositions would be provided in a combined amount effective to kill or inhibit proliferation of the cell. This process may involve contacting the cells with the expression construct and the agent(s) or multiple factor(s) at the same time. This may be achieved by contacting the cell with a single composition or pharmacological formulation that includes both agents, or by contacting the cell with two distinct compositions or formulations, at the same time, wherein one composition includes the expression construct and the other includes the second agent(s).

Tumor cell resistance to chemotherapy and radiotherapy agents represents a major problem in clinical oncology. One goal of current cancer research is to find ways to improve the efficacy of chemo- and radiotherapy by combining it with gene therapy. For example, the herpes simplex-thymidine kinase (HS-tK) gene, when delivered to brain tumors by a retroviral vector system, successfully induced susceptibility to the antiviral agent ganciclovir (Culver, et al., 1992). In the context of the present invention, it is contemplated that chimeric molecules could be used similarly in conjunction with chemotherapeutic, radiotherapeutic, gene therapy, or immunotherapeutic intervention, in addition to other pro-apoptotic or cell cycle regulating agents.

Alternatively, the therapy may precede or follow the other agent treatment by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations.

Various combinations may be employed, wherein chimeric molecule therapy is “A” and the secondary agent, such as radio- or chemotherapy, for example, is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of the therapeutic expression constructs of the present invention to a patient will follow general protocols for the administration of chemotherapeutics, taking into account the toxicity, if any, of the vector. It is expected that the treatment cycles would be repeated as necessary. It also is contemplated that various standard therapies, as well as surgical intervention, may be applied in combination with the described hyperproliferative cell therapy.

A. Chemotherapy

Cancer therapies also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein tansferase inhibitors, transplatinum, 5-fluorouracil, vincristin, vinblastin and methotrexate, or any analog or derivative variant of the foregoing.

B. Radiotherapy

Other factors that cause DNA damage and have been used extensively include what are commonly known as 7-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

C. Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy, thus, could be used as part of a combined therapy, in conjunction with gene therapy. The general approach for combined therapy is discussed below. Generally, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

D. Genes

In yet another embodiment, the secondary treatment is a gene therapy in which a therapeutic polynucleotide is administered before, after, or at the same time as a chimeric polypeptide of the present invention. Delivery of a chimeric polypeptide in conjuction with a second vector encoding one of the following gene products will have a combined anti-hyperproliferative effect on target tissues. Alternatively, a single vector encoding both genes may be used. A variety of proteins are encompassed within the invention, some of which are described below.

1. Inducers of Cellular Proliferation

The proteins that induce cellular proliferation further fall into various categories dependent on function. The commonality of all of these proteins is their ability to regulate cellular proliferation. For example, a form of PDGF, the sis oncogene, is a secreted growth factor. Oncogenes rarely arise from genes encoding growth factors, and at the present, sis is the only known naturally-occurring oncogenic growth factor. In one embodiment of the present invention, it is contemplated that anti-sense mRNA directed to a particular inducer of cellular proliferation is used to prevent expression of the inducer of cellular proliferation.

The proteins FMS, ErbA, ErbB and neu are growth factor receptors. Mutations to these receptors result in loss of regulatable function. For example, a point mutation affecting the transmembrane domain of the Neu receptor protein results in the neu oncogene. The erbA oncogene is derived from the intracellular receptor for thyroid hormone. The modified oncogenic ErbA receptor is believed to compete with the endogenous thyroid hormone receptor, causing uncontrolled growth.

The largest class of oncogenes includes the signal transducing proteins (e.g., Src, Abl and Ras). The protein Src is a cytoplasmic protein-tyrosine kinase, and its transformation from proto-oncogene to oncogene in some cases, results via mutations at tyrosine residue 527. In contrast, transformation of GTPase protein ras from proto-oncogene to oncogene, in one example, results from a valine to glycine mutation at amino acid 12 in the sequence, reducing ras GTPase activity.

The proteins Jun, Fos and Myc are proteins that directly exert their effects on nuclear functions as transcription factors.

2. Inhibitors of Cellular Proliferation

The tumor suppressor oncogenes function to inhibit excessive cellular proliferation. The inactivation of these genes destroys their inhibitory activity, resulting in unregulated proliferation. The tumor suppressors p53, p16 and C-CAM are described below.

High levels of mutant p53 have been found in many cells transformed by chemical carcinogenesis, ultraviolet radiation, and several viruses. The p53 gene is a frequent target of mutational inactivation in a wide variety of human tumors and is already documented to be the most frequently mutated gene in common human cancers. It is mutated in over 50% of human NSCLC (Hollstein et al., 1991) and in a wide spectrum of other tumors.

The p53 gene encodes a 393-amino acid phosphoprotein that can form complexes with host proteins such as large-T antigen and EIB. The protein is found in normal tissues and cells, but at concentrations which are minute by comparison with transformed cells or tumor tissue

Wild-type p53 is recognized as an important growth regulator in many cell types. Missense mutations are common for the p53 gene and are essential for the transforming ability of the oncogene. A single genetic change prompted by point mutations can create carcinogenic p53. Unlike other oncogenes, however, p53 point mutations are known to occur in at least 30 distinct codons, often creating dominant alleles that produce shifts in cell phenotype without a reduction to homozygosity. Additionally, many of these dominant negative alleles appear to be tolerated in the organism and passed on in the germ line. Various mutant alleles appear to range from minimally dysfunctional to strongly penetrant, dominant negative alleles (Weinberg, 1991).

Another inhibitor of cellular proliferation is p16. The major transitions of the eukaryotic cell cycle are triggered by cyclin-dependent kinases, or CDK's. One CDK, cyclin-dependent kinase 4 (CDK4), regulates progression through the G1. The activity of this enzyme may be to phosphorylate Rb at late G1. The activity of CDK4 is controlled by an activating subunit, D-type cyclin, and by an inhibitory subunit, the p161NK4 has been biochemically characterized as a protein that specifically binds to and inhibits CDK4, and thus may regulate Rb phosphorylation (Serrano et al., 1993; Serrano et al., 1995). Since the p161NK4 protein is a CDK4 inhibitor (Serrano, 1993), deletion of this gene may increase the activity of CDK4, resulting in hyperphosphorylation of the Rb protein. p16 also is known to regulate the function of CDK6.

p161NK4 belongs to a newly described class of CDK-inhibitory proteins that also includes p16B, p19, p21WAF1, and p27KIP1. The p161NK4 gene maps to 9p21, a chromosome region frequently deleted in many tumor types. Homozygous deletions and mutations of the p161NK4 gene are frequent in human tumor cell lines. This evidence suggests that the p161NK4 gene is a tumor suppressor gene. This interpretation has been challenged, however, by the observation that the frequency of the p161NK4 gene alterations is much lower in primary uncultured tumors than in cultured cell lines (Caldas et al., 1994; Cheng et al., 1994; Hussussian et al., 1994; Kamb et al., 1994; Kamb et al., 1994; Mori et al., 1994; Okamoto et al., 1994; Nobori et al., 1995; Orlow et al., 1994; Arap et al., 1995). Restoration of wild-type p16NK4 function by transfection with a plasmid expression vector reduced colony formation by some human cancer cell lines (Okamoto, 1994; Arap, 1995).

Other genes that may be employed according to the present invention include Rb, APC, DCC, NF-1, NF-2, WT-1, MEN-I, MEN-II, zacl, p73, VHL, MMAC1/PTEN, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf, erb, fms, trk, ret, gsp, hst, abl, E1A, p300, genes involved in angiogenesis (e.g., VEGF, FGF, thrombospondin, BAI-1, GDAIF, or their receptors) and MCC.

3. Regulators of Programmed Cell Death

Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl 2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl 2 protein now is recognized to be a member of a family of related proteins, which can be categorized as death agonists or death antagonists.

Subsequent to its discovery, it was shown that Bcl 2 acts to suppress cell death triggered by a variety of stimuli. Also, it now is apparent that there is a family of Bcl 2 cell death regulatory proteins which share in common structural and sequence homologies. These different family members have been shown to either possess similar functions to Bcl 2 (e.g., BclXL, BCiW, BclS, Mcl-1, Al, Bfl-1) or counteract Bcl 2 function and promote cell death (e.g., Bax, Bak, Bik, Bim, Bid, Bad, Harakiri).

E. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies. The chimeric molecule of the present invention may be employed as neoadjuvant surgical therapy, such as to reduce tumor size prior to resection, or it may be employed as postadjuvant surgical therapy, such as to sterilize a surgical bed following removal of part or all of a tumor.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

F. Other Agents

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adehesion, or agents that increase the sensitivity of the hyperproliferative cells to apoptotic inducers. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL would potentiate the apoptotic inducing abililties of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyerproliferative efficacy of the treatments. Inhibitors of cell adehesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

XIII. Kits of the Invention

Any one or more of the compositions described herein may be comprised in a kit. In a non-limiting example, a chimeric molecule, the chimeric molecule components and/or one or more additional agents may be comprised in a kit. The kits will thus comprise, in suitable container means, a chimeric molecule, the chimeric molecule components and/or an additional agent of the present invention.

The kits may comprise a suitably aliquoted chimeric molecule, chimeric molecule components and/or additional agent compositions of the present invention, whether labeled or unlabeled, as may be used for treatment of one or more individuals with cancer. The one or more components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the chimeric molecule, the chimeric molecule components and/or additional agent, and any other reagent containers in close confinement for commercial sale. Such containers may include injection or blow molded plastic containers into which the desired vials are retained.

Therapeutic kits of the present invention are kits comprising the chimeric molecule, the chimeric molecule components, or pharmaceutically acceptable salts thereof. Such kits will generally contain, in suitable container means, a pharmaceutically acceptable formulation of a chimeric molecule protein, polypeptide, peptide, domain, inhibitor, and/or a gene and/or vector expressing any of the foregoing in a pharmaceutically acceptable formulation. The kit may have a single container means, and/or it may have distinct container means for each compound.

When the components of the kit are provided in one and/or more liquid solutions, the liquid solution may be an aqueous solution, with a sterile aqueous solution being particularly preferred. The chimeric molecule and the chimeric molecule component compositions may also be formulated into a syringeable composition. In this case, the container means may itself be a syringe, pipette, and/or other such like apparatus, from which the formulation may be applied to an infected area of the body, injected into an animal, and/or even applied to and/or mixed with the other components of the kit.

However, the components of the kit may be provided as dried powder(s). When reagents and/or components are provided as a dry powder, the powder can be reconstituted by the addition of a suitable solvent. The solvent may be aqueous or organic. It is envisioned that the solvent may also be provided in another container means.

The container means will generally include at least one vial, test tube, flask, bottle, syringe and/or other container means, into which the chimeric molecule and/or the chimeric molecule components being a protein, gene and/or inhibitory formulation are placed, preferably, suitably allocated. The kits may also comprise a second container means for containing a sterile, pharmaceutically acceptable buffer and/or other diluent.

Irrespective of the number and/or type of containers, the kits of the invention may also comprise, and/or be packaged with, an instrument for assisting with the injection/administration and/or placement of the ultimate chimeric molecule protein and/or gene composition within the body of an animal. Such an instrument may be a syringe, pipette, forceps, and/or any such medically approved delivery vehicle, for example.

### EXAMPLES

The following examples are included to demonstrate preferred embodiments of the invention. It should be appreciated by those skilled in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1

### Exemplary Material and Methods for Examples 2-8

Cell Lines and Culture

Four human pancreatic cancer cell lines (AsPc-1, Capan-1, Capan-2, and L3.6pl) were grown in Dulbecco's modified Eagle's medium (DMEM, Life Technologies Inc., Rockville, Md.) supplemented with 10% heat-inactivated fetal bovine serum (FBS), 100 units/ml penicillin and 100 μg/ml streptomycin.

Chemotherapeutic Agents and scFv23/TNF Fusion Construct

5-fluorouracil (5-FU) was from Roche Laboratories (Nutley, N.J.). Cisplatin and Etoposide (VP-16) were from Bristol Laboratories (Princeton, N.J.). Doxorubicin was from Cetus Corporation (Emeryville, Calif.). Gemcitabine was from Eli Lilly Co. (Indianapolis, Ind.). The scFv23/TNF fusion construct was produced in a bacterial expression host, purified to homogeniety and assessed for biological activity as previously described (Rosenblum et al., 1995).

Antibodies

Monoclonal anti-HER-2/neu antibody (Ab), rabbit polyclonal anti-HER-1 Ab, rabbit polyclonal anti-TNFR-1 Ab, rabbit polyclonal anti-TNFR-2 Ab, rabbit polyclonal anti-caspase-8 Ab, monoclonal anti-caspase-3 Ab, and monoclonal anti-PARP Ab were obtained from Santa Cruz Biotechnology, Santa Cruz, Calif. Rabbit polyclonal anti-phospho Akt Ab, and rabbit polyclonal anti-Akt Ab (Cell Signaling Technology, Beverly, Mass.) were used for Western blot analysis. The caspase-3 inhibitor, n-Acetyl-Asp-Glu-Val-Asp-al, (Ac-DEVD-CHO) was purchased from Sigma-Aldrich Co. (St. Louis, Mo.).

In Vitro Cytotoxicity Assays and Combination Studies

All human pancreatic cancer cells were seeded (1×10⁴/well) in flat-bottom 96-well microtiter plates (Becton Dickinson Labware, Franklin Lakes, N.J.) and 24 hr later, scFv23/TNF, TNF, and five chemotherapeutic agents (5-fluorouracil, cisplatin, etoposide, doxorubicin, and gemcitabine,) were added in triplicate wells. For combination studies, scFV23/TNF and each of five chemotherapeutic agents were combined at their individual IC₂₅ concentrations. To examine the potential mediation of the synergistic effect of combination treatments by activation of caspase-3 cells were pretreated with or without 100 μM caspase-3 inhibitor (Ac-DEVD-CHO) for 3 hr and then treated with their individual IC₂₅ concentrations. After incubation for an additional 72 hr, remaining adherent cells were stained by adding 50 μl of crystal violet solution (0.5% w/v in 20% MeOH/H₂O). Dye-stained cells were solubilized by addition of 100 μl of Sorenson's buffer [100 mM sodium citrate (pH 4.2) in 50% ethanol], and absorbance was measured at 630 nm using an ELISA plate reader (Bio-Tek Instruments, Inc., Winooski, Vt.).

The synergistic, additive or antagonistic effects of drug combinations were assessed according to the median effect principle as described by Chou and Talalay (Chou and Talalay, 1984): fa/fu=(D/Dm)m, where D is the dose of the drug, Dm is the IC50, fa is the fraction affected by the dose, fu is the fraction unaffected and m is a coefficient that determines the sigmoidicity of the curve.

Western Blot Analysis

To check the status of HER-1, HER-2/neu, TNF receptor-1, TNF receptor-2, and p-Akt, four human pancreatic cancer cell lines (AsPc-1, Capan-1, Capn-2, and L3.6pl) were washed two times with phosphate buffered saline (PBS) and lysed on ice for 20 min in 0.3 ml of lysis buffer (10 mM Tris-HCl, pH 8, 60 mM KCl, 1 mM EDTA, 1 mM DTT, 0.2% NP-40). Cell lysates (50 mg) were fractionated by 8-15% SDS-PAGE and transferred on Immobilon-P nitrocellulose membranes (Schleicher & Schuell Inc., Keene, N.H.). Membranes were blocked for 2 hours in Tris-buffered saline (TBS) containing 3% bovine serum albumin. Monoclonal anti-HER-2/neu Ab (Oncogene Research Products, San Diego, Calif.), rabbit polyclonal anti-HER-1 Ab, rabbit polyclonal anti-TNFR-1 Ab, rabbit polyclonal anti-TNFR-2 Ab, rabbit polyclonal anti-phospho Akt Ab, rabbit polyclonal anti-Akt, and goat anti-β-actin Ab were used for immunoblotting. Goat anti-mouse/goat anti-rabbit or swain anti-goat antibodies conjugated with horseradish peroxidase (Bio-Rad Laboratories, Hercules, Calif.) were used to visualize immunoreactive proteins at a 1:4000 dilution using ECL detection reagent (Amersham Pharmacia Biotech Inc., Piscataway, N.J.).

Detection of Apoptosis

Apoptosis was detected by TUNEL assay. To assess apoptosis, L3.6pl cells were plated on cover glass, allowed to adhere overnight, and then treated with 200 nM TNF or 200 nM scFv23/TNF for 48 hr. The cells were washed with PBS, permeabilized (0.1% Triton X-100, 0.1% sodium citrate), and then fixed in 4% paraformaldehyde. Fixed cells were stained with in situ cell death detection kit (Roche). Cells undergoing apoptosis were identified by fluorescence microscopy.

### Example 2

### Status of HER-2/neu, HER-1, TNFR-1, TNFR-2, and p-Akt in Four Human Pancreatic Cancer Cell Lines

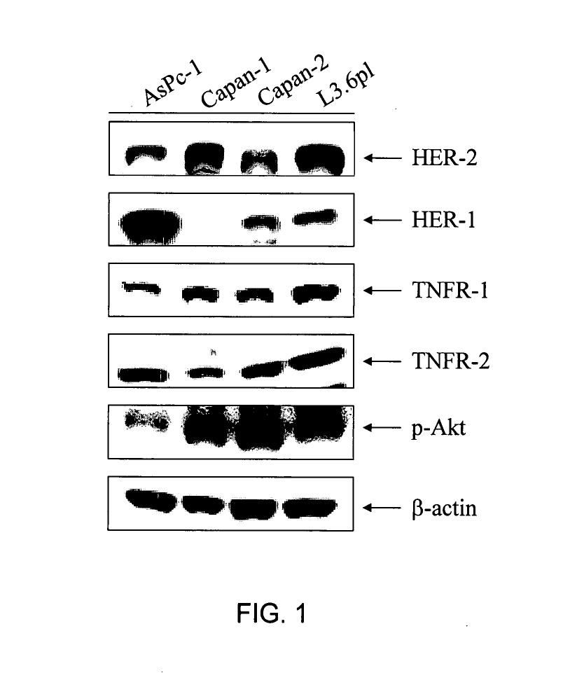

HER-2/neu has previously been found to be overexpressed in pancreatic tumor biopsy specimens and HER-2/neu expression has been proposed as a negative prognostic marker in pancreatic intraepithelial neoplasia (Tomaszewska et al., 1998). HER-2/neu expression was determined in four pancreatic cancer cell lines. All four pancreatic cancer cell lines (AsPc-1, Capan-1, Capan-2, and L3.6pl) expressed HER-2/neu, TNFR-1, TNFR-2, and phospho-Akt. Compared with AsPc-1 cells, L3.6pl cells expressed 3.7 fold higher levels of HER-2/neu, 3.1 fold higher levels of TNFR-1, and 1.6 fold higher levels of TNFR-2. Three of four pancreatic cell lines (Capan-1, Capan-2, and L3.6pl) also displayed elevated baseline levels of activated Akt. Compared with AsPc-1 cells, Capan-1 cells were found to express the highest levels of p-Akt (FIG. 1 and Table 1).

The receptor for epidermal growth factor (HER-1) has been previously found to be over-expressed on 33% of human pancreatic carcinomas (Thybusch-Bernhardt et al., 2001). Thus, the status of HER-1 in all four pancreatic cancer cell lines was evaluated. HER-1 expression was detectable in three pancreatic cancer cell lines (AsPc-1, Capan-2, and L3.6pl). Capan-1 cells expressed virtually no HER-1 (EGFR) while AsPc-1 cells expressed the highest levels of the four lines tested (FIG. 1 and Table 1).

TABLE 1
Comparative Expression of Various Signaling Proteins
on Human Pancreatic Cancer Cell Lines

| Cell line | HER-2/neu | HER-1 | TNFR-1 | TNFR-2 | p-Akt (fold) |
|---|---|---|---|---|---|
| AsPc-1 | 1 | 1 | 1 | 1 | 1 |
| Capan-1 | 3.5 | 0 | 2.7 | 0.8 | 5.7 |
| Capan-2 | 1.2 | 0.3 | 1.2 | 0.7 | 2.9 |
| L3.6pl | 3.7 | 0.4 | 3.1 | 1.6 | 3.8 |

### Example 3

### Effect of scFv23/TNF, TNF, and Chemotherapeutic Agents on Growth of Human Pancreatic Cancer Cell Lines

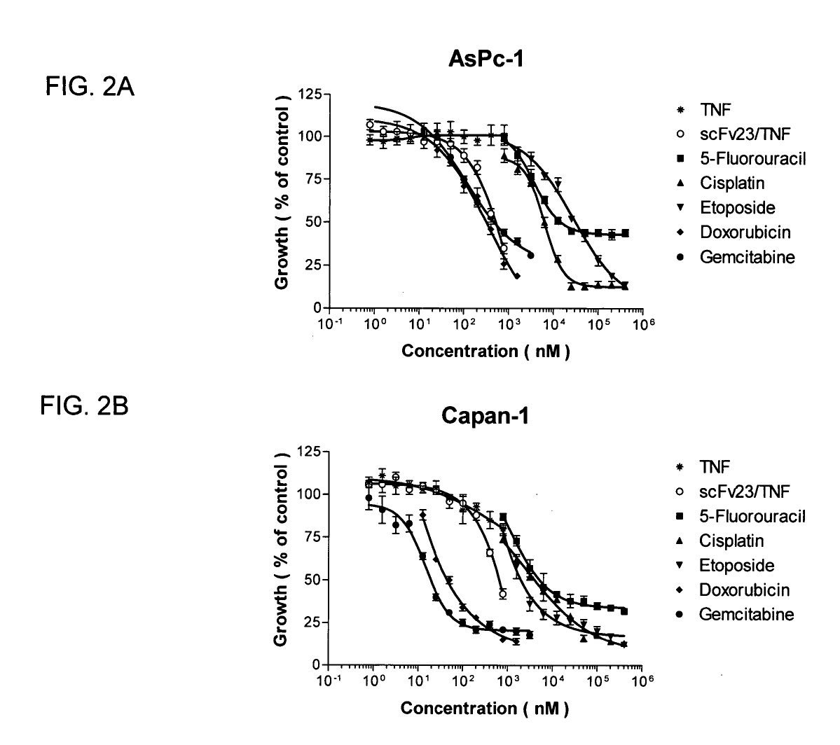

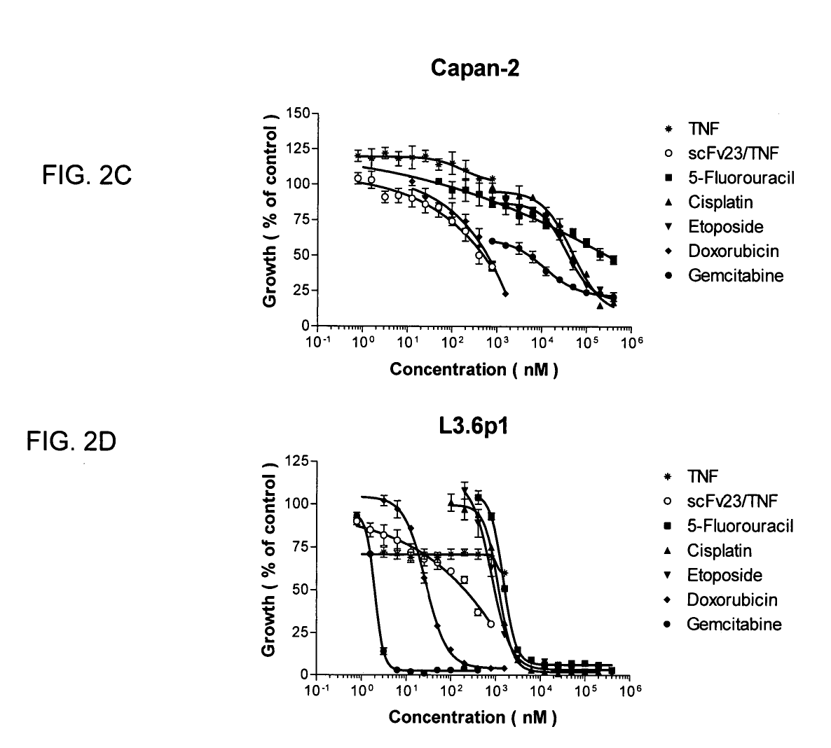

The ability of these chemotherapeutic agents to inhibit cell proliferation in vitro was markedly different among the four cell lines tested. All pancreatic cancer cell lines were highly resistant to the cytotoxic effects of TNF (IC₅₀>1600 nM). 5-fluorouracil, cisplatin, and etoposide showed IC₅₀ values between 1 and 300 mM whereas doxorubicin, gemcitabine and scFv23/TNF were comparatively more active with IC₅₀ values ranging between 6 and 700 nM (FIG. 2A-2D and Table 2).

TABLE 2
IC₅₀ of Various Agents Against Four Exemplary
Human Pancreatic Cancer Cell Lines

| Drug | AsPc-1 | Capan-1 | Capan-2 | L3.6pl |
|---|---|---|---|---|
| 5-Fluorouracil (5-FU) | 7.5 | 6 | 300 | 1 |
| Cisplatin (CIS) | 14 | 4.5 | 50 | 3.6 |
| Etoposide (ETO) | 28 | 2 | 40 | 2 |
| Doxorubicin (DOX) | 0.32 | 0.06 | 0.5 | 0.03 |
| Gemcitabine (GEM) | 0.2 | 0.02 | 0.15 | 0.006 |
| scFv23/TNF | 0.5 | 0.7 | 0.4 | 0.15 |
| TNF | >1.6* | >1.6* | >1.6* | >1.6* |

*Highest concentration achieved.

The IC₅₀ values were determined after 72 hr of exposure to the drugs and were defined as the concentration causing 50% growth inhibition in treated cells compared to control cells.

Interestingly, L3.6pl cells expressing the highest levels of HER-2/neu, TNFR-1, TNFR-2 were the most sensitive to the tested drugs, whereas Capan-2 cells expressing comparatively lower levels of HER-2/neu, TNFR-1, and TNFR-2 were the most resistant to the tested drugs.

### Example 4

### Effect of scFv23/TNF in Combination with Various Chemotherapeutic Agents on Growth of Four Human Pancreatic Cancer Cell Lines

Studies combining scFv23/TNF and various chemotherapeutic agents demonstrated a synergistic cytotoxic effect of scFv23/TNF with 5-fluorouracil and an antagonistic effect of scFv23/TNF with doxorubicin in all pancreatic cancer cell lines. However, the addition of cisplatin or gemcitabine to scFv23/TNF resulted in antagonistic cytotoxic effects in 3/4 cell lines tested whereas the addition of etoposide to scFv23/TNF resulted in synergistic effect in 3/4 pancreatic cancer cell lines (Table 3).

TABLE 3
Analysis of Cytotoxicity Induced by scFv23/TNF in
Combination with Other Chemotherapeutic Agents

| | Combination Index (CI) | | | |
|---|---|---|---|---|
| Treatment | AsPc-1 | Capan-1 | Capan-2 | L3.6pl |
| 5-FU + | SYN (0.632) | SYN (0.611) | SYN (0.548) | SYN (0.366) |
| scFv23/TNF | | | | |
| CIS + | ANT (2.250) | SYN (0.566) | ANT (1.532) | ANT (4.667) |
| scFv23/TNF | | | | |
| ETO + | SYN (0.498) | SYN (0.869 | SYN (0.664) | ANT (1.640) |
| scFv23/TNF | | | | |
| DOX + | ANT (1.805) | ANT (1.203) | ANT (2.084) | ANT (3.578) |
| scFv23/TNF | | | | |
| GEM + | ANT (2.375) | ANT (1.250) | SYN (0.703) | ANT (2.833) |
| scFv23/TNF | | | | |

To analyze the cellular interaction between the 2 agents, the tested combination of the 2 agents combination index (CI) values were calculated as proposed by Chou and Talalay (1984), for mutually exclusive drugs: CI=(D)1/(Dx)1+(D)₂/(Dx)₂. Where (D)1 and (D)₂ in combination kill X % of cells, and (Dx)1 and (Dx)₂ are the estimated dose of the drug alone capable of producing the same effect of the combined drugs. If CI near to 1 indicates additive effect (ADD), CI>1 indicates antagonism (ANT), CI<1 indicates synergism (SYN).

These results indicate that targeting HER-2/neu and TNFR-1 expressing tumor cells using the scFv23/TNF fusion toxin may be an effective therapy for pancreatic cancer especially when utilized in combination with specific chemotherapeutic agents such as 5-FU.

### Example 5

### Effect of 5-Fluorouracil, scFv23/TNF, and 5-FU Plus scFv2/TNF on Akt Phosphorylation

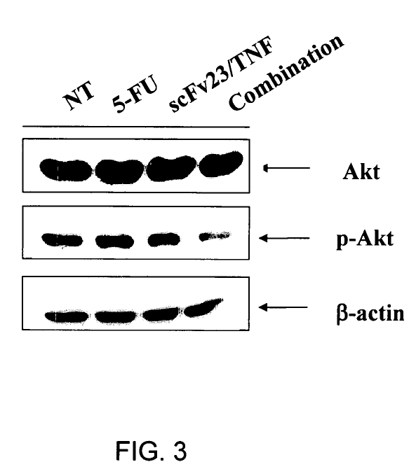

HER-2/neu over-expression results in activation of different downstream pathways such as the Akt kinase pathway, which leads to cell proliferation and cell survival. To determine whether 5-FU or scFv23/TNF affects this survival pathway, L3.6pl cells were treated with IC₂₅ doses of 5-fluorouracil, scFv23, or 5-FU+scFv23/TNF. The activation of Akt kinase was then assessed by Western blot analysis using antibodies to Akt and to phospho-Akt. As shown in FIG. 3, treatment of cells with 5-FU, scFv23/TNF as single agents, or combinations had no impact on the total levels of Akt, while the combination 5-FU+scFv23/TNF inhibited phosphorylation of the Akt protein by 64%. These results indicate that 5-FU+scFv23/TNF-induced cytotoxicity may be mediated, at least in part, by an inhibitory effect on Akt phosphorylation events.

### Example 6

### Effect of 5-Fluorouracil, scFv23/TNF, and 5-FU Plus scFv2/TNF on Bcl-2 Expression

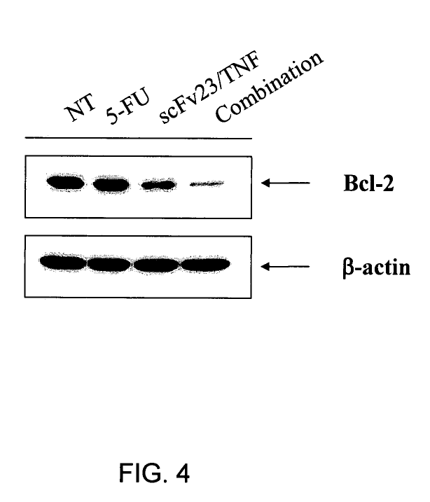

Increased levels of the anti-apoptotic protein Bcl-2 contribute to cellular resistance of tumor cells to a variety of chemotherapeutic agents including cyclophosphamide, methotrexate, anthracycline, cytarabine, paclitaxel, and corticosteroids (Wachter et al., 1999). To determine whether 5-FU, scFv23/TNF or 5-FU+scFv23/TNF effects are mediated through changes in cellular levels of Bcl-2, L3.6pl cells were treated with IC₂₅ doses of 5-fluorouracil, scFv23, TNF, or 5-FU+scFv23/TNF. As shown in FIG. 4, treatment of cells with 5-FU had no impact on cellular levels of Bcl-2 while scFv23/TNF and 5-FU+scFv23/TNF inhibited Bcl-2 expression levels 44% and 74%, respectively. These results suggest that 5-FU+scFv23/TNF-induced cytotoxicity may be mediated by inhibition of Bcl-2 expression.

### Example 7

### Effects of 5-Fluorouracil, scFv23/TNF, and 5-FU Plus scFv2/TNF on Apoptosis, Caspase-8, Caspase-3, and PARP Cellular Levels

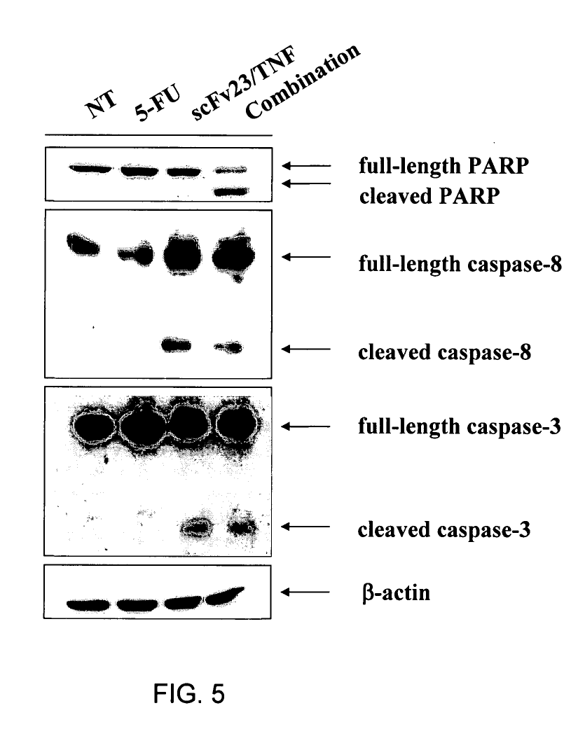

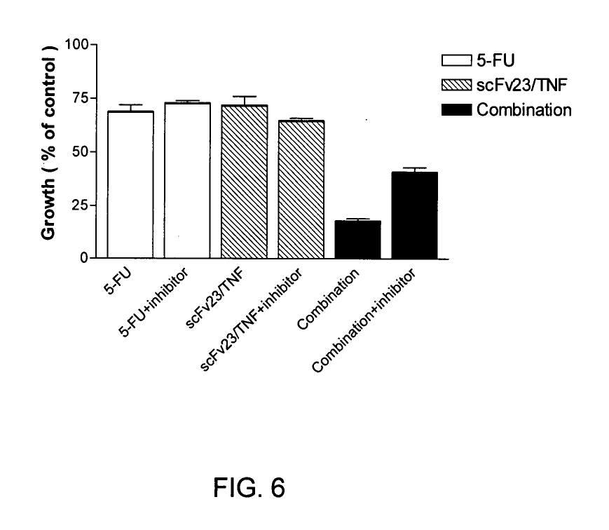

To determine whether the cytotoxic effect of scFv23/TNF was associated with apoptosis, L3.6pl cells were assayed for apoptosis by TUNEL staining. The 5-FU+scFv23/TNF-treated cells demonstrated induction of apoptotic cell death within 48 hrs after treatment. The caspase series of proteins is known to be a central mediator of the apoptotic effects of TNF and other cytokines. To determine whether caspase-8 and caspase-3 were activated in L3.6pl cells during 5-FU+scFv23/TNF-induced cell death, the cleavage of caspase-8, caspase-3, and its substrate poly (ADP)-ribose polymerase (PARP) was investigated. Treatment with 5-FU had no effect on caspase-8, caspase-3, and PARP cleavage, whereas exposure of the cells to the scFv23/TNF or scFv23/TNF plus 5-FU combination resulted in cleavage of caspase-8, and caspase-3. In addition, scFv23/TNF plus 5-FU combination induced PARP cleavage at 48 hr (FIG. 5). To determine whether 5-FU+scFv23/TNF-induced apoptosis was dependent on activation of the caspase-3 pathway, the effect of a caspase-3 inhibitor on the cytotoxicity of 5-FU+scFv23/TNF was examined against L3.6pl cells. As shown in FIG. 6, treatment with the caspase-3 inhibitor had no effect on the cytotoxic effects of 5-FU or scFv23/TNF as single agents. However, pre-treatment with the inhibitor followed by combination treatment (5-FU+scFv23/TNF) was able to partially reverse the synergistic cytotoxic effects observed. This suggests that the synergistic cytotoxic effects of the combination may depend, at least in part on a caspase-driven pathway.

### Example 8

### Significance of Targeting TNF in Pancreatic Cancer

Human epidermal growth factor receptor-2 (HER-2/erbB-2) belongs to a family of four transmembrane receptors (HER-1, HER-3, and HER-4) (Lohrisch and Piccart, 2001; Yarden, 2001; Rubin and Yarden, 2001) and it plays a key role in the HER family signaling events, cooperating with other HER receptors via a complex signaling network to regulate cell growth, differentiation, and survival. Over-expression of HER-2/neu has been observed in several cancers where it is associated with multiple drug resistance, higher metastatic potential, and decreased patient survival times (Tomaszewska et al., 1998; Hynes and Stern, 1994; Singleton and Strickler, 1992; Stancovski et al., 1994; Torre et al., 1997; Safran et al., 2001). To evaluate the influence of HER-2/neu expression in pancreatic cancer as it relates to clinical response to therapeutic agents, a variety of groups have used several HER-2/neu targeting strategies including using HER-2/neu targeted ribozymes (Irie et al., 200; Thybusch-Bernhardt et al., 2001; Aigner et al., 2000; Suzuki et al., 2000) humanized anti-HER-2/neu antibody (Herceptin), and combination chemotherapeutic treatment regimens with Herceptin (Waldmann et al., 2000; Buchler et al., 2001; Butera et al., 1998).

The approach of the particular embodiment of the present invention was to utilize HER-2/neu expression on the surface of tumor cells as a therapeutic target employing the anti-HER-2/neu single chain antibody to deliver TNF directly to tumor cells (Rosenblum et al., 2000) Previous studies by the inventor demonstrated that this approach can be highly effective in directing TNF in vivo to tumor cells, and it was further demonstrated that fusion constructs containing TNF were highly cytotoxic even to tumor cells resistant to TNF itself. The mechanistic effects of the scFv23/TNF construct were examined on a panel of four pancreatic cancer cell lines, which were characterized for various levels of oncogene expression and comparative response to chemotherapeutic agents.

The chemotherapeutic agents utilized in this exemplary study were selected to present a spectrum of different cellular targets and are representative of the major classes of agents with therapeutic value. The potential combinations of tumor-targeted delivery of TNF in combination with chemotherapeutic agents have not been previously examined. Combining scFv23/TNF and various chemotherapeutic agents clearly demonstrated a uniform synergistic effect of scFv23/TNF and 5-FU in all pancreatic tumor cell lines. There was a correlation between expression of HER-2/neu and TNFR-1 and response for HER-2/neu-overexpressing cells such as L3.6pl to be more sensitive to chemotherapeutic agents in combination with scFv23/TNF. Pegram et al. reported that 5-FU has an antagonistic effect in vitro in combination with anti-HER-2/neu monoclonal antibodies, whereas cisplatin, etoposide, and doxorubicin previously showed a synergistic or an additive effect in combination with Herceptin (Pegram et al., 2000). However, the present inventors found a uniform synergistic effect of scFv23/TNF in combination with 5-FU and an antagonistic effect of scFv23/TNF in combination with doxorubicin against all four pancreatic cancer cell lines (Table 3).

Over-expression of HER-2/neu is known to activate the Akt pathway and to confer resistance to apoptosis induced by many therapeutic drugs (Kneufermann et al., 2003). Three of four human pancreatic adenocarcinoma cell lines displayed elevated baseline levels of activated Akt, and there was a correlation between HER-2/neu and p-Akt expression. Especially Capan-1 and L3.6pl cell lines have high levels of HER-2/neu and activated Akt. Treatment of L3.6pl cells with combination 5-FU+scFv23/TNF resulted in significant reduction in Akt phosphorylation. This indicates that 5-FU+scFv23/TNF-induced cytotoxicity may be mediated, at least in part, by the inhibition of Akt survival signaling pathway.

Overexpression of Bcl-2 has been shown to contribute to the cellular resistance of a variety of chemotherapeutic drugs, including cyclophosphamide, methotrexate, anthracycline, cytarabine, paclitaxel, and corticosteroids. (Wuchter et al., 1999) Sasaki et al reported that the level of Bcl-2 in cancer cells was an indicator of 5-FU efficacy (Sasaki et al., 2003). The present inventors determined that scFv23/TNF and 5-FU+scFv23/TNF inhibited 44% and 74%, respectively; however, treatment of cells with 5-FU had no impact on the levels of Bcl-2. Down-regulation of Bcl-2 by scFv23/TNF may have induced the sensitization of L3.6pl cells to be more sensitive to 5-FU. Therefore, scFv23/TNF in combination with 5-FU accelerates the inhibition of Bcl-2 expression.

The present inventors also determined that another critical factor in the mediation of scFv23/TNF cytotoxicity is the caspase activation cascade. Binding of TNF to TNFR-1 can induce the formation of signaling complexes, TNF-R1-TRADD-FADD-pro-caspase-8, resulting in the activation of caspase-8 (Nagata, 1997). The activation of caspase-8 is thought to result in proteolytic activation of the other caspases (Medema et al., 1997). The activation of caspase-3 contributes to paclitaxel-induced apoptosis in HER-2/neu-overexpressing SKOV3.1p159 and immunotoxin-induced apotosis (Keppler-Hafkemeyer et al., 1998). Treatment with scFv23/TNF alone and combination 5-FU+scFv23/TNF resulted in activation of caspase-8 with eventual cleavage of caspase-3 and PARP. The present inventors determined that the combination 5-FU+scFv23/TNF can cause a synergistic cytotoxicity through activation of caspase-8, caspase-3, and PARP cleavage.

The signal transduction effects of 5-FU, scFv23/TNF, and 5-FU+scFv23/TNF are summarized in Table 4.

TABLE 4
Summary of Signal Transduction Effects of
5-Fluorouracil, scFv23/TNF, and Combination
Thereof on Exemplary L3.6pl Cells

| Signal | 5-fluorouracil | scFv23/TNF | 5-FU + scFv23/TNF |
|---|---|---|---|
| Akt | NE | NE | NE |
| p-Akt | NE | NE | ↓ |
| Bcl-2 | NE | ↓ | ↓ |
| Caspase-8 cleavage | NE | ↑ | ↑ |
| Caspase-3 cleavage | NE | ↑ | ↑ |
| PARP cleavage | NE | NE | ↑ |

*NE represents no effect.

Thus, delivery of the cytokine TNF to HER-2/neu expressing tumor cells using the scFv23/TNF fusion toxin is an effective therapy for pancreatic cancer especially when utilized in combination with chemotherapeutic agents.

### Example 9

### Methods and Materials for Examples 10-14

Materials

Monoclonal anti-HER-2/neu antibody (Ab), rabbit polyclonal anti-TNFR1 Ab, rabbit polyclonal anti-TNFR2 Ab, rabbit polyclonal anti-caspase-8 Ab, monoclonal anti-Caspase-3 Ab, monoclonal anti-PARP Ab, rabbit polyclonal anti-TRADD Ab, rabbit polyclonal anti-TRAF2 Ab, rabbit polyclonal anti-IκB-α Ab were all obtained from Santa Cruz Biotechnology, Santa Cruz, Calif. Rabbit polyclonal anti-phospho Akt Ab, and rabbit polyclonal anti-Akt Ab (Cell Signaling Technology, Beverly, Mass.) were used for Western blot analysis. For neutralization assays, monoclonal anti-TNFR-1 Ab was purchased from Oncogene Research Products (San Diego, Calif.). N-Acetyl-Asp-Glu-Val-Asp-al (Ac-DEVD-CHO) was purchased from Sigma-Aldrich Co. (St. Louis, Mo.). The cell growth XTT assay kit was purchased from Roche Diagnostics Co. (Indianapolis, Ind.).

Cell Lines and Culture

SKBR-3 cells were grown in McCoy's 5A modified medium, supplemented with 10% heat-inactivated fetal bovine serum (FBS), 2 mM L-glutamine, 100 units/ml penicillin and 100 μg/ml streptomycin. SKBR-3 low passage cells expressing high amounts of HER-2/neu (SKBR-3/H) used in our study were between passage 5 and 8 while the SKBR-3 high passage cells used were between passage 40 and 45 and displayed comparatively lower levels of HER-2/neu (SKBR-3/L).

In Vitro Cytotoxicity Assays

SKBR-3 cells were seeded (1×10⁴/well) in flat-bottom 96-well microtiter plates (Becton Dickinson Labware, Franklin Lakes, N.J.) and 24 hrs later scFv23, TNF, and scFv23/TNF were added in triplicate wells. To examine the effect of caspase-3 inhibitor on the cytotoxicity of scFv23/TNF, SKBR-3/H cells were pretreated with or without 100 μM caspase-3 inhibitor (Ac-DEVD-CHO) for 3 hr and then treated with various concentration of scFv23/TNF. After 72 hr, 50 μl of XTT labeling mixture (Roche) was added to each well, after which the cells incubated for another 4 hr. The spectrophotometric absorbance was measured at 450 nm using an ELISA reader (Bio-Tek Instruments, Inc., Winooski, Vt.).

Neutralizing Effect of Anti-TNFR-1 Antibody on scFv23/TNF-treated SKBR-3 Cell Growth

SKBR-3 cells were seeded (1×10⁴/well) in flat-bottom 96-well microtiter plates (Becton Dickinson Labware, Franklin Lakes, N.J.), 24 hrs later pretreated with various concentrations of anti-TNFR-1 antibody for 2 hr, and then TNF and scFv23/TNF were added in triplicate wells. After 72 hr, cell viability was detected by XTT assay (Roche).

Detection of Apoptosis

Apoptosis was detected by DNA fragmentation and TUNEL assay. To evaluate DNA fragmentation, SKBR-3/H cells were seeded at 5×10⁵ cells/60 mm petri-dish, allowed to grow overnight, and then treated with 200 nM TNF or 200 nM scFv23/TNF. After 24 hr and 48 hr, cells were washed with PBS, resuspended in a DNA extraction buffer containing 5 mM Tris-HCl, pH 8, 50 mM EDTA, 10 μg/ml RNAse, and 0.25% SDS and then incubated for 1 hr at 37° C. To remove protein, resuspended cell lysates were treated with 100 μg/ml proteinase K for 3 hr at 50° C. DNA was extracted using phenol and chloroform followed by ethanol precipitation. The genomic DNA was resuspended in Tris-EDTA (pH 8) and was fractionated by electrophoresis on a 1% agarose gel containing ethidium bromide.

To assess apoptosis, SKBR-3/H cells were plated on cover glass, allowed to adhere overnight, and then treated with 200 nM TNF or 200 nM scFv23/TNF for 24 hr and 48 hr. The cells were washed with PBS, permeabilized (0.1% Triton X-100, 0.1% sodium citrate), and then fixed in 4% paraformaldehyde. Fixed cells were stained with in situ cell death detection kit (Roche). Cells undergoing apoptosis were identified by fluorescence microscopy.

Western Blot Analysis

SKBR-3/H cells were seeded at 5×10⁵ cells/60 mm petri-dish, allowed to grow overnight, and then treated with 200 nM scFv23, 200 nM TNF or 200 nM scFv23/TNF. After treatment, cells were washed twice with phosphate buffered saline (PBS) and lysed on ice for 20 min in 0.3 ml of lysis buffer (10 mM Tris-HCl, pH 8, 60 mM KCl, 1 mM EDTA, 1 mM DTT, 0.2% NP-40). Cell lysates (50 μg) were fractionated by 8-15% SDS-PAGE and electrophoretically transferred to Immobilon-P nitrocellulose membranes (Schleicher & Schuell Inc., Keene, N.H.). Membranes were blocked 2 hrs in Tris-buffered saline (TBS) containing 3% bovine serum albumin and then probed with various antibodies (monoclonal anti-HER-2/neu Ab, rabbit polyclonal anti-TNFR1 Ab, rabbit polyclonal anti-TNFR2 Ab, rabbit polyclonal anti-caspase-8 Ab, monoclonal anti-caspase-3 Ab, monoclonal anti-PARP Ab, rabbit polyclonal anti-TRADD Ab, rabbit polyclonal anti-TRAF2 Ab, rabbit polyclonal anti-IκB-α Ab, rabbit polyclonal anti-phospho Akt Ab, and rabbit polyclonal anti-Akt Ab). Goat anti-mouse/goat anti-rabbit or swain anti-goat antibodies conjugated with horseradish peroxidase (Bio-Rad Laboratories, Hercules, Calif.) were used to visualize immunoreactive proteins at a 1:4000 dilution using ECL detection reagent (Amersham Pharmacia Biotech Inc., Piscataway, N.J.).

### Example 10

### Correlation Between Signaling Protein Expression and Sensitivity to scFv23/TNF

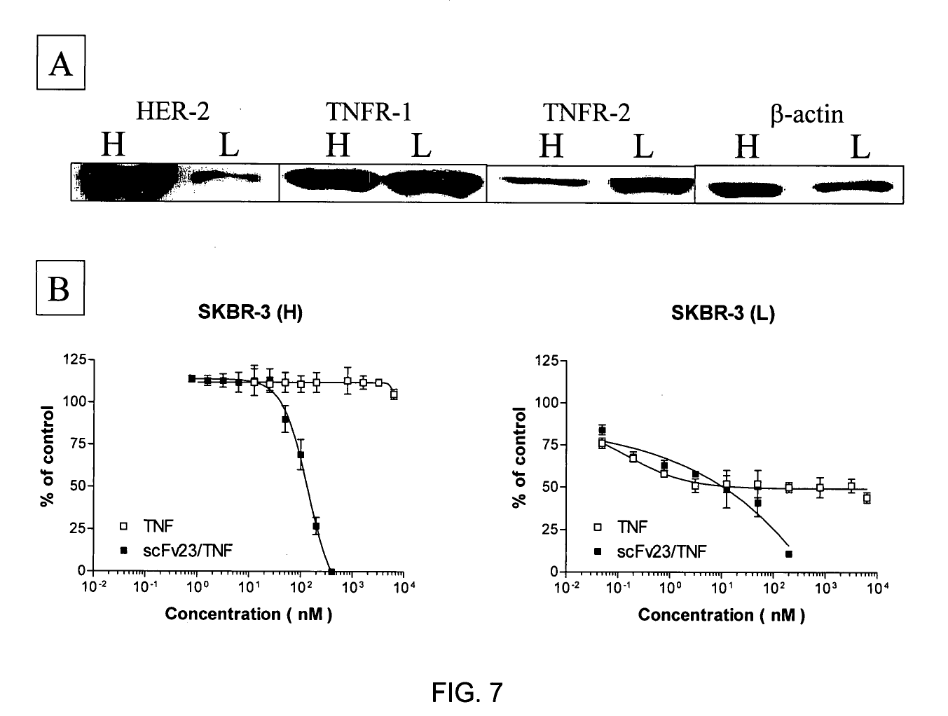

To determine whether a correlation exists between HER-2/neu, TNFR-1, and TNFR-2 expression levels and cytotoxic effects of TNF and scFv23/TNF, we examined the relative levels of expression of HER-2/neu, TNF receptor-1 (TNFR-1), and TNF receptor-2 (TNFR-2) on SKBR-3/H and 3/L cell lines by Western blot analysis (FIG. 7A) and quantitated by densitometric analysis in Table 5. As shown in FIG. 7A and Table 5, both SKBR-3 cell lines expressed HER-2/neu, TNFR-1, and TNFR-2.

TABLE 5
Comparative sensitivity and expression of signaling
proteins in SKBR-3 human breast cancer cell lines

| | IC₅₀ | | | Relative Expression (fold) | | |
|---|---|---|---|---|---|---|
| Cell line | scFv23 | TNF | scFv23/TNF | HER-2 | TNFR-1 | TNFR-2 |
| SKBR-3/ | NE* | NE | 150 nM | 3.3 | 1 | 1 |
| H | | | | | | |
| SKBR-3/ | NE | 10 nM | 4 nM | 1 | 2.3 | 4 |
| L | | | | | | |

*NE represents no effect.

Interestingly, SKBR-3/H cells expressed 3.3-fold higher level of HER-2/neu than the SKBR-3/L cells. On the other hand, SKBR-3/L cells expressed 2.3 fold and 4 fold higher levels of TNF receptor-1 and TNF respector-2, respectively compared to SKBR-3/H cells. SKBR-3/H cells expressing higher levels of HER-2/neu were completely resistant to TNF itself at doses up to 6 μM although these cells were sensitive to the cytotoxic effects of scFv23/TNF (IC₅₀=150 nM). Treatment with scFv23 itself on either cell line had no effect. However, SKBR-3/L cells expressing lower levels of HER-2/neu and high levels of TNFR-1 and TNFR-2 demonstrated similar IC₅₀ values to TNF itself and scFv23/TNF (10 nM and 4 nM, respectively) (FIG. 7B). These results indicate that continual culture of the SKBR-3 cell line result in an up regulation of the TNFR-1 and TNFR-2 receptors and a concomitant down regulation of HER-2/neu. These data indicate that the scFv23/TNF fusion construct can overcome HER-2/neu-induced TNF resistance.

### Example 11

### Neutralizing Effect of TNF Receptor-1 on scFv23/TNF-Induced Growth Inhibition

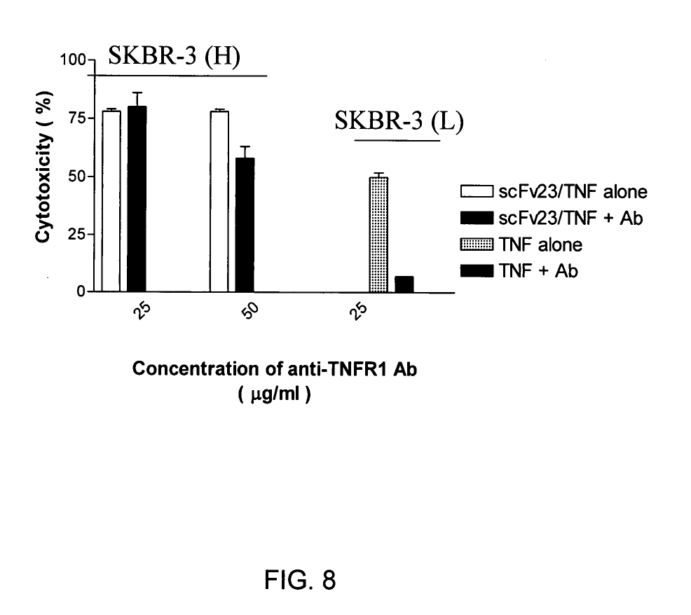

To determine whether the cytotoxic effects of the scFv23/TNF construct were mediated entirely through interaction with cell-surface TNFR-1 receptors, the effect of an anti-TNFR-1 neutralizing antibody on the cytotoxicity of scFv23/TNF against SKBR-3 cells was examined. As shown in FIG. 8, treatment of SKBR-3/L cells with a dose of 25 μg/ml antibody was able to completely ablate the TNF-induced cytotoxicity. In contrast, the cytotoxicity of scFv23/TNF against SKBR-3/H cells was not affected until concentrations of 50 μg/ml anti-TNFR-1 Ab were attained. Complete abrogation of the cytotoxic effects of scFv23/TNF against SKBR-3/H cells could not be achieved by addition of anti-TNFR-1 neutralizing antibody.

### Example 12

### Effect of scFv23/TNF, TNF and scFv23 on the IκB-α and Akt Pathway

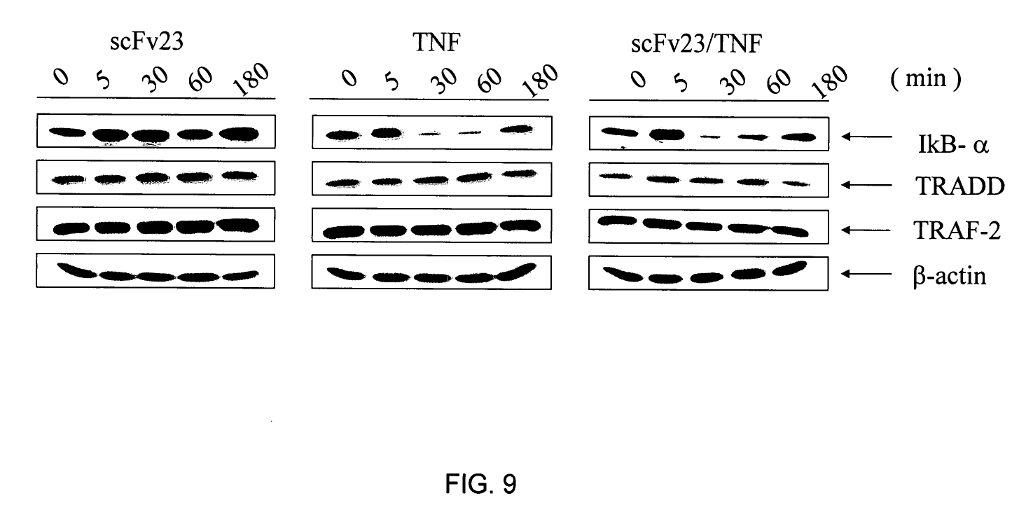

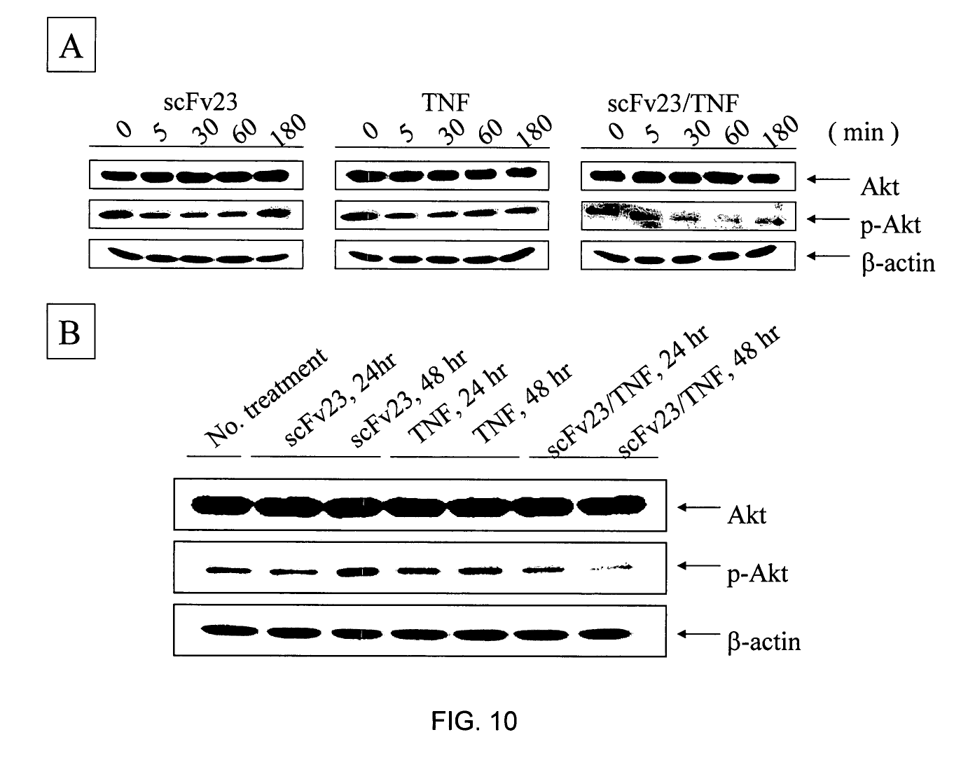

After binding to TNF receptors on the tumor cell surface, the cytotoxic effects of TNF-α can be mediated directly by activating signaling pathways that initiate programmed cell death. To determine whether scFv23/TNF induced apoptosis followed a similar signal transduction process compared to native TNF, the effects of these three agents on IκB-α, TRADD, and TRAF2 expression were examined. HER-2/neu-overexpressing SKBR-3/H cells were treated with 200 nM of scFv23, TNF, or scFv23/TNF for various times, and then the cell lysates were harvested and subjected to Western blot analysis. As shown in FIG. 9, treatment of cells with scFv23, TNF, or scFv23/TNF had no impact on the levels of TRAF2. Treatment with scFv23/TNF for 180 min resulted in a modest decrease in TRADD. After 30 minutes, IκB-α was degraded in both TNF- and scFv23/TNF-treated cells whereas treatment with scFv23 had no effect on IκB-α degradation. Three hours after addition of either TNF or scFv23/TNF, levels of IκB-α increased in basal levels. This indicates that IκB-α pathway may be involved in scFv23/TNF-mediated signaling transduction but this was not an effect of the scFv23 component. HER-2/neu over-expression results in activation of different downstream pathways such as Akt kinase pathway, which leads to cell proliferation and cell survival. To determine whether scFv23/TNF affects on this Akt survival pathway, SKBR-3/H cells were treated with scFv23, TNF, or scFv23/TNF. The activation of Akt kinase was then assessed by Western blot analysis using antibodies to Akt and to phospho-Akt. As shown in FIGS. 10A and 10B, treatment with either scFv23 or TNF activated phosphorylation of Akt at 48 hr of exposure. On the other hand, treatment of cells with scFv23/TNF resulted in down-regulation of phosphorylated Akt early after drug administration (30 min) and much later (48 hr). This indicates that HER-2/neu-induced TNF resistance is mediated, at least in part, by the Akt survival signaling pathway and scFv23/TNF-induced cytotoxicity is mediated by inhibition of Akt phosphorylation.

### Example 13

### Effects of scFv23/TNF and TNF on Apoptosis, Caspase-8, Caspase-3, and PARP Cleavage

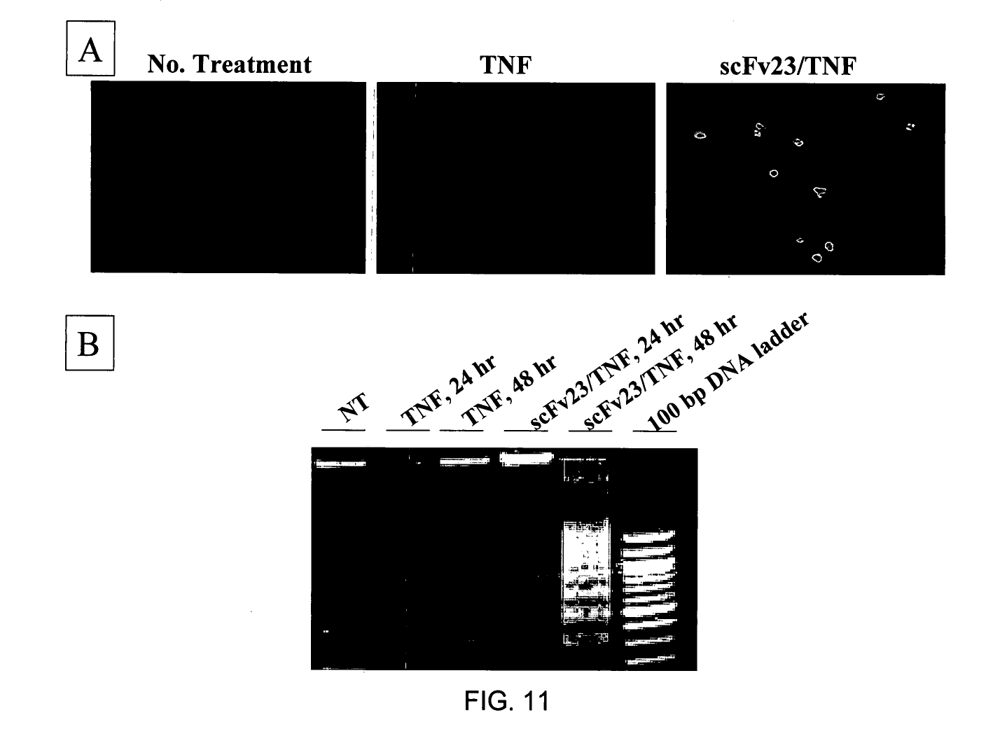

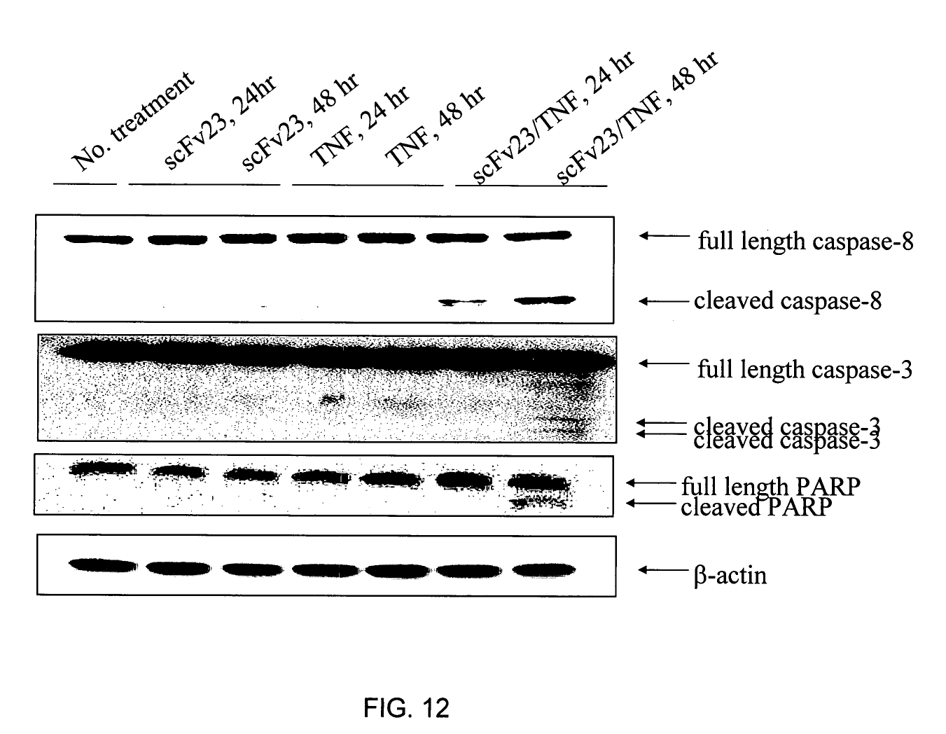

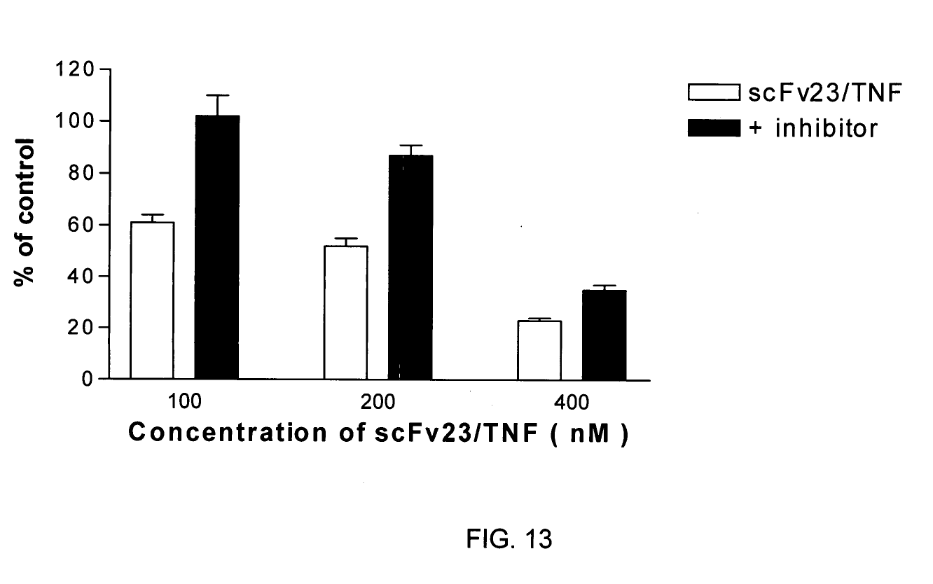

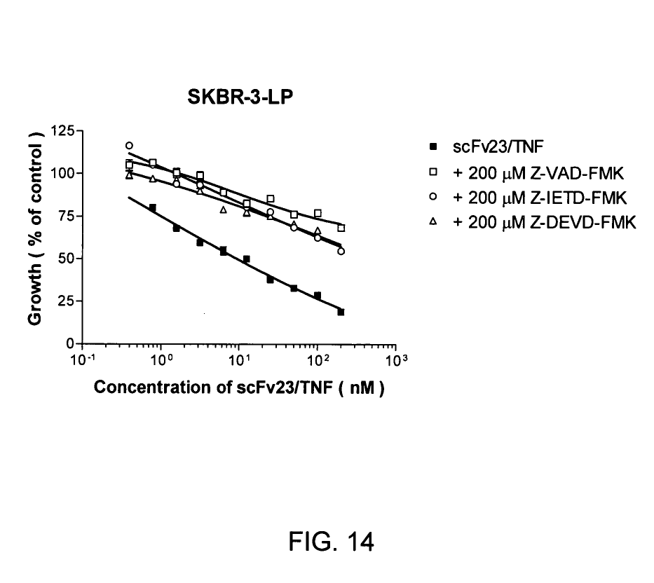

To determine whether the cytotoxic effect of scFv23/TNF was associated with apoptosis, DNA was extracted from SKBR-3/H cells at 24 hr and 48 hr after exposure to either 200 nM TNF or scFv23/TNF. The DNA was subjected to electrophoresis on a 1% agarose gel. A DNA fragmentation pattern characteristic of apoptosis was detected in scFv23/TNF-treated but not TNF-treated SKBR-3/H cells (FIG. 11A). SKBR-3/H cells were also assayed for apoptosis by TUNEL staining. As shown in FIG. 11B, scFv23/TNF-treated cells showed DNA fragmentation as well as nuclear condensation typical of apoptotic cell death at 48 hr of exposure. The caspase series of proteins is known to be a central mediator of the apoptotic effects of TNF and other cytokines. To determine whether caspase-8 and caspase-3 were activated in SKBR-3/H cells during scFv23/TNF-induced cell death, the cleavage of caspase-8, caspase-3, and its substrate poly (ADP)-ribose polymerase (PARP) was studied. Treatment with TNF had no effect on caspase-8, caspase-3, and PARP cleavage. In contrast, treatment with scFv23/TNF resulted in cleavage of caspase-8, caspase-3, and PARP at 48 hr (FIG. 12). To determine whether scFv23/TNF-induced apoptosis was dependent on activation of the caspase-3 pathway, the effect of a caspase-3 inhibitor on the cytotoxicity of scFv23/TNF against SKBR-3/H cells was examined. FIG. 13 shows that scFv23/TNF-induced cytotoxicity was inhibited by a caspase-3 inhibitor (Ac-DEVD-CHO). To determine whether scFv23/TNF-induced apoptosis was dependent on activation of the caspase-8 and -3 pathway, the effect of caspase inhibitors on the cytotoxicity of scFv23/TNF against SKBR-3-LP cells was examined. FIG. 14 shows scFv23/TNF-induced cytotoxicity was inhibited by general caspase inhibitor (Z-VAD-FMK), caspase-8 inhibitor (Z-IETD-FMK), and caspase-3 inhibitor (Z-DEVD-FMK). This result demonstrates that scFv23/TNF elicits an apoptotic response that appears to be mediated, at least in part, through a caspase-8 and -3 dependent cascade.

### Example 14

### Significance of Unique Apoptotic Signaling of scFv23/TNF in HER-2/Neu-Overexpressing Cells

The use of antibodies for the specific delivery of cytokines to tumor cells has been demonstrated by numerous groups using TNF, interferon, IL-2, and lymphotoxin (Rosenblum et al., 1991; Zuckerman et al., 1987; Reisfeld et al., 1996). Several of these studies have been demonstrated that the antibody-targeted cytokine was more effective than the original cytokine. Utilizing an antibody construct containing TNF, the present inventors initially demonstrated that delivery of TNF to tumor cells using recombinant single-chain antibody fusion constructs containing TNF and targeting gp240 and HER-2/neu could overcome resistance of tumor cells to TNF in vitro (Rosenblum et al., 1995; Rosenblum et al., 2000).

Overexpression of HER-2/neu appears to be associated with a survival advantage and with TNF resistance at least in breast, ovarian, and HER-2-transfected cell lines (Tang et al., 1994; Lichtenstein et al., 1990; Hudziak et al., 1988). On the other hand, down-regulation of HER-2/neu has been shown to confer enhanced sensitivity to the cytotoxicity of TNF in doxorubicin-resistant tumor cell lines (Sleijfer et al., 1998). Studies have additionally demonstrated that EGF signaling in breast and cervical carcinoma cells can also modulate the cytotoxic effects of TNF (Hoffmann et al., 1998).

The present inventors determined that continual culture of the SKBR-3 cell line resulted in down-regulation of HER-2/neu and conferred TNF sensitivity to low amounts of HER-2/neu-expressing SKBR-3/L cells, and scFv23/TNF composed of the anti-HER-2/neu single chain antibody fused to TNF can overcome HER-2-induced TNF resistance in HER-2/neu-overexpressing SKBR-3/H cells. Two important factors for contributing the scFv23/TNF-induced cytotoxicity are the Akt and caspase(s). The serine/threonine protein kinase Akt has been shown to have a pivotal role in cell cycle progression (Brennan et al., 1997; Muise-Helmericks et al., 1998; Gill and Downward, 1999), angiogenesis (Jiang et al., 2000), inhibition of apoptosis (Sabbatini and McCormick, 1999; Zhou et al., 2000), and cell growth (Verdu et al., 1999). Over-expression of HER-2/neu is known to activate the Akt pathway and to confer resistance to apoptosis induced by many therapeutic drugs (Yu and Hung, 2000; Knuefermann et al., 2003). SKBR-3/H cells that over-express HER-2/neu had endogenous levels of p-Akt and Akt. Treatment of these cells with TNF or scFv23 antibody alone had no effect on cell growth but was shown to induce Akt phosphorylation 48 hrs after exposure. In contrast to the effects of TNF, scFv23/TNF treatment resulted in significant reduction in Akt phosphorylation. The result suggests that Akt phosphorylation plays an important role in conferring a TNF-resistance on HER-2/neu-overexpressing SKBR-3/H cells and scFv23/TNF-induced cytotoxicity may be mediated, at least in part, by the inhibition of Akt survival signaling pathway.

Another critical factor in the mediation of scFv23/TNF cytotoxicity is the caspase activation cascade. Binding of TNF to TNF-R1 can induce the formation of signaling complexes, TNF-R1-TRADD-FADD-pro-caspase-8, resulting in the activation of caspase-8 (Nagata, 1997). The activation of caspase-8 is thought to result in proteolytic activation of the other caspases (Medema et al., 1997). The activation of caspase-3 contributes to paclitaxel-induced apoptosis in HER-2/neu-overexpressing SKOV3.1pl (Ueno et al., 2000) and immunotoxin-induced apotosis (Keppler-Hafkemeyer et al., 1998). Treatment with scFv23/TNF resulted in activation of caspase-8 in a time-dependent manner and with eventual cleavage of caspase-3 and PARP at 24 and 48 hrs, respectively. The data indicate that the scFv23/TNF-induced cytotoxic mechanism was accompanied by inducing the apoptotic cascade through activation of caspase-8, caspase-3, and PARP cleavage.

In one embodiment, a correlation between scFv23/TNF-induced cytotoxicity and expression of TNF receptor(s) is because the fusion construct physically interacts with the TNFR-1 in a manner different from that of native TNF. Alternatively, or in addition, since the scFv23 antibody effectively internalizes into cells, this could deliver TNF to the cytoplasm where it would be available for interaction with intracellular TNFR-1.

The observed differences in signaling events between TNF and scFv23/TNF are provided in Table 6.

TABLE 6
Summary of signal transduction effects of scFv23,
TNF, and scFv23/TNF on Exemplary
HER-2-overexpressing SKBR-3/H cells

| Signal | scFv23 | TNF | scFv23/TNF |
|---|---|---|---|
| IκB-α | NE* | ↓ | ↓ |
| TRADD | NE | NE | ↓ |
| TRAF-2 | NE | NE | NE |
| Akt | NE | NE | NE |
| p-Akt | ↑ | ↑ | ↓ |
| Caspase-8 cleavage | NE | NE | ↑ |
| Caspase-3 cleavage | NE | NE | ↑ |
| PARP cleavage | NE | NE | ↑ |
| DNA fragmentation | NE | NE | ↑ |

The in vitro mechanistic studies indicate that scFv23/TNF is an effective cytotoxic agent against HER-2/neu-overexpressing cancer cells that are resistant to TNF.

### Example 15

### Exemplary Materials and Methods for Examples 16-20

Materials

Monoclonal anti-HER-2/neu antibody (Ab), rabbit polyclonal anti-TNF-R1 Ab, rabbit polyclonal anti-TNF-R2 Ab, rabbit polyclonal anti-caspase-8 Ab, monoclonal anti-caspase-3 Ab, monoclonal anti-PARP Ab, rabbit polyclonal anti-TRADD Ab, rabbit polyclonal anti-TRAF2 Ab, and rabbit polyclonal anti-IκB-α Ab were all obtained from Santa Cruz Biotechnology, Santa Cruz, Calif. Rabbit polyclonal anti-phospho Akt Ab, and rabbit polyclonal anti-Akt Ab (Cell Signaling Technology, Beverly, Mass.) were used for Western blot analysis. For inhibition assays, recombinant human TNF-R1:Fc fusion protein was purchased from Alexis (San Diego, Calif.). The general caspase inhibitor (Z-VAD-FMK), caspase-8 inhibitor (Z-IETD-FMK), and caspase-3 inhibitor (Z-DEVD-FMK) were purchased from R&D Systems (Minneapolis, Minn.). Herceptin was purchased form Genentech (South San Francisco, Calif.). The cell growth XTT assay kit was purchased from Roche Diagnostics Co. (Indianapolis, Ind.).

Cell Lines and Culture

SKBR-3 cells were grown in McCoy's 5A modified medium (DMEM, Life Technologies Inc., Rockville, Md.) supplemented with 10% heat-inactivated fetal bovine serum (FBS), 2 mM L-glutamine, 100 units/ml penicillin and 100 μg/ml streptomycin. SKBR-3 low passage cells expressing high amounts of HER-2/neu (SKBR-3-LP) used in our study were between passage 5 and 8 while the SKBR-3 high passage cells used were between passage 40 and 45 and displayed comparatively lower levels of HER-2/neu (SKBR-3-HP). The L3.6pl human pancreatic cancer cell line was kindly provided by Dr. Killian (M.D. Anderson Cancer Center, Houston, Tex.) and was grown in Dulbecco's modified Eagle's medium (DMEM, Life Technologies Inc.) supplemented with 10% heat-inactivated FBS, 100 units/ml penicillin and 100 μg/ml streptomycin.

In Vitro Cytotoxicity Assays

SKBR-3 cells were seeded (1×10⁴/well) in flat-bottom 96-well microtiter plates (Becton Dickinson Labware, Franklin Lakes, N.J.) and 24 hrs later scFv23, TNF, scFv23/TNF, or Herceptin (Genentech) were added in triplicate wells. To examine the effect of caspase inhibitor on the cytotoxicity of scFv23/TNF, SKBR-3-LP cells were pretreated with or without 200 μM general caspase inhibitor (Z-VAD-FMK), caspase-8 inhibitor (Z-IETD-FMK), or caspase-3 inhibitor (Z-DEVD-FMK) (R&D) for 2 hr and then treated with various concentration of scFv23/TNF. After 72 hr, 50 μl of XTT labeling mixture (Roche) was added to each well, after which the cells were incubated for another 4 hr. The spectrophotometric absorbance was measured at 450 nm using an ELISA reader (Bio-Tek Instruments, Inc., Winooski, Vt.).

Assessment of the Role of TNF-R1 in scFv23/TNF Cytotoxicity

SKBR-3 cells were seeded (1×10⁴/well) in flat-bottom 96-well microtiter plates (Becton Dickinson Labware, Franklin Lakes, N.J.) and 24 hrs later were pretreated with recombinant human TNF-R1:Fc fusion protein (Alexis) for 2 hr, and then treated with TNF, Herceptin (Genentech), or scFv23/TNF added in triplicate wells. After incubation for 72 hr, cell viability was detected by XTT assay (Roche).

Detection of Apoptosis

The development of apoptotic cell death was detected by DNA fragmentation and by TUNEL assay. To evaluate DNA fragmentation, SKBR-3-LP cells were seeded at 5×10⁵ cells/60 mm petri-dish, allowed to adhere overnight and then treated with 200 nM TNF or 200 nM scFv23/TNF. After 24 hr and 48 hr of exposure, cells were washed with PBS, resuspended in a DNA extraction buffer containing 5 mM Tris-HCl, pH 8, 50 mM EDTA, 10 □g/ml RNAse, and 0.25% SDS and then incubated for 1 hr at 37° C. To remove protein, resuspended cell lysates were treated with 100 μg/ml proteinase K for 3 hr at 500C. DNA was extracted using phenol and chloroform followed by ethanol precipitation. The genomic DNA was resuspended in Tris-EDTA (pH 8) and was fractionated by electrophoresis on a 1% agarose gel containing ethidium bromide.

To assess apoptosis using TUNEL assay, SKBR-3-LP cells were plated on glass cover slips, allowed to adhere overnight and then treated with 200 nM TNF or 200 nM scFv23/TNF for 24 hr and 48 hr. The cells were washed with PBS, permeabilized (0.1% Triton X-100, 0.1% sodium citrate), and then fixed in 4% paraformaldehyde. Fixed cells were stained with an in situ cell death detection kit (Roche). Cells undergoing apoptosis were identified by fluorescence microscopy (Nikon, Japan).

Western Blot Analysis

SKBR-3 and L3.6pl cell lines were seeded at 5×10⁵ cells/60 mm petri-dish, allowed to grow overnight, and then treated with 200 nM scFv23, 200 nM TNF, 200 nM scFv23/TNF or 10 mg/ml of Herceptin. After treatment, cells were washed twice with phosphate buffered saline (PBS) and lysed on ice for 20 min in 0.3 ml of lysis buffer (10 mM Tris-HCl, pH 8, 60 mM KCl, 1 mM EDTA, 1 mM DTT, 0.2% NP-40). Cell lysates (50 μg) were fractionated by 8-15% SDS-PAGE and electrophoretically transferred to Immobilon-P nitrocellulose membranes (Schleicher & Schuell Inc., Keene, N.H.). Membranes were blocked for 2 hrs in Tris-buffered saline (TBS) containing 3% bovine serum albumin and then probed with various antibodies (monoclonal anti-HER-2/neu Ab, rabbit polyclonal anti-TNF-R1 Ab, rabbit polyclonal anti-TNF-R2 Ab, rabbit polyclonal anti-caspase-8 Ab, monoclonal anti-caspase-3 Ab, monoclonal anti-PARP Ab, rabbit polyclonal anti-TRADD Ab, rabbit polyclonal anti-TRAF2 Ab, rabbit polyclonal anti-IκB-α Ab, rabbit polyclonal anti-phospho Akt Ab, and rabbit polyclonal anti-Akt Ab). Goat anti-mouse/goat anti-rabbit or swain anti-goat antibodies conjugated with horseradish peroxidase (Bio-Rad Laboratories, Hercules, Calif.) were used to visualize immunoreactive proteins at a 1:4000 dilution using ECL detection reagent (Amersham Pharmacia Biotech Inc., Piscataway, N.J.). Data are presented as the relative density of protein bands normalized to β-actin. The intensity of the bands was quantified using Histogram.

### Example 16

### Sensitivity to scFv23/TNF and Correlation with HER-2/Neu, TNF-R1, and TNF-R2 Expression

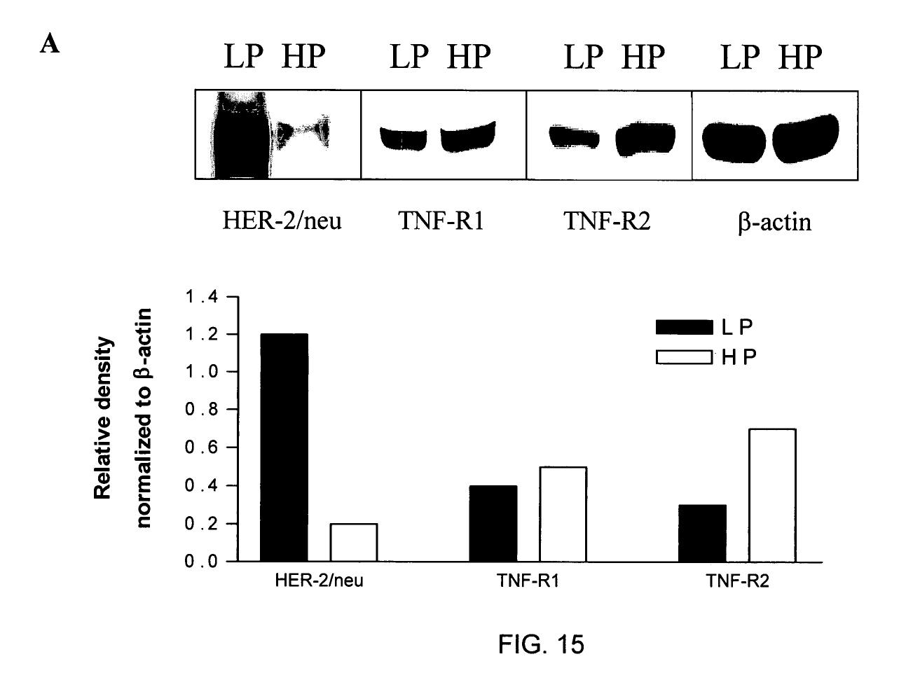

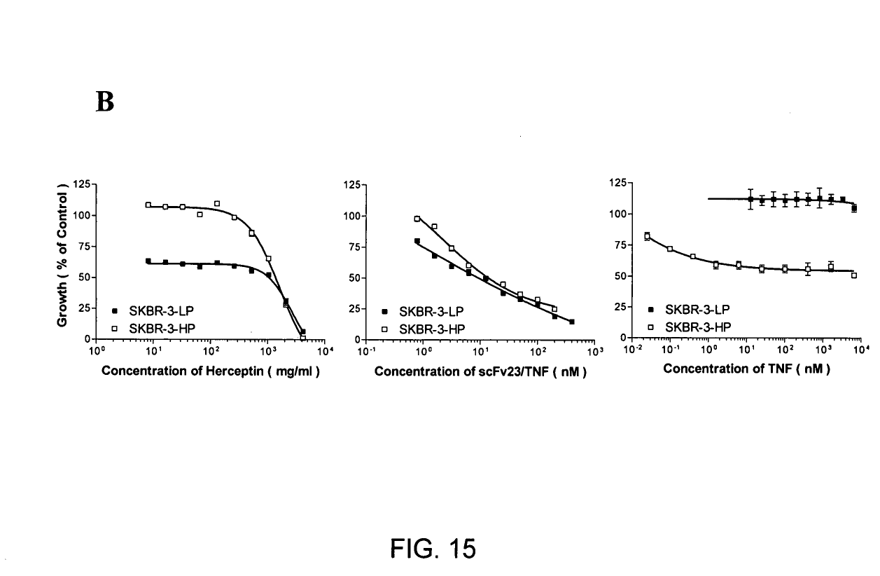

Studies in the inventor's lab have previously shown that the human breast cancer cell line SKBR-3 appears to down-regulate HER-2/neu cellular expression after prolonged passage in vitro. Western blot analysis (FIG. 15A) confirms that high passage cells (SKBR-3-HP, passage >40) express 6 fold lower levels of HER-2/neu compared to lower passage cells (SKBR-3-LP, less than passage 10). In addition, SKBR-3-HP cells also expressed 2.3 fold higher levels of TNF-R2 but equivalent levels of TNF-R1. The inventor next evaluated the response of these two cell lines to the cytotoxic effects of Herceptin, scFv23/TNF, or TNF. Compared to SKBR-3-HP cell lines expressing low levels of HER-2/neu, SKBR-3-LP cells expressing higher levels of HER-2/neu were more sensitive to the cytotoxic effects of Herceptin. On the other hand, SKBR-3-HP cells were more sensitive to the cytotoxic effects of TNF compared to SKBR-3-LP cells thus confirming previous studies suggesting that HER-2/neu over-expression correlates with resistance to TNF. In contrast, both SKBR-3 cell lines demonstrated virtually identical sensitivity to scFv23/TNF (FIG. 15B). These results indicate that continual culture of the SKBR-3 cell line results in down-regulation of HER-2/neu and a concomitant up-regulation of the TNF-R2. Studies by other groups (Sacca et al,. 1998; Amar et al., 1995) indicate that TNF-R1 is primarily responsible for mediating a TNF cytotoxic signal. It is unclear whether these observations are causally related or correlated with cellular resistance to TNF cytotoxic effects in SKBR-3 cells. However, these data indicate that the scFv23/TNF immunocytokine can overcome TNF cellular resistance associated with HER-2/neu over-expression. Furthermore, the significant differences observed in biological activity between scFv23/TNF and TNF itself on SKBR-3-LP cells afforded an opportunity to compare mechanistic pathways that may be responsible for these observations.

### Example 17

### The Role of TNF-R1 on scFv23/TNF-Induced Growth Inhibition

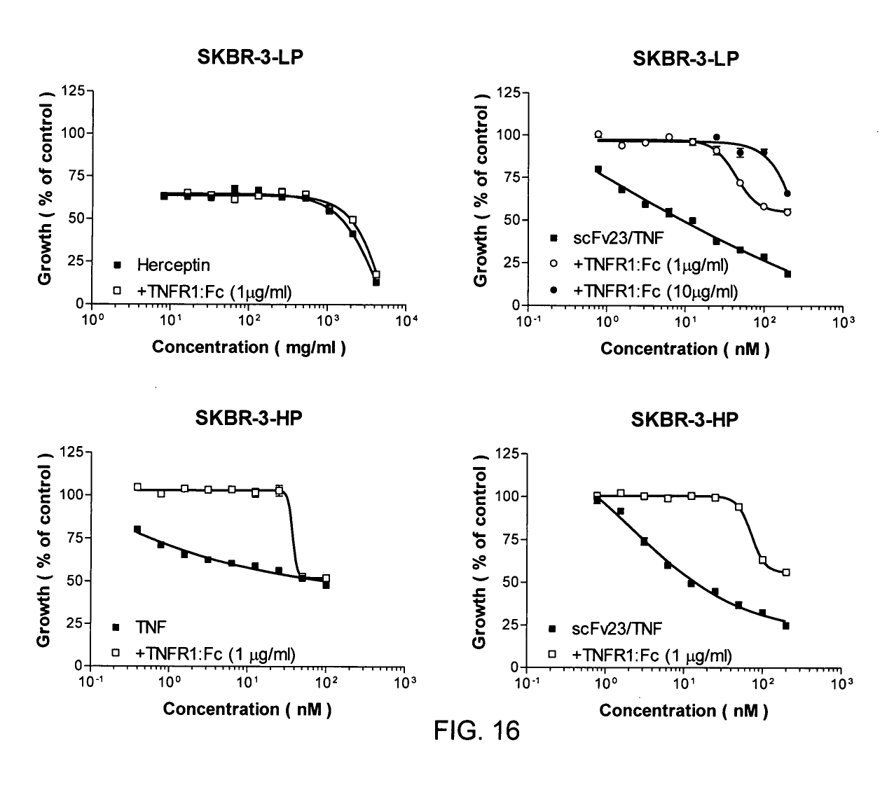

To determine whether the cytotoxic effects of the scFv23/TNF were mediated through interaction with the cell-surface TNF-R1, the inventor specifically blocked the binding of the TNF component of the scFv23/TNF fusion construct to TNF-R1 using TNF-R1:Fc fusion protein. As shown in FIG. 16, addition of TNF-R1:Fc was able to abrogate scFv23/TNF or TNF-induced cytotoxicity but not Herceptin-induced cytotoxicity on SKBR-3-LP or -HP cells. Inhibition of the cytotoxic effects of scFv23/TNF was directly dependent on the concentration of TNF-R1:Fc fusion protein added. The results indicate that in specific embodiments of the invention scFv23/TNF-induced cytotoxicity is mediated principally by interaction with the cell surface TNF-R1.

### Example 18

### Effect of scFv23/TNF on TNF-R Expression

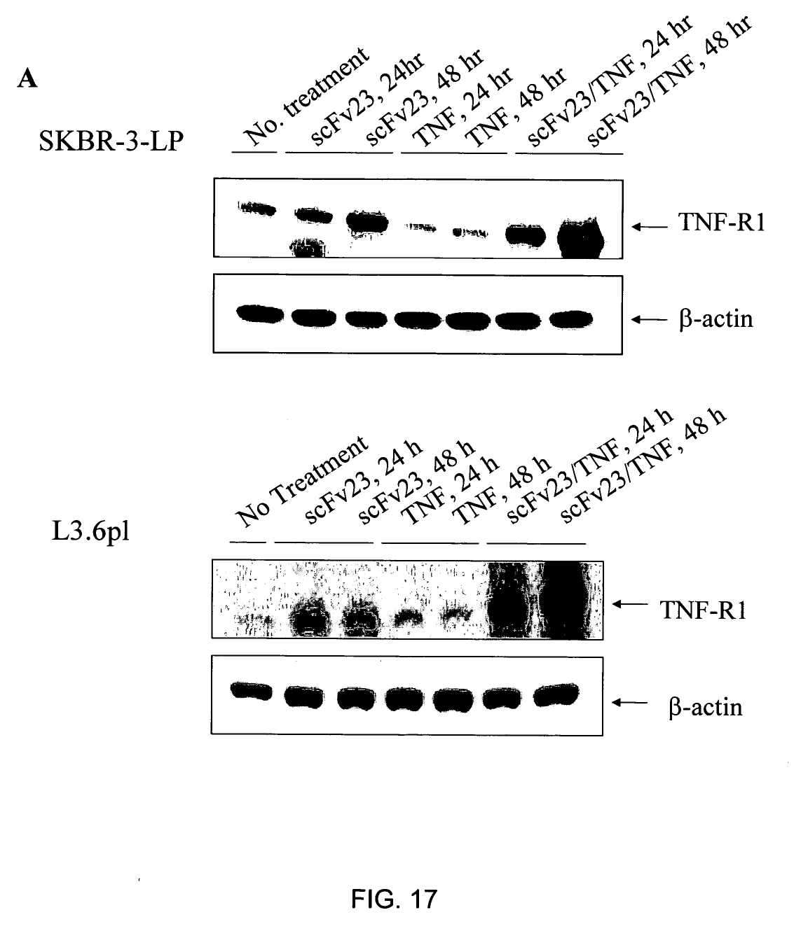

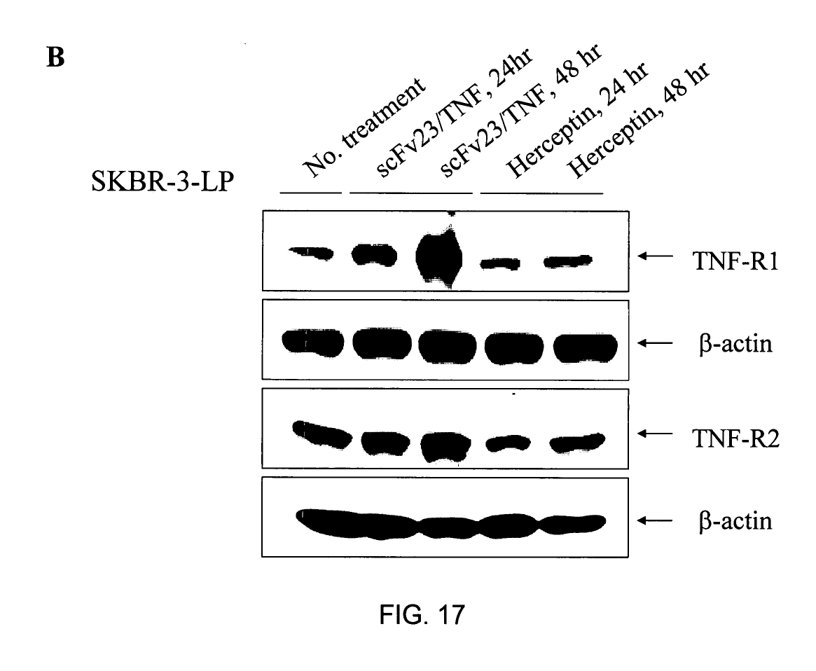

It was next examined whether the immunocytokine scFv23/TNF can modulate the cellular expression of TNF-R1. SKBR-3-LP cells were treated with scFv23, TNF, scFv23/TNF, or Herceptin. Treatment of SKBR-3-LP cells with either scFv23/TNF or scFv23 antibody alone induced up-regulation of TNF-R1 expression in a time-dependent fashion. This appeared to be an effect of the scFv23 component since TNF treatment reduced the levels of TNF-R1. Since scFv23/TNF treatment was found to induce a 5-fold increase in TNF-R1 expression in SKBR-3-LP cells, we next investigated whether the scFv23/TNF-mediated up-regulation of TNF-R1 was restricted to a particular type of tumor cell. The effect of scFv23/TNF on TNF-resistant, HER-2/neu-overexpressing L3.6pl human pancreatic cancer cells was examined. Treatment of L3.6pl cells with scFv23/TNF was also found to dramatically induce up-regulation of TNF-R1 in a manner identical to that found for SKBR-3-LP cells (FIG. 17A). These results indicate that the cellular expression level of TNF-R1 is directly correlated with scFv23/TNFcytotoxic effect in TNF-resistant, HER-2/neu-overexpressing tumor cell lines, in particular aspects of the invention.

In addition to the use of scFv23/TNF, it was next examined whether other HER-2/neu targeting molecules such as Herceptin can modulate the expression of TNF-R1 or TNF-R2 on HER-2/neu-overexpressing SKBR-3-LP cells. As shown in FIG. 17B, the inventor found that treatment with scFv23/TNF or Herceptin had no effect on the expression of TNF-R2, whereas Herceptin induced 1.8-fold and scFv23/TNF induced 7.3-fold higher expression of TNF-R1 compared to controls. This result indicates that TNF-R1 but not TNF-R2 expression and function is involved in TNF resistance in HER-2/neu-overexpressing SKBR-3-LP cells.

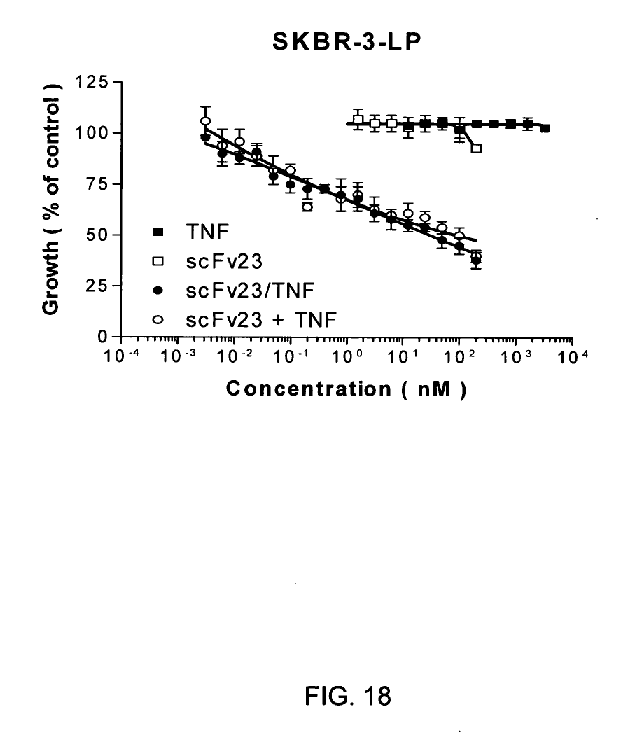

While the data showed that the anti-HER-2/neu single-chain antibody (scFv23) can induce up-regulation of TNF-R1, the effect of scFv23 on cell growth was examined. The inventor treated SKBR-3-LP cells with scFv23 alone or in combination with TNF and compared this with TNF or scFv23/TNF cytotoxicity. The combination of scFv23 and TNF was much more cytotoxic than TNF alone and was similar to the effect of the scF23/TNF fusion construct against TNF-resistant SKBR-3-LP (FIG. 18). Therefore, the results indicate that the induction of TNF-R1 expression by treatment with either scFv23 or scFv23/TNF plays a crucial role in regulating TNF sensitivity in HER-2/neu-overexpressing cancer cells.

### Example 19

### Effect of scFv23/TNF on Survival Pathways

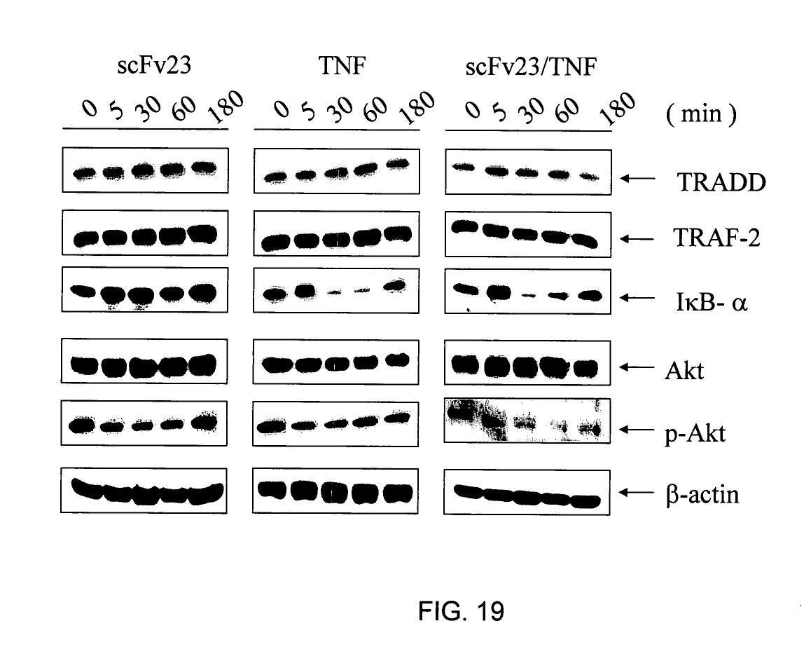

After binding to TNF-R1, TNF exerts dualistic biological functions by activating both survival pathways and apoptotic pathways. Activation of TNF-R1 results in an activation of NF-κB (degradation of IκB-α) and induction of NF-κB-regulated anti-apoptotic factors by a pathway including TRADD and TRAF2 (Wajant et al., 1999). To determine whether the TNF component of scFv23/TNF can activate anti-apoptotic pathways compared to native TNF, the inventor treated HER-2/neu-overexpressing SKBR-3-LP cells with 200 nM of scFv23, TNF, or scFv23/TNF for various times, harvested cells and subjected cell lysates to Western blot analysis. As shown in FIG. 19, treatment with scFv23/TNF for 180 min resulted in a modest decrease in TRADD. Treatment of cells with scFv23, TNF, or scFv23/TNF had no impact on the levels of TRAF2. On the other hand, after 30 minutes of treatment, IκB-α was degraded in both TNF- or scFv23/TNF-treated cells; whereas treatment with scFv23 had no effect on IκB-α degradation. This indicates an effect related to TNF component of the scFv23/TNF construct. Three hours after addition of either TNF or scFv23/TNF, levels of IκB-α appeared to increase back to basal levels. These results indicate that the IκB-α pathway may be involved in scFv23/TNF-mediated signaling transduction and this effect did not appear to be an effect of the scFv23 component of the scFv23/TNF construct.

Over-expression of HER-2/neu has been shown to result in activation of different downstream pathways such as Akt kinase pathway, which leads to cell proliferation and cell survival. To determine whether scFv23/TNF treatment affects the Akt survival pathway, SKBR-3-LP cells were treated with scFv23, TNF, or scFv23/TNF. The activation of Akt kinase was then assessed by Western blot analysis using antibodies to Akt and to phospho-Akt. As shown in FIG. 19, treatment with either scFv23 or TNF had no effect on the total cellular content or the phosphorylation Akt. On the other hand, treatment of cells with scFv23/TNF resulted in down-regulation of phosphorylated Akt after 30 min of drug administration. This indicates that scFv23/TNF can apparently modulate this survival pathway. This appears to be specific for the scFv23/TNF construct since neither scFv23 nor TNF treatment alone.

### Example 20

### Significance of scFv23/TNF Effects

Over-expression of HER-2/neu appears to be associated with a survival advantage and with TNF resistance in breast, ovarian, and HER-2/neu-transfected cell lines (Tang et al., 1994; Lichtenstein et al., 1990; Hudziak et al., 1988). On the other hand, down-regulation of HER-2/neu has been shown to confer enhanced sensitivity to the cytotoxicity of TNF in doxorubicin-resistant tumor cell lines (Sleijfer et al., 1998). Studies have additionally demonstrated that EGF signaling in breast and cervical carcinoma cells can also modulate the cytotoxic effects of TNF (Hoffmann et al., 1998).

In the study, scFv23/TNF composed of the anti-HER-2/neu single chain antibody fused to TNF can overcome HER-2/neu-induced TNF resistance in HER-2/neu-overexpressing SKBR-3-LP cells. This indicates that TNF-R1 expression, caspase activation, and Akt phosphorylation are three critical factors that contribute to scFv23/TNF-induced cytotoxicity in TNF-resistant HER-2/neu-overexpressing SKBR-3-LP cells.

First, a critical factor in the mediation of scFv23/TNF cytotoxicity appears to be modulation of TNF receptor-1. Amplification of the HER-2/neu oncogene has been shown to lead to resistance of NIH3T3 cells to TNF and this correlates with down-regulation of TNF receptor binding (Hudziak et al., 1988). The down-regulation of TNF-binding capacity by protein kinase C has also been shown to be associated with a decrease in TNF sensitivity (Unglaub et al., 1987). Therefore, the effect of scFv23/TNF on the expression of TNF-R1 was examined. scFv23/TNF could induce up-regulation of TNF-R1 in time-dependent fashion and the blocking of the binding of scFv23/TNF to TNF receptor-1 was able to abrogate scFv23/TNF-induced cytotoxicity, indicating that the immunocytokine, scFv23/TNF, sensitizes TNF-resistant HER-2/neu-overexpressing SKBR-3-LP cells to TNF via the modulation of TNF receptor-1. The TNF-mediated down-regulation of HER-2/neu in pancreatic tumor cells has been shown to be associated with an increase in TNF sensitivity (Kalthoff et al., 1993). In the invention, treatment of SKBR-3-LP cells with scFv23/TNF resulted in the inhibition of HER-2/neu phosphorylation at 48 hr of exposure whereas the treatment with TNF had no effect on the HER-2/neu phosphorylation (Data not shown). In specific embodiments, therefore, the down-regulation of HER-2/neu phosphorylation by scFv23/TNF leads to the up-regulation of TNF receptor-1.

Second, a critical factor in the mediation of scFv23/TNF cytotoxicity appeared to be the involvement of various caspases. TNF-induced apoptosis is mainly mediated by TNF-R1 (Tartaglia et al., 1993). Binding of TNF to TNF-R1 can induce the formation of signaling complexes, TNF-R1-TRADD-FADD-pro-caspase-8, resulting in the activation of caspase-8 (Nagat, 1997). The activation of caspase-8 is thought to result in proteolytic activation of the other caspases (Medema et al., 1997). The activation of caspase-3 contributes to paclitaxel-induced apoptosis in HER-2/neu-overexpressing SKOV3.1pl (Ueno et al., 2000) and immunotoxin-induced apoptosis (Keppler-Hafkemeyer et al., 1998). Treatment with scFv23/TNF resulted in activation of caspase-8, caspase-3, and PARP cleavage in a time-dependant manner. The data indicate that the scFv23/TNF-induced cytotoxic mechanism was accompanied by inducing the apoptotic cascade through activation of caspase-8, caspase-3, and PARP cleavage via TNF-R1.

Finally, another important factor in the mediation of scFv23/TNF cytotoxicity is the modulation of Akt phosphorylation. The serine/threonine protein kinase Akt has been shown to have a pivotal role in cell cycle progression (Brennan et al., 1997; Muise-Helmericks et al., 1998; Gille and Downward, 1999), angiogenesis (Jiang et al., 2000), inhibition of apoptosis (Sabbatini and McCormick, 1999; Zhou et al., 2000), and cell growth (Verdu et al., 1999). Over-expression of HER-2/neu is known to activate the Akt pathway and to confer resistance to apoptosis induced by many therapeutic drugs (Yu and Hung, 2000; Kneufermann et al., 2003). SKBR-3-LP cells which over-express HER-2/neu had endogenous levels of p-Akt and Akt. Treatment with either scFv23 or TNF had no effect on the total cellular content or the phosphorylation Akt. On the other hand, treatment of cells with scFv23/TNF resulted in down-regulation of phosphorylated Akt. The result indicates that Akt phosphorylation plays an important role in conferring a TNF-resistance on HER-2/neu-overexpressing SKBR-3-LP cells and scFv23/TNF-induced cytotoxicity may be mediated, at least in part, by the inhibition of Akt survival signaling pathway.

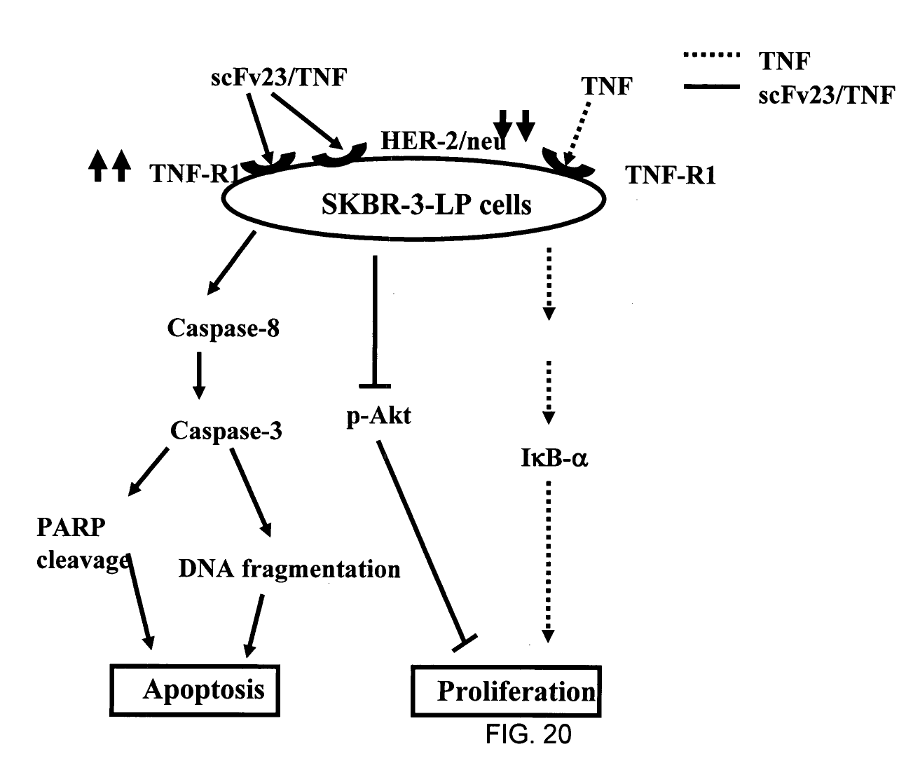

Taken together, the inventor observed that treatment of SKBR-3-LP cells with the immunocytokine scFv23/TNF resulted in up-regulation of TNF-R1 expression, down-regulation of Akt phosphorylation, and TNF-induced apoptosis through cleavage of caspase-8, caspase-3, and poly ADP-ribose polymerase. The observed differences in signaling events between TNF and scFv23/TNF are summarized in FIG. 20. The in vitro mechanistic studies indicate that the scFv23/TNF sensitizes TNF-resistant HER-2/neu-overexpressing SKBR-3-LP cells to TNF-induced apoptosis via the over-expression of TNF receptor-1, and in specific embodiments scFv23/TNF targeting the HER-2/neu may be an effective cytotoxic agent against HER-2/neu-overexpressing cancer cells that are inherently resistant to TNF. In addition, the immunocytokine scFv23/TNF was more cytotoxic than TNF itself against MCF-7 breast tumor cell lines expressing intermediate levels of HER-2/neu (Data not shown). In particular aspects of the invention, the scFv23/TNF immunocytokine can not only overcome TNF resistance in HER-2/neu-overexpressing cells but also it may be an excellent candidate for all breast cancer tumors, even those expressing modest amounts of HER-2/neu. In vivo pharmacokinetic, tissue disposition and xenograft therapeutic studies, for example, assist in the clinical development of this agent.

### Example 21

### Exemplary Materials and Methods for Examples 22-32

Cell Lines

The human melanoma cell lines A375-M and AAB-527 were obtained from Dr. I. J. Fidler (University of Texas M. D. Anderson Cancer Center, UTMDACC, Houston, Tex.) and Dr. B. Giovanella (the Stehlin Foundation, Houston, Tex.). A375-M cells were grown in Dulbecco's Modified Eagle's medium (DMEM) supplemented with 10% fetal bovine serum (FBS), sodium pyruvate (1 mM), non-essential amino acids (0.01 mM), glutamine (2 mM), MEM vitamins. AAB-527 cells were cultured in DMEM with 10% FBS, sodium pyruvate (1 mM). The human neuroglioma H4 cell was obtained from Dr. Bryant Darnay (UTMDACC). H4 cells were cultured in DMEM supplemented with 10% FBS, 4.5 g/L glucose. The human breast cancer cell line SK-BR3 was purchased from the American Tissue and Cell Culture Collection (ATCC, Rockville, Md.). The cells were grown in McCoy's 5A Medium Modified with 10% FBS, glutamine (2 mM) and maintained in log phase by passage twice weekly.

Expression and Purification of scFvMEL/TNF Fusion Proteins

The scFvMEL/TNF fusion gene was constructed using PCR-based construction methods. The fusion gene was finally cloned into a bacterial expression vector pET32a (+) and soluble fusion protein was expressed and purified as previously described (Mujoo et al., 1995). The final purified protein was stored at 4° C.

SDS-PAGE and Western Blot Analysis of Expressing scFvMEL/TNF Protein

Protein samples were analyzed by electrophoresis on a 10% SDS-PAGE under reducing conditions and visualized by staining with Coomassie Blue. Western analysis was performed using either rabbit anti-scFvMEL antibody (generated from the MDACC Core Facility) or using rabbit anti-huTNFa antibody (Sigma, St. Louis, Mo.), and then incubated with horseradish peroxidase (HRP)-labeled goat anti-rabbit IgG (1:5000 dilution), detected by enhanced chemiluminescence (ECL, Amersham Pharmacia Biotech) detection system and exposed to x-ray film.

Cytotoxicity Assay

The cytotoxicity of various agents against human cells in log-phase culture was assessed by crystal violet staining (Rosenblum et al., 1991). Briefly, cells were plated into 96-well plates at a density of 4×10³ cells per well and allowed to adhere for 24 h at 37° C. in 5% CO₂. After 24 h, the medium was replaced with medium containing different concentrations of either TNF or purifed scFvME/TNF. After 72 h, the effect of TNF and scFvMEL/TNF on the growth of tumor cells in culture was determined by crystal violet staining and the optical densities of the stained wells were measured at 595 nm using a 96-well multiscanner autoreader.

Determination of IκBα by Western Blot

Cells were treated with scFvMEL/TNF or TNF at various times, the cells were washed and lysed by lysis buffer containing 50 mM Tris, 150 mM NaCl, 5 mM EDTA, 100 mM DTT, 1% Triton X-100, 2 μg/ml leupeptin and 2 μg/ml apropnin. Equal amounts of total protein were loaded on 8.5% SDS-PAGE and standard Western blot assays were carried out and detected using anti-IκBα antibodies at 1:3000 dilution. The membranes were washed with PBST and treated with a secondary antibody conjugated to HRP. The antigen-antibody reaction was visualized by ECL detection system.

p38-MAPK Activation Assay

Cells were treated with doses of either scFvMEL/TNF or TNF for various times and the cells were harvested and lysed by lysis buffer. Equal amounts (50 μg total protein) were loaded onto 12% SDS-PAGE gels, and standard Western blotting was performed and detected using antibodies to MKK3, phospho-MKK3/MKK6, p38 MAP Kinase, phospho-p38 MAP Kinase (Thr180/Try182), ATF-2 or phospho-ATF-2 antibody (New England Biolabs) raised in rabbits (1:3000 dilution). Western blots were then visualized using HRP-goat anti rabbit IgG (1:5000 dilution) followed by ECL detection.

SAPK/JNK Activation Assay

Log-phase cells were treated with the same concentrations of TNF or scFvMEL/TNF for different times at 37° C. Cell lysates were extracted with cell lysis buffer. A 50-μg aliquot of protein was resolved on each lane on 10% SDS-PAGE, electrotransferred onto nitrocellulose membrane, and probed with rabbit polyclonal antibodies (1:3000 dilution) to MKK4, phosphospecific anti-SEK1 MKK4, SAPK/JNK, phosphospecific anti-p54/46 SAPK/JNK (Thr183/Tyr185), c-Jun or phospho-c-Jun (New England Biolabs). The membranes were then incubated with HRP-goat anti-rabbit IgG (1:5000 dilution), and bands were detected using an ECL detection system.

Analysis of PARP Cleavage

Briefly, 2×10⁶ cells/ml were treated with TNF for 24 h at the concentrations of 1 nM on A375-M and 200 nM on AAB-527, respectively, or treated with scFvMEL/TNF for 24 h at the concentrations of 0.1 nM for A375-M and 20 nM for AAB-527 cells. Following incubation, cell extracts were prepared by incubating cells for 30 min on ice in 0.05 ml of cell lysis buffer. A 50-μg protein sample from each supernatant was resolved by SDS-PAGE using a 7.5% gel and transferred onto a nitrocellulose membrane. The transferred proteins were probed with anti-PARP antibody (Roche Molecular Biochemicals, Indianapolis, Ind.) and detected by HRP-goat anti-mouse IgG and visualized by ECL. PARP degradation was represented by detection of both cleaved (86 kDa) and uncleaved (116 kDa) proteins recognized by the antibody.

Caspase-3 Activation

Cells were treated with scFvMEL/TNF or TNF at the I.C.₅₀ concentrations for various times (1 h, 4 h, 8 h, 16 h and 24 h). The same amount of total protein (50 μg) was then loaded onto 12% SDS-PAGE gels and a standard Western Blot was performed detected by a monoclonal antibody to cleaved caspase-3 (New England Biolabs), then further incubated with HRP-goat anti mouse IgG, detected by ECL detection system.

In Situ Cell Death Detection (TUNEL)

Cells (10,000 cell per well) in 16-well chamber slide (Nunc) were treated with scFvMEL/TNF or TNF at I.C.₅₀ for 24 h and washed briefly with PBS. Cells were fixed by addition of 3.7% formaldehyde at room temperature for 10 min, and permeabilized by 0.1% Triton X-100, 0. % sodium citrate on ice for 2 min. Cells were incubated with a TUNEL reaction mixture (Roche Molecular Biochemicals, Indianapolis, Ind.) at 37° C. for 60 min. After the final wash step, the cells were analyzed under a Nikon Eclipse TS-100 fluorescence microscope.

Detection of TNF Receptors and TNF Receptor Signaling Related Proteins on Melanoma Cells by Western Blotting

The same amount total proteins of cell lysate were loaded onto SDS-PAGE and standard Western blotting analysis was performed. The proteins were probed with primary antibodies to detect TNF R1, TNF R2, TRADD, TRAF2, RIP and β-actin (Santa Cruz Biotech, Santa Cruz, Calif.) then incubated with HRP-conjugated secondary antibody, finally detected with ECL detection system and exposed to X-ray film.

Internalization of scFvMEL/TNF

Cells were plated into 16-well chamber slide (1×10⁴ cells per well), and were pre-blocked for 1 h by addition of 25 μg/ml anti-TNFR1 antibody then treated with I.C.₅₀ concentrations of scFvMEL/TNF for 4 h, 1 h, etc. Cell surface binding was removed by exposure to glycine buffer (500 mM NaCl, 0.1 M Glycine, pH 2.5) and neutralized with 50 mM Tris, pH 7.5, followed by immunofluorenscence double staining. Briefly, cells were fixed with 3.7% formaldehyde and permeabilized with 0.1% Triton X-100 in PBS. After blocking with 0.5% BSA, cells were incubated with rabbit anti-hu TNF or rabbit anti scFvMEL antibody for 30 min at RT, then incubated with FITC-conjugate anti rabbit IgG (1:100 dilution, Sigma) containing propidium iodide (PI, 2.5 μg/ml) for 30 min at RT in dark. Samples were washed with PBS and air-dried. The slide was mounted with antifade DABCO mounting medium containing 1 mg/ml PI and analyzed under a Nikon Eclipse TS-100 fluorescence microscope.

RNA Preparation for Microarray Analysis

Human melanoma AAB527 cells were treated with scFvMEL/TNF or TNF at I.C.₅₀ concentrations for 24 h and untreated cells were used as a control. RNA was isolated from approximately 1×10⁷ cells with TRIzol Reagent (Life Technologies, Inc., Gaithersburg, Md.) according to manufactures instructions. The quality of total RNA was evaluated by denaturing formaldehyde/agarose gel electrophoresis. Microarray analyses were performed by the Cancer Genomics Core Lab and the Bioinformatics Section of the M. D. Anderson Cancer Center, Houston, Tex.

### Example 22

### Expression of the Plasmid Encoding scFvMEL/TNF Fusion Protein

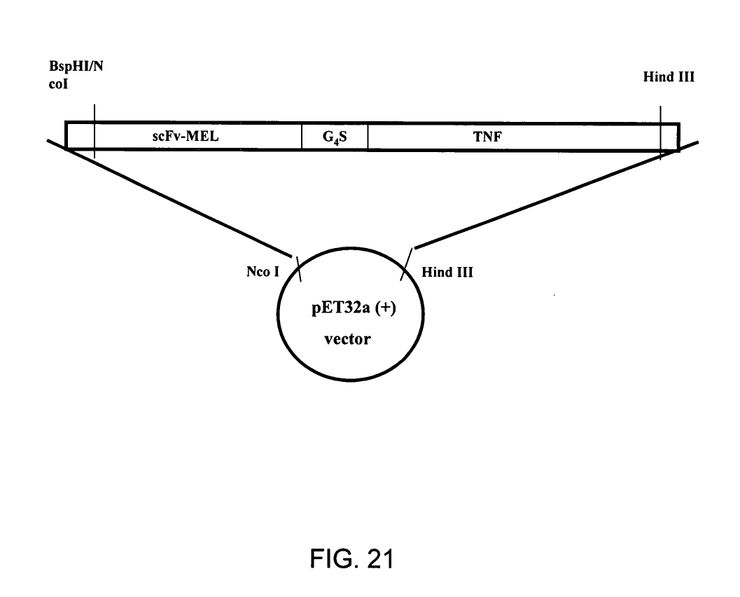

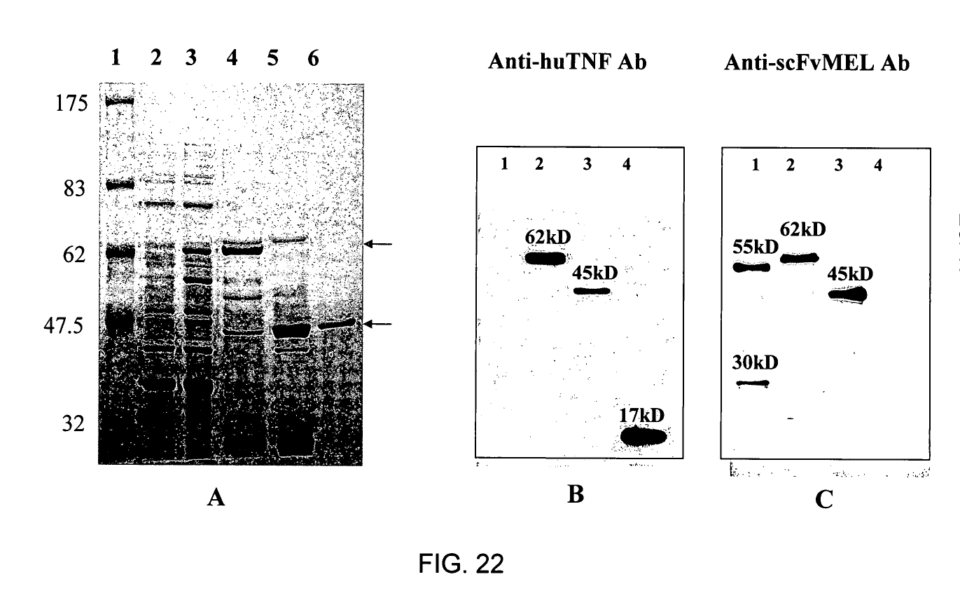

The scFvMEL/TNF fusion gene was constructed by PCR and ligated into the bacterial protein expression vector pET32 (FIG. 21). The fusion protein was expressed using E. coli strain AD494 (DE₃) plysS under the control of a T7 promoter, and synthesis of the target protein was induced by addition of IPTG. Soluble protein was purified by TALON-metal affinity chromatography and the His-tag was cleaved from the target protein by exposure to recombinant enterokinase (rEK). The fusion construct was then further polished through Q-Sepharose FF ion exchange chromatography. SDS-PAGE showed that Talon chromatography highly purified the target protein migrating at the expected molecular weight of 62 kDa. The 17 kDa tag was enzymatically cleaved leaving the native scFvMEL/TNF protein migrating at 45 kDa under reducing conditions (FIG. 22A). Composition of the final fusion protein was confirmed by Western blot using either rabbit anti-huTNF (FIG. 22B) or rabbit anti-scFvMEL antibodies (FIG. 22C). Structure of the construct was additionally confirmed by DNA sequence analysis.

### Example 23

### Cytotoxic Effects of scFvMEL/TNF and TNF in Melanoma Cells in Culture

The cytotoxicity of the scFvMEL/TNF was assessed against log-phase antigen-positive, TNF-sensitive human melanoma A375-M cells and antigen-positive, TNF-resistant human melanoma AAB-527 cells, respectively. These effects were compared with antigen-negative, TNF-sensitive human breast cancer SK-BR3-HP and antigen-negative, TNF-resistant human neuroglioma H4 cells. The results showed that against antigen-positive A375-M cells, scFvMEL/TNF (I.C.₅₀ 0.1 nM) appeared to be approximately 10 fold more active than native TNF (I.C.₅₀ 1.4 nM) (p<0.0001). Against TNF-sensitive, antigen-negative SK-BR3-HP cells, the cytotoxicity of scFvMEL/TNF (I.C.₅₀ 2.5 nM) showed a dose-response curve similar to that of authentic TNF (I.C.₅₀ 2.7 nM) (p>0.05). Against antigen-negative, TNF-resistant H4 cells, the scFvMEL/TNF demonstrated no cytotoxic effects at doses up to 100 nM. However against antigen-positive, TNF-resistant human melanoma AAB-527 cells, the scFvMEL/TNF showed significant dose-related cytotoxic effects (I.C.₅₀ 20 nM). In contrast, these AAB-527 cells were resistant to the cytotoxic effects of TNF at concentrations of up to 5000 nM (Table 7).

TABLE 7
Cytotoxicity^{a} of scFvMEL/TNF vs TNF on different cell lines

| | | I.C.50^{b} scFvMEL/TNF | I.C.50^{b} | Targeting | |
|---|---|---|---|---|---|
| Cell lines | Cell features | (nM) | TNF (nM) | Index^{d} | p Value |
| Human melanoma | Antigen gp240 (+) | 0.098 ± 0.013 | 1.370 ± 0.020 | 15 | <0.0001 |
| A375-M | TNF-sensitive | | | | |
| Human melanoma | Antigen gp240 (+) | 20.620 ± 1.125 | >5000^{c} | >250 | — |
| AAB-527 | TNF-resistant | | | | |
| Human breast | Antigen gp240 (−) | 2.549 ± 0.085 | 2.710 ± 0.014 | 1 | >0.05 |
| cancer SKBR3- | TNF-sensitive | | | | |
| HP | | | | | |
| Human | Antigen gp240 (−) | >100^{c} | >5000^{c} | — | — |
| neuroglioma H4 | TNF-resistant | | | | |

^{a}72 h treatment
^{b}data from 3 independent experiments
^{c}no cytotoxic effect at the Indicated concentrations
^{d}Targeting Index = IC50 of TNF/IC50 of scFvMEL/TNF

### Example 24

### Both scFvMEL/TNF and TNF Can Induce Degradation of IκBA

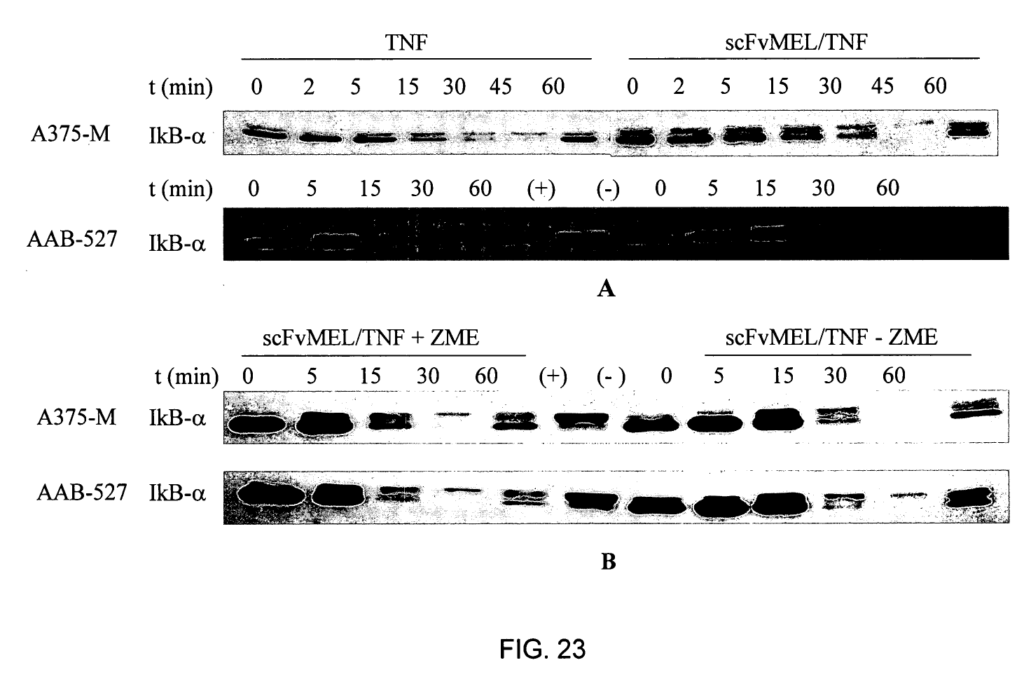

Western blot analysis was used to investigate the degradation of IκBα. NF-κB is present in its inactive state in the cytoplasm where it is bound to IκB-α. The degradation of IκB-α is critical for NF-κB activation and translocation to the nucleus. Western blot analysis of the levels of IκB-α in the cytoplasm before and after scFvMEL/TNF treatment showed that on A375-M and AAB-527 cells, scFvMEL/TNF treatment caused the degradation of IκB-α starting at 15 min. It was completely degraded at 30 min and was re-synthesized by 60 min. A similar profile was observed on these cells after treatment with TNF alone (FIG. 23A). Antibody ZME-018 is the parental murine antibody for the scFvMEL recombinant fragment. Both agents recognize the same antigenic domain on the gp240 target antigen presenting on human melanoma cell surface (Burger and Dayer, 2002; Boris and Steinke, 2003; Bharti and Aggarwal, 2002; Orlowski and Baldin, 2002; Sun and Andersson, 2002). When A375-M and AAB-527 cells were pretreated with ZME-018 for 4 h and then treated with scFvMEL/TNF and detected the levels of IκBα in the cytoplasm, antibody pre-treatment had no significant effect on scFvMEL/TNF induced degradation of IκBA (FIG. 23B).

### Example 25

### TNF and scFvMEL/TNF Effects on the P38-Map Kinase Pathway

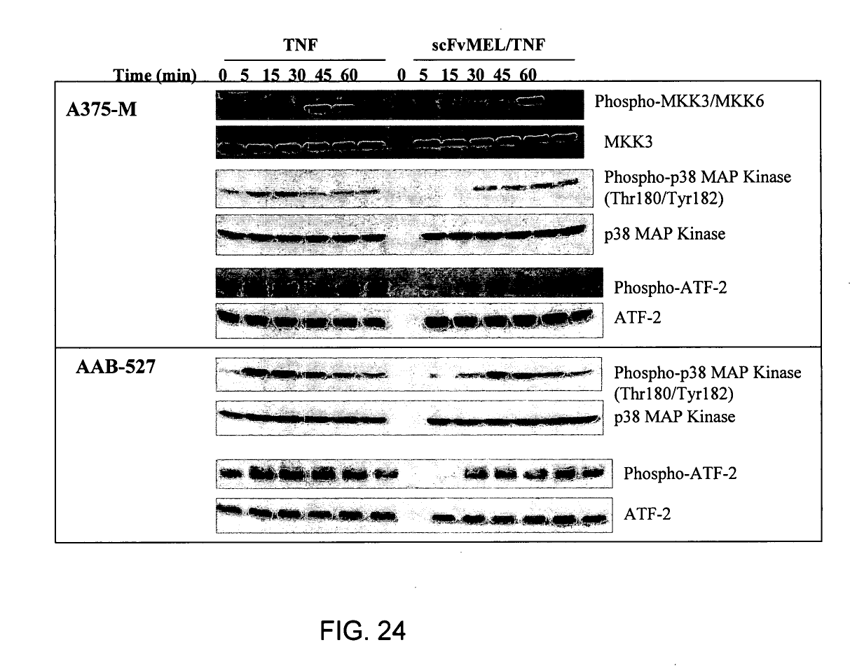

The p38 MAP kinase pathway was activated by both scFvMEL/TNF and TNF exposure on either A375-M or AAB527 cells. The activation events observed with scFvMEL/TNF treatment occurred somewhat later than that observed with TNF treatment. MKK3 was activated by TNF by 30 min and by scFvMEL/TNF at 45 min on A375-M cells. MKK3 activates p38 MAP kinase by phosporylation at Thr 180 and Tyr 182. Activated p38 MAP Kinase has been shown to phosphorylate the transcription factor ATF-2, etc. (FIG. 24).

### Example 26

### SAPK/JNK Pathway Activation by TNF but Not by scFvMEL/TNF

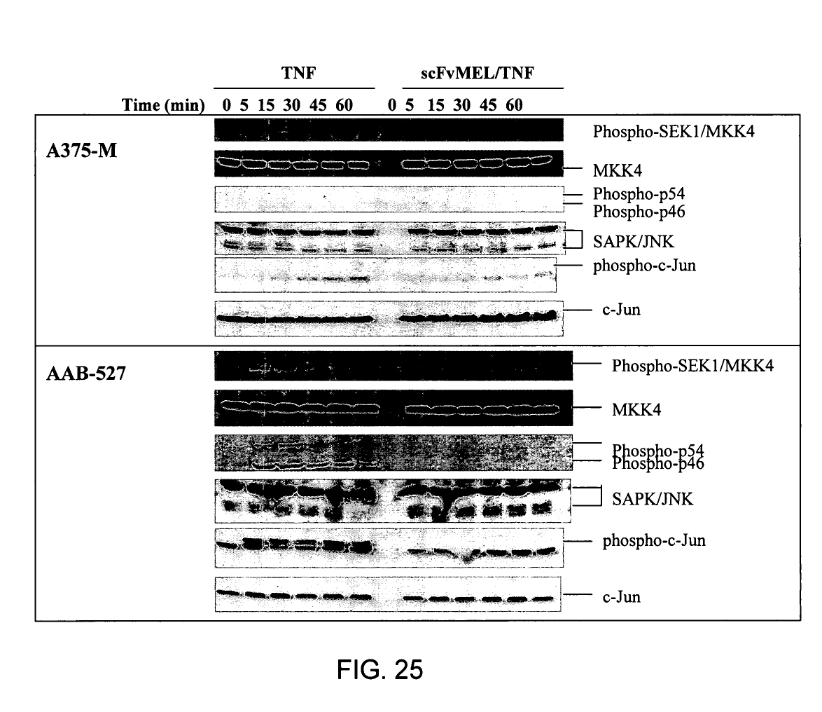

Exposure to TNF rapidly stimulated SAPK/JNK activation in both human melanoma A375-M and AAB-527 cells. In AAB527 cells, MKK4 was phosphorylated by TNF treatment starting at 5 min after exposure. Activated MKK4 was shown to activate SAPK/JNK by phosporylation at Thr 183 and Tyr 185 displaying cytoplasmic p54/p46 also at 5 min. The activated SAPK/JNK binds to the N-terminal region of c-Jun transcription factors and resulted in phosphorylation of c-Jun. TNF induced phosphorylation signals of p54/p46 were much lower in TNF sensitive A375-M than in TNF resistant AAB527 cells. However, when either AAB-527 or A-375M melanoma cells were treated with scFvMEL/TNF, no phosphorylation of MKK4, SAPK/JNK or c-Jun was observed (FIG. 25).

### Example 27

### Treatment with scFvMEL/TNF but Not TNF Induces Apoptosis on Antigen-Positive, TNF-Resistant Melanoma Cells

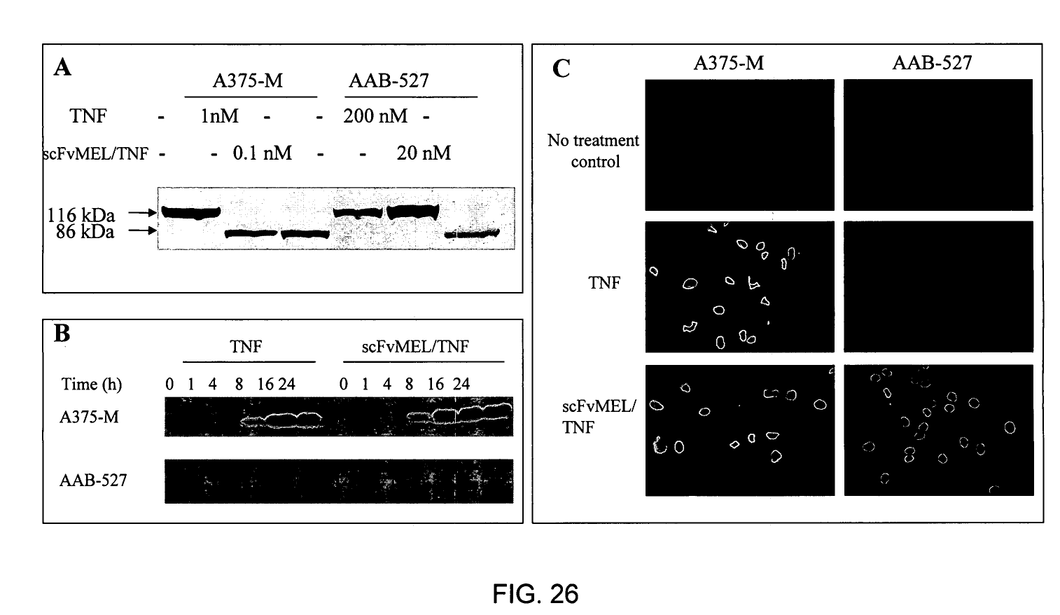

Antigen-positive, TNF-sensitive A375-M melanoma cells, when treated with either TNF (1 nM) or scFvMEL/TNF (0.11 nM) for 24 h, were growth inhibited and demonstrated PARP cleavage. In contrast, TNF-resistant AAB-527 melanoma cells, when treated with TNF at concentrations up to 200 nM for 24 h demonstrated no PARP cleavage. In contrast 24 hr after treatment of these cells with scFvMEL/TNF (20 nM), PARP cleavage was clearly observed (FIG. 26A). Treatment with both TNF and scFvMEL/TNF resulted in activation of caspase-3 on A375-M cells after 4 h treatment. Treatment with scFvMEL/TNF but not TNF resulted in caspase-3 activation in AAB-527 cells after 8 h treatment (FIG. 26B). TUNEL assay demonstrated apoptotic nuclei in either scFvMEL/TNF or TNF treated A375-M cells after 24 h, whereas positive apoptotic nuclei were only found on AAB-527 cells treated with scFvMEL/TNF but not after treatment with TNF (FIG. 26C).

### Example 28

### Both Human Melanoma A375-M and AAB-527 Cells Express TNF Receptors and TNF Receptor Signaling-Related Proteins

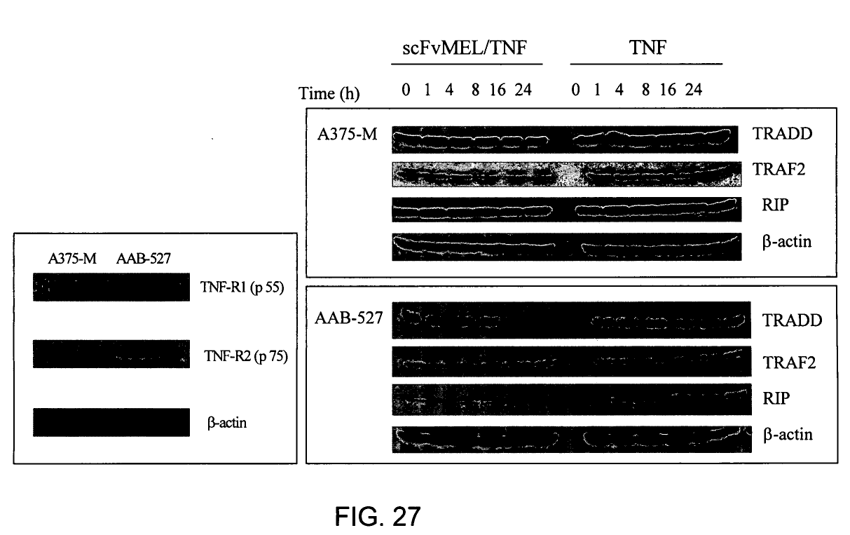

Both TNF R1 and TNF R2 were detected on A375-M and AAB-527 by Western blotting. TRADD, TRAF2 and RIP were also detected on melanoma cells. The levels of TRAF2 decreased when A375-M cells were treated with scFvMEL/TNF after 16 h and TNF after 24 h. The Western results showed that TRADD and RIP decreased on AAB-527 cells treated with scFvMEL/TNF after 16 h, but no significant changes were observed when those cells were treated with TNF (FIG. 27).

### Example 29

### Effect of Neutralizing Anti-TNFR1 Antibodies on the Cytotoxicity of scFvMEL/TNF on A375-M Cells

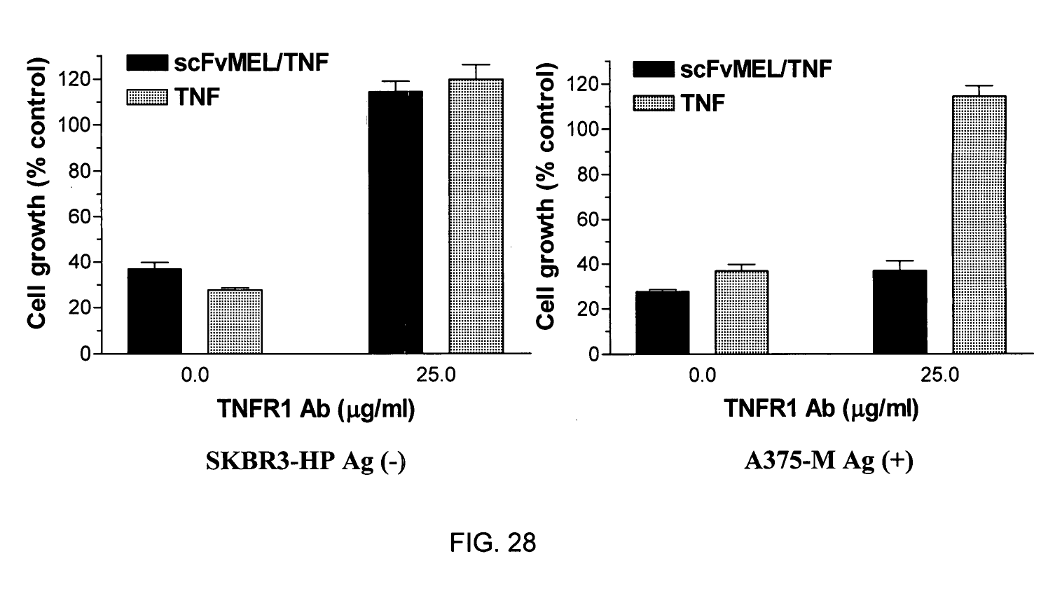

An anti-TNFR1 antibody (25 μg/ml, Alexis Biochemicals) was used to neutralize TNF receptor-induced cytotoxicity on either antigen-positive, TNF-sensitive A375-M cells or antigen-negative, TNF-sensitive SKBR3-HP cells. The results (FIG. 28) showed that anti-TNFR1 can neutralize TNF induced cytotoxicity on either SKBR3-HP (100%) or A375-M cells (100%). Importantly, anti-TNFR1 can neutralize the cytotoxicity of scFvMEL/TNF on SKBR3-HP (100%), but was unable to neutralize the cytotoxicity of scFvMEL/TNF on A375-M. This indicates that the cytotoxic effects of the scFvMEL/TNF construct observed on target cells may not occur solely through an interaction with the TNFR1 receptor.

### Example 30

### Internalization of scFvMEL/TNF into Antigen-Positive A375-M Cells as Assessed by Confocal Microscopy

The TNF moiety of scFvMEL/TNF was effectively delivered into the cytosol of A375-M cells after treatment with scFvMEL/TNF for as little as 1 h exposure as shown by immunofluorescence microscopy. The fluorescent signal intensity in cytosol increased after exposure for 4 h and remained constant for up to 24 h thereafter.

### Example 31

### Effects of scFvMEL/TNF and TNF Assessed by Microarray Analysis

Analysis of cDNA microarray was employed to identify genes exhibiting differential expression between samples of untreated TNF-resistant AAB527 cells and those treated for 24 h with either TNF or scFvMEL/TNF. Array analysis of 2500 genes indicated that there were 67 genes down regulated and 63 genes up regulated by both scFvMEL/TNF and TNF treatment. In addition, 155 genes were down regulated and 132 genes up regulated uniquely by scFvMEL/TNF. Those identified genes are primarily involved in cell-surface receptor-linked signaling, intracellular signaling cascade events, stress responses and intracellular protein trafficking and transport. The scFvMEL/TNF fusion protein was shown to down-regulate specific genes involved in cell cycle and cell proliferation as well as genes regulating nucleotide metabolism (Table 8).

TABLE 8
Genes down-regulated or up-regulated by scFvMEL/TNF but
not by TNF on AAB-527 cells after microarray analysis

| Genes down-regulated by scFvMEL/TNF only | Genes up-regulated by scFvMEL/TNF only |
|---|---|
| Cell surface receptor linked signaling | |
| Human putative endothelin receptor type B-like | Apoptosis inducing protein (TRICK2B) |
| protein | (TNFRSF10B) |
| Human rag A protein mRNA | Putative chemokine receptor (GTP-binding |
| | protein HM74) |
| Guanine nucleotide binding protein beta | Regulator of G-protein signaling 16 |
| polypeptide 1 (GNB1) | mRNA(RGS16) |
| Platelet-derived growth factor alpha polypeptide | Insulin-like growth factor 2 (somatomedin |
| (PDGFA) | A) (IGF2) |
| Intracellular signaling cascade | |
| JNK activating kinase I | Signal transducer and activator of |
| | transcription 4 (STAT4) |
| Signal transducer and activator of transcription- | Human protein kinase (MLK-3) mRNA |
| α/β (STAT1) | (MAP3K11) |
| Human c-jun proto oncogene | Human Rad mRNA |
| Human Hou mRNA (NMI) | Formyl peptide receptor 1 (FPR1) |
| Cell division cycle 42 (GTP-binding protein 25 kD) | |
| Cell cycle and cell proliferation | |
| RFC4 and RFC1 | V-myb avian myeloblastosis viral ncogene |
| | homolog-like 2 |
| VEGF | Replication factor C (activator 1) 3 (38 kD) |
| | (RFC3) |
| Cyclin G2 (CCNG2) | Human kinase suppressor of ras-1 (KSR1) |
| Cell division cycle 27 | |
| Cell division cycle 2 G1 to S and G2 to M | |
| (CDC2) | |
| Stress response | |
| C4/C2 activating component of Ra-reactive | |
| factor (MASP1) | |
| Major histocompatibility complex enhancer- | Human tumor necrosis factor type I receptor |
| binding protein MAD3 (NFKBIA) | associated protein (TRAP1) mRNA |
| Human transcription factor NFATx mRNA | |
| (NFATC3) | |
| Intracellular protein traffic and transport | |
| Human ubiquitin-related protein SUMO-1 | Human TBP-associated factor TAFII80 |
| mRNA | mRNA |
| Human (HepG2) glucose transporter gene | RAB31 member RAS oncogene family |
| | mRNA |
| Solute carrier family 2 (facilitated glucose | |
| transporter) member3 | |
| Nucleobase metabolism | |
| Transcription elongation factor S-II (TCEA1) | SRF accessory protein 1 B (SAP-1) (ELK4) |
| Human histone stem-loop binding protein | Human Dnase 1-like III protein (DNAS1L3) |
| (SLBP) | mRNA |
| Uridine monophosphate synthetase (UMPS) | |
| Ubiquitin-conjugating enzyme E2B and E2A | |
| (UBE2B) (UBE2A) | |
| Methyl-CpG binding domain protein 1 (MBD1) | |
| Beta catenin gene (CTNNB1) | |
| Cell adhension | |
| | Cadherin 3 (P-cadherin) |
| | Integrin beta 8 and integrin beta 4 subunit |

### Example 32

### Significance of Overcoming TNF Resistance by scFvMEL/TNF

Malignant melanoma is a primary example of a cancer that is highly metastatic and that responds poorly to various treatments, including chemotherapy and γ-irradiation (Bian et al., 2002). The present inventors previously reported (Mujoo et al., 1995) a fusion construct designated scFvMEL/TNF composed of the antibody scFvMEL that recognizes the surface domain of the gp240 antigen present on 80% of human melanoma cell. The antibody-specific delivery of TNF to the cell surface of melanoma cells resulted in augmented cytotoxicity compared to TNF alone. In addition, the present inventors demonstrated that antibody-TNF chemical conjugates and fusion constructs were capable of delivering TNF to tumors in vivo (Tamanini et al., 2003). Moreover, the chemical conjugate of TNF was highly cytotoxic to melanoma cells resistant to TNF (Rosenblum et al., 1991; Tamanini et al., 2003). However, the present inventors confirm that scFvMEL/TNF demonstrates cytotoxicity against human melanoma cells resistant to TNF alone and was more active against sensitive cells compared to native TNF. As expected, no differences were identified between the cytotoxicity of scFvMEL/TNF and that of TNF on antigen-negative cells.

Mechanistic studies were undertaken to identify signaling events important to understanding how scFvMEL/TNF is capable of overcoming TNF cellular resistance. Previous studies (Adams and Schier, 1999) have demonstrated that a key signaling event in TNF-induced cytotoxicity is activation of NF-κB and MAP kinase activation leading to the induction of apoptosis. Under normal conditions, NF-κB is present in the cytoplasm in its inactive state as a heterotrimer consisting of p50, p65, and IκB-α. Upon activation, several growth-regulatory genes such as ICAM-1, VCAM-1, matrix metalloprotease-9, cIAP2 (cellular inhibitor for apoptosis) are all regulated by NF-κB activation (Lyu et al., submitted; Minami et al., 2003; Harimaya et al., 2000; Schoemaker et al., 2002). Therefore, NF-κB appears to be a central regulator of homeostasis and a potential target for cancer drug development (Lin, 2003; Xia et al., 1995). However, the activation of NF-κB is initiated by a wide variety of stress stimuli, which themselves can cause apoptosis (Marti et al., 1997). The present inventors demonstrated that scFvMEL/TNF and TNF both induced the degradation of IκB-α on both TNF-sensitive and TNF-resistant human melanoma cells within 30 min. Moreover, the adaptor proteins for NF-κB activation such as TRAF2 or RIP were both decreased on A375-M cells after treatment with scFvMEL/TNF or TNF for 16 h or on AAB-527 cells treated with scFvMEL/TNF after 16 h, respectively. This suggests that an early and transient NF-κB activation can be induced by TNF even in resistant cells. The activation of NF-κB by scFvMEL/TNF was found to occur through TNF ligand-receptor interaction since co-administration of ZME-018 with scFvMEL/TNF was unable to prevent activation of IκB-α. Studies have suggested that NF-κB transcription factors can both promote cell survival and induce apoptosis depending on cell type, and that NF-κB activation and apoptosis are directly linked, however, constitutive activation of NF-κB can cause resistance to apoptosis (Hehlgans and Mannel, 2002). Therefore, apoptosis induced by scFvMEL/TNF was compared with native TNF on human melanoma cells in relation to caspase-3 and PARP cleavage. PARP is a substrate of caspase 3 (CPP-32) and specific cleavage of PARP is a hallmark of cellular apoptosis (Lippke et al., 1996). The scFvMEL/TNF construct induced PARP cleavage and apoptosis (TUNEL) in both TNF-sensitive and TNF-resistant human melanoma cells. However, native TNF induced PARP cleavage only in TNF-sensitive but not in TNF-resistant melanoma cells. This indicates that the fusion construct can overcome TNF resistance, in part, through signaling related to the apoptotic pathway.

The mechanisms of cytotoxicity of the scFvMEL/TNF fusion protein were further characterized by investigating the activation of the p38MAP kinase and SAPK/JNK survival pathways. Even though both scFvMEL/TNF and TNF activated the p38 MAP kinase pathway, TNF rapidly induced the phosphorylation of cytoplasmic MKK4 early (within 5 min), resulting in activation/phosphorylation of SAPK/JNK. Moreover, as expected, the TNF-induced phosphorylation of SAPK/JNK was higher in TNF-resistant melanoma cells than in TNF-sensitive cells. On the other hand, treatment with the scFvMBL/TNF construct resulted in reduced activation of the SAPK/JNK pathway suggesting that activation of SAPK/JNK survival pathway might contribute to the observed cellular resistance to TNF cytotoxicity by increasing cell survival with TNF exposure. These studies strongly indicate that cytotoxicity of scFvMEL/TNF fusion construct differs from that of TNF primarily in its effects on SAPK/JNK survival signaling.

The activation of opposing pathways is well-known in the TNF literature (Lin, 2003). In several cell lines, exposure to TNF results in the activation of the SAPK/JNK cascade and the initiation of programmed cell death. Studies by Marti et al. (Marti et al., 1997) suggest that SAPK can significantly affect TNF-induced signaling in human melanoma cell lines although up-regulation of the JNK/SAPK pathway is associated with a variety of effects that are largely determined by the cell type and situation. In specific embodiments of the present invention, reduction in the SAPK/JNK activity resulting in apoptosis is an important mechanism by which the fusion construct demonstrates improved cytotoxicity against TNF-sensitive cells as well as activity against TNF-resistant cells.

In specific embodiments of the present invention, such distinct differences in signaling events between TNF and the exemplary fusion constructs described herein is that the construct could interact with the TNFR1 surface receptor in a manner somewhat different from that of TNF. The present inventors showed that anti-TNFR1 antibodies can efficiently neutralize TNF induced cytotoxicity on either antigen negative or antigen positive cells. Although this neutralizing antibody neutralized the cytotoxicity of scFvMEL/TNF on antigen negative cells, it was unable to neutralize cytotoxicity on antigen positive cells. Alternatively, this data may suggest that the cytotoxic effects of scFvMEL/TNF may not completely occur through interaction with the cell surface TNF receptor. Internalization studies of scFvMEL/TNF showed that the TNF moiety of the fusion construct was delivered into the cytosol of human melanoma cells after exposure for 1 h. Given the different effects on cytotoxicity, intracelluar signaling and the unique profile of genes induced by scFvMEL/TNF compared to native TNF, one possible explanation for these differential effects is that TNF delivered to the intracellular compartment might be able to interact with intracelluar TNFR1 or other intracellular proteins to transduce a unique signal. This embodiment is supported by the observation that many cells (including A-375 and AAB527 melanoma cells) display high intracellular pool levels of TNFR1 (Hehlgans and Mannel, 2002) and that the microarray analysis identified unique genes regulated by the scFvMEL/TNF fusion construct compared to TNF.

Thus, the scFvMEL/TNF fusion protein was more cytotoxic on melanoma cells in vitro compared with native TNF alone and is cytotoxic against cells resistant to TNF. The fusion construct simultaneously induced apoptotic events and down-regulated SAPK/JNK survival pathways. This is distinct from the actions of TNF itself that induced both survival and apoptotic events. The antibody-mediated delivery of TNF to cells activates numerous different intracellular pathways compared to TNF alone, as assessed by microarray analysis, and in specific embodiments of the present invention, this difference is due at least in part to interaction with the TNF receptors in a manner different from that of TNF.

### Example 33

### Exemplary Materials and Methods for Examples 34-36

Cell Lines

A375-M (human melanoma, gp240 antigen positive, TNF-sensitive), AAB-527 (human melanoma, gp240 antigen positive, TNF-resistant), SKBR3-HP (human breast cancer, gp240 negative, TNF-sensitive), H4 (human neuroglioma, gp240 negative, TNF-resistant) were maintained in culture using Dulbecco's MEM (DMEM) with 10% fetal bovine serum (FBS) containing antibiotics (0.05 mg/ml), added glutamine (200 mM) and sodium pyruvate (100 mM) for AAB-527 and A375-M specifically, and also added nonessential amino acids (10 mM) and MEM vitamins for A375-M cells specifically. Tissue culture media and supplements were purchased from Life Technologies Inc., (Rockville, Md.).

Transfection and Stable A375GFP Cell Lines

The plasmid pcDNA3-EGFP was produced by inserting Hind III/XhoI fragment containing the enhanced green fluorescent protein (EGFP) coding sequence from pCMV-EGFP into the same sites of pcDNA3.1. Cells were cultured for 24 h in six-well plates with 1 ml/well of DMEM medium with 10% FBS until 60-70% confluence was reached. The liposomal DNA (Lipofectamine-pcDNA3-GFP complex) or nonliposomal DNA (pcDNA3) was directly added into the culture plates at a ratio of 2 μg of DNA/10⁶ cells. To produce A375 cells that stably express EGFP, G418 (400 μg/ml) selection was started 24 h after transfection. After 10 days of selection with G418, the surviving cells were examined by fluorescence microscopy. Fluorescent colonies were picked and expanded. Cellular expression of GFP was evaluated using a fluorescence/visible light microscope set-up to directly assess the percentage of total cells fluorescing.

Production of scFvMEL/TNF, scFvMEL, and Recombinant Human TNF

The scFvMEL/TNF fusion gene was constructed using PCR-based construction methods. The fusion gene was finally cloned into a bacterial expression vector pET32a (+) and soluble fusion protein was expressed and purified as previously described (Liu et al., 2004).

Human recombinant TNF gene from plasmid pET32scFvMEL/TNF was subcloned into pET32a (+) vector and protein was expressed in E. coli Origami (DE3) (Novagen, Madison, Mich.). The expression and purification of TNF were the same as that of the fusion protein scFvMEL/TNF. The biologic activity of TNF was determined by using a standard assay, which depends on cytotoxicity in L-929 murine fibroblast cells as previously described (Rosenblum et al., 1991). The rHuTNFα from Roche Molecular Biochemicals (Indianapolis, Ind.) was used as a standard. The biological activity of the final purified huTNF was 2×10⁷ U/mg protein.

The scFvMEL gene was amplified by PCR from plasmid pET32scFvMEL/TNF and the genes were cloned into pET21b vector and formed plasmid pET21scFvMEL. The protein scFvMEL was expressed in E. coli AD494 (DE3) plysS. The biologically functional scFvMEL protein obtained by refolding from inclusion body based on a recipe by Steinle A, et al. (Steinle et al., 2001). The final purified scFvMEL protein has the specific binding activity detected by ELISA.

In Vitro Cytotoxicity of scFvMEL/TNF and TNF

To examine the cytotoxicity of scFvMEL/TNF and TNF, cells were plated into 96-well plates at a density of 3000 cells per well and allowed to adhere for 24 h at 37° C. in 5% CO₂. After 24 h, the medium was replaced with medium containing different concentrations of either scFvMEL/TNF or TNF. The effect of scFvMEL/TNF and TNF on the growth of tumor cells in culture was determined by crystal violet staining as previously described (Rosenblum et al., 1991). Cell plates were read at 630 nm (Bio-Tek Instruments, Winooski, Vt.). The absorbance was compared with control wells (medium alone).

Protein Labeling Using P-Iodobenzoate

Proteins were labeled with ¹²⁵I (Dupont, Wilmington, Del.) by the P-iodobenzoate method as previously described (Rosenblum et al,. 1995).

Animal Model Studies

Pharmacokinetic Study

BALB/c female mice, 4-6 weeks old, were injected with 2 μCi per mouse, 5 μg total protein in 200 μl of normal saline. 1, 2, 4, 8, 24, 48, 72 h after injection, two mice at each assay time were sacrificed by cervical dislocation. Blood samples were removed from chest cavity, weighed and counted to determine total radioactivity in a gamma counter (Packard, model 5360). The blood samples were also centrifuged and plasma was decanted and counted to determine radioactivity. Results from plasma determinations of radioactivity were analyzed by a least-square nonlinear regression (PK Analyst from MicroMath, Inc.) program.

In Vivo Toxicity Study

Five groups of female BALB/c mice (4-6 weeks old, 5 mice per group) were injected (i.v., tail vein) once daily for 5 days with either saline (vehicle control, Group 1) or with four different doses of drug (Groups 2-5). The total dose delivered in each group was 1, 2, 3, and 4 mg/kg which corresponded to 25, 50, 75, and 100% of an established maximum tolerated dose (MTD). Seven days after the last injection (Day 12), animals on study were sacrificed with carbon dioxide, terminally bled for hematological parameters including a complete blood count (CBC) and clinical chemistry analysis including Total Bilirubin, Phosphorus, AST (SGOT), ALT (SGPT) Total protein, Albumin, Globulin, Calcium, Sodium, Potassium, Chloride, Alk Phosphatase, Creatinene, BUN, and subjected to a complete necropsy including heart, lungs, spleen, kidneys, and liver, etc. were fixed by immersion in neutral-buffered 10% Formalin solution. The tissues were embedded in paraffin blocks from which 2- to 4-μm sections were cut and stained with H & E, which were carried out by the Department of Veterinary Medicine and Surgery of the University of Texas M. D. Anderson Cancer Center.

In Vivo Efficacy Study

BABL/c nude (nu/nu) mice, 4-6 weeks old, were injected with 3×10⁶ A375GFP log-phase melanoma cells subcutaneously in the right flank. The tumors were allowed to establish for 2 weeks prior to the start of therapy and the mice were divided into four groups. Each group had five mice with 30- to 50-mm3 established tumors. The mice were injected (i. v. tail vein) daily for 5 days with saline, scFvMEL (2.5 mg/kg), scFvMEL (0.2 mg/kg) plus TNF (0.2 mg/kg) or scFvMEL/TNF (2.5 mg/kg). The other set of group had 5 mice with 100- to 200-mm3 well-established tumors. The mice were injected (i. v. tail vein) daily for 5 days with scFvMEL/TNF at the same dosage. At the end of therapy, the tumors were monitored by Xenogen IVIS 200 Imaging System weekly and by caliper every 2 or 3 days.

### Example 34

### Cytotoxicity of scFvMEL/TNF In Vitro

The cytotoxicity of the scFvMEL/TNF was assessed against log-phase antigen-positive, TNF-sensitive human melanoma A375-M cells and antigen-positive, TNF-resistant human melanoma AAB-527 cells, respectively. We also compared these effects with antigen-negative, TNF-sensitive human breast cancer SKBR3-HP and antigen-negative, TNF-resistant human neuroglioma H4 cells. The results showed that against antigen-positive A375-M cells, scFvMEL/TNF (I.C.₅₀ 0.1 nM) appeared to be approximately 10 fold more active than native TNF (I.C.₅₀ 1.4 nM) (p<0.0001). Against TNF-sensitive, antigen-negative SKBR3-HP cells, the cytotoxicity of scFvMEL/TNF (I.C.₅₀ 2.5 nM) showed a dose-response curve similar to that of authentic TNF (I.C.₅₀ 2.7 nM) (p>0.05). Against antigen-negative, TNF-resistant H4 cells, the scFvMEL/TNF demonstrated no cytotoxic effects at doses up to 1000 nM. However against antigen-positive, TNF-resistant human melanoma AAB-527 cells, the scFvMEL/TNF showed significant dose-related cytotoxic effects (I.C.₅₀ 20 nM). In contrast, these AAB-527 cells were resistant to the cytotoxic effects of TNF at concentrations of up to 5000 nM (Table 9).

TABLE 9
Cytotoxicity* of scFvMEL/TNF vs TNF on Various Human Cell Lines

| | I.C.₅₀ | |
|---|---|---|
| I.C.₅₀ | | Targeting |

| | | | | | |
|---|---|---|---|---|---|
| Cell lines | Cell Features | scFvMEL/TNF(nM) | TNF(nM) | Index** | p value |

| | | | | | |
|---|---|---|---|---|---|
| Melanoma | Gp240 (+) | 0.1 ± 0.013 | 1.37 ± 0.020 | 15 | <0.0001 |
| A375-M | TNF-sensitive | | | | |
| Melanoma | Gp240 (+) | 20.62 ± 1.125 | >5000*** | >250 | — |
| AAB-527 | TNF-resistant | | | | |
| Breast Cancer | Gp240 (−) | 2.55 ± 0.085 | 2.71 ± 0.014 | 1 | >0.05 |
| SKBR3-HP | TNF-sensitive | | | | |
| Neuroglioma | Gp240 (−) | >1000*** | >5000*** | — | — |
| H4 | TNF-resistant | | | | |

*72 h treatment
**Targeting Index = I.C.₅₀ of TNF/I.C.₅₀ of scFvMEL/TNF
***No cytotoxic effects at the indicated concentrations

### Example 35

### In Vivo Pharmacokinetics of scFvMEL/TNF

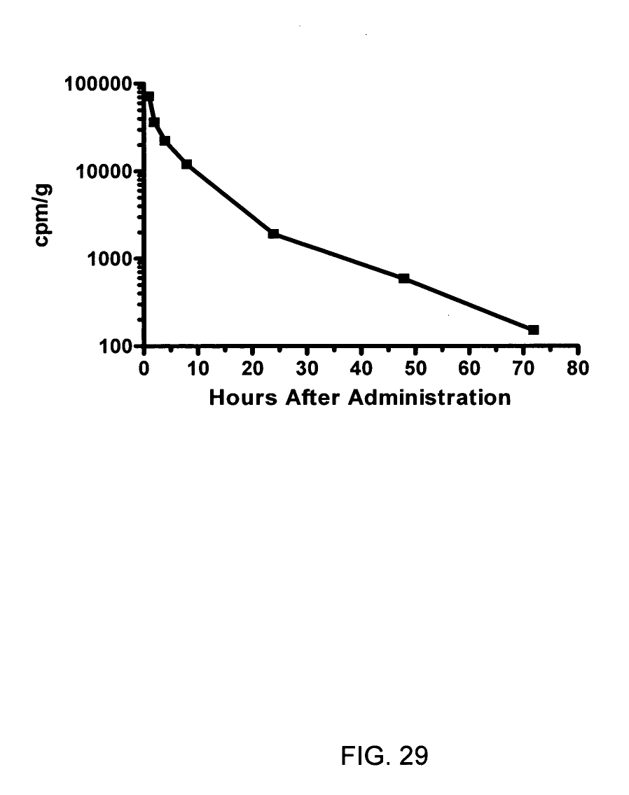

As described in Example 34, scFvMEL/TNF was radiolabeled using the p-iodobenzoate method described previously. The figure shows the mean±SEM of the data at each time-point. The curve represents the least-squares, best-fit line through these points. The data demonstrated a triphasic curve fit with calculated half-lives of 0.38 h, 3.9 h and 17.6 h for the α-, β- and γ-phases respectively (FIG. 29). However, the plasma clearance of radio-labeled TNF, chemical conjugate ZME-TNF as well as intact antibody ZME was biphasic and closely fit (γ2>0.94) an open, two-compartment mathematical model. Comparatively, as shown in Table 10, the half-lives for full-length antibody ZME-018 and chemical conjugate ZME-TNF were similar in this model with β-phase half-lives of 1.39 h and 1.2 h respectively.

TABLE 10
Pharmacokinetic Parameters of ¹²⁵I-ZME,
ZME-TNF, TNF, and scFvMEL/TNF

| | | ZME-TNF | | ScFvMEL/TNF |
|---|---|---|---|---|
| | ZME | Chemical | | Fusion |
| Parameters | Full-length | Conjugate | TNF | Construct |
| Plasma Half Life (α) | 1.39 | 1.20 | 0.45 | 0.38 |
| (hrs) | | | | |
| Plasma Half Life (β) | 41.30 | 36.05 | 2.68 | 3.93 |
| (hrs) | | | | |
| Plasma Half Life (γ) | — | — | — | 17.59 |
| (hrs) | | | | |
| Vd (ml) | 1.91 | 11.68 | 3.95 | 19.50 |
| C × t (μ Ci/ml × | 139.60 | 12.61 | 3.51 | 96.82 |
| min) | | | | |
| Clp (ml/kg × min) | 0.16 | 1.09 | 3.38 | 1.03 |

Vd: volume of distribution
C × t: the area under the concentration curve
Clp: plasma clearance rate

In addition, the half-lives of β-phase was also similar at 41.3 h and 36.1 h respectively. In contrast, the clearance of free TNF in this model was relatively rapid with α- and β-phase half-lives of 27.1 min and 2.7 h respectively. The immediately apparent volume of distribution (Vd) for ZME-018 alone approximated the blood volume (1.9 ml) while TNF alone had a somewhat larger Vd (3.9 ml), whereas, the ZME-TNF chemical conjugate displayed a higher Vd (11.6 ml) than either ZME-018 or TNF, suggesting a greater distribution outside the vasculature. The fusion construct scFvMEL/TNF demonstrated the highest Vd (19.5 ml) among all, suggesting the most extensive extra vascular disposition in all of its component agents. The area under the concentration curve (c×t) for TNF was substantially lower than that of ZME-018 alone (3.5 compared to 139.6 μCiml-1 min) because of its relatively short plasma half-life. The c×t for fusion construct scFvMEL/TNF was substantially larger than those of both TNF and chemical conjugate ZME-TNF, and lower than that of ZME-018 because of its relatively greater distribution outside the vasculature.

### Example 36

### Toxicity Studies of scFvMEL/TNF in BALB/c Mice

Mortality, Gross Pathology and Organ Weight:

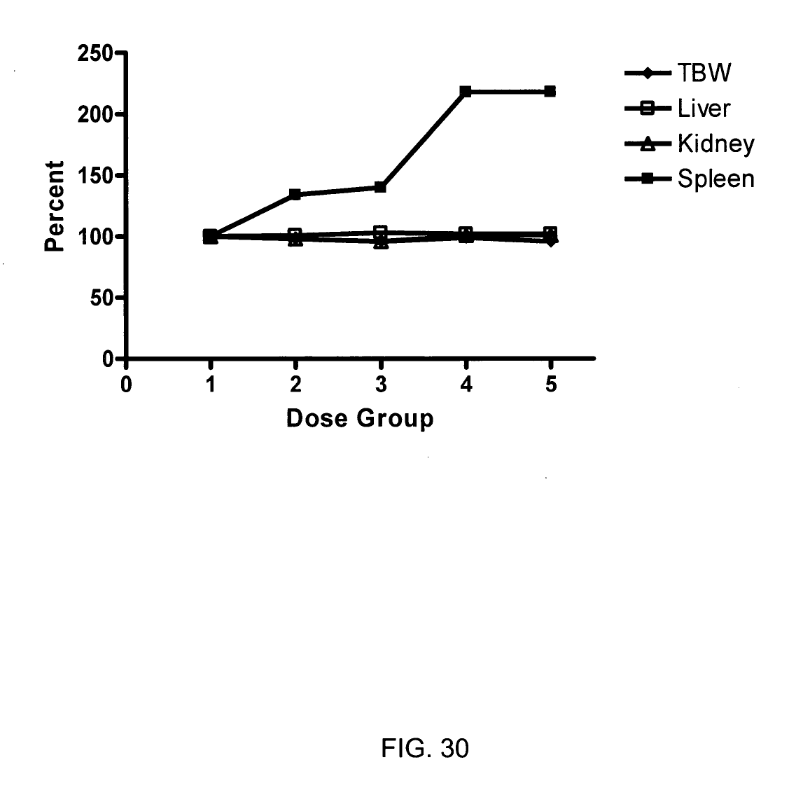

No deaths occurred on this study. No dose-related gross pathology findings were observed. A few gross lesions were observed that were considered incidental and not related to administration of scFvMEL/TNF. ScFvMEL/TNF causes a dose-related increase in the relative spleen weights (relative to body weight) at doses of 1, 2, 3, and 4 mg/kg (FIG. 30). The magnitude of the increase plateaus at 3 mg/kg. The increase in spleen weight correlates with increased extramedullar hematopoiesis in the red pulp and follicular hyperplasia in the white pulp.

Clinical Pathology:

No test substance-related alterations were noted in the hematology parameters (Table 11) and clinical chemistry parameters (Table 12).

TABLE 11
Group Means for Hematology Parameters

| | HGB | | WBC | RBC | Plat. | | | | | |
|---|---|---|---|---|---|---|---|---|---|---|
| Groups | g/dl | HCT % | 10\hat{ }3/ul | 10\hat{ }6/ul | 10\hat{ }3/ul | SEGS % | LYMS % | MON % | EOS % | BASO % |
| 1. Saline | 14.7 | 41.0 | 10.8 | 9.4 | 768 | 12.2 | 81.3 | 1.7 | 3.6 | 1.2 |
| 2. 25% MTD (1 mg/kg) | 13.1 | 35.9 | 7.8 | 8.3 | 914 | 15.7 | 74.7 | 4.7 | 1.8 | 3.3 |
| 3. 50% MTD (2 mg/kg) | 13.8 | 38.0 | 7.9 | 8.8 | 944 | 20.4 | 75.6 | 1.6 | 1.4 | 0.9 |
| 4. 75% MTD (3 mg/kg) | 13.7 | 39.0 | 7.1 | 9.0 | 406 | 24.6 | 69.8 | 2.7 | 1.5 | 1.5 |
| 5. 100% MTD (4 mg/kg) | 12.7 | 36.0 | 4.8 | 8.2 | 584 | 30.6 | 57.2 | 7.3 | 1.8 | 3.1 |
| Reference Range | 12.8-16.4 | 35.9-48.2 | 1.4-8.9 | 7.2-9.8 | 615-1802 | 1-35 | 54.8-92.4 | 1.5-9.7 | 1-6.6 | 0 |

TABLE 12
Group Means for Clinical Chemistry Parameters

| | T. | Na | K | | | | | | | | | | | |
|---|---|---|---|---|---|---|---|---|---|---|---|---|---|---|
| | BIL | mEq/ | mEq/ | Cl | Cr | BUN | Ca+ | PO4 | AST | ALT | Alk | T.P. | Alb | Glob |
| Groups | mg/dl | L | L | mmol/L | mg/dl | mg/dl | mg/dl | mg/dl | IU/L | IU/L | IU/L | gm/dl | gm/dl | gm/dl |
| 1. saline | 0.4 | 153.1 | 7.1 | 111.4 | 0.2 | 21.7 | 10.3 | 8.8 | 133 | 58 | 140 | 5.5 | 3.8 | 1.7 |
| 2. 25% | 0.2 | 152.5 | 7.7 | 113.2 | 0.2 | 18.6 | 10.5 | 8.8 | 115 | 76 | 117 | 5.5 | 3.7 | 1.8 |
| MTD | | | | | | | | | | | | | | |
| (1 mg/ | | | | | | | | | | | | | | |
| kg) | | | | | | | | | | | | | | |
| 3. 50% | 0.3 | 151.6 | 7.9 | 110.0 | 0.2 | 17.6 | 10.3 | 8.8 | 216 | 179 | 123 | 5.6 | 3.8 | 1.9 |
| MTD | | | | | | | | | | | | | | |
| (2 mg/ | | | | | | | | | | | | | | |
| kg) | | | | | | | | | | | | | | |
| 4. 75% | 0.3 | 151.8 | 7.8 | 109.8 | 0.2 | 13.4 | 10.8 | 9.5 | 258 | 112 | 111 | 5.9 | 3.7 | 2.2 |
| MTD | | | | | | | | | | | | | | |
| (3 mg/ | | | | | | | | | | | | | | |
| kg) | | | | | | | | | | | | | | |
| 5. 100% | 0.3 | 150.2 | 8.8 | 110.4 | 0.2 | 18.7 | 10.9 | 10.7 | 376 | 199 | 134 | 5.9 | 3.7 | 2.2 |
| MTD | | | | | | | | | | | | | | |
| (4 mg/ | | | | | | | | | | | | | | |
| kg) | | | | | | | | | | | | | | |
| Reference | 0-0.5 | 146-152 | 7-11 | 103-112 | 0-0.4 | 18-33 | 8.9-12.1 | 8.9-13.6 | 66-410 | 0-344 | 111-224 | 5.5-6.1 | 2.9-3.4 | 2.5-2.8 |
| Range | | | | | | | | | | | | | | |

There is very slight increase in the mean AST and ALT levels in groups of 2, 3, and 4 mg/kg when compared to the control group mean, but still within the reference range for this laboratory. These minimal elevations in the mean in these treated groups were due to single animals in each group. No histopathologic correlate was observed in any of these animals, suggesting the elevations in these three animals may be spurious.

Histopathology:

ScFvMEL/TNF systemic administration results in apparent dose dependent lesions in the liver, lung, and spleen of Balb/c female mice (Table 13).

TABLE 13
Summary Incidence: scFvMEL/TNF-Related Lesions*
and Lesion Average Grades**

| | | | Lung- | | | | | |
|---|---|---|---|---|---|---|---|---|
| | Liver- | | Thrombus, | | Spleen- | | | |
| | Extramedullary | | fibrosed/re- | | Extramedullary | | Spleen- | |
| | Hematopoiesis, | | cannalized, | | Hematopoiesis, | | Follicular Cell | |
| | increased | | singular | | increased | | Hyperplasia | |
| Groups | #P/T*** | Grade | #P/T | Grade | #P/T | Grade | #P/T | Grade |
| 1. Saline | 2/5 | 1 | 0/5 | | 2/5 | 1 | 2/5 | 1 |
| 2. 25% | 1/5 | 1 | 0/5 | | 5/5 | 1.5 | 5/5 | 2.8 |
| MTD | | | | | | | | |
| (1 mg/kg) | | | | | | | | |
| 3. 50% | 4/5 | 1 | 0/5 | | 5/5 | 2 | 5/5 | 3.2 |
| MTD | | | | | | | | |
| (2 mg/kg) | | | | | | | | |
| 4. 75% | 5/5 | 1 | 0/5 | | 5/5 | 4 | 5/5 | 3.4 |
| MTD | | | | | | | | |
| (3 mg/kg) | | | | | | | | |
| 5. 100% | 5/5 | 1 | 3/5 | 1 | 5/5 | 4 | 5/5 | 4 |
| MTD | | | | | | | | |
| (4 mg/kg) | | | | | | | | |

*Organ morphologic diagnosis
**The incidence of the lesion in the group
Grade 1 = modest, rare 5-10%
Grade 2 = mild, infrequent 10-20%
Grade 3 = moderate, frequent 20-50%
Grade 4 = severe, extensive >50%
***#P/T, Numbers of positive mice/total mice

The incidence and severity of these lesions appear dose-dependent and include increased extramedullary hematopoiesis of the liver and spleen at doses of 2, 3, and 4 mg/kg scFvMEL/TNF and greater, pulmonary fibrin thrombi, lung at doses of 4 mg/kg scFvMEL/TNF and greater, and follicular hyperplasia of the splenic white pulp at doses of 1, 2, 3, and 4 mg/kg scFvMEL/TNF and greater. The most sensitive indicator of scFvMEL/TNF-related effects is follicular lymphoid hyperplasia of the spleen. The no-observed-adverse-effect level (NOAEL) for scFvMEL/TNF under the conditions of this study is 3 mg/kg.

In Vivo Antitumor Effects of scFvMEL/TNF

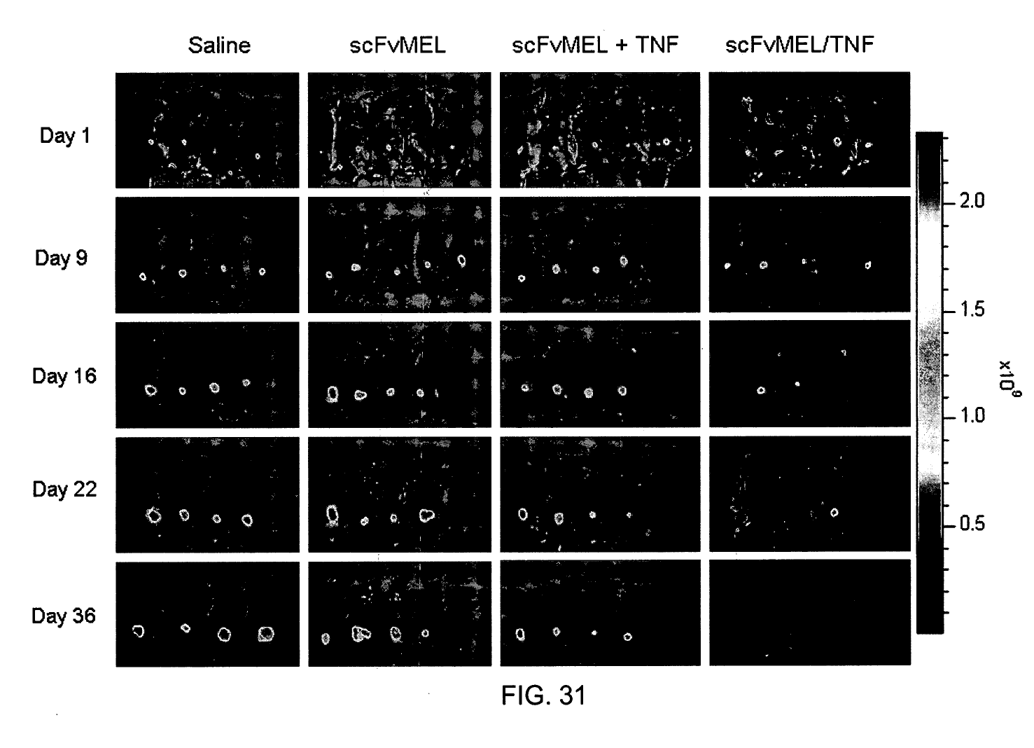

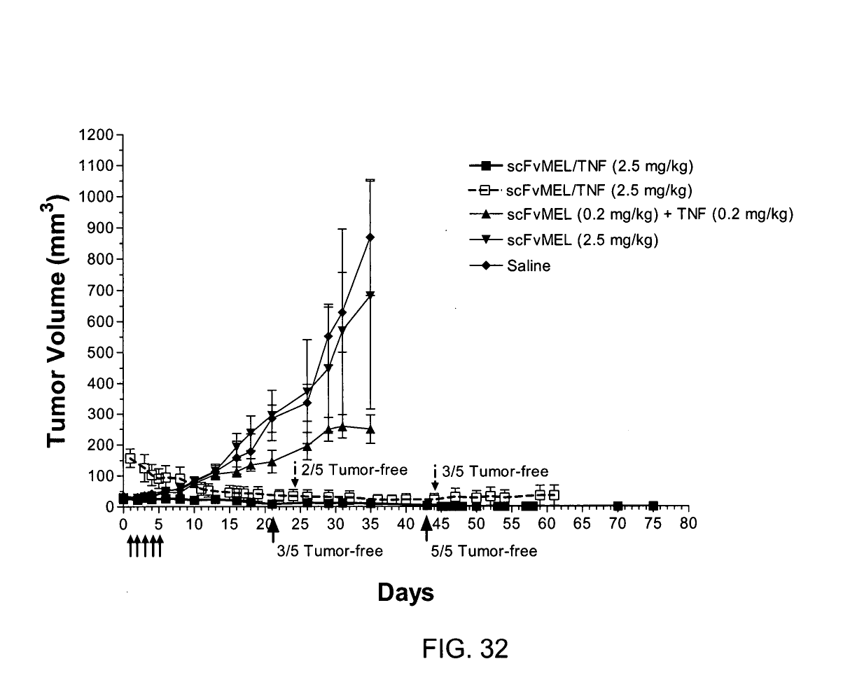

Groups of mice bearing established (30-50 mm³) A375GFP xenografts were treated (i. v. tail vein) daily (day 1-day 5) for 5 days with saline, scFvMEL (2.5 mg/kg), scFvMEL (0.2 mg/kg) plus TNF (0.2 mg/kg) or scFvMEL/TNF (2.5 mg/kg). Mice were observed and their tumors were imaged by Xenogen IVIS 200 imaging system (FIG. 31) and measured by caliper (FIG. 32) every 2 or 3 days. Treatment of scFvMEL/TNF at 2.5 mg/kg dosage at early stage (50 mm³) demonstrated potent antitumor activity indicating three of 5 mice tumor-free on day 21, and complete tumor regression, five of 5 mice tumor-free on day 43 when monitored by caliper measurement. While all mice treated with either saline, scFvMEL alone, or scFvMEL plus human recombinant TNF had rapid tumor growth. Mice treated at later stage (150 mm³), also showed tumor regression (3/5 tumor free on day 44). There was no subsequent outgrowth of tumors from mice rendered tumor free. These data show that scFvMEL/TNF can target melanoma cells in vivo and can result in pronounced anti-melanoma effects.

### Example 37

### Significance of Effects of scFvMEL/TNF

Malignant melanoma is a primary example of a cancer that is highly metastatic and which responds poorly to various treatments, including chemotherapy and γ-irradiation (Helmbach et al., 2001). Novel therapeutic strategies targeting melanoma are currently under development in several laboratories (Leong, 2003). The inventor previously reported (Liu et al., 2004) a fusion construct designated scFvMEL/TNF composed of the antibody scFvMEL which recognizes the surface domain of the gp240 antigen present on 80% of human melanoma cell. The antibody-specific delivery of TNF to the cell surface of melanoma cells resulted in augmented cytotoxicity compared to TNF alone. In addition, it was demonstrated that antibody-TNF chemical conjugates and fusion constructs were capable of delivering TNF to tumors in vivo (Liu et al., 2004; Rosenblum et al., 1995). In this study, we further demonstrated that the fusion construct scFvMEL/TNF has potent antitumor activity after in vivo administration. In addition, the MTD estimated for the scFvMEL/TNF fusion construct (4 mg/kg) was found to be almost 10 fold higher than the MTD reported for TNF itself (0.3 mg/kg) (Kuroda et al., 2000) suggesting that the targeted construct is capable of directing the active TNF cytokine to tumor cells and away from tissue sites which cause toxicity.

Tumor necrosis factor (TNF) is known to not only possess direct cytotoxicity against tumor cells, but it also induces tumor vessel disruption (Watanabe et al,. 1988). However, systemic administration of TNF protein has been shown to result in significant host toxicity without demonstrating significant antitumor effect (Moritz et al., 1989; Blick et al., 1987). A variety of strategies have been suggested to utilize the anti-tumor properties of this agent and simultaneously reduce the systemic side effects, including antibody-mediated delivery (Liu et al., 2004; Scherf et al., 1996) or transfer of the TNF gene into tumor cells (Koshita et al,. 1995).

TNF as a therapeutic payload for targeted therapeutics has undergone extensive pre-clinical evaluation by our group and others (Liu et al,. 2004; Rosenblum et al,. 2000; Rosenblum et al,. 1991; Cumis et al., 2004; Hoogenboom et al,. 1991; Rosenblum et al,. 1995). More recently, the novel immunocytokine scFv23/TNF targeted Her-2/neu over-expressing malignancies has been shown to sensitize TNF-resistant Her-2/neu over-expressing breast cancer cells to TNF-induced apoptosis (Lyu and Rosenblum, 2005). These data indicate that TNF targeted to tumor cells may have fundamental differences in the cellular effects exerted by the fusion construct compared to that of TNF itself.

Antibodies for targeted delivery of cytokines provide not only for enhanced localization to tumor tissue after in vivo administration, they also have the potential to increase the plasma half-life of therapeutic agents (Mihara et al., 1991). As shown in this study, TNF has a relatively short serum half-life compared to that of a monoclonal antibody (half lives typically 20-40 hrs). The chemical conjugate ZMB-TNF consisting of full-length IgG antibody ZME-018 chemically coupled to TNF demonstrated a much longer serum half-life thereby increasing the circulating time of biological active TNF.

Because effective clinical management of solid tumors such as melanoma generally requires prolonged treatment regimens, the immunogenicity of this molecule is of concern. The immunogenicity of antibody-cytokine containing full-length antibodies is due, at least in part, to the large size of the antibody itself, and the long circulation time of the constructs. The serum half-life of fusion construct scFvMEL/TNF was much shorter than that of chemical conjugate, suggesting that the single-chain immunocytokine, by comparison, should be significantly less immunogenic due to its small size and relatively rapid clearance kinetics from the circulation. In addition to these properties, scFvMEL/TNF demonstrated the highest Vd (19.5 ml) and substantial larger c×t (96 μCi/ml×min) than those of both TNF and chemical conjugate ZME-TNF suggesting its relatively greater distribution outside the vasculature. Tissue distribution studies (Liu et al,. 2004) of the scFvMEL/TNF construct indicated that tumor localization of the construct occurs efficiently and that by 72 h, concentrations of the fusion construct are highest in tumor tissues compared to that of free TNF. The tumor-targeting capability of the construct may account for the increase in the apparent volume of distribution (Vd) of the construct compared to that of TNF. In addition, the smaller size of the scFvMEL/TNF construct compared to the larger ZME-TNF conjugate may be responsible for its comparatively facile extra-vascular disposition. The short half-life observed with this construct suggests that dosing intervals of 24 or 48 h appear to be optimal to achieve maximal concentration of the agent in tumor tissue.

Of critical importance to the eventual clinical development of scFvMEL/TNF is examination of maximum tolerated dose (MTD) toxicity profile and efficacy studies of this fusion construct in well-characterized human melanoma xenograft models. The MTD of the fusion construct scFvMEL/TNF was 0.8 mg per kg of body weight daily for 5 consecutive days and that corresponded to an MTD of 4 mg/kg total dose. It is important to note that the impressive antitumor effects observed in vivo with the fusion construct were obtained at a dose of 2.5 mg/kg which is approximately 60% of the MTD dose and was also below the NOAEL dose (3 mg/kg) determined from our toxicology studies.

The results indicate that neither significant adverse effects nor organ toxicity were associated with i. v. injections of scFvMEL/TNF at doses up to 100% of the MTD (4 mg/kg). The scFvMEL/TNF construct could be safely administered at this schedule at doses up to 75% of the MTD (3.0 mg/kg). Even though dose-related extramedullary hematopoiesis was noted in the liver and spleen, and follicular cell (lymphoid) hyperplasia was noted in the spleen, none of these lesions was considered adverse effects. In addition, many of these effects were noted in the saline-treated controls. In addition, there were no significant changes in hematology parameters or changes in clinical chemistry parameters notes in mice even when treated at 100% of the MTD. A single re-canalized thrombus in lung tissue was observed in 3 out of 5 mice in MTD dose level. These thrombi occurred in only one vessel, and did not compromise pulmonary function, however, they are considered biologically adverse and for this reason, the no observed adverse effect (NOAEL) was assessed at the 3 mg/kg (75% MTD) dose level.

The MTD of scFvMEL/TNF in mice was found to be significantly higher than that of rhuTNF alone (0.3 mg/kg) (Kuroda et al,. 2000). However, rhuTNF induced severe toxicity at the MTD since intravenously administered TNF leads to destruction of normal tissue microvasculature (Kuroda et al,. 2000; Kuroda et al,. 1995). In the present study, scFvMEL/TNF showed no significant normal organ injury in mice despite its higher antitumor potency. Thus, scFvMEL/TNF appears to have more selective activity towards tumors compared with a rather non-specific toxicity profile for rhTNF.

Thus, careful assessment of pharmacokinetics of the scFvMEL/TNF construct has provided a rationale for designing an optimal administration schedule. Assessment of the therapeutic efficacy of this schedule demonstrates impressive, long-term antitumor effects against melanoma xenografts against both small, established lesions and tumors that are much more advanced. These efficacy studies combined with histopathology, clinical chemistry and effects on clinical chemistry parameters provide a rationale for designing Phase I trials in patients with gp240 positive tumors.

### Example 37

### Exemplary Materials and Methods for Examples 38-42

Cell Culture

Human melanoma cells were obtained from Dr. I. J. Fidler of M. D. Anderson Cancer Center, Houston, Tex. TXM-1, TXM-13, TXM-18L cells were cultured in minimum essential medium (MEM) and A375-M were cultured in Dulbecco's MEM containing 10% fetal bovine serum (FBS), added sodium pyruvate (100 mM), nonessential amino acids (10 mM), glutamine (200 nM) and MEM vitamins. MEL-526 were cultured in RPMI 1640 containing 10% FBS. All cells were routinely grown at a density of 7×10⁶ cells/T-75 flask, subcultured twice per week and were routinely tested and found to be free of Mycoplasma contamination using the Gen-Probe assay kit.

Elisa

Ninety-six well ELISA plates containing adherent melanoma cells (5×10⁴ cells per well) were blocked by addition of a solution containing 5% bovine serum albumin (BSA) for 1 h. For detection gp240 antigen, cells were incubated with monoclonal antibody ZME-018 IgG2a followed by incubation with goat anti-mouse/horseradish peroxidase conjugate (HRP-GAM). For binding activity of GrB/scFvMEL, cells were incubated with purified GrB/scFvMEL at various concentrations for 1 h at room temperature (RT). After they were washed, the cells were incubated with rabbit anti-scFvMEL antibody, followed by addition of goat anti-rabbit/HRP conjugate (HRP-GAR) antibody. Finally, the substrate (2,2′-azino-bis-3-ethylbenzthiazoline 6-sulfonic acid, ABTS) solution containing 1 μl/ml 30% H₂O₂ was added to the wells. Absorbance at 405 nm was measured after 30 min.

Antigen gp240 Staining and FACS Analysis

Samples consisting of 1×10⁶ cells were first treated with monoclonal antibody ZME-018 IgG2a for 20 min at 4° C., then stained with allophycocyanin (APC)-conjugated Goat-Anti-Mouse antibody (BD Immunocytometry System, CA) for another 20 min at 4° C., both resuspended in 100 μl FACS staining buffer (2% FCS/DPBS). As a negative staining control, cells were stained with an isotype-matched control antibody of irrelevant specificity (Mouse IgG2a, PharMingen, San Diego, Calif.) at the same concentration as the antibody against gp240. Following staining, cells were washed twice with DPBS, then resuspended in 500 μl of 1% paraformaldehyde solution and stored on ice in the dark. FACS analysis was carried out right afterward on a FACS Caliber cytometer (Becton Dickinson, San Jose, Calif.). APC fluorescence was detected in the FL-4 channel. For each cell line, 10,000 events were acquired. Analysis was performed with the CellQuest Pro™ software ((Becton Dickinson).

Cytotoxicity Assays In Vitro Against Melanoma Cells

Samples (GrB/scFvMEL or scFvMEL/rGel) were assayed using a standard 72-h cell proliferation assay with melanoma cell monolayers and using crystal violet staining procedures as described previously (Nishikawa et al., 1992). The percent of control refers to the percentage of cells in the drug-treated wells compared to that of control (untreated) wells.

Combination Studies of GrB/scFvMEL with Chemotherapeutic Agents

Cells in exponential growth phase were plated into 96-well plates. After 24 hr, the cells were treated with drug-containing medium. At the end of the indicated incubation period, growth inhibition was assessed by crystal violet staining. In order to determine the effects of sequencing, cells were treated with different two sequences.

Sequence I (C1): cells were pretreated with chemotherapeutic agent for 6 h, and then co-administered with chemotherapeutic agent and GrB/scFvMEL for 72 h.

Sequence II (C2): cells were pretreated with GrB/scFvMEL for 6 h, and then co-administered with chemotherapeutic agent and GrB/scFvMEL for 72 h.

Sequence III: cells were pretreated with GrB/scFvMEL for 6 h, followed by treatment with chemotherapeutic agents for 72 h.

Sequence IV: cells were treated with various chemotherapeutic agents for 72 h without GrB/scFvMEL pretreatment.

Chemotherapeutic agents include doxorubicin (DOX), vincristine (VCR), etoposide (VP-16), cisplantin (CDDP), cytarabine (Ara C) and 5-FU.

Combination Studies of GrB/scFvMEL with Radiation Therapy

Cells were seeded in triplicate at the density of 4000 cells per well. The different doses of γ-irradiation were delivered by ¹³⁷Cs source at a dose rate of 2Gy, 4Gy and 6Gy respectively. The cells were irradiated either alone or after addition of various concentrations of the GrB/scFvMEL fusion construct to determine whether one sensitizes to the other. After 72 hrs, cells were stained by crystal violet.

Animal Model Studies

Establish A375-M Xenograft Model

Athymic (nu/nu) mice, 4-6 weeks old, were obtained from Harlan Sprague Dawley, Indianapolis. Ind. The animals were maintained under specific-pathogen-free conditions and were used at 6-8 weeks of age. Animals were injected subcutaneously, (right flank) with 3×10⁶ log-phase A375-M melanoma cells and tumors were allowed to establish. Once tumors were measurable (˜30-50 mm³), animals were treated (i.v. via tail vein) with either saline (control) or GrB/scFvMEL fusion construct.

Localization of GrB/scFvMEL After Systematic Administration

Mice bearing A375-M xenograft tumors were administered GrB/scFvMEL. Twenty-four hours later, animals were sacrificed and representative tissue sections were removed and formalin fixed and stained by H & E and immunohistochemical staining for GrB/scFvMEL detected by either anti-GrB or anti-scFvMEL antibody.

TUNEL Assay to Detect Apoptosis

Tumor tissue sections were stained by TUNEL using an in situ cell death detection kit (Roche Molecular Biochemicals, Mannhein, Germany). Briefly, pretreatment of paraffin-embedded tissue was performed to dewax, rehydrate and then incubate with proteinase K followed by fixation and permeabilization. The tissue sections were incubated with a terminal deoxynucleotidyl transferase-mediated nick end labeling (TUNEL) reaction mixture in a humidified chamber for 60 min at 37° C. and then rinse the slides 3 times with PBS. Samples were analyzed under Nikon Eclipse TS 100 fluorescent microscope and photographs were taken with a scope-mounted Nikon digital camera (Tokyo, Japan).

In Vivo Cytotoxicity Studies

Once tumors establishes to measurable size (˜30-50 mm³), animals were treated (i. v. via tail vein) with either saline (control) or GrB/scFvMEL fusion construct (37.5 mg/kg) for 5 times at every other day. Animals were monitored, and tumors were measured for an additional 28 days.

### Example 38

### Antigen GP240 Expression on Different Melanoma Cells (A375-M, TXM-18, TXM-13, MEL526 AND TXM-1)

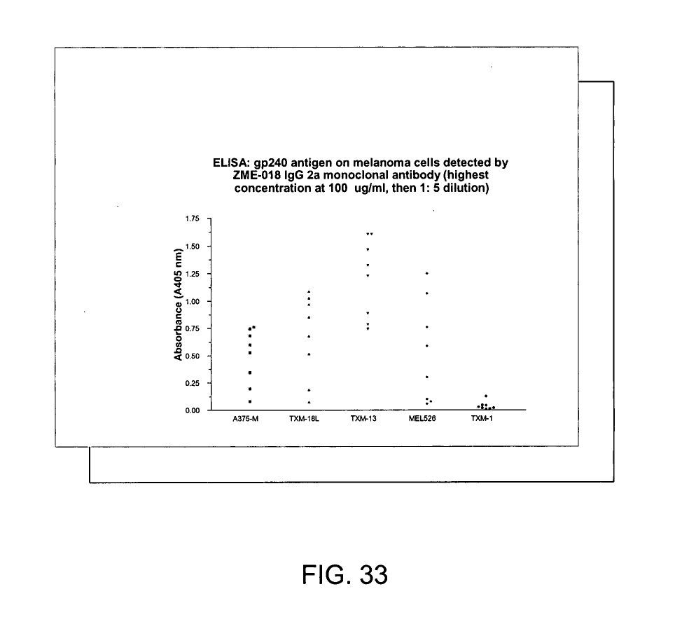

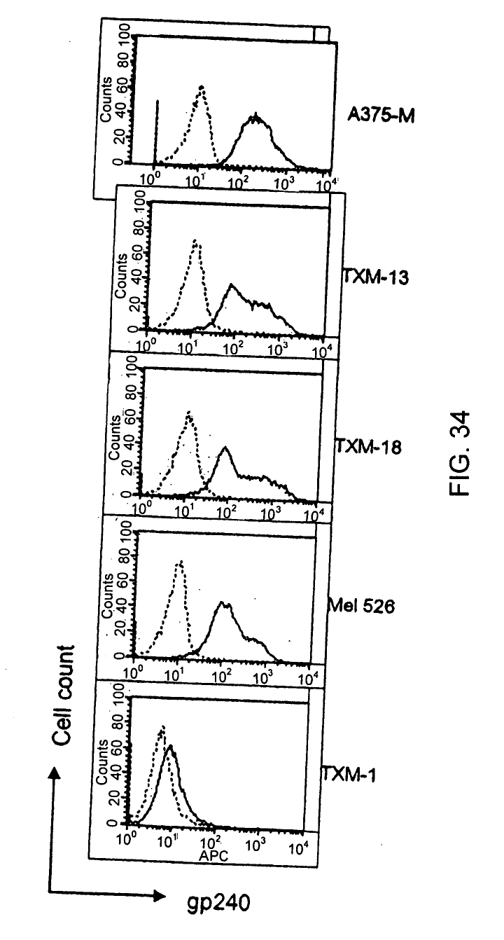

To examine the expression of gp240 antigen on melanoma A375-M, TXM-18, TXM-13, MEL 526 and TXM-1 cells, parental monoclonal antibody ZME-018 IgG2a that specifically binds to gp240 antigen was used in ELISA (FIG. 33) and flow cytometry (FIG. 34). The results demonstrated that gp240 antigen presents on A375-M, TXM-18L, TXM-13, and MEL-526 cells, however, there was very low level of expression on TXM-1 cells.

### Example 39

### Binding Activity of scFvMEL Moiety of GrB/scFvMEL Fusion Protein by Elisa

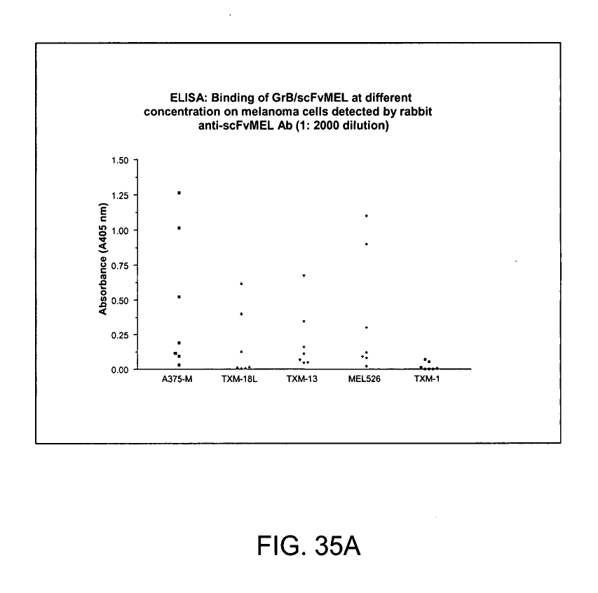

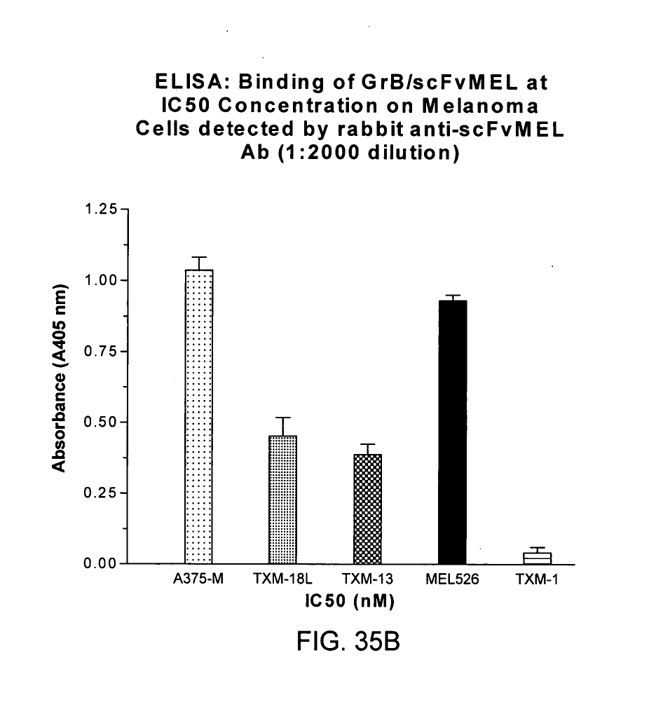

An ELISA was performed to determine the binding activity of the GrB/scFvMEL fusion construct to melanoma cells. GrB/scFvMEL bound to high-level gp240 antigen expressing melanoma A375-M, TXM-18L, TXM-13 and MEL-526 cells. Moreover, the binding activity was stronger in A375-M and MEL-526 followed by TXM-18L and TXM-13. However, the protein did not bind to TXM-1 in which has very low-level expression of gp240 antigen as detected by an anti-scFvMEL rabbit monoclonal antibody (FIGS. 35A and 35B).

### Example 40

### In Vitro Cytotoxicity of GrB/scFvMEL Against Different Melanoma Cells (IC₅₀ Comparison)

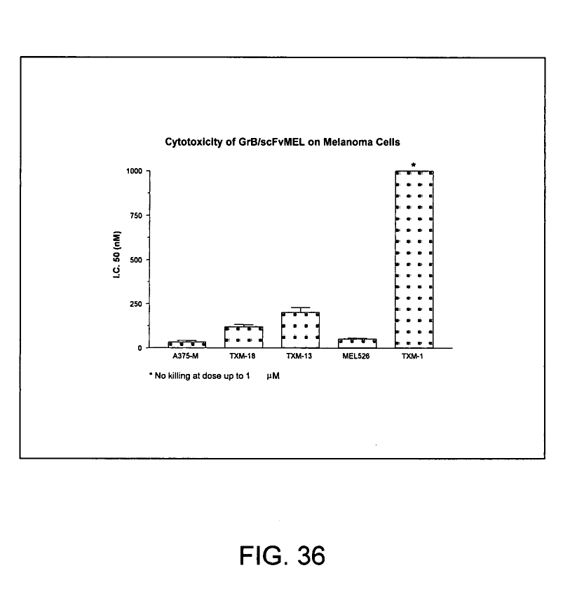

The cytotoxicity of GrB/scFvMEL was assessed against log-phase A375-M, TXM-18L, TXM-13, MEL-526 and TXM-1 cells in culture. A 50% growth inhibitory effects were found at concentrations of 20 nM on A375-M cells, 50 nM on MEL-526 cells, ˜100 nM on TXM-18L, 200 nM on TXM-13, respectively. However, no cytotoxic effects were found on TXM-1 cells at doses of up to 1 μM (FIG. 36). By comparison, the cytotoxic effects of GrB/scFvMEL were approximately the same as that of another fusion toxin, MEL sFv/rGel on these melanoma cells (Table 14).

TABLE 14
Cytotoxicities of GrB/scFvMEL vs MEL sFv/rGel against
Different Human Melanoma Cell Lines (I.C.₅₀, nM)

| Cell lines | GrB/scFvMEL | MEL sFv/rGel |
|---|---|---|
| A375-M | 32.1 ± 7.95 | 22.03 ± 3.08 |
| MEL-526 | 50.0 ± 4.32 | 40.0 ± 5.55 |
| TXM-18L | 158.3 ± 12.45 | 144.3 ± 9.56 |
| TXM-13 | 200.0 ± 17.92 | 180.3 ± 15.30 |
| TXM-1 | >1 μM | >1 μM |

These data demonstrated that the extent of binding activity of GrB/scFvMEL to gp240 antigen positive cells incorporate with the cytotoxicity of the fusion protein.

### Example 41

### Combination Studies of GrB/scFvMEL with Conventional Chemotherapeutic Agents on A375-M

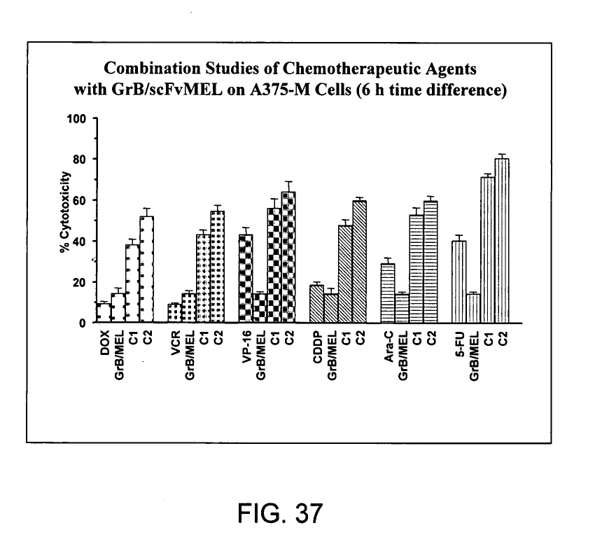

Co-administration GrB/scFvMEL and chemotherapeutic agents (doxorubicin, vincristine sulfate, etoposide, cisplatin or cytorabine) to A375 cells for 72 hours, demonstrated synergistic antitumor activity with adriamycin, vincristine or cisplatin and additive effects in combination with etoposide or cytorabine. Pre-treatment with GrB/scFvMEL for 6 h followed by co-exposure to these chemotherapeutic agents for 72 hours (sequence II-C2) showed significantly inhibited growth as compared to pre-treatment with drugs followed by co-exposure the fusion construct (sequence I-C1) (FIG. 37). Moreover, the cytotoxicities of various chemotherapeutic agents significantly increased when A375 cells were pre-treated with GrB/scFvMEL for 6 h followed by treatment with chemotherapeutic agents for 72 h (sequence III) compare to without GrB/scFvMEL pretreatment (sequence IV) (p<0.01) (Table 15).

TABLE 15
Cytotoxicities of Chemotherapeutic Agents against A375-M Cells
after 72 h Exposure: Pre-treatment with GrB/scFvMEL for 6 h Can
Significantly Increase the Cytotoxicity of Chemotherapeutic Agents

| Treatment* | MEAN** ± SEM | p value*** |
|---|---|---|
| DOX (Seq. III) | 33.73 ± 1.590 | |
| DOX (Seq. IV) | 30.33 ± 1.093 | 0.1528 |
| VCR (Seq. III) | 57.00 ± 2.454 | |
| VCR (Seq. IV) | 24.57 ± 2.987 | 0.001 |
| VP-16 (Seq. III) | 59.80 ± 1.986 | |
| VP-16 (Seq. IV) | 51.27 ± 1.102 | 0.0198 |
| CDDP (Seq. III) | 54.47 ± 0.233 | |
| CDDP (Seq. IV) | 43.80 ± 0.586 | 0.0001 |
| Ara C (Seq. III) | 68.70 ± 0.950 | |
| Ara C (Seq. IV) | 62.10 ± 1.054 | 0.0097 |
| 5Fu (Seq. III) | 44.43 ± 2.118 | |
| 5Fu (Seq. IV) | 21.90 ± 0.794 | 0.0006 |

*Treatment:
Seq. III vs. Seq. IV.
Seq. III: Cells pretreated with GrB/scFvMEL for 6 h, followed by treatment with chemotherapeutic agents for 72 h
Seq. IV: Cells were treated with various chemotherapeutic agents for 72 h without GrB/scFvMEL pretreatment.
**% cytotoxicity
***p < 0.05 statistically significant difference

The results indicated that the effects of chemotherapeutic agents could be sensitized by pretreatment with GrB/scFvMEL for 6 h on gp240 antigen positive targeted cells.

### Example 42

### In Vivo Animal Studies of GrB/scFvMEL (A375-M Xenograft Model)

Mice bearing A375-M xenograft tumors were administered GrB/scFvMEL. Twenty-four hours later, animals were sacrificed and representative tissue sections were removed and formalin fixed and stained by H & E and TUNEL. Tumor tissue displayed apoptotic neuclei in treatment group.

Immunohistochemical staining for GrB/scFvMEL detected by either anti-GrB or anti-scFvMEL antibody were performed. Localization or internalization of GrB/scFvMEL was observed in tumor tissue.

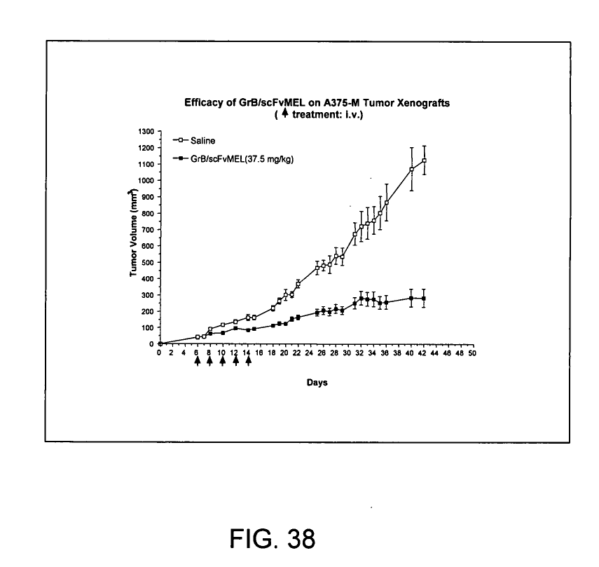

To examine the in vivo anti-tumor effects of GrB/scFvMEL, the studies were performed on A375-M human melanoma tumor xenografts. Mice bearing the tumors were treated (iv tail vein) 5× every other day with either GrB/scFvMEL or saline. Tumor volumes were measured for 42 days. The saline-treated control tumors increased from 50 mm³ to 1200 mm³ over this period. Tumors treated with GrB/scFvMEL (37.5 mg/kg) increased from 50 mm³ to 200 mm³ (FIG. 38).

### Example 43

### Exemplary Materials and Methods for Examples 44-50

The present example provides exemplary materials and methods related to the exemplary GrB/scFvMEL chimeric molecule.

Cell Culture

Human melanoma cells were obtained from Dr. I. J. Fidler (M. D. Anderson Cancer Center, Houston, Tex.). TXM-1, TXM-18L cells were cultured in minimum essential medium (MEM) and A375-M were cultured in Dulbecco's MEM containing 10% fetal bovine serum (FBS), added sodium pyruvate (100 mM), nonessential amino acids (10 mM), glutamine (200 nM) and MEM vitamins. MEL-526 were cultured in RPMI 1640 containing 10% FBS. All cells were routinely grown at a density of 7×10⁶ cells/T-75 flask, subcultured twice per week and were routinely tested and found to be free of Mycoplasma contamination using the Gen-Probe assay kit.

Expression and Purification of GrB/scFvMEL

The construction, expression, and purification of GrB/scFvMEL have been previously described (Liu et al., 2003). The fusion protein was stored in sterile 150 mM NaCl at −20° C.

Antigen gp240 Staining and FACs Analysis

Samples consisting of 1×10⁶ cells were first treated with ZME-018 IgG2a for 20 min at 4° C., then stained with allophycocyanin (APC)-conjugated Goat-Anti-Mouse antibody (BD Immunocytometry System, CA) for another 20 min at 4° C., both resuspended in 100 μl FACs staining buffer (2% FCS/DPBS). As a negative staining control, cells were stained with an isotype-matched control antibody of irrelevant specificity (Mouse IgG2a, PharMingen, San Diego, Calif.) at the same concentration as the antibody against gp240. Following staining, cells were washed twice with DPBS, then resuspended in 500 μl of 1% paraformaldehyde solution and stored on ice in the dark. FACs analysis was performed immediately thereafter on a FACs Caliber cytometer (Becton Dickinson, San Jose, Calif.). APC fluorescence was detected in the FL-4 channel. For each cell line, 10,000 events were acquired. Analysis was performed with the CellQuest Pro™ software ((Becton Dickinson).

ELISA Assays

Ninety-six well ELISA plates containing adherent melanoma cells (5×10⁴ cells per well) were used as described previously (Rosenblum et al., 1991). To detect the binding activity of GrB/scFvMEL, cells were incubated with purified GrB/scFvMEL at various concentrations for 1 h at room temperature (RT). After they were washed, the cells were incubated with rabbit anti-scFvMEL antibody, followed by addition of goat anti-rabbit/HRP conjugate (HRP-GAR) antibody. Finally, the substrate (2,2′-azino-bis-3-ethylbenzthiazoline-6-sulfonic acid, ABTS) solution containing 1 μl/ml 30% H₂ O₂ was added to the wells. Absorbance at 405 nm was measured after 30 min.

Internalization Analysis by Immunofluorescence

Cells were plated into 16-well chamber slides (Nalge Nunc International, Naperville, Ill.) at a density of 1×10⁴ cells per well. Cells were treated with GrB/scFvMEL (40 nM) for 1 hr. Proteins binding to the cell surface were removed by brief incubation with glycine buffer (0.5 M NaCl, 0.1 M glycine, pH 2.5) followed by immunofluorescent staining, as described previously (Liu et al., 2003). Briefly, cells were fixed in 3.7% formaldehyde and permeabilized in 0.2% Triton X-100. Samples were blocked with 3% BSA, incubated with goat anti-GrB mAb, and then incubated with FITC-coupled anti-goat IgG and propidium iodide (PI, 2.5 μg/ml). The slides were mounted with DABCO containing 1 μg/ml of PI and analyzed under a Nikon Eclipse TS-100 fluorescence microscope. Photographs were taken with a scope-mounted camera.

In Vitro Cytotoxicity Assays

To examine the cytotoxicity of GrB/scFvMEL or MEL/sFv/rGel, melanoma cells were plated on 96-well plates at a density of 4×10³ cells per well and allowed to adhere for 24 hr at 37° C. in 5% CO₂. After 24 hr, the medium was replaced with medium that contained various concentrations of fusion proteins. The effects on the growth of tumor cells in culture were determined by crystal violet (0.5% in 20% methanol) staining and solublized with Sorenson's buffer (0.1 M sodium citrate, pH 4.2 in 50% ethanol) as described previously[19]. The percent of control refers to the percentage of cells in the drug-treated wells compared to that of control (untreated) wells.

Combination Studies of GrB/scFvMEL with Chemotherapeutic Agents

Cells in exponential growth phase were plated into 96-well plates. After 24 hr, the cells were treated with drug-containing medium. At the end of the indicated incubation period, growth inhibition was assessed by crystal violet staining. I.C.₂₅ dose of either GrB/scFvMEL or chemotherapeutic agents have been used in combination studies. In order to determine the effects of sequencing, cells were treated with two different sequences: Sequence I: cells were pretreated with chemotherapeutic agent for 6 h, and then followed by co-administration with GrB/scFvMEL for 72 h. Sequence II: cells were pretreated with GrB/scFvMEL for 6 h, and then followed by co-administration with chemotherapeutic agent for 72 h. Chemotherapeutic agents include doxorubicin (DOX), vincristine (VCR), etoposide (VP-16), cisplatin (CDDP), cytarabine (Ara C) and 5-fluorouracil (5-FU).

Clonogenic Survival Assay:

The effectiveness of the combination of GrB/scFvMEL and ionizing radiation was assessed by clonogenic assay. Melanoma cells were either treated with PBS or pretreated with GrB/scFvMEL (10 nM) for 16 hours. Then, cells were irradiated with various doses of ionizing radiation and then processed for clonogenic cell survival assay. Following treatment, cells were trypsinized and counted. Known numbers were replated in triplicate and returned to the incubator to allow macroscopic colony development. Colonies were stained with crystal violet solution and counted after ˜14 days. The percentage plating efficiency and fraction surviving a given treatment was calculated based on the survival of non-irradiated cells treated with the agent in question.

Tumor Cell Invasion (Matrigel) Assay:

Subconfluent A375 DR cells were collected by trypsinization, resuspended in culture medium, and seeded in 20 μl (100,000 cells) on the lid of a culture dish. The lid was then placed on a dish filled with 2 ml of culture medium and incubated at 37° C. for 48 h. Matrigel solution (100 μl, 2.7 mg/ml) was pipetted onto the bottom of wells of a 24-well culture dish and left to set at 37° C. Cell aggregates were transferred over the cushion and then overlaid with additional of 100 μl of Matrigel. The aggregates into Matrigel were covered with 400 μl culture medium in the absence or in the presence of GrB/scFvMEL (50 nM). The aggregates were then observed daily under a light microscope, and at the end of the incubation time pictures of the aggregates were taken. The densities of cells invaded into matrigel surrounding the aggregates was analyzed by AlphaEase®FC software (Alpha Innotech, San Leandro, Calif.) and the percent of invasion was calculated based on the cell densities of two groups and standardized by the value of non-treatment control group as 100% invasion.

Animal Model Studies

(1) A375-M Xenograft Model and Therapy Study Design

Athymic (nu/nu) mice, 4-6 weeks old, were obtained from Harlan Sprague Dawley, Indianapolis. Ind. The animals were maintained under specific-pathogen-free conditions and were used at 6-8 weeks of age. Animals were injected subcutaneously, (right flank) with 3×10⁶ log-phase A375-M melanoma cells and tumors were allowed to establish. Once tumors were measurable (30-50 mm3), animals were treated via i.v. tail vein with either saline (control) or GrB/scFvMEL fusion construct. Fusion protein GrB/scFvMEL was given intravenously in a 0.2 ml volume. Doses mentioned in this paper are total doses administered once every other day for 5 doses (qod). The doses are reported as mg/kg based on a mouse of average weight of 20 g. For example, a 20 g mouse given 750 μg of GrB/scFvMEL would receive a dose of 37.5 mg/kg.

(2) Localization of GrB/scFvMEL after Systematic Administration

Mice bearing A375-M xenograft tumors were administered GrB/scFvMEL (37.5 mg/kg). Twenty-four hours later, animals were sacrificed and representative tissue sections were removed and formalin fixed and stained by hematoxylin and eosin (H & E) and immunohistochemical staining for GrB/scFvMEL detected by either anti-GrB or anti-scFvMEL antibody.

(3) TUNEL Assay to Detect Apoptosis

Tumor tissue sections were stained by TUNEL using an in situ cell death detection kit (Roche Molecular Biochemicals, Mannhein, Germany). Briefly, pretreatment of paraffin-embedded tissue was performed to dewax, rehydrate and then incubate with proteinase K followed by fixation and permeabilization. The tissue sections were incubated with a terminal deoxynucleotidyl transferase-mediated nick end labeling (TUNEL) reaction mixture in a humidified chamber for 60 min at 37° C. and then rinse the slides 3 times with PBS. Samples were analyzed under Nikon Eclipse TS 100 fluorescent microscope and photographs were taken with a scope-mounted Nikon digital camera (Tokyo, Japan).

(4) In Vivo Cytotoxicity Studies

Once tumors establishes to measurable size (˜30-50 mm3), animals were treated (i. v. via tail vein) with either saline (control) or GrB/scFvMEL fusion construct for 5 times at every other day (37.5 mg/kg). Tumor growth was monitored by caliper-measuring two perpendicular tumor diameters every 2 or 3 days for additional 28 days, and the volume of the tumor was calculated from the formula: tumor volume=(width)²×length/2 (Osborne et al., 1985).

Statistical Analysis

Data were analyzed using the paired t test (Prism 3.0). Data are presented as means±SE. A difference was regarded as significant if p<0.05.

### Example 44

### Antigen gp240 Expression on Melanoma Cells, Cell Binding Activity of the scFvMEL Component of GrB/scFvMEL and Internalization of GrB/scFvMEL

To examine the expression of gp240 antigen on melanoma cell lines, melanoma cells consisting of 1×10⁶ were stained with parental monoclonal antibody ZME-018 IgG 2a that specifically binds to gp240 antigen for 20 min and APC conjugated goat-anti-mouse antibody for another 20 min at 4° C. As a negative staining control, cells were stained with an isotype-matched control antibody of irrelevant specificity (mouse IgG2a) at the same concentration as the antibody against gp240. Expression level of gp240 was indicated as assessed by FACs assay as the solid lines for melanoma cell lines A375-M, MEL-526, TXM-18 and TXM-1 on which the percent of gp240 positive (% positive) was 98.9, 97.8, 40.9, and 4.6, respectively. These data demonstrated that gp240 antigen much higher expressed on A375-M, MEL-526 cells, higher expressed on TXM-18 cells, however very low level expressing of gp240 antigen on TXM-1 cells showing that the solid lines were overlaid by the dot lines representing the isotype control (FIG. 34).

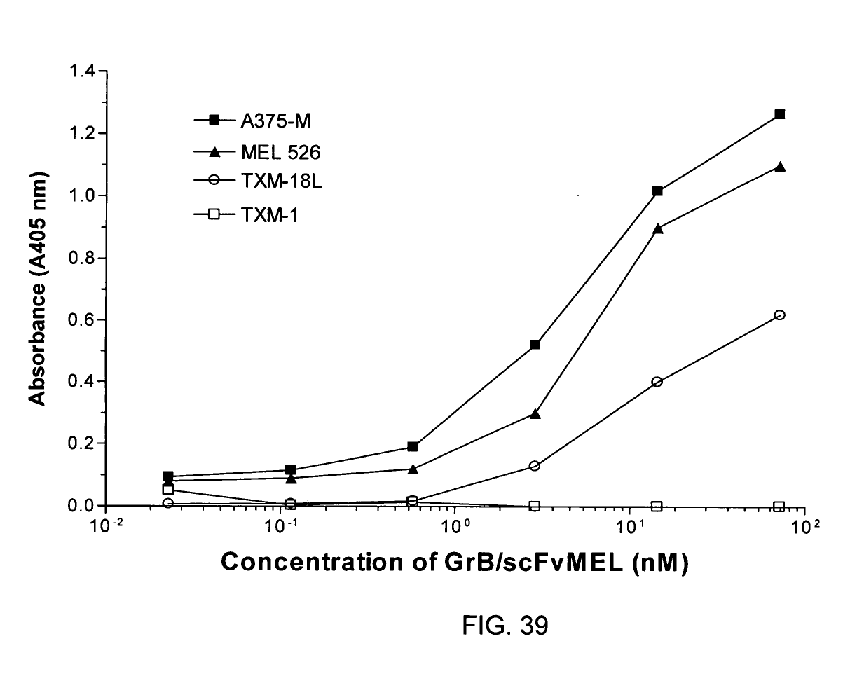

Since gp240 antigen highly presents on melanoma A375-M, MEL-526, TXM-18L cells but not TXM-1 cells, we further examined if the GrB/scFvMEL fusion construct could bind to the antigen positive cells. An ELISA was performed to determine the binding activity of the GrB/scFvMEL fusion construct to melanoma cells. The results demonstrated that GrB/scFvMEL bound to high-level gp240 antigen expressing melanoma A375-M, MEL-526, and TXM-18L. Moreover, the binding activity was stronger in A375-M and MEL-526 followed by TXM-18L. However, the protein did not bind to TXM-1 in which has very low-level expression of gp240 antigen as detected by an anti-scFvMEL rabbit monoclonal antibody (FIG. 39).

The GrB moiety of the fusion construct was efficiently delivered into the cytosol of gp240 antigen positive A375-M, MEL-526, TXM-18 melanoma cells after treatment with GrB/scFvMEL for 1 h as assessed by immunofluorescent staining detected using goat anti-GrB antibody. This effectively demonstrates that the gp240 expression on the tumor cell surface and the binding of the construct to gp240 antigen are responsible for internalization of the GrB/scFvMEL fusion construct.

### Example 45

### In Vitro Cytotoxicity of GrB/scFvMEL Against Various Melanoma Cell Lines

The cytotoxicity of GrB/scFvMEL was assessed against log-phase A375-M, MEL-526, TXM-18L and TXM-1 cells in culture. The 50% growth inhibitory effects were found at concentrations of 30 nM on A375-M cells, ˜50 nM on MEL-526 cells, ˜150 nM on TXM-18L, respectively. However, no cytotoxic effects were found on TXM-1 cells at doses of up to 1 μM (FIG. 36, Table 16). By comparison, the cytotoxic effects of GrB/scFvMEL on target cells were similar to those of the previous described MEL sFv/rGel (Rosenblum et al., 2003) (Table 16).

TABLE 16
Cytotoxicity of GrB/scFvMEL vs MEL sFv/rGel
against Various Human Melanoma Cell Lines*

| | Gp240 Antigen | I.C.₅₀ (nM) | I.C.₅₀ (nM) |
|---|---|---|---|
| Cell line | % positive** | GrB/scFvMEL | scFvMEL/rGel |
| A375-M | 98.9 | 32.1 ± 7.95 | 22.03 ± 3.08 |
| MEL-526 | 97.8 | 50.0 ± 4.32 | 40.0 ± 5.55 |
| TXM-18L | 40.9 | 158.3 ± 12.45 | 144.3 ± 9.56 |
| TXM-1*** | 4.6 | >1000 | >1000 |

*Samples (GrB/scFvMEL, MEL sFv/rGel) were assayed using a standard 72-h cell proliferation assay and using crystal violet staining. I.C.₅₀ values were calculated and demonstrated as means ± SEM (nM).
**Percent of positive (% positive) FACS analysis.
***No cytotoxic effect was observed at doses up to 1000 nM.

These data indicate that there appears to be a correlation between high expression of gp240 leading to improved cellular binding of GrB/scFvMEL and resulting in a comparatively greater cytotoxicity of the fusion protein. In addition, studies in the laboratory of the inventor demonstrate that a companion construct MEL sFv/rGel generates a cytotoxic effect against cells through a necrotic rather than an apoptotic process. These comparative studies demonstrate that the cytotoxic/apoptotic effects of the GrB payload can match the robust cytotoxic/necrotic effects of rGel-containing fusion toxins.

### Example 46

### Combination Studies of GrB/scFvMEL with Conventional Chemotherapeutic Agents on A375-M

Since cellular apoptotic processes appear to mediate response/resistance to chemotherapy (Simstein et al., 2003; Soengas and Lowe, 2003), the inventor next examined the effects of GrB/scFvMEL in combination with various chemotherapeutic agents on target cells. Co-administration GrB/scFvMEL and various classes of chemotherapeutic agents to A375-M cells for 72 hours, demonstrated synergistic antitumor activity with adriamycin (DOX), vincristine (VCR) or cisplatin (CDDP) and additive effects in combination with etoposide (VP16), cytorabine (Ara-C) or 5-Fu. Pre-treatment with GrB/scFvMEL for 6 h followed by co-exposure to these chemotherapeutic agents for 72 hours (sequence II) showed significantly inhibited growth as compared to pre-treatment with drugs followed by co-exposure the fusion construct (sequence I) (FIG. 37). The results indicated that the cytotoxic effects of chemotherapeutic agents could be markedly enhanced by pretreatment with GrB/scFvMEL for 6 h on gp240 antigen-positive melanoma cells. GrB/scFvMEL was not cytotoxic to antigen negative TXM-1 cells at concentrations up to 1 μM. The inventor pretreated TXM-1 cells with GrB/scFvMEL at 1 μM for 6 hr, then co-administered various chemotherapeutic agents at I.C.₅₀ or I. C.₂₅ doses. As expected, the cytotoxic effects of various chemotherapeutic agents were not enhanced by pre-treatment with GrB/scFvMEL for 6 hr.

### Example 47

### Cytotoxicity of GrB/scFvMEL Against A375-Doxorubicin Resistant (A375DR) Subline

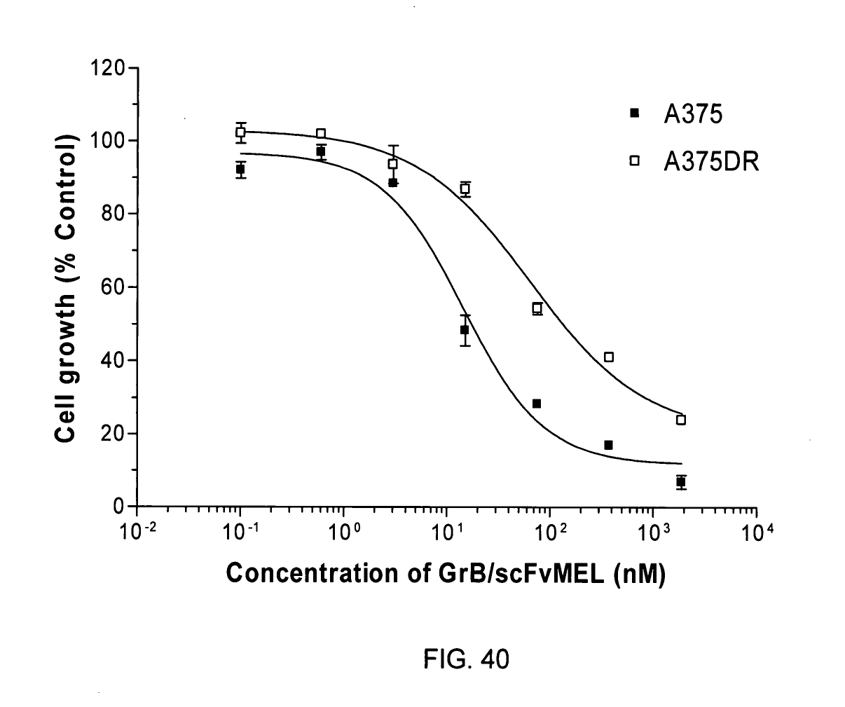

To examine if cellular resistance to doxorubicin also causes cross-resistance to GrB/scFvMEL fusion construct, an A375-doxorubicin resistant (A375DR) cell line was first established by continuous exposure of log-phase parental A375-M cells to increasing concentrations of doxorubicin followed by serial dilution and clonal selection of colonies. One clone designated A375DR was 400 fold resistant to doxorubicin compared to the parental A375 cell line (I.C.₅₀: 200 nM for A375DR vs. I.C.₅₀: 0.5 nM for the parental A375 cell line). Treatment of the A375DR cells demonstrated an I.C.₅₀ only 4.5 fold higher compared to the parental A375 cells (I.C.₅₀: 63.6 nM for A375DR vs I.C.₅₀: 14.5 nM for A375) (FIG. 40, Table 17). Expression of the gp240 antigen was assessed by ELISA using ZME-018 antibody specifically recognizing gp240 antigen. This study demonstrated that the relative gp240 expression was virtually identical on parental A375 and A375DR cells (data not shown). The data demonstrated that cellular resistance to doxorubicin is associated with a marginal cross-resistance to GrB/scFvMEL.

TABLE 17
Comparison of cytotoxic effects of GrB/scFvMEL and Doxorubicin on
parental A375 cells and A375 doxorubicin-resistant (A375DR) sublines

| | I.C.₅₀ (nM) | I.C.₅₀ (nM) |
|---|---|---|
| Cells | Doxorubicin | GrB/scFvMEL |
| A375 | 0.5 ± 0.19 | 14.5 ± 3.93 |
| A375DR | 200.0 ± 16.28 | 63.6 ± 4.01 |
| Resistance Index* | 400.0 | 4.5 |

*Resistance Index is defined as (I.C.₅₀ on A375DR)/(I.C.₅₀ on A375)

### Example 48

### Effects of Pre-Treatment with GrB/scFvMEL on Radiosensitivity in Human Melanoma A375-M Cells

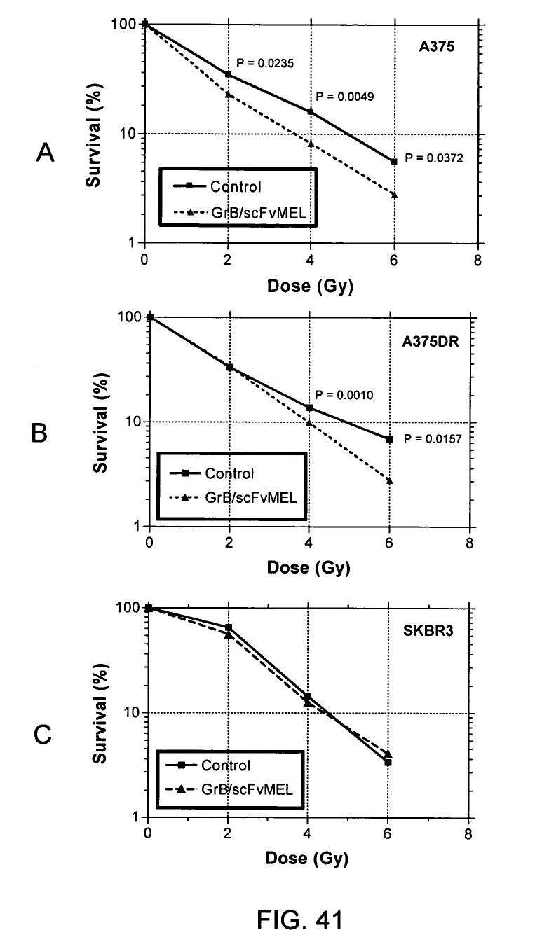

To determine if induction of apoptosis by GrB/scFvMEL can impact the sensitivity of melanoma cells to external-beam ionizing radiation, A375, A375DR and antigen negative SKBR3 cells were first pretreated with 10 nM of GrB/scFvMEL for 16 hours, the cells were then irradiated and plated for clonogenic cell survival. FIG. 41 showed that GrB/scFvMEL treatment suppressed the clonogenic survival of both A375 and A375DR cells. The numbers of surviving colonies at radiation doses of 2, 4 and 6 Gy were reduced from approximately 34.8±0.23%, 16.0±0.06% and 5.6±0.55% respectively in the A375 radiation-alone control group to 23.1±1.59%, 8.2±0.49% and 2.8±0.52% in the GrB/scFvMEL plus radiation treatment group respectively (FIG. 41A, Table 18A).

TABLE 18A
Clonogenic survival (% Survival) of A375 cells treated with irradiation
alone (control) vesus irradiation and treatment with GrB/scFvMEL.

| | 0 Gy | 2 Gy | 4 Gy | 6 Gy |
|---|---|---|---|---|
| Control | 100 | 34.8 ± 0.23 | 16.0 ± 0.06 | 5.6 ± 0.55 |
| GrB/scFvMEL | 100 | 23.1 ± 1.59 | 8.2 ± 0.49 | 2.8 ± 0.52 |
| p value | >0.05 | *<0.05 | *<0.05 | *<0.05 |

The observed sensitization was statistically significant (p<0.05) at 2, 4 and 6 Gy dosage groups. After treatment of A375DR cells with irradiation alone, the colony survival at doses 4 and 6 Gy was 13.8±0.32% and 6.9±0.64% respectively compared to 9.4±0.29% and 2.8±0.12%, respectively in the groups co-treated with GrB/scFvMEL and radiation (FIG. 41B, Table 18B).

TABLE 18B
Clonogenic survival (% Survival) of A375DR cells treated with irradiation
alone (control) vesus irradiation and treatment with GrB/scFvMEL.

| | 0 Gy | 2 Gy | 4 Gy | 6 Gy |
|---|---|---|---|---|
| Control | 100 | 33.4 ± 0.09 | 13.8 ± 0.32 | 6.9 ± 0.64 |
| GrB/scFvMEL | 100 | 34.0 ± 1.07 | 9.4 ± 0.29 | 2.8 ± 0.12 |
| p value | >0.05 | >0.05 | *<0.05 | *<0.05 |

The observed sensitization in the A375DR groups was statistically significant with p<0.05 at 4 and 6 Gy, respectively. However, no statistically-significant sensitization was observed in antigen-negative SKBR3 cells treated with irradiation compared with GrB/scFvMEL-treated cells followed by irradiation at 2, 4, and 6 Gy (p>0.05) (FIG. 41C, Table 18C). Therefore, treatment with GrB/scFvMEL sensitizes both antigen-positive A375 and A375DR doxorubicin-resistant cells to ionizing radiation. This radio-sensitization appears to be dependant on expression of gp240 antigen since we did not observe radiation sensitization with antigen-negative cells.

TABLE 18C
Clonogenic survival (% Survival) of SKBR3-HP cells treated
with irradiation alone (control) vesus irradiation and
treatment with GrB/scFvMEL.

| | 0 Gy | 2 Gy | 4 Gy | 6 Gy |
|---|---|---|---|---|
| Control | 100 | 59.8 ± 6.90 | 12.5 ± 4.45 | 3.4 ± 0.13 |
| GrB/scFvMEL | 100 | 55.9 ± 0.27 | 12.2 ± 1.43 | 4.1 ± 0.23 |
| p value | >0.05 | >0.05 | >0.05 | >0.05 |

### Example 49

### Effect of GrB/scFvMEL on the Metastatic Potential of A375DR Cells

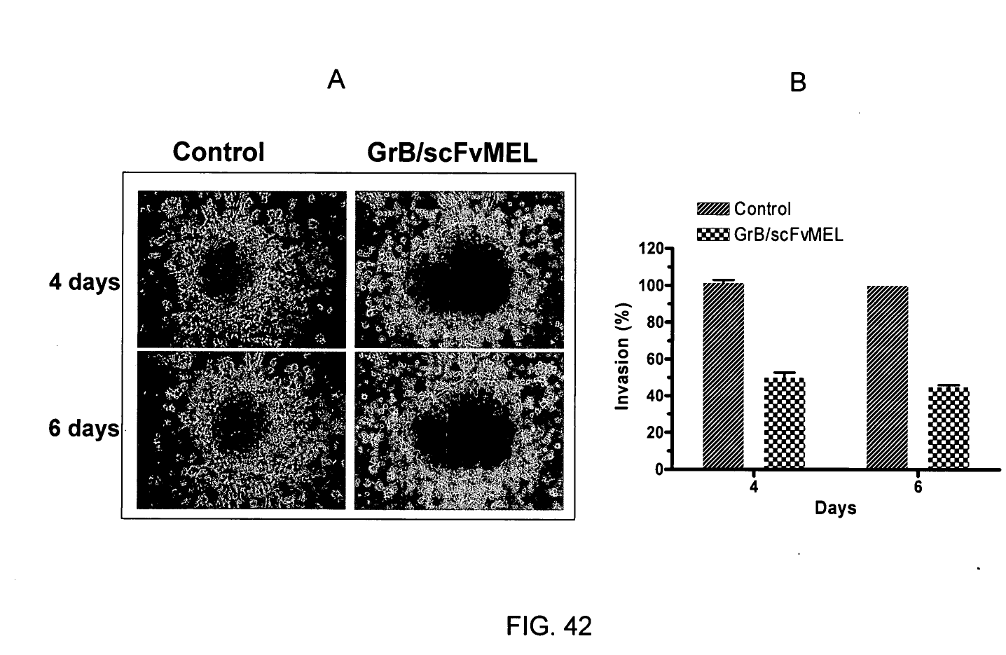

It was then examined whether the GrB/scFvMEL fusion construct might affect the metastatic potential of melanoma cells since the cellular apoptotic posture is one of the factors known to be responsible for mediating cellular invasion and metastatic potential (Glinsky and Glinsky, 1996; Glinsky et al., 1997; Rubio et al., 2001; Simstein et al., 2003). The effects of the GrB/scFvMEL on the ability of A375DR cells to invade a matrix of a reconstituted basement membrane (Matrigel) was studied. A375DR cells spontaneously form cell aggregates in Matrigel, when prepared by the hanging-drop technique. A375DR cells actively leave the aggregate and invade the Matrigel preparation at 4 and 6 days (FIG. 42A). The densities of cells invaded into matrigel surrounding the aggregates was analyzed by AlphaEase®FC software (Alpha Innotech, San Leandro, Calif.) and the percent of invasion was calculated based on the cell densities of two groups and standardized by the value of non-treatment control group as 100% invasion. The treatment of A375DR cells with GrB/scFvMEL inhibits A375DR invasion of Matrigel. The capability of invasion of cells significantly decreased to 50.0±2.89% at 4 days and 45.0±1.16% at 6 days (FIGS. 42A and 42B).

### Example 50

### In Vivo Animal Studies of GrB/scFvMEL (A375-M Xenograft Model)

The internalization or localization of the GrB/scFvMEL fusion construct in mice bearing A375-M xenograft tumors was first characterized. After administration of GrB/scFvMEL 24 hr later, mice were sacrificed and representative tissue sections were removed and formalin fixed and stained by hematoxylin and eosin (H and E) and detected by either anti-GrB antibody or anti-scFvMEL antibody. Localization or internalization of GrB/scFvMEL was observed in tumor tissue.

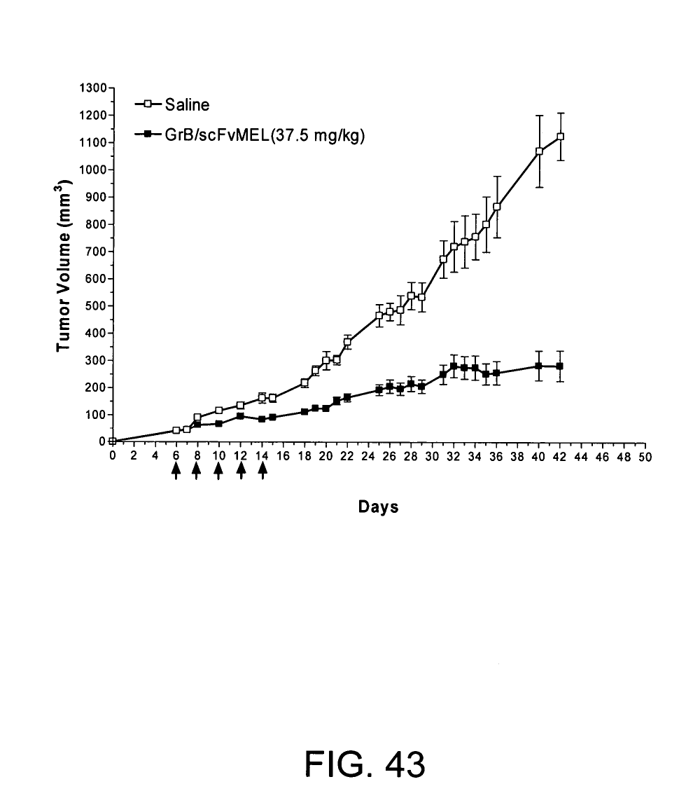

To examine the in vivo anti-tumor effects of GrB/scFvMEL, the studies were performed on A375-M human melanoma tumor xenografts. Mice bearing the tumors were treated (iv tail vein) 5 times every other day with either GrB/scFvMEL or saline. The saline treatment control group tumors increased 24 fold (from 50 mm³ to 1200 mm³) over 28 days. In contrast, GrB/scFvMEL treated tumors increased 4 fold (from 50 mm³ to 200 mm³) (FIG. 43). The GrB/scFvMBL fusion construct demonstrates impressive antitumor activity. The tumor tissue nuclei were stained by TUNEL assay. The results clearly demonstrated that tumor tissue displayed apoptotic nuclei in GrB/scFvMEL treatment group (FIG. 40).

### Example 51

### Exemplary Clinical Studies

The present example regards scFvMEL/TNF in particular yet exemplary embodiments, but one of skill in the art recognizes that the exemplary protocol may be employed with other chimeric molecules of the invention. Furthermore, one of skill in the art recognizes that this exemplary protocol may be optimized when adapting the protocol for another chimeric molecule, which is done routinely in the art.

In specific embodiments of the invention, the safety and tolerability of a chimeric molecule of the invention, such as scFvMEL/TNF, for example, is evaluated, such as in patients with gp240-positive advanced malignancies, including melanoma and lobular breast cancer, for example. In additional embodiments, the pharmacokinetic profile of a chimeric molecule of the invention, such as scFvMEL/TNF, is evaluated when administered to individuals, such as patients with gp-240 malignancies. In further embodiments, the maximum tolerated dose (MTD) and dose limiting toxicities (DLT) of a chimeric molecule of the invention is established, such as scFvMEL/TNF, for example.

ScFvMEL is a murine antibody directed against the gp240 surface glycoprotein found on malignant melanoma, lobular breast carcinomas and possibly other types of tumors. Tumor necrosis factor (TNF) is a 17, 000 MW cytotoxic polypeptide and mediates a wide spectrum of systemic and cellular responses, including tumor necrosis and apoptosis. TNF is cytostatic or cytotoxic to a number of human tumor cells in vitro. The use of the TNF in cancer therapy is restricted by severe toxicity. A variety of strategies have been suggested to utilize the antitumor properties of this agent and simultaneously reduce the systemic side effects including antibody-mediated delivery. ScFvMEL/TNF is a fusion protein consisting of a single chain murine monoclonal antibody (scFvMEL) fused at its C-terminal end to recombinant TNF. ScFvMEL/TNF can deliver cytotoxic amounts of TNF to the approximately 80% of malignant melanoma tumor cells that express gp240. This novel immunocytokine constitutes an attractive and promising compound for development in clinical trials.

Exemplary eligibility criteria include the following: 1) adults older than 12 years of age with diagnosis of malignant melanoma confirmed by histopathology; 2) more than 20% of the tumor cells in the available pathologic sample must demonstrate some detectable staining in either the membrane or cytoplasm; 3) mental and emotional ability of participant subject to understand and sign written informed consent forms; 4) adequate performance status. (Karnofsky scale 60%/ECOG≦2); 5) at least three weeks since previous therapy such as major surgery, cytotoxic chemotherapy, immunotherapy interleukin-2 or interferon, or anti-cancer vaccination; and/or 6) adequate function of renal, hepatic, and hematological systems as reflected by the following parameters: creatinine <2.0 mg/dL; AST/ALT <2.5 times the normal upper limits of references laboratory performing the assay; AST or ALT <6× normal upper range if liver metastases have been documented; total bilirubin <2.5 mg/dL AST or ALT <6× normal upper range if liver metastases have been documented; platelet counts in peripheral blood >100,000 cells/uL; absolute neutrophil counts >1.5 cells/uL; patients must have at least one measurable or evaluable lesion that can be followed throughout the study. Patients with lesions that are not measurable but still evaluable (e.g. small volume multiple cutaneous deposits) may be entered on study. Excluded individuals include the following: women who are pregnant or lactating; dementia or other mental disturbance that would impede adequate understanding and decision-making during consent process; concomitant use of immunosuppressant medications (i.e., corticosteroids, cyclosporine, tacrolimus, etc.); history of other concomitant primary malignancies; and/or concomitant use of cytotoxic or immunological agents (hydroxyurea, tamoxifen, etc.).

All cycles of scFvMEL/TNF will be administered in the outpatient setting. The initial cohort of patients will receive 3 mg/m² given as a 1 hr IV infusion on days 1-5 and 8-12 (MON-FRI of each of two successive weeks) of each 28 day cycle. Therapy may be subsequently continued if the subject does not develop unacceptable toxicity (grade 3-4) or progression of disease during treatment. Assessment of the response of visible or palpable tumor will be repeated at the end of each cycle; assessment of the response of tumors detectable only by imaging will be carried out at the end of every two cycles. Further treatment with study drug will be at the discretion of the principal investigator and will be based on an evaluation of all safety and efficacy data available at the time.

ScFvMEL/TNF is a Highly Selective Immunocytokine

The fusion construct scFvMEL/TNF immunocytokine was shown specifically high-level binding to gp240 antigen positive melanoma cells but not or low-level binding to gp240 antigen-negative other cells as assessed by ELISA. Scatchard analysis of binding of radiolabeled-scFvMEL/TNF to gp240 antigen positive, TNF-sensitive human melanoma A375-M cells revealed the presence of two binding sites on cell surface, one with a dissociation constant (Kd) of 1.9 nM and approximately 4000 binding sites per cells, the other with a Kd of 15.6 nM and approximately 1.6×10⁵ binding sites per cells (Liu et al., 2004).

ScFvMEL/TNF is a trimer (135,000 dalton) in solution. Analysis of scFvMEL/TNF by size-exclusion FPLC showed that the scFvMEL/TNF fusion construct migrated at an apparent size of approximately 135 kDa at 93% of the injected protein. This agrees closely with the predicted size of a trimeric structure of the 45 kDa fusion construct. Therefore, the fusion construct appears to self-aggregate in solution into a trimeric structure in a fashion similar to that of the native TNF. A small peak comprising 7% of the injected material was observed migrating with an apparent molecular weight of approximately 45 kDa. This is the expected size of scFvMEL/TNF monomer (Liu et al., 2004).

TNF moiety of scFvMEL/TNF was Efficiently Internalized

The binding and internalization of scFvMEL/TNF was evaluated by immunoflourescence of A375-M cells. Cells pre-blocked with anti-TNFR1 antibody and then treated with scFvMEL/TNF at 37° C. At the designated times, cell surfaces were washed and stripped by low pH (pH 2.5) glycine buffer to remove excess fusion protein. Cells were fixed and permeabilized followed by blocking nonspecific binding. Further cells were incubated with rabbit anti-huTNF antibody and detected with FITC-coupled antirabbit IgG. The signals of TNF were observed in the cytosol of 1-hr-treated cells but not in that of non-treated cells under fluorescent microscope. The amount of TNF in the cytosol was dependent on the duration of exposure to the construct.

The Biological Activity of the TNF Component in the Fusion Construct is the Same as that of Native TNF

The biologic activity of TNF was determined by using a standard assay which depends on cytotoxicity in L-929 cells. Native TNF had a specific activity of 26.7×10⁶ units/mg protein. The biologic activity of the TNF component of the fusion construct scFvMEL/TNF remained essentially intact because the specific activity of scFvMEL/TNF (10×10⁶ units/mg protein) was approximately the same as that of native TNF. No loss of activity occurred when TNF was fused to the antibody structure.

Immunocytokine Specificity

The scFvMEL/TNF purified fusion protein was tested for specific cytotoxicity against an antigen-positive, TNF-sensitive human melanoma A375-M cells, an antigen-positive, TNF-resistant human melanoma AAB527 cells, an antigen-negative, TNF-sensitive human breast cancer SKBR3-HP cells, and an antigen-negative, TNF-resistant human neuroglioma H4 cells compared with native TNF alone. The cytotoxic effect of the immunocytokine against antigen-positive, TNF-sensitive A375-M cells was 10 fold more active than native TNF. Against antigen-negative, TNF-sensitive SKBR3-HP cells, the cytotoxicity of scFvMEL/TNF showed a dose-response curve similar to that of native TNF. Against antigen-negative, TNF-resistant cells, the scFvMEL/TNF demonstrated no cytotoxic effects at doses up to 100 nM. However, against antigen-positive, TNF-resistant AAB-527 cells, the scFvMEL/TNF showed significant dose-related cytotoxic effects. The scFvMEL/TNF can overcome TNF resistance.

Pre-Clinical Pharmacology Studies of scFvMEL/TNF in Mice

Pharmacology studies of scFvMEL/TNF in mice show a tri-phasic clearance curve with calculated plasma half-lives of 0.38, 3.9 and 17.6 hours for the alpha, beta and gamma half-lives respectively. The immediate apparent volume of distribution (Vda) of scFvMEL/TNF was 19.50 ml. The area under the concentration curve (AUC) was calculated to be 4.0 μg/ml×hr. The mean residence time was found to be 8.3 hrs.

LD, MTD and Toxicology Studies of scFvMEL/TNF in Mice

Mice were treated (iv) with total doses of scFvMEL/TNF ranging from 0.5 to 16.7 mg/kg in six divided treatments every day for 5 days. A dose of 8.3 mg/kg was found to be the LD₁₀₀ while the LD₂₅ was found to be 4.8 mg/kg. The highest non-lethal dose defining the MTD was found to be 4 mg/kg total dose at this schedule.

Toxicity studies in Balb/c mice were conducted at various doses including 100% of the MTD (4 mg/kg), 75% MTD (3 mg/kg), 50% MTD (2 mg/kg) and 25% MTD (1 mg/kg). There were no significant effects noted on clinical chemistry parameters at these doses. There were no significant effects noted on hematological effects. The no-observed adverse effect level (NOAEL) was determined to be 3 mg/kg (9 mg/m²). Therefore, the starting dose in patients will be 1/3 the NOAEL dose or 3 mg/m2 (total dose given as 0.3 mg/m2/day on days 1-5 and 8-12 of each 28-day cycle).

In Vivo Efficacy of scFvMEL/TNF

Treatment of scFvMEL/TNF at 2.5 mg/kg dosage at early stage (50 mm3) demonstrated potent antitumor activity indicating 3 of 5 mice tumor-free on day 21, and complete tumor regression, 5 of 5 mice tumor-free on day 43 when monitor by caliper measurement. Mice treated at later stage (200 mm³), also showed tumor regression, 3 of 5 tumor-free on day 44. There was no subsequent outgrowth of tumors from mice rendered tumor free. These data showed that scFvMEL/TNF can target melanoma cells in vivo and can result in pronounced anti-melanoma effects.

Study Design

This is a Phase I, open-label, dose escalation study to assess the safety, tolerability, PK and efficacy of scFvMEL/TNF. Once the MTD has been defined, additional patients will be entered at this dose to further refine the recommended Phase I dose. The starting dose will be 0.3 mg/m² (1/3 of the mouse NOAEL dose) given on each of days 1-5 and 8-12 of a 28-day cycle.

Dose Escalation

Dose escalation will proceed as follows: 100% escalation until any biological activity (except mild nausea and vomiting) is observed; 50% escalation thereafter until a grade 2 adverse event (AE) is observed; 25% escalation thereafter until the dose at which limiting toxicity occurs is encountered. This is defined as the dose at which 2 or more of 6 patients suffer a grade 3 non-hematological or a grade 4 hematological adverse event. The maximum tolerated dose (MTD) is defined as the next lower dose level. Once the MTD has been ascertained, at least 3 patients will receive at least 3 cycles at that dose to ascertain whether there is cumulative toxicity. At least 6 patients will be studied at the maximum tolerated dose.

One patient will be treated per dose level until a grade 2 adverse event (AE) develops; thereafter 3 patients will be enrolled per dose level. If 1 of 3 patients at any given dose level suffers a grade ≧3 or greater AE then 3 more patients will be entered at that dose level. If >2 of 6 patients suffer a grade 3 non-hematological or a grade 4 hematological AE no further dose escalation will be undertaken. This dose level will be defined as the maximum tolerated dose (MTD).

Once a dose has been reached that requires the entry of 3 patients per dose level, patients may be entered at the next higher dose level once 1 of 3 patients at the preceding dose level has received 10 infusions over 14 days plus 2 weeks follow-up and the remaining 2 patients at the preceding dose level have received at least 5 infusions and are eligible for their sixth infusion, all without the occurrence of dose limiting toxicity (DLT).

Dose escalation is permitted in the same patient during subsequent cycles provided no AE grade >1 was observed during the preceding cycle. However, if a grade 2 or greater AE occurred, and the patient is otherwise eligible to receive additional cycles, the patient will continue to receive scFvMEL/TNF without further dose escalation.

All decisions about dose escalation will be made by the Principal Investigator in consultation with the medical monitor. The planned dose escalation levels may be changed as information from the trial becomes available.

Patients are eligible to received additional cycles of scFvMEL/TNF if there is evidence of improvement (stable disease or a decrease in tumor size, symptom relief) and non-hematologic adverse events were no greater than Grade 2. Patients may receive up to a total of 5 additional cycles at the discretion of the study investigator. The dose to be used in subsequent cycles will be determined by the study investigator based on all information available at the time.

Dose adjustments for individual patients receiving >1 cycle will be based on adverse events. If the patient suffers a study drug-related grade 3 AE on the preceding cycle subsequent doses will be held until the AE resolves to pretreatment baseline.

Definition of Dose Limiting Toxicity (DLT)

DLT is defined as: “an adverse reaction which is probably or definitely related to study drug” that meet the following NCI Common Toxicity Criteria: Hematologic Toxicity: grade 4 neutropenia ≧5 days, Grade 4 thrombocytopenia, Neutropenic infection; Non-Hematologic Toxiticy: any grade 3 or 4 non-haematologic toxicity, excluding nausea, vomiting, diarrhea and alopecia.

Statistical Analysis

Given the nature of this clinical trial, regular descriptive statistics will be used for its analysis.

Treatment Plan

All cycles of scFvMEL/TNF will be administered in the outpatient setting. The initial cohort of patients will receive 3 mg/m² given as a 1 hr IV infusion on days 1-5 and 8-12 (MON-FRI of each of two successive weeks) of each 28-day cycle. Therapy may be subsequently continued if the subject does not develop unacceptable toxicity (grade 3-4) or progression of disease during treatment. Assessment of the response of visible or palpable tumor will be repeated at the end of each cycle; assessment of the response of tumors detectable only by imaging will be carried out at the end of every two cycles. Further treatment with study drug will be at the discretion of the Principal Investigator and will be based on an evaluation of all safety and efficacy data available at the time.

Patients will be seen in clinic weekly at which time and change in their symptoms and signs, observable tumor and concomitant medications will be noted and blood obtained for determination of hematologic parameters and serum chemistries. A repeat tumor biopsy will be obtained at the beginning of the third week of the first cycle (day 16±3). Imaging studies to assess tumor size will be repeated at the end of every other cycle.

Blood Sampling for Pharmacokinetics

All patients will undergo blood sampling for determination of study drug levels and measurement of antibody levels on each cycle. Intensive sampling will be done on cycle 1; less intense sampling will be done on each subsequent cycle. Each blood sample is to consist of 10 ml of blood drawn into a heparinized tube. All blood samples should be immediately centrifuged at 4° C. and the plasma removed and frozen in 3 aliquots. The timings of these samples may be modified based on analysis of the first few patients in order to acquire optimal pharmacokinetic parameters.

For Cycle 1, Day 1: Just prior to the start of study drug infusion, right at the end of study drug infusion and at the following times after the end of drug infusion: 5, 10, 20, 40, 80, 160 and 320 min. For Days 2-5 and days 8-12: just before and immediately at the end of each daily drug infusion. For Cycle 2 and all subsequent cycles For Days 1-5 and days 8-12: just before and immediately at the end of each daily drug infusion.

Histologic Evaluation of Tumor Biopsies

A biopsy of accessible tumor will be obtained prior to the first infusion of study drug and again at the beginning of the third week of the first cycle (day 16±3). In addition to routine pathologic examination the following parameters will be assessed on both the pre- and post-treatment sample using immunohistochemical and in situ hybridization assays: expression of gp240, proliferation rate (anti Ki67), and degree of apoptosis (with TUNEL technique), tumor content of TNF and extent of necrosis.

All data will be collected in a semiquantitative manner and compared using the chi-square test and Student t-test.

Evaluation of Toxicity

Toxicity will be monitored and graded according to the Cancer Therapy Evaluation Program Common Toxicity Criteria version 2.0 (CTCv2.0). Adverse events not included in the CTCv2.0 should be reported and graded under the “other” adverse events within the appropriate category. A copy of the CTCv2.0 can be downloaded from the CTEP homepage.

Criteria for Response

Tumor response will be a prospective secondary end point of this clinical study. The objective tumor responses in this study will be evaluated using the Response Evaluation Criteria in Solid Tumors Group (RECIST) (Therasse, Arbuck et al.) Easily observable skin lesions will be measured with calipers; tumor masses internal to the body will be assessed using CT, MRI or ultrasound using protocol guidelines developed by the Department of Radiology at M. D. Anderson Cancer Center. The same method of assessment and the same technique will be used to characterize each identified and reported lesions at baseline and during follow-up.

Definitions: At baseline, Tumor Lesions will be Categorized as Follows.

Measurable: All lesions that can be accurately measured in at least one direction [longest diameter as recorded] as =20 mm with conventional techniques or as =10 mm with spiral C T scan. Measurable disease is defined by the presence of at least one lesion that this description.

Non-measurable: All lesions with longest diameter <20 mm with conventional techniques or <10 mm with spiral CT scan, and truly non-measurable lesions (leptomeningeal disease, ascites, pleural or pericardial effusions, bone disease, lymphangitis.

Target and non-target lesions: All measurable lesions up to a maximum of five lesions per organ and 10 lesions in total, representative of all involved organs, should be identified as target lesions and recorded and measured at baseline. Target lesions should be selected by size (those with the longest diameter) and their suitability for accurate repeated measurements (either by imaging studies or clinically). A sum of the longest diameter for all target lesions will be calculated and reported as the baseline sum longest diameter. The baseline sum longest diameter will be used as the reference to characterize the objective tumor response. All other lesions should be identified as non-target lesions and recorded at baseline.

Overall Responses

Evaluation of target lesions: complete response occurs when disappearance of all target lesions occurs; partial response occurs when imaging studies or clinical evaluation demonstrates at least 30% decrease in the sum of the longest diameter of target lesions, taking as reference the baseline sum longest diameter; progressive disease occurs when at least 20% increase in the sum of the longest diameter of target lesions, taking as reference the smallest sum longest diameter recorded since treatment started or the appearance of one or more new lesions; stable disease occurs whenever neither sufficient shrinkage to qualify for partial response nor sufficient increase in size to qualify for progressive disease is present.

Evaluation of non-target lesions: complete response: is the disappearance of all non-target lesions; Incomplete response/stable disease is the persistence of one or more non-target lesions; progressive disease is the appearance of one or more new lesions and/or unequivocal progression of the existing non-target lesions; Evaluation of best overall response: the best overall response is the best response recorded from the start of treatment until disease progression/recurrence (taking as reference for progressive disease the smallest measurements recorded since the treatment started). Patients with deterioration of their health status requiring discontinuation of therapy without objective evidence of disease progression at that time should be classified as having “symptomatic deterioration.” Every effort should be made to document the objective disease progression, even after discontinuation of treatment. If complete resolution of a lesion occurs and it is difficult to distinguish residual disease from normal tissue, a histological evaluation of the residual abnormality must be investigated with a fine needle aspirate before confirming the complete response status.

Pharmacokinetic Studies of scFvMEL/TNF in Mice

The exemplary pharmacokinetics of scFvMEL/TNF in mice was determined. An exemplary method employing ¹²⁵I-radiolabeled scFvMEL/TNF using P-iodobenzoate was utilized. This method conjugates N-succinimidyl p-iodobenzoate to the protein. Briefly, 37.5 μl 1% HOAc/MeOH, 10 μl 1 mg/ml N-chlorosuccinimide in MeOH, and 10 μl PBS were sequentially added to a reaction vial fitted with a rubber septum containing N-succinimidyl 4-tri-n-butylstannlbenzoate) Neorx Corp., Seattle, Wash.) (12.5 mg) in 12.5 μl HOAc/MeOH. A 1-mCi aliquot of ¹²⁵I (Dupont) was added to the reaction solution and after 5 min, the reaction was quenched by addition of 10 μl 0.1M NaHSO₃. The MeOH solvent was evaporated under a N₂ stream for 10 min. A 500 μg sample of protein in 100 ml PBS was mixed with 100 μl 0.5 M borate buffer (pH 9.3) and then added to the reaction vial. The conjugation was allowed to proceed for 5 min at RT. Unreacted radioiodine was removed by chromatography on a Sephadex G-25 (PD-10) column (Pharmacia LKB Biotechnology, Piscataway, N.J.). The radiochemical yield was 40%-60%. Incorporation of radiolabel into protein measured by trichloroacetic acid precipitation was greater than 90%. The specific activity of radiolabeled proteins was 0.4 μCi/μg.

An exemplary pharmacokinetics study is as follows. BALB/c mice, 4-6 weeks old, were injected with 2 μCi per mouse, 5 μg total protein in 200 μl of normal saline. One hour, 2 h, 4 h, 8 h, 24 h, 48 h, 72 h after injection, two mice at each assay time were sacrificed by cervical dislocation. Blood samples were removed from chest cavity, weighed and counted to determine total radioactivity in a gamma counter (Packard, model 5360). The blood samples were also centrifuged and plasma was decanted and counted to determine radioactivity. Results from plasma determinations of radioactivity were analyzed by a least square nonlinear regression (RSTRIP from MicroMath, Inc.) program to determine pharmacokinetic parameters.

The data was graphed, and the mean±SEM of the data at each timepoint was determined. The data demonstrated that the scFvMEL/TNF construct clears from the circulation with a terminal hase half-life of 17.6 hrs after iv administration. The immediate apparent volume of districution (Vda) of scFvMEL/TNF was 19.50 ml. The area under the concentration curve (AUC) was calculated to be 4.0 mg/ml×hr. The mean residence time was determined to be 8.3 hrs.

Determination of Maximal Tolerated Dose (MTD) and Lethal Dose (LD) for scFvMEL/TNF Immunocytokine in Nude Mice

The MTD and LD of scFvMEL/TNF Immunocytokine was determined. Nude mice (nu/nu) were assigned to 6 different cages and scFvMEL/TNF (2 mg/ml) immunocytokine was administered by tail vein daily×5. The LD₂₅ of scFvMEL/TNF in mice is approximately 4.8 mg/kg. The LD₁₀₀ of scFvMEL/TNF in mice is approximately 8.3 mg/kg. The MTD of scFvMEL/TNF in mice is approximately 4.0 mg/kg.

Formulation Studies to Determine Optimal Storage Conditions for Maintenance of Biological Activity of scFvMEL/TNF

The optimal buffer composition, pH, storage temperature and additional protein content to maintain optimal biological activity of scFvMEL/TNF was determined.

Samples of scFvMEL/TNF were added to sodium phosphate buffer (100 mM) at pH 6.5, 7.0 and 7.5. Duplicate samples containing 0.1% HSA were also prepared and all six samples were stored at 4° C. or −20° C. Over time (8 weeks), samples were removed and tested for comparative cytotoxicity against A375-M cells in culture as follows:

Cells were plated into 96-well plates at a density of 4×10³ cells/well and allowed to adhere overnight. Then, the medium was replaced with medium containing different concentrations of scFvMEL/TNF. After 72 h, the effects of scFvMEL/TNF on the growth of tumor cells in culture were determined using crystal violet staining. Briefly, cells were fixed and stained by the addition of 0.5% crystal violet in 20% methanol (0.05 ml/well) for 30 min. The plates were rinsed in deionized water and crystal violet was extracted from adherent cells by the addition of 0.2 ml Sorenson's buffer/well (0.1 M sodium citrate, pH 4.2, in 50% ethanol). Cell plates were vortexed for 30 min at room temperature and the absorbance was read at 630 nm (Bio-Tek Instruments, Winooski, Vt.) and compared with control wells medium alone).

There were no effective differences in cytotoxic effects of scFvMEL/TNF between samples stored at 4° C. in buffer at pH 6.5, 7.0, or 7.5. There were no effective differences in cytotoxic effects of scFvMEL/TNF between samples formulated at pH 6.5, 7.0 or 7.5 and stored at −20° C. Addition of 0.1% HSA, in the formulations at pH 6.5, 7.0 and 7.5 and stored at 4° C. or −20° C. had no effect. Therefore, based on this data, optimal formulation conditions for scFvMEL/TNF are pH 7.5 in phosphate buffered saline (PBS). Optimal storage conditions for this formulation are −20° C. for up to 3 months without detectable loss of biological activity or degradation of protein structure.

Dose Rangefinder: Toxicity of scFvMEL/TNF in Femal Balb/c Mice afer 5 Daily Intravenous Injections

A multi-dose intravenous rangefinder study with scFvMel/TNF was conducted in Balb/c female mice. Five female mice per group were injected intravenously daily for 5 days with 0.2, 0.4, 0.6 and 0.8 mg/kg/day scFvMel/TNF (Groups 2-5). The total dose delivered was 1, 2, 3, and 4 mg/kg that corresponded to 25, 50, 75, and 100% of a previously established maximum tolerated dose (MTD). The vehicle control group (Group 1) consisted of saline. Seven days after the last injection (Day 12), animals on study were killed with carbon dioxide, terminally bled for hematology and clinical chemistry analyses, and subjected to a complete necropsy.

Under the conditions of this study, the major findings were that no deaths occurred; no test substance-related alterations in hematology or clinical chemistry parameters were noted; no test substance-related gross alterations of tissue specimens were noted; dose-related increase in relative spleen weights was noted in all dose groups and correlated with an increase in extramedullary hematopoiesis of the red pulp and follicular cell hyperplasia of the white pulp; microscopic test substance-related findings were present in the liver, spleen and lung. Dose-related extramedullary hematopoiesis was noted in the liver and spleen, and follicular cell (lymphoid) hyperplasia was noted in the spleen. Neither of these lesions was considered adverse. A single re-canalized thrombus was observed in 3/5 mice in the highest dose level. These thrombi occurred in only one vessel, and did not compromise pulmonary function. However, they are considered biologically adverse.

A no-observed-adverse-effect level (NOAEL) was 0.6 mg/kg/day for female mice under the conditions of this study. This dose level delivers a total dose of 3 mg/kg that is equivalent to 75% of a previously established MTD.

Exemplary materials and methods for this study are as follows. On scheduled sacrifice day 12, animals were killed with carbon dioxide, terminally bled from the heart, and subjected to a complete necropsy examination. For clinical pathology, the blood was collected into EDTA containers for a complete blood count (CBC), and the whole blood was placed in a clot tube for harvesting of serum to perform clinical chemistries.

Hematology: CBC

| | | |
|---|---|---|
| Clinical Chemistries: | Total Bilirubin | Calcium |
| | Phosphorus | Sodium |
| | AST (SGOT) | Potassium |
| | ALT (SGPT) | Chloride |
| | Total Protein | Alk Phosphatase |
| | Albumin | Creatinine |
| | Globulin | BUN |

For necropsy issues, a complete necropsy is defined as examination of the external surface of the body, all orifices, the cranial, thoracic, and abdominal cavities and their contents. The following tissues were collected from all mice sacrificed by design: digestive system including liver, esophagus, stomach, duodenum, jejunum, ileum, cecum, colon, rectum, salivary glands, pancreas; cardiovascular system including heart and aorta; respiratory system including trachea and lungs; urinary system including kidneys and urinary bladder; hematopoietic system including spleen, thymus, mesenteric lymph node, mandibular lymph node, bone marrow (femur/sternum); endocrine system including pituitary gland, thyroid gland, parathyroid glands, adrenal glands; nervous system including brain (cerebrum, midbrain, cerebellum, medulla/pons), spinal cord (cervical, thoracic, lumbar), sciatic nerve; musculoskeletal system including skeletal muscle, femur/knee joint, sternum; reproductive system including testes, epididymides, prostate, seminal vesicles, ovaries, uterus (cervix, body, horns) and other including skin, mammary gland, eyes, injection site and gross lesions

All tissues were placed in 10% neutral buffered fomalin as the fixative. Mice sacrificed by design had the following organs* weighed: liver, kidneys, and spleen. Relative organ weights (percent of final body weight) were calculated. All tissues collected from Groups 1 and 5 were processed to slides, stained with hematoxylin and eosin, and examined microscopically. Liver, kidneys, lung and spleen from Groups 2-4 were processed to slides, stained with hematoxylin and eosin, and examined microscopically.

### CONCLUSION

One of skill in the art recognizes that similar routine procedures may be performed for any chimeric molecule of the invention.

### Patents and Patent Applications

U.S. Pat. No. 4,677,064

U.S. Pat. No. 5,247,070

U.S. Pat. No. 5,422,104

U.S. Pat. No. 5,519,119

U.S. Pat. No. 5,606,023

U.S. Pat. No. 5,652,353

U.S. Pat. No. 5,652,353

U.S. Pat. No. 5,773,582

WO 93/21232

WO 97/22364

WO 97/46259

WO 99/45128

WO 99/49059

### PUBLICATIONS

A. P. Simoes-Wust, T. Schurpf, J. Hall, R. A. Stahel and U. Zangemeister-Wittke, Bcl-2/bcl-xL bispecific antisense treatment sensitizes breast carcinoma cells to doxorubicin, paclitaxel and cyclophosphamide, Breast Cancer Res. Treat. 76, pp. 157-166 (2002).
A. Sierra, X. Castellsague, A. Escobedo, B. Lloveras, M. Garcia-Ramirez, A. Moreno and A. Fabra, Bcl-2 with loss of apoptosis allows accumulation of genetic alterations: a pathway to metastatic progression in human breast cancer, Int. J. Cancer 89, pp. 142-147 (2000).
Adams, G. P., and Schier, R. Generating improved single-chain Fv molecules for tumor targeting. J. Immunol. Methods, 231: 249-260, Review, 1999.
Aggarwal B B and Natarajan K. Tumor necrosis factors: developments during the last decade. Eur Cytokine Netw 1996; 7:93-124.
Aigner A, Hsieh S S, Malerczyk C, Czubayko F. Reversal of HER-2 over-expression renders human ovarian cancer cells highly resistant to taxol. Toxicology 2000; 144:221-28.
Amar S, Van Dyke T E, Eugster H P, Schultze N, Koebel P, Bluethmann H. Tumor necrosis factor (TNF)-induced cutaneous necrosis is mediated by TNF receptor-1. J Inflamm 1995; 47: 180-9.
Aqeilan R, Yarkoni S, Lorberboum-Galski H. Interleukin 2-Bax: a novel prototype of human chimeric proteins for targeted therapy. FEBS Lett. 1999 Aug. 27; 457(2):271-6.
Atwell J L, Breheney K A, Lawrence L J, McCoy A J, Kortt A A, Hudson P J. scFv multimers of the anti-neuraminidase antibody NC10: length of the linker between VH and VL domains dictates precisely the transition between diabodies and triabodies. Protein Eng 1999; 12:597-604.
Awada A, Cardoso F, Atalay G, Giuliani R, Mano M, and Piccart M J (2003). The pipeline of new anticancer agents for breast cancer treatment in 2003. Crit Rev Oncol Hematol 48, 45-63.
B. Bucci, E. Carico, A. Rinaldi, F. Froio, Y. M. Puce, I. D'Agnano, A. Vecchione and E. Brunetti, Biological indicators of aggressiveness in Ti ductal invasive breast cancer, Anticancer Res. 21, pp. 2949-2955 (2001).
Bajetta E, Del Vecchio M, Bernard-Marty C, Vitali M, Buzzoni R, Rixe O, Nova P, Aglione S, Taillibert S, and Khayat D (2002). Metastatic melanoma: chemotherapy. Semin Oncol 29, 427-445.
Bargman C I, Hung M C, Weinberg R A. The neu oncogene encodes an epidermal growth factor-related protein. Nature 1986; 319: 226-30.
Belka C, Heinrich V, Marini P, Faltin H, Schulze-Osthoff K, Bamberg M, and Budach W (1999). Ionizing radiation and the activation of caspase-8 in highly apoptosis-sensitive lymphoma cells. Int J Radiat Biol 75, 1257-1264.
Berchuck A, Kamel A, Whitaker R, et al. Over expression of HER-2/neu is associated with poor survival in advanced epithelial ovarian cancer. Cancer Res 1990; 50: 4087-91.
Bharti, A. C., and Aggarwal, B. B. Nuclear factor-kappa B and cancer: its role in prevention and therapy. Biochem. Pharmacol., 64: 883-888, 2002.
Bian, X., Opipari, A. W. Jr., Ratanaproeksa, A. B., Boitano, A. E., Lucas, P. C., and Castle, V. P. Constitutively active NFkappa B is required for the survival of S-type neuroblastoma. J. Biol. Chem., 277: 42144-42150, 2002.
Bird R E, Hardman K D, Jacobson J W, Johnson S, Kaufman B M, Lee S M, Lee T, Pope S H, Riordan G S, Whitlow M. Single-chain antigen-binding proteins. Science 1988; 242:423-426.
Blick M, Sherwin S A, Rosenblum M, Gutterman J. Phase I study of recombinant tumor necrosis factor in cancer patients. Cancer Res 1987; 47:2986-2989.
Boris, L. C., and Steinke, J. W. Cytokines and chemokines. J. Allergy Clin. Immunol., 111: S460-475, Review, 2003.
Borish L C and Steinke J W. 2. Cytokines and chemokines. J Allergy Clin Immunol 2003; 111:S460-S475.
Boz G, De Paoli A, Innocente R., Rossi C., Tosolini G., Pederzoli P, Talamini, R, Trovo, M G. Radiotherapy and continuous infusion 5-fluorouracil in patients with nonresectable pancreatic carcinoma. Int J Radiat Oncol Biol Phys 2001; 51:736-40.
Brennan P, Babbage J W, Burgering B M, Groner B, Reif K, Cantrell D A. Phosphatidylinositol 3-kinase couples the interleukin-2 receptor to the cell cycle regulation. Immunity 1997; 7: 679-89.
Buchler P, Reber H A, Buchler M C, Roth M A, Buchler, M W, Friess H, Isacoff, W H, Hines, O J. Therapy for pancreatic cancer with a recombinant humanized anti-HER2 antibody (herceptin). J Gastrointest Surg 2001; 5:139-46.
Buchsbaum D J, Bonner J A, Grizzle W E, Stackhouse M A, Carpenter M, Hicklin D J, Bohlen, P, Raisch, K P. Treatment of pancreatic cancer xenografts with Erbitux (IMC-C225) anti-EGFR antibody, gemcitabine, and radiation. Int J Radiat Oncol Biol Phys 2002; 54:1180-93.
Burger, D., and Dayer, J. M. Cytokines, acute-phase proteins, and hormones: IL-1 and TNF-alpha production in contact-mediated activation of monocytes by T lymphocytes. Ann. N.Y. Acad. Sci., 966: 464-473, Review, 2002.
Butera J, Malachovsky M, Rathore R, Safran H. Novel approaches in development for the treatment of pancreatic cancer. Front Biosci 1998; 3:E226-E229.
C. Botti, S. Buglioni, M. Benevolo, D. Giannarelli, P. Papaldo, F. Cognetti, P. Vici, F. Di Filippo, F. Del Normo, F. M. Venanzi, P. G. Natali and M. Mottolese, Altered expression of FAS system is related to adverse clinical outcome in stage I-II breast cancer patients treated with adjuvant anthracycline-based chemotherapy, Clin. Cancer Res. 10, pp. 1360-1365 (2004).
C. D. Archer, M. Parton, I. E. Smith, P. A. Ellis, J. Salter, S. Ashley, G. Gui, N. Sacks, S. R. Ebbs, W. Allum, N. Nasiri and M. Dowsett, Early changes in apoptosis and proliferation following primary chemotherapy for breast cancer, Br. J. Cancer 89, pp. 1035-1041 (2003).
Cappello, P., Novelli, F., Formi, G., and Giovarelli, M. Death receptor ligands in tumors. J. Immunother., 25: 1-15, 2002.
Carmichael J. Clinical response benefit in patients with advanced pancreatic cancer. Role of gemcitabine. Digestion 1997; 58:503-7.
Cascinu S, Silva R R, Barni S, Labianca R, Frontini L, Piazza E, Pancera, G, Giordani, P, Giuliodori, L, Pessi, M A, Fusco, V, Luporini, G, et al. A combination of gemcitabine and 5-fluorouracil in advanced pancreatic cancer, a report from the Italian Group for the Study of Digestive Tract Cancer (GISCAD). Br J Cancer 1999; 80:1595-98.
Chan M C and Murphy R M. Kinetics of cellular trafficking and cytotoxicity of 9.2.27-gelonin immunotoxins targeted against the high-molecular-weight melanoma-associated antigen. Cancer Immunol Immunother 1999; 47:321-329.
Chou T C, Talalay P. Quantitative analysis of dose-effect relationships: the combined effects of multiple drugs or enzyme inhibitors. Adv Enyme Regul 1984; 22:27-55.
Classen C F, Falk C S, Friesen C, Fulda S, Herr I, and Debatin K M (2003). Natural killer resistance of a drug-resistant leukemia cell line, mediated by up-regulation of HLA class I expression. Haematologica 88, 509-521.
Clement M V, Haddad P, Soulie A, Legros-Maida S, Guillet J, Cesar E, and Sasportes M (1990). Involvement of granzyme B and perforin gene expression in the lytic potential of human natural killer cells. Res Immunol 141, 477-489.
Cocher, D., Bird, R. E., Roselli, M., Hardman, K. D., Hohnson, S., Pope, S., Dodd, S. W., Pantoliano, M.-W., Milenic, D. E., and Schlom, J. In vivo tumor targeting of a recombinant single-chain antigen-binding protein. J. Natl. Cancer Inst., 82: 1191-1197, 1990.
Colcher D, Bird R, Roselli M, Hardman K D, Johnson S, Pope S, Dodd S W, Pantoliano M W, Milenic D E, Schlom J. In vivo tumor targeting of a recombinant single-chain antigen-binding protein. J Natl Cancer Inst 1990; 82:1191-1197.
Colcher D, Pavlinkova G, Beresford G, Booth B J, Choudhury A, Batra S K. Pharmacokinetics and biodistribution of genetically-engineered antibodies. Q J Nucl Med 1998; 42:225-241.
Cooke, S. P., Pedley, R. B., Boden, R., Begent, R. H., and Chester, K. A. In vivo tumor delivery of a recombinant single chain Fv:: tumor necrosis factor—alpha fusion protein. Bioconjug. Chem., 13: 7-15, 2002.
Cosman, D. A family of ligands for the TNF receptor superfamily. Stem Cell, 12: 440-445, 1994.
Coussens L, Yang-Feng T L, Liao Y C, et al. Tyrosine kinase receptor with extensive homology to EGF receptor shares chromosomal location with neu oncogene. Science 1985; 230: 1132-9.
Cowan, K. J., and Storey, K. B. Mitogen-activated protein kinases: new signaling pathways functioning in cellular responses to environmental stress. J. Exp. Biol., 206: 1107-1115, 2003.
Curnis F, Gasparri A, Sacchi A, Longhi R, Corti A. Coupling tumor necrosis factor-alpha with alphaV integrin ligands improves its antineoplastic activity. Cancer Res 2004; 64:565-571.
D. L. Newton, H. J. Hansen, H. Liu, D. Ruby, M. S. Iordanov, B. E. Magun, D. M. Goldenberg and S. M. Rybak, Specifically targeting the CD22 receptor of human B-cell lymphomas with RNA damaging agents, Crit Rev. Oncol. Hematol. 39, pp. 79-86 (2001).
Dadachova E, Nosanchuk J D, Shi L, Schweitzer A D, Frenkel A, Nosanchuk J S, Casadevall A. Dead cells in melanoma tumors provide abundant antigen for targeted delivery of ionizing radiation by a mAb to melanin. Proc Natl Acad Sci USA 2004; 101:14865-14870.
Davis, C. B., and Gillies, S. D. Immunocytokines: amplification of anti-cancer immunity. Cancer Immunol. Immunother., 52: 297, 2003.
Dell'Erba L, Calo-Gabrieli G, Caruso M L, Thomas R, Cortino G, Valentini A M, Muto P, Albrizio M, Pastena M I, Lastoria S. Immunohistochemical reactivity of anti-melanoma monoclonal antibody 225.28S in human breast cancer biopsies. Anticancer Res 2001; 21:925-930.
Dergham S T, Dugan M C, Joshi U S, Chen Y C, Du W, Smith D W, Arlauskas, P, Crissman, J D, Vaitkevicius, V K, Sarkar, F H. The clinical significance of p21 (WAF1/CIP-1) and p53 expression in pancreatic adenocarcinoma. Cancer 1997; 80:372-81.
Ducreux M, Rougier P. The French experience with infusional 5-FU in gastric and pancreatic cancer. J Infus Chemother 1996; 6:203-05.
E. K. Rowinsky, Signal events: Cell signal transduction and its inhibition in cancer, Oncologist. 8 Suppl 3, pp. 5-17 (2003).
F. Martinez-Arribas, M. J. Nunez-Villar, A. R. Lucas, J. Sanchez, A. Tejerina and J. Schneider, Immunofluorometric study of Bcl-2 and Bax expression in clinical fresh tumor samples from breast cancer patients, Anticancer Res. 23, pp. 565-568 (2003).
Frey C, Twomey P, Keehn R, Elliott D, Higgins G. Randomized study of 5-FU and CCNU in pancreatic cancer: report of the Veterans Administration Surgical Adjuvant Cancer Chemotherapy Study Group. Cancer 1981; 47:27-31.
Gille H, Downward J. Multiple ras effector pathways contribute to G1 cell cycle progression. J Biol Chem 1999; 274: 22033-40.
Gjertsen M K, Gaudernack G. Mutated Ras peptides as vaccines in immunotherapy of cancer. Vox Sang 1998; 74(2):489-95.
Glinsky G V, Glinsky V V, Ivanova A B, and Hueser C J (1997). Apoptosis and metastasis: increased apoptosis resistance of metastatic cancer cells is associated with the profound deficiency of apoptosis execution mechanisms. Cancer Lett 115, 185-193.
Graf L H, Jr. and Ferrone S. Human melanoma-associated antigens. Immunol Ser 1989; 43:643-679.
Gumbs A A, Moore P S, Falconi M, Bassi C, Beghelli S, Modlin I, Scarpa, A. Review of the clinical, histological, and molecular aspects of pancreatic endocrine neoplasms. J Surg Oncol 2002; 81:45-53.
Gunzburg W H, Salmons B. Novel clinical strategies for the treatment of pancreatic carcinoma. Trends Mol Med 2001; 7:30-7.
Hahn S A, Kern S E. Molecular genetics of exocrine pancreatic neoplasms. Surg Clin North Am 1995; 75:857-69.
Hanada, T., and Yoshimura, A. Regulation of cytokine signaling and inflammation. Cytokine Growth Factor Rev., 13: 413-421, Review, 2002.
Harimaya, K., Tanaka, K., Matsumoto, Y., Sato, H., Matsuda, S., and Iwamoto, Y. Antioxidants inhibit TNFalpha-induced motility and invasion of human osteosarcoma cells: possible involvement of NFkappaB activation. Clin. Exp. Metastasis, 8:121-129, 2000.
Haycox A, Lombard M, Neoptolemos J, Walley T. Review article: current treatment and optimal patient management in pancreatic cancer. Aliment Pharmacol Ther 1998; 12:949-64.
Hehlgans, T., and Mannel, D. N. The TNF-TNF receptor system. Biol. Chem., 383: 1581-1585, 2002.
Helmbach H, Rossmann E, Kern M A, Schadendorf D. Drug-resistance in human melanoma. Int J Cancer 2001; 93:617-622.
Hoffmann M, Schmidt M, Wels W. Activation of EGF receptor family members suppresses the cytotoxic effects of tumor necrosis factor-alpha. Cancer Immunol Immunother 1998; 47: 167-75.
Hoogenboom H R, Raus J C, Volckaert G. Targeting of tumor necrosis factor to tumor cells: secretion by myeloma cells of a genetically engineered antibody-tumor necrosis factor hybrid molecule. Biochim Biophys Acta 1991; 1096:345-354.
Hudziak R M, Lewis G D, Shalaby M R, et al. Amplified expression of the HER/ERBB2 oncogene induces resistance to tumor necrosis factor α in NIH 3T3 cells. Proc Natl Acad Sci USA 1988; 85: 5102-6.
Humbert M, Devergne O, Cerrina J, Rain B, Simonneau G, Dartevelle P, Duroux P, Galanaud P, and Emilie D (1992). Activation of macrophages and cytotoxic cells during cytomegalovirus pneumonia complicating lung transplantations. Am Rev Respir Dis 145, 1178-1184.
Hung M C, Lau Y K. Basic science of HER-2/neu: a review. Semin Oncol 1999; 26: 51-9.
Hynes N E and Stern D F. The biology of erbB-2/neu/HER-2 and its role in cancer. Biochim Biophys Acta 1994; 1198:165-184.
Ignatoski K M, Maehama T, Markwart S M, Dixon J E, Livant D L, Ethier S P. ERBB-2 over expression confers PI 3′ kinase-dependent invasion capacity on human mammary epithelial cells. Br J Cancer 2000; 82: 666-74.
Irie A, Kashani-Sabet M, Scanlon K J, Uchida T, Baba S. Hammerhead ribozymes as therapeutic agents for bladder cancer. Mol Urol 2000; 4: 61-6.
Jaffee E M, Hruban R H, Biedrzycki B, Laheru D, Schepers K, Sauter P R, Goemann, M, Coleman, J, Grochow, L, Donehower, R C, Lillemoe, K D, O'Reilly, S, et al. Novel allogeneic granulocyte-macrophage colony-stimulating factor-secreting tumor vaccine for pancreatic cancer: a phase I trial of safety and immune activation. J Clin Oncol 2001; 19: 145-56.
Jansen B, Wacheck V, Heere-Ress E, Schlagbauer-Wadl H, Hoeller C, Lucas T, Hoermann M, Hollenstein U, Wolff K, and Pehamberger H (2000). Chemosensitisation of malignant melanoma by BCL2 antisense therapy. Lancet 356, 1728-1733.
Jiang B H, Zheng J Z, Aoki M, Vogt P K. Phosphatidylinositol 3-kinase signaling mediates angiogenesis and expression of vascular endothelial growth factor in endothelial cells. Proc Natl Acad Sci USA 2000; 97: 1749-53.
Jose M D, Ikezumi Y, van Rooijen N, Atkins R C, and Chadban S J (2003). Macrophages act as effectors of tissue damage in acute renal allograft rejection. Transplantation 76, 1015-1022.
K. M. Debatin, Activation of apoptosis pathways by anticancer drugs, Adv. Exp. Med. Biol. 457, pp. 237-244 (1999).
Kadison, A. S., and Morton, D. L. Immunotherapy of malignant melanoma. Surg. Clin. North Am., 83 (2): 343-370, Review, 2003.
Kalthoff H, Roeder C, Gieseking J, Humburg I, Schmiegel W. Inverse regulation of ERBB2 and epidermal growth factor receptors by tumor necrosis factor α. Proc Natl Acad Sci USA 1993; 90: 8972-6.
Kantor R R, Albino A P, Ng A K, Ferrone S. Biosynthesis and intracellular processing of four human melanoma associated antigens. Cancer Res 1986; 46:5223-5228.
Kantor R R, Ng A K, Giacomini P, Ferrone S. Analysis of the NIH workshop monoclonal antibodies to human melanoma antigens. Hybridoma 1982; 1:473-482.
Kaufman H L, Di V J, Jr, Horig H. Immunotherapy for pancreatic cancer: current concepts. Hematol Oncol Clin North Am 2002; 16:159-97.
Keppler-Hafkemeyer A, Brinkmann U, Pastan I. Role of caspases in immunotoxin-induced apoptosis of cancer cells. Biochemistry 1998; 37:16934-942.
Knuefermann C, Lu Y, Liu B, Jin W, Liang K, Wu L, Schmidt M, Milles G B, Mendelson J, Fan Z. HER2/PI-3K/Akt activation leads to a multidrug resistance in human breast adenocarcinoma cells. Oncogene 2003; 22:3205-12.
Koizumi, M., Endo, K., Watanabe, Y., Saga, T., Sakahara, H., and Konishi, J. Immunoscintigraphy and pharmacokinetics of indium-111-labeled ZME-018 monoclonal antibody in patients with malignant melanoma. Jpn. J. of Cancer Res., 79: 973-981, 1988.
Koshita Y, Lu Y, Fujii S, Neda H, Matsuyama T, Satoh Y, Itoh Y, Takahashi M, Kato J, Sakamaki S,. Efficacy of TNF-alpha gene-transduced tumor cells in treatment of established in vivo tumor. Int J Cancer 1995; 63:130-135.
Kraan M C, Haringman J J, Weedon H, Barg E C, Smith M D, Ahem M J, Smeets T J, Breedveld F C, and Tak P P (2004). T cells, fibroblast-like synoviocytes, and granzyme B+ cytotoxic cells are associated with joint damage in patients with recent onset rheumatoid arthritis. Ann Rheum Dis 63, 483-488.
Kreitman R J (1999). Immunotoxins in cancer therapy. Curr Opin Immunol 11, 570-578.
Kulke M H. Metastatic pancreatic cancer. Curr Treat Options Oncol 2002; 3:449-57.
Kuroda K, Miyata K, Fujita F, Koike M, Fujita M, Nomura M, Nakagawa S, Tsutsumi Y, Kawagoe T, Mitsuishi Y, Mayumi T. Human tumor necrosis factor-alpha mutant RGD-V29 (F4614) shows potent antitumor activity and reduced toxicity against human tumor xenografted nude mice. Cancer Lett 2000; 159:33-41.
Kuroda K, Miyata K, Shikama H, Kawagoe T, Nishimura K, Takeda K, Sakae N, Kato M. Novel muteins of human tumor necrosis factor with potent antitumor activity and less lethal toxicity in mice. Int J Cancer 1995; 63:152-157.
Kurschus F C, Kleinschmidt M, Fellows E, Dorumair K, Rudolph R, Lilie H, and Jenne D E (2004). Killing of target cells by redirected granzyme B in the absence of perforin. FEBS Lett 562, 87-92.
L. M. Veenendaal, H. Jin, S. Ran, L. Cheung, N. Navone, J. W. Marks, J. Waltenberger, P. Thorpe and M. G. Rosenblum, In vitro and in vivo studies of a VEGF121/rGelonin chimeric fusion toxin targeting the neovasculature of solid tumors, Proc. Natl. Acad. Sci. U.S.A 99, pp. 7866-7871 (2002).
L. Serrone and P. Hersey, The chemoresistance of human malignant melanoma: an update, Melanoma Res. 9, pp. 51-58 (1999).
Lemoine N R, Hughes C M, Barton C M, Poulsom R, Jeffery R E, Kloppel G, Hall, P A, Gullick, W J. The epidermal growth factor receptor in human pancreatic cancer. J Pathol 1992; 166:7-12.
Leong, S. P. Future perspectives on malignant melanoma. Surg. Clin. North Am., 83(2): 453-456, X, Review, 2003.
Lichtenstein A, Berenson J, Gera J F, Waldburger K, Martinez-Maza O, Berek J S. Resistance of human ovarian cancer cells to tumor necrosis factor and lymphokine-activated killer cells: correlation with expression of HER2/neu oncogenes. Cancer Res 1990; 50: 7364-70.
Lichtenstein A, Gera J F, Andrews J, Berenson J, Ware C F. Inhibitors of ADP-ribose polymerase decrease the resistance of HER2/neu-expressing cancer cells to the cytotoxic effects of tumor necrosis factor. J Immunol 1991; 146: 2052-8.
Lin, A. Activation of the JNK signaling pathway: breaking the brake on apoptosis. Bioessays, 25: 17-24, Review, 2003.
Lippke, J. A., Gu, Y., Sarnecki, C., Caron, P. R., and Su, M. S-S. Identification and characterization of CPP32/Mch2 homolog 1, a novel cysteine protease similar to CPP32. J. Biol. Chem., 271: 1825-1828, 1996.
Liu Y, Cheung L H, Hittelman W N, Rosenblum M G. Targeted delivery of human pro-apoptotic enzymes to tumor cells: In vitro studies describing a novel class of recombinant highly cytotoxic agents. Mol Cancer Ther 2003; 2:1341-1350.
Liu Y, Cheung L H, Marks J W, Rosenblum M G. Recombinant single-chain antibody fusion construct targeting human melanoma cells and containing tumor necrosis factor. Int J Cancer 2004; 108:549-557.
Liu Y, Cheung L H, Thorpe P, and Rosenblum M G (2003). Mechanistic studies of a novel human fusion toxin composed of vascular endothelial growth factor (VEGF)121 and the serine protease granzyme B: directed apoptotic events in vascular endothelial cells. Mol Cancer Ther 2, 949-959.
Liu, Y., Cheung, L., and Rosenblum, M. G. The fusion constructs targeted to human melanoma cells can overcome resistance to TNFa. Proc. Of AACR (Abstract # 1237)41:194, 2000.
Locksley R M, Killeen N, Lenardo M J. The TNF and TNF receptor superfamilies intergrating mammalian biology. Cell 2001; 104: 487-501.
Lohrisch C, Piccart M. An overview of HER2. Semin Oncol 2001; 28:3-11.
Lokich J J, Skarin A T. Combination therapy with 5-fluorouracil (5-FU; NSC-19893) and 1,3-bis(2-chloroethyl)-1-nitrosourea (BCNU; NSC-409962) for disseminated gastrointestinal carcinoma. Cancer Chemother Rep 1972; 56:653-57.
Lyu M A and Rosenblum M G. The immunocytokine scFv23/TNF sensitizes HER-2/neu-overexpressing SKBR-3 cells to tumor necrosis factor (TNF) via up-regulation of TNF receptor-1. Mol Cancer Ther 2005; 4:1205-1213.
M. G. Rosenblum, L. H. Cheung, Y. Liu and J. W. Marks, III, Design, expression, purification, and characterization, in vitro and in vivo, of an antimelanoma single-chain Fv antibody fused to the toxin gelonin, Cancer Res. 63, pp. 3995-4002 (2003).
M. J. Serrano, P. Sanchez-Rovira, I. Algarra, A. Jaen, A. Lozano and J. J. Gaforio, Evaluation of a gemcitabine-doxorubicin-paclitaxel combination schedule through flow cytometry assessment of apoptosis extent induced in human breast cancer cell lines, Jpn. J. Cancer Res. 93, pp. 559-566 (2002).
MacDonald G, Shi L, Vande V C, Lieberman J, and Greenberg A H (1999). Mitochondria-dependent and -independent regulation of Granzyme B-induced apoptosis. J Exp Med 189, 131-144.
Macey D J, Denardo S J, Denardo G L, Goodnight J K, Unger M W. Uptake of indium-111-labeled monoclonal antibody ZME-018 as a function of tumor size in a patient with melanoma. Am J Physiol Imaging 1988; 3:1-6.
Magee C J, Ghaneh P, Neoptolemos J P. Surgical and medical therapy for pancreatic carcinoma. Best Pract Res Clin Gastroenterol 2002; 16:435-55.
Marti, A., Luo, Z., Cunningham, C., Ohta, Y., Hartwig, J., Stossel, T. P., Kyriakis, J. M., and Avruch, J. Actin-binding protein-280 binds the stress-activated protein kinase (SAPK) activator SEK-1 and is required for tumor necrosis factor-alpha activation of SAPK in melanoma cells. J. Biol. Chem., 272: 2620-2628, 1997.
Matsuno S, Egawa S, Arai K. Trends in treatment for pancreatic cancer. J Hepatobiliary Pancreat Surg 2001; 8:544-48.
McGrath M S, Rosenblum M G, Philips M R, and Scheinberg D A (2003). Immunotoxin resistance in multidrug resistant cells. Cancer Res 63, 72-79.
Medema J P, Scaffidi C, Kischkel F C, et al. FLICE is activated by association with the CD95 death-inducing signaling complex (DISC). EMBO J. 1997; 16: 2794-804.
Michael M, Moore M. Clinical experience with gemcitabine in pancreatic carcinoma. Oncology (Huntingt) 1997; 11:1615-22.
Mihara M, Koishihara Y, Fukui H, Yasukawa K, Ohsugi Y. Murine anti-human IL-6 monoclonal antibody prolongs the half-life in circulating blood and thus prolongs the bioactivity of human IL-6 in mice. Immunology 1991; 74:55-59.
Minami, T., Abid, M. R., Zhang, J., King, G., Kodama, T., and Aird, W. C. Thrombin stimulation of vascular adhesion molecule-1 in endothelial cells is mediated by protein kinase C (PKC)-delta-NF-kappa B and PKC-zeta-GATA signaling pathways. J. Biol. Chem., 278: 6976-6984, 2003.
Moritz T, Niederle N, Baumann J, May D, Kurschel E, Osieka R, Kempeni J, Schlick E, Schmidt C G. Phase I study of recombinant human tumor necrosis factor alpha in advanced malignant disease. Cancer Immunol Immunother 1989; 29:144-150.
Muise-Helmericks R C, Grimes H L, Bellacosa A, Malstrom S E, Tsichlis P N, Rosen N (1998) Cyclin D expression is controlled post-transcriptionally via phosphatidylinositlo 3-kinase dependent pathway. J Biol Chem 273: 29864-29872.
Muise-Helmericks R C, Grimes H L, Bellacosa A, Malstrom S E, Tsichlis P N, Rosen N. Cyclin D expression is controlled post-transcriptionally via phosphatidylinositlo 3-kinase dependent pathway. J Biol Chem 1998; 273: 29864-72.
Mujoo K, Cheung L, Murray J L, Rosenblum M G. Pharmacokinetics, tissue distribution, and in vivo antitumor effects of the antimelanoma immunotoxin ZMB-gelonin. Cancer Immunol Immunother 1995; 40:339-345.
Murphy J R, Lakkis F G, vanderSpek J C, Anderson P, Strom T B. Protein engineering of diphtheria toxin. Development of receptor-specific cytotoxic agents for the treatment of human disease. Targeted Diagn Ther 1992; 7:365-382.
Mustika R, Budiyanto A, Nishigori C, Ichihashi M, and Ueda M (2005). Decreased expression of Apaf-1 with progression of melanoma. Pigment Cell Res 18, 59-62.
Nagata S. Apoptosis by death factor. Cell 1997; 88: 355-65.
Niitsu, Y., Watanabe, N., Sone, H., Neda, H., Yamauchi, N., and Urushizaki, I. Mechanisms of the cytotoxic effect of tumor necrosis factor. Jpn. J. Cancer Res., 76: 1193-1197, 1985.
Nishikawa, K., Rosenblum, M. G., Newman, R. A., Pandita, T. K., Hittelman, W. N., and Donato, N.J. Resistance of human cervical carcinoma cells to tumor necrosis factor correlates with their increased sensitivity to cisplatin: evidence of a role for DNA repair and epidermal growth factor receptor. Cancer Res., 52: 4758-4765, 1992.
Orlowski, R. Z., and Baldwin, A. S. Jr. NF-kappaB as a therapeutic target in cancer. Trends Mol. Med., 8: 385-389, Review, 2002.
Orth K, Chinnaiyan A M, Garg M, Froelich C J, and Dixit V M (1996). The CED-3/ICE-like protease Mch2 is activated during apoptosis and cleaves the death substrate lamin A. J Biol Chem 271, 16443-16446.
Osborne C K, Hobbs K, and Clark G M (1985). Effect of estrogens and antiestrogens on growth of human breast cancer cells in athymic nude mice. Cancer Res 45, 584-590.
Ozawa S, Ueda M, Ando N, Abe O, Shimizu N. Epidermal growth factor receptors in cancer tissues of esophagus, lung, pancreas, colorectum, breast and stomach. Jpn J Cancer Res 1988; 79:1201-07.
P. A. Ellis, I. E. Smith, S. Detre, S. A. Burton, J. Salter, R. A'Hern, G. Walsh, S. R. Johnston and M. Dowsett, Reduced apoptosis and proliferation and increased Bcl-2 in residual breast cancer following preoperative chemotherapy, Breast Cancer Res. Treat. 48, pp. 107-116 (1998).
P. Lipponen, Apoptosis in breast cancer: relationship with other pathological parameters, Endocr. Relat Cancer 6, pp. 13-16 (1999).
Pak B J, Lee J, Thai B L, Fuchs S Y, Shaked Y, Ronai Z, Kerbel R S, and Ben David Y (2004). Radiation resistance of human melanoma analyzed by retroviral insertional mutagenesis reveals a possible role for dopachrome tautomerase. Oncogene 23, 30-38.
Pastan I and Kreitman R J. Immunotoxins in cancer therapy. Curr Opin Investig Drugs 2002; 3:1089-1091.
Pastan I, Willingham M C, FitzGerald D J (1986) Immunotoxins. Cell 47: 641-648.
Pastan I. Immunotoxins containing Pseudomonas exotoxin A: a short history. Cancer Immunol Immunother 2003; 52:338-341.
Pegram M D, Lopez A, Konecny G, Slamon D J. Trastuzumab and chemotherapeutics: drug interactions and synergies. Semin Oncol 2000; 27: 21-5.
Permert J, Hafstrom L, Nygren P, Glimelius B. A systematic overview of chemotherapy effects in pancreatic cancer. Acta Oncol 2001; 40:361-70.
Prpic M L, Kastelan M, Gruber F, Laskarin G, Sotosek T, V, Strbo N, Zamolo G, Zauhar G, and Rukavina D (2004). Perforin expression in peripheral blood lymphocytes and skin-infiltrating cells in patients with lichen planus. Br J Dermatol 151, 433-439.
R. A. Campbell, P. Bhat-Nakshatri, N. M. Patel, D. Constantinidou, S. Ali and H. Nakshatri, Phosphatidylinositol 3-kinase/AKT-mediated activation of estrogen receptor alpha: a new model for anti-estrogen resistance, J. Biol. Chem. 276, pp. 9817-9824 (2001).
Rao, K. M. MAP kinase activation in macrophages. J. Leukoc. Biol., 69: 3-10, Review, 2001.
Reisfeld R A, Becker J C, Gillies S D. Immunocytokines: a new approach to immunotherapy of melanoma. Melanoma Res 1997; 7 Suppl 2:S99-106.
Reisfeld R A, Gillies S D, Mendelson J, Varki N M, Becker J C. Involvement of B lymphocytes in the growth inhibition of human pulmonary melanoma metastases in athymic nu/nu mice by an antibody-lymphotoxin fusion protein. Cancer Res 1996; 56: 1707-12.
Rieux-Laucat F, Le Deist F, and De Saint B G (2005). Autoimmune lymphoproliferative syndrome and perforin. N Engl J Med 352, 306-307.
Ro J S, E1-Naggar A, Ro J Y, et al. C-erbB-2 amplification in node-negative human breast cancer. Cancer Res 1989; 49: 6941-4.
Rocha S, Soengas M S, Lowe S W, Glanzmann C, Fabbro D, Winterhalter K, Bodis S, and Pruschy M (2000). Protein kinase C inhibitor and irradiation-induced apoptosis: relevance of the cytochrome c-mediated caspase-9 death pathway. Cell Growth Differ 11, 491-499.
Rockmann H, and Schadendorf D (2003). Drug resistance in human melanoma: mechanisms and therapeutic opportunities. Onkologie 26, 581-587.
Rosenblum M. Immunotoxins and toxin constructs in the treatment of leukemia and lymphoma. Adv Pharmacol 2004; 51:209-228.
Rosenblum M G, Cheung L, Kim S K, Mujoo K, Donato N J, Murray J L. Cellular resistance to the antimelanoma immunotoxin ZME-gelonin and strategies to target resistant cells. Cancer Immunol Immunother 1996; 42:115-121.
Rosenblum M G, Cheung L, Mujoo K, Murray J L. An antimelanoma immunotoxin containing recombinant human tumor necrosis factor: tissue disposition, pharmacokinetic, and therapeutic studies in xenograft models. Cancer Immunol Immunother 1995; 40:322-328.
Rosenblum M G, Cheung L, Murray J L, Bartholomew R. Antibody-mediated delivery of tumor necrosis factor (TNF-alpha): improvement of cytotoxicity and reduction of cellular resistance. Cancer Commun 1991; 3:21-7.
Rosenblum M G, Cheung L H, Liu Y, Marks J W, III. Design, expression, purification, and characterization, in vitro and in vivo, of an antimelanoma single-chain Fv antibody fused to the toxin gelonin. Cancer Res 2003; 63:3995-4002.
Rosenblum M G, Horn S A, Cheung L H. A novel recombinant fusion toxin targeting HER-2/neu-overexpressing cells and containing human tumor necrosis factor. Int J Cancer 2000; 88: 267-73.
Rosenblum M G, Levin B, Roh M, Hohn D, McCabe R, Thompson L, Cheung L, Murray J L. Clinical pharmacology and tissue disposition studies of 131I-labeled anticolorectal carcinoma human monoclonal antibody LiCO 16.88. Cancer Immunol Immunother 1994; 39:397-400.
Rosenblum M G, Marks J W, Cheung L H. Comparative cytotoxicity and pharmacokinetics of antimelanoma immunotoxins containing either natural or recombinant gelonin. Cancer Chemother Pharmacol 1999; 44:343-348.
Rosenblum M G, Murray J L, Cheung L, Rifkin R, Salmon S, Bartholomew R. A specific and potent immunotoxin composed of antibody ZME-018 and the plant toxin gelonin. Mol Biother 1991; 3:6-13.
Rosenblum M G, Verschraegen C F, Murray J L, Kudelka A P, Gano J, Cheung L, Kavanagh J J. Phase I study of 90Y-labeled B72.3 intraperitoneal administration in patients with ovarian cancer: effect of dose and EDTA coadministration on pharmacokinetics and toxicity. Clin Cancer Res 1999; 5:953-961.
Rosenblum, M. G., Cheung, L., Kim, S. K., Mujoo, K., Donato, N.J., and Murray, J. L. Cellular resistance to the antimelanoma immunotoxin ZME-gelonin and strategies to target resistant cells. Cancer Immunol. Immunother., 42: 115-121, 1996.
Ross J S, Fletcher J A. The HER-2/neu oncogene in breast cancer: prognostic factor, predictive factor, and target for therapy. Oncologist 1998; 3: 237-52.
Rubin I, Yarden Y. The basic biology of HER2. Ann Oncol 2001; 12(11):S3-S8.
Rubio N, Espana L, Fernandez Y, Blanco J, and Sierra A (2001). Metastatic behavior of human breast carcinomas overexpressing the Bcl-x(L) gene: a role in dormancy and organospecificity. Lab Invest 81, 725-734.
S. Abramovitch and H. Werner, Functional and physical interactions between BRCA1 and p53 in transcriptional regulation of the IGF-R gene, Horm. Metab Res. 35, pp. 758-762 (2003).
S. Inoue, A. E. Salah-Eldin and K. Omoteyama, Apoptosis and anticancer drug resistance, Hum. Cell 14, pp. 211-221 (2001).
S. J. Park, C. H. Wu and A. R. Safa, A P-glycoprotein- and MRP1-independent doxorubicin-resistant variant of the MCF-7 breast cancer cell line with defects in caspase-6, -7, -8, -9 and -10 activation pathways, Anticancer Res. 24, pp. 123-131(2004).
S. Syed and E. Rowinsky, The new generation of targeted therapies for breast cancer, Oncology (Huntingt) 17, pp. 1339-1351 (2003).
Sabbatini P, McCormick F. Phosphoinositide 3-OH kinase (P13K) and PKB/Akt delay the onset of p53-mediated, transcriptionally dependent apoptosis. J Biol Chem 1999; 274: 24263-9.
Sacca R, Cuff C A, Lesslauer W, Ruddle N H. Differential activities of secreted Lymphotoxin-α₃ and membrane Lymphotoxinα₁β₂ in Lymphotoxin-induced inflammation: Critical role of TNF receptor 1 signaling. J Immunol 1998; 160: 485-91.
Safran H, Steinhoff M, Mangray S, Rathore R, King T C, Chai L, Berzein, K, Moore, T, Lannitti, D, Reiss, P, Pasquariello, T, Akerman, P, et al. Overexpression of the HER-2/neu oncogene in pancreatic adenocarcinoma. Am J Clin Oncol 2001; 24:496-99.
Sakorafas G H, Lazaris A, Tsiotou A G, Koullias G, Glinatsis M T, Golematis B C. Oncogenes in cancer of the pancreas. Eur J Surg Oncol 1995; 21:251-53.
Saks S, Rosenblum M. Recombinant human TNF-alpha: preclinical studies and results from early clinical trial. Immunol Ser 1992; 56: 567-87.
Sasaki M, Kumazaki T, Tanimoto K, and Nishiyama M. Bcl-2 in cancer and normal tissue cells as a predication marker of response to 5-fluorouracil. 2003; 22:181-86.
Schafer J, Bachtler J, Engling A, Little J B, Weber K J, and Wenz F (2002). Suppression of apoptosis and clonogenic survival in irradiated human lymphoblasts with different TP53 status. Radiat Res 158, 699-706.
Scherf U, Benhar I, Webber K O, Pastan I, Brinkmann U. Cytotoxic and antitumor activity of a recombinant tumor necrosis factor-B1(Fv) fusion protein on LeY antigen-expressing human cancer cells. Clin Cancer Res 1996; 2:1523-1531.
Schoemaker, M. H., Ros, J. E., Homan, M., Trautwein, C., Liston, P., Poelstra, K., van Goor, H., Jansen, P. L., and Moshage, H. Cytokine regulation of pro- and anti-apoptotic genes in rat hepatocytes: NF-kappaB-regulated inhibitor of apoptosis protein 2 (cIAP2) prevents apoptosis. J. Hepatol., 36: 742-750, 2002.
Shi L, Kraut R P, Aebersold R, and Greenberg A H (1992). A natural killer cell granule protein that induces DNA fragmentation and apoptosis. J Exp Med 175, 553-566.
Shiohara M, Gombart A F, Berman J D, Koike K, Komiyama A, Koeffler H P. Cytostatic effect of TNFalpha on cancer cells is independent of p21 WAF1. Oncogene 1997; 15:1605-1609.
Simstein R, Burow M, Parker A, Weldon C, and Beckman B (2003). Apoptosis, chemoresistance, and breast cancer: insights from the MCF-7 cell model system. Exp Biol Med (Maywood) 228, 995-1003.
Singleton T P, Strickler J G. Clinical and pathologic significance of the c-erbB-2 (HER-2/neu) oncogene. Pathol Annu 1992; 27:165-90.
Slamon D J, Clark G M, Wong S G, Levin W J, Ullrich A, McGuire W L. Human breast cancer: correlation of relapse and survival with amplification of the HER-2/neu oncogene. Science 1987; 235: 177-82.
Slamon D J, Godolphin W, Jones L A, et al. Studies of the HER-2/neu proto-oncogene in human breast and ovarian cancer. Science 1989; 244: 707-12.
Sleijfer S, Asschert J G W, Timmer-Bosscha H, Mulder N H. Enhanced sensitivity to tumor necrosis factor-α in doxorubicin-resistant tumor cell lines due to down-regulated c-erbB2. Int J Cancer 1998; 77: 101-6.
Smith, R. A., and Baglioni, C. The active form of tumor necrosis factor is atrimer. J. Biol. Chem., 262: 6951-6954, 1987.
Smyth M J, and Trapani J A (1995). Granzymes: exogenous proteinases that induce target cell apoptosis. Immunol Today 16, 202-206.
Smyth M J, Browne K A, Thia K Y, Apostolidis V A, Kershaw M H, and Trapani J A (1994). Hypothesis: cytotoxic lymphocyte granule serine proteases activate target cell endonucleases to trigger apoptosis. Clin Exp Pharmacol Physiol 21, 67-70.
Smyth M J, O'Connor M D, and Trapani J A (1996). Granzymes: a variety of serine protease specificities encoded by genetically distinct subfamilies. J Leukoc Biol 60, 555-562.
Soengas M S, and Lowe S W (2003). Apoptosis and melanoma chemoresistance. Oncogene 22, 3138-3151.
Spaeny-Dekking E H, Hanna W L, Wolbink A M, Wever P C, Kummer A J, Swaak A J, Middeldorp J M, Huisman H G, Froelich C J, and Hack C E (1998). Extracellular granzymes A and B in humans: detection of native species during CTL responses in vitro and in vivo. J Immunol 160, 3610-3616.
Stancovski I, Sela M, Yarden Y. Molecular and clinical aspects of the Neu/ErbB-2 receptor tyrosine kinase. Cancer Treat Res 1994; 71:161-91.
Steinle A, Li P, Morris D L, Groh V, Lanier L L, Strong R K, Spies T. Interactions of human NKG2D with its ligands MICA, MICB, and homologs of the mouse RAE-1 protein family. Immunogenetics 2001; 53:279-287.
Sugarman, B. J., Aggarwal, B. B., Hass, P. E., Figarci, I. S., Palladino, M. A., and Shepard, H. M. Recombinant tumor necrosis factor-alpha: effect on proliferation of normal and transformed cells in vitro. Science, 230: 943-945, 1985.
Sun, Z., and Andersson, R. NF-kappaB activation and inhibition: a review. Shock, 18: 99-106, 2002.
Sutton V R, Davis J E, Cancilla M, Johnstone R W, Ruefli A A, Sedelies K, Browne K A, and Trapani J A (2000). Initiation of apoptosis by granzyme B requires direct cleavage of bid, but not direct granzyme B-mediated caspase activation. J Exp Med 192, 1403-1414.
Suzuki T, Anderegg B, Ohkawa T, Irie A, Engebraaten O, Halks-Miller M, Holm, P S, Curiel, D T, Kashani-Sabet, M, Scanlon, K J. Adenovirus-mediated ribozyme targeting of HER-2/neu inhibits in vivo growth of breast cancer cells. Gene Ther 2000; 7: 241-48.
T. Lin, L. Zhang, J. Davis, J. Gu, M. Nishizaki, L. Ji, J. A. Roth, M. Xiong and B. Fang, Combination of TRAIL gene therapy and chemotherapy enhances antitumor and antimetastasis effects in chemosensitive and chemoresistant breast cancers, Mol. Ther. 8, pp. 441-448 (2003).
Takada T, Kato H, Sasaki M, Matsushiro T, Yamauchi H, Kajihara T, Watanabe, G, Hanaue, H. Prospective randomized trial comparing modified FAM (5-fluorouracil (5-FU)+adriamycin+mitomycin C) versus 5-FU alone for the treatment of non-resectable pancreatic and biliary tract carcinomas (the 1st trial in non-resectable patients). Study Group of Surgical Adjuvant Therapy for Carcinomas of the Pancreas and Biliary Tract. Gan To Kagaku Ryoho 1992; 19:1295-1301.
Tamanini, A., Rolfini, R., Nicolis, E., Melotti, P., and Cabrini, G. MAP kinases and NF-kappaB collaborate to induce ICAM-1 gene expression in the early phase of adenovirus infection. Virology, 307: 228-242, 2003.
Tang P, Hung M C, Klostergaard J. TNF cytotoxicity: effects of HER-2/neu expression and inhibitors of ADP-ribosylation. Lymphokine Cytokine Res 1994; 13: 117-23.
Tartaglia L A, Rothe M, Hu Y F, Goeddel D V. Tumor necrosis factor's cytotoxic activity is signaled by the p55 TNF receptor. Cell 1993; 73: 213-6.
Thybusch-Bernhardt A, Aigner A, Beckmann S, Czubayko F, Juhl H. Ribozyme targeting of HER-2 inhibits pancreatic cancer cell growth in vivo. Eur J Cancer 2001; 37: 1688-94.
Thybusch-Bernhardt A, Beckmann S, Juhl H. Comparative analysis of the EGF-receptor family in pancreatic cancer: expression of HER-4 correlates with a favourable tumor stage. Int J Surg Investig 2001; 2:393-400.
Tomaszewska R, Okon K, Nowak K, Stachura J. HER-2/Neu expression as a progression marker in pancreatic intraepithelial neoplasia. Pol J Pathol 1998; 49:83-92.
Torre E A, Salimbeni V, Fulco R A. The erbB 2 oncogene and chemotherapy: a mini-review. J Chemother 1997; 9:51-5.
Trapani J A, Browne K A, Smyth M J, and Jans D A (1996). Localization of granzyme B in the nucleus. A putative role in the mechanism of cytotoxic lymphocyte-mediated apoptosis. J Biol Chem 271, 4127-4133.
Tsujimoto, M., Yip, Y. K., and Vilceck, J. Tumor necrosis factor, specific binding and internalization in sensitive and resistant cells. Proc. Natl. Acad. Sci. USA, 82: 7626-7630, 1985.
Tyson F L, Boyer C M, Kaufman R, et al. Expression and amplification of the HER-2/neu (c-erbB-2) protooncogene in epithelial ovarian tumors and cell lines. Am J Obstet Gynecol 1991; 165: 640-6.
Tzahar E, Yarden Y. The ErbB-2/HER2 oncogenic receptor of adenocarcinomas: from orphan hood to multiple stromal ligands. Biochem Biophys Acta 1998; 1377: M25-M37.
Uchida H, Shinoura N, Kitayama J, Watanabe T, Nagawa H, and Hamada H (2002). Caspase-8 gene transduction augments radiation-induced apoptosis in DLD-1 cells. Biochem Biophys Res Commun 292, 347-354.
Ueno N T, Bartholomeusz C, Herrmann J L, Estrov Z, Shao R, Andreeff M, Price J, Paul R W, Anklesaria P, Yu D, Hung M C. E1A-mediated paclitaxel sensitization in HER-2/neu-overexpressing ovarian cancer SKOV3.1pl through apoptosis involving the caspase-3 pathway. Clin Cancer Res 2000; 6:250-59.
Unglaub R, Maxeiner B, Thoma B, Thoma B, Pfizenmaier K, Scheurich P. Downregulation of tumor necrosis factor (TNF) sensitivity via modulation of TNF binding capacity by protein kinase C activators. J Exp Med 1987; 166: 1788-97.
van Moorsel C J, Peters G J, Pinedo H M. Gemcitabine: Future Prospects of Single-Agent and Combination Studies. Oncologist 1997; 2:127-34.
Veenendaal L M, Jin H, Ran S, Cheung L, Navone N, Marks J W, Waltenberger J, Thorpe P, Rosenblum M G. In vitro and in vivo studies of a VEGF121/rGelonin chimeric fusion toxin targeting the neovasculature of solid tumors. Proc Natl Acad Sci USA 2002; 99:7866-7871.
Verdu J, Buratovich M A, Wilder E L, Birnbaum M J. Cell-autonomous regulation of cell and organ growth in Drosophila by Akt/PKB. Nat Cell Biol 1999; 1: 500-6.
Volm M, and Koomagi R (1999). The implications of proliferation and apoptosis for lung cancer metastasis. Oncol Rep 6, 373-376.
W. Wu, S. Chaudhuri, D. R. Brickley, D. Pang, T. Karrison and S. D. Conzen, Microarray analysis reveals glucocorticoid-regulated survival genes that are associated with inhibition of apoptosis in breast epithelial cells, Cancer Res. 64, pp. 1757-1764 (2004).
Wajant H, Grell M, Scheurich P. TNF receptor associated factors in cytokine signaling. Cytokine Growth Factor Rev 1999; 10: 15-26.
Waldmann T A, Levy R, Coller B S. Emerging Therapies: Spectrum of Applications of Monoclonal Antibody Therapy. Hematology (Am Soc Hematol Educ Program) 2000; 394-408.
Wallach D, Varfolomeev E E, Malinin N L, Goltsev Y V, Kovalenko A V, Boldin M P. Tumor necrosis factor receptor and Fas signalin mechanisms. Annu Rev Immunol 1999; 17:331-67.
Ward S, Morris E, Bansback N, Calvert N, Crellin A, Forman D, Larvin, M, Radstone, D. A rapid and systematic review of the clinical effectiveness and cost-effectiveness of gemcitabine for the treatment of pancreatic cancer. Health Technol Assess 2001; 5:1-70.
Watanabe N, Niitsu Y, Umeno H, Kuriyama H, Neda H, Yamauchi N, Maeda M, Urushizaki I. Toxic effect of tumor necrosis factor on tumor vasculature in mice. Cancer Res 1988; 48:2179-2183.
Williams T M, Weiner D B, Greene M I, Maguire H C, Jr. Expression of c-erbB-2 in human pancreatic adenocarcinomas. Pathobiology 1991; 59:46-52.
Wowk M E, and Trapani J A (2004). Cytotoxic activity of the lymphocyte toxin granzyme B. Microbes Infect 6, 752-758.
Wuchter C, Karawajew L, Ruppert V, Buchner T, Schoch C, Haferlach T,
Ratei R, Dorken B, and Ludwig W D. Clinical significance of CD95, Bcl-2 and Bax expression and CD95 function in adult de novo acute myeloid leukemia in context of P-glycoprotein function, maturation stage, and cytogenetics. Leukemia 1999: 13:1943-1953.
Xiang R, Lode R N, Dolman C S, Dreier T, Varki N M, Qian X, Lo K M, Lan Y, Super M, Gillies S D, Reisfeld R A. Elimination of established murine colon carcinoma metastases by antibody-interleukin 2 fusion protein therapy. Cancer Res 1997; 57:4948-4955.
Xiong H Q, Abbruzzese J L Epidermal growth factor receptor-targeted therapy for pancreatic cancer. Semin Oncol 2002; 29:31-7.
Yagita H, Nakata M, Kawasaki A, Shinkai Y, and Okumura K (1992). Role of perforin in lymphocyte-mediated cytolysis. Adv Immunol 51, 215-242.
Yamamoto T, Ikawa S, Akiyama T, et al. Similarity of protein encoded by the human c-erbB-2 gene to epidermal growth factor receptor. Nature 1986; 319: 230-4.
Yamanaka Y. The immunohistochemical expressions of epidermal growth factors, epidermal growth factor receptors and c-erbB-2 oncoprotein in human pancreatic cancer. Nippon Ika Daigaku Zasshi 1992; 59:51-61.
Yarden Y. Biology of HER2 and its importance in breast cancer. Oncology 2001; 61(2):1-13.
Yokoyama M, Yamanaka Y, Friess H, Buchler M, Korc M. p53 expression in human pancreatic cancer correlates with enhanced biological aggressiveness. Anticancer Res 1994; 14:2477-83.
Yu D, Hung M C. Role of erbB2 in breast cancer chemosensitivity. BioEssays 2000; 2: 673-80.
Zhao J, Zhang L H, Jia L T, Zhang L, Xu Y M, Wang Z, Yu C J, Peng W D,
Wen W H, Wang C J, Chen S Y, and Yang A G (2004). Secreted antibody/granzyme B fusion protein stimulates selective killing of HER2-overexpressing tumor cells. J Biol Chem 279, 21343-21348.
Zhou B P, Hu M C T, Miller S A, et al. HER-2/neu blocks tumor necrosis factor-induced apoptosis via the Akt/NF-κB pathway. J Biol Chem 2000; 275: 8027-31.
Zouboulis C C, Schroder K, Garbe C, Krasagakis K, Kruger S, Orfanos C E. Cytostatic and cytotoxic effects of recombinant tumor necrosis factor-alpha on sensitive human melanoma cells in vitro may result in selection of cells with enhanced markers of malignancy. J Invest Dermatol 1990; 95:223S-230S.
Zuckerman J E, Murray J L, Rosenblum M G. Preparation and biological activity of recombinant leukocyte interferon A (rIFN-α A) conjugated to an anti-melanoma murine monoclonal antibody (ZME-018). Pro Amer Assoc Cancer Res 1987; Abstract No. 1060.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A method of conferring or restoring chemosensitivity to one or more chemotherapy-resistant cancer cells in an individual, comprising delivering to the individual a therapeutically effective amount of a chimeric molecule comprising a cell-specific targeting moiety and an anti-cell proliferation moiety.

2. The method of claim 1, wherein the cell-specific targeting moiety is further defined as a cancer cell-targeting moiety.

3. The method of claim 2, wherein the cancer cell-targeting moiety is further defined as an antibody, a growth factor, a hormone, a peptide, an aptamer, or a cytokine.

4. The method of claim 3, wherein the antibody is further defined as a full-length antibody, chimeric antibody, Fab′, Fab, F(ab′)2, single domain antibody (DAB), Fv, single chain Fv (scFv), minibody, diabody, triabody, or a mixture thereof.

5. The method of claim 4, wherein the antibody is a scFv.

6. The method of claim 3, wherein the antibody is an anti-HER-2/neu antibody.

7. The method of claim 6, wherein the HER-2/neu antibody is scFv23.

8. The method of claim 3, wherein the antibody is an anti-gp240 antigen antibody.

9. The method of claim 8, wherein the anti-gp240 antigen antibody comprises scFvMEL.

10. The method of claim 3, wherein the cancer cell-targeting moiety comprises one or more growth factors.

11. The method of claim 10, wherein the growth factor is transforming growth factor, epidermal growth factor, insulin-like growth factor, fibroblast growth factor, heregulin, platelet-derived growth factor, vascular endothelial growth factor, or hypoxia inducible factor.

12. The method of claim 3, wherein the cancer cell-targeting moiety comprises one or more hormones.

13. The method of claim 12, wherein the hormone is human chorionic gonadotropin, gonadotropin releasing hormone, an androgen, an estrogen, thyroid-stimulating hormone, follicle-stimulating hormone, luteinizing hormone, prolactin, growth hormone, adrenocorticotropic hormone, antidiuretic hormone, oxytocin, thyrotropin-releasing hormone, growth hormone releasing hormone, corticotropin-releasing hormone, somatostatin, dopamine, melatonin, thyroxine, calcitonin, parathyroid hormone, glucocorticoids, mineralocorticoids, adrenaline, noradrenaline, progesterone, insulin, glucagon, amylin, erythropoitin, calcitriol, calciferol, atrial-natriuretic peptide, gastrin, secretin, cholecystokinin, neuropeptide Y, ghrelin, PYY₃₋₃₆, insulin-like growth factor-1, leptin, thrombopoietin, angiotensinogen, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, or, IL-36.

14. The method of claim 3, wherein the cancer cell-targeting moiety comprises one or more cytokines.

15. The method of claim 14, wherein the cytokine is IL1, IL2, IL3, IL4, IL5, IL6, IL7, IL8, IL9, IL10, IL11, IL12, IL13, IL14, IL15, IL-16, IL-17, IL-18, granulocyte-colony stimulating factor, macrophage-colony stimulating factor, granulocyte-macrophage colony stimulating factor, leukemia inhibitory factor, erythropoietin, granulocyte macrophage colony stimulating factor, oncostatin M, leukemia inhibitory factor, IFN-γ, IFN-α, IFN-β, LT-β, CD40 ligand, Fas ligand, CD27 ligand, CD30 ligand, 4-1BBL, TGF-β, IL 1α, IL-1β, IL-1 RA, MIF, IGIF, or a mixture thereof.

16. The method of claim 1, wherein the anti-cell proliferation moiety is further defined as an apoptosis-inducing moiety or a cytotoxic agent.

17. The method of claim 16, wherein the apoptosis-inducing moiety is a granzyme, a Bcl-2 family member, cytochrome C, or a caspase.

18. The method of claim 17, wherein the granzyme is granzyme A, granzyme B, granzyme C, granzyme D, granzyme E, granzyme F, granzyme G, granzyme H, granzyme I, granzyme J, granzyme K, granzyme L, granzyme M, or granzyme N.

19. The method of claim 18, wherein the granzyme is granzyme B.

20. The method of claim 17, wherein the Bcl-2 family member is Bax, Bak, Bcl-Xs, Bad, Bid, Bik, Hrk, or Bok.

21. The method of claim 17, wherein the caspase is caspase-1, caspase-2 caspase-3, caspase-4, caspase-5, caspase-6, caspase-7, caspase-8, caspase-9, caspase-10, caspase-11, caspase-12, caspase-13, or caspase-14.

22. The method of claim 16, wherein the cytotoxic agent is TNF-α, gelonin, Prodigiosin, a ribosome-inhibiting protein (RIP), Pseudomonas exotoxin, Clostridium difficile Toxin B, Helicobacter pylori VacA, Yersinia enterocolitica YopT, Violacein, diethylenetriaminepentaacetic acid, irofulven, Diptheria Toxin, mitogillin, ricin, botulinum toxin, cholera toxin, or saporin 6.

23. The method of claim 16, wherein the cytotoxic agent is recombinant.

24. The method of claim 1, wherein the cell-specific targeting moiety and the anti-cell proliferation moiety are chemically conjugated.

25. The method of claim 1, wherein the cell-specific targeting moiety and the anti-cell proliferation moiety are comprised in a fusion polypeptide.

26. The method of claim 1, wherein the cell-specific targeting moiety and the anti-cell proliferation moiety are connected by a linker.

27. The method of claim 1, wherein the chemotherapy-resistant cancer cell is further defined as HER-2/neu overexpressing, resistant to TNF-α, Nf-κB-overexpressing, Nf-κB signaling-defective, or a combination thereof.

28. The method of claim 1, wherein the chemotherapy-resistant cells are resistant to one or more classes of chemotherapeutic agents.

29. The method of claim 28, wherein the classes of chemotherapeutic agents are selected from the group consisting of alkylating agents, nitrosoureas, antimetabolites, antitumor antibiotics, plant alkyloids, taxanes, and hormonal agents.

30. The method of claim 1, wherein the chemotherapy-resistant cells are resistant to one or more of 5-fluorouracil, cisplatin, etoposide, doxorubicin, or gemcitabine.

31. The method of claim 1, further comprising an additional cancer therapy for the individual.

32. The method of claim 31, wherein the additional cancer therapy is chemotherapy, surgery, radiation, gene therapy, hormone therapy, immunotherapy, or a combination thereof.

33. The method of claim 32, wherein the chemotherapy and the chimeric molecule are administered concomitantly.

34. The method of claim 32, wherein the chemotherapy and the chimeric molecule are administered in succession.

35. The method of claim 34, wherein the chimeric molecule is administered prior to the chemotherapy.

36. The method of claim 34, wherein the chimeric molecule is administered subsequent to the chemotherapy.

37. The method of claim 32, wherein the chemotherapy and the chimeric molecule provide a synergistic effect on the cancer cell.

38. The method of claim 32, wherein the chemotherapy and the chimeric molecule provide an additive effect on the cancer cell.

39. The method of claim 32, wherein the chimeric molecule is further defined as neoadjuvant surgical therapy.

40. The method of claim 32, wherein the chimeric molecule is further defined as postadjuvant surgical therapy.

41. The method of claim 1, wherein the chimeric molecule is scFvMEL/GrB.

42. The method of claim 1, wherein the chimeric molecule is scFv23/TNF-α.

43. The method of claim 1, wherein the chimeric molecule is scFvMEL/TNF-α.

44. A method of sensitizing one or more cancer cells in an individual to a chemotherapy, comprising administering to the individual a therapeutically effective amount of a chimeric molecule, said chimeric molecule comprising a cell-targeting moiety and an anti-cell proliferation moiety.

45. A method of inducing apoptosis in one or more TNF-resistant cancer cells in an individual, comprising administering to the individual a therapeutically effective amount of a chimeric molecule, said chimeric molecule comprising a cell-specific targeting moiety and an anti-cell proliferation moiety.

46. A method of inducing apoptosis in one or more HER-2/neu overexpressing cancer cells in an individual, comprising administering to the individual a therapeutically effective amount of a chimeric molecule, said chimeric molecule comprising a cell-specific targeting moiety and an anti-cell proliferation moiety.

47. A method of inducing apoptosis in one or more gp240 antigen-positive cells in an individual, comprising administering to the individual a therapeutically effective amount of a chimeric molecule, said chimeric molecule comprising a cell-specific targeting moiety and an anti-cell proliferation moiety.

48. A method of treating cancers in an individual that are Her-2/neu overexpressing and Nf-κB overexpressing, comprising administering to the individual a therapeutically effective amount of a chimeric molecule, said chimeric molecule comprising a cell-specific targeting moiety and an anti-cell proliferation moiety.

49. A method of treating cancer in an individual, comprising administering to the individual a therapeutically effective amount of at least one chemotherapeutic agent, wherein said agent acts by interrupting NF-κB signaling, and a chimeric molecule, said chimeric molecule comprising a cell-specific targeting moiety and an anti-cell proliferation moiety.
